SEQUENCE LISTING (581 sequences)

SEQ ID NO: 1 - protein, 879 aa, Homo sapiens
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2 - protein, 731 aa, Homo sapiens
PSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 3 - protein, 611 aa, Homo sapiens
VATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 4 - protein, 485 aa, homo sapien
SPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 5 - protein, 343 aa, Homo sapiens
KPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 6 - protein, 217 aa, Homo sapiens
VRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 7 - protein, 66 aa, homo sapiens
SKPVFITVKMDVLNAFKYPLLIGVGLSTVIGLLSCLIGYCSSHWCCKKEVQETRRERRRLMSMEMD

SEQ ID NO: 8 - protein, 21 aa, Homo sapiens
MGRLASRPLLLALLSLALCRG

SEQ ID NO: 9 - protein, 385 aa, Homo sapiens
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 10 - protein, 247 aa, Homo sapiens
PGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 11 - protein, 168 aa, Homo sapiens
HGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 12 - protein, 66 aa, homo sapiens
SHLAALWPFLGIVAEVLVLVTIIFIYEKRRKPEDVLDDDDAGSAPLKSSGQHQNDKGKNVRQRNSS

SEQ ID NO: 13 - protein, 18 aa, homo sapiens
MAAALFVLLGFALLGTHG

SEQ ID NO: 14 - protein, 613 aa, homo sapiens
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 15 - protein, 456 aa, homo sapiens
APPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 16 - protein, 320 aa, Homo sapiens
AHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 17 - protein, 179 aa, homo sapiens
REEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 18 - protein, 24 aa, Homo sapiens
VALVTGATVLGTITCCFMKRLRKR

SEQ ID NO: 19 - protein, 27 aa, Homo sapiens
MGALRPTLLPPSLPLLLLLMLGMGCWA

SEQ ID NO: 20 - protein, 1195 aa, homo sapiens
MKCFFPVLSCLAVLGVVSAQRQVTVQEGPLYRTEGSHITIWCNVSGYQGPSEQNFQWSIYLPSSPEREVQIVSTMDSSFPYAIYTQRVRGGKIFIERVQGNSTLLHITDLQARDAGEYECHTPSTDKQYFGSYSAKMNLVVIPDSLQTTAMPQTLHRVEQDPLELTCEVASETIQHSHLSVAWLRQKVGEKPVEVISLSRDFMLHSSSEYAQRQSLGEVRLDKLGRTTFRLTIFHLQPSDQGEFYCEAAEWIQDPDGSWYAMTRKRSEGAVVNVQPTDKEFTVRLETEKRLHTVGEPVEFRCILEAQNVPDRYFAVSWAFNSSLIATMGPNAVPVLNSEFAHREARGQLKVAKESDSVFVLKIYHLRQEDSGKYNCRVTEREKTVTGEFIDKESKRPKNIPIIVLPLKSSISVEVASNASVILEGEDLRFSCSVRTAGRPQGRFSVIWQLVDRQNRRSNIMWLDRDGTVQPGSSYWERSSFGGVQMEQVQPNSFSLGIFNSRKEDEGQYECHVTEWVRAVDGEWQIVGERRASTPISITALEMGFAVTAISRTPGVTYSDSFDLQCIIKPHYPAWVPVSVTWRFQPVGTVEFHDLVTFTRDGGVQWGDRSSSFRTRTAIEKAESSNNVRLSISRASDTEAGKYQCVAELWRKNYNNTWTRLAERTSNLLEIRVLQPVTKLQVSKSKRTLTLVENKPIQLNCSVKSQTSQNSHFAVLWYVHKPSDADGKLILKTTHNSAFEYGTYAEEEGLRARLQFERHVSGGLFSLTVQRAEVSDSGSYYCHVEEWLLSPNYAWYKLAEEVSGRTEVTVKQPDSRLRLSQAQGNLSVLETRQVQLECVVLNRTSITSQLMVEWFVWKPNHPERETVARLSRDATFHYGEQAAKNNLKGRLHLESPSPGVYRLFIQNVAVQDSGTYSCHVEEWLPSPSGMWYKRAEDTAGQTALTVMRPDASLQVDTVVPNATVSEKAAFQLDCSIVSRSSQDSRFAVAWYSLRTKAGGKRSSPGLEEQEEEREEEEEEEEDDDDDDPTERTALLSVGPDAVFGPEGSPWEGRLRFQRLSPVLYRLTVLQASPQDTGNYSCHVEEWLPSPQKEWYRLTEEESAPIGIRVLDTSPTLQSIICSNDALFYFVFFYPFPIFGILIITILLVRFKSRNSSKNSDGKNGVPLLWIKEPHLNYSPTCLEPPVLSIHPGAID

SEQ ID NO: 21 - protein, 798 aa, homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 22 - protein, 1032 aa, homo sapiens [sequence omitted]

SEQ ID NO: 23 - protein, 660 aa, homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTETGFHHVSQADIEFLTSIDPTASASGSAGITGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYA

SEQ ID NO: 24 - protein, 1023 aa, homo sapiens
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 25 - protein, 1020 aa, homo sapiens
MGRGAGREYSPAATTAENGGGKKKQKEKELDELKKEVAMDDHKLSLDELGRKYQVDLSKGLTNQRAQDVLARDGPNALTPPPTTPEWVKFCRQLFGGFSILLWIGAILCFLAYGIQAAMEDEPSNDNLYLGVVLAAVVIVTGCFSYYQEAKSSKIMDSFKNMVPQQALVIREGEKMQINAEEVVVGDLVEVKGGDRVPADLRIISSHGCKVDNSSLTGESEPQTRSPEFTHENPLETRNICFFSTNCVEGTARGIVIATGDRTVMGRIATLASGLEVGRTPIAMEIEHFIQLITGVAVFLGVSFFVLSLILGYSWLEAVIFLIGIIVANVPEGLLATVTVCLTLTAKRMARKNCLVKNLEAVETLGSTSTICSDKTGTLTQNRMTVAHMWFDNQIHEADTTEDQSGATFDKRSPTWTALSRIAGLCNRAVFKAGQENISVSKRDTAGDASESALLKCIELSCGSVRKMRDRNPKVAEIPFNSTNKYQLSIHEREDSPQSHVLVMKGAPERILDRCSTILVQGKEIPLDKEMQDAFQNAYMELGGLGERVLGFCQLNLPSGKFPRGFKFDTDELNFPTEKLCFVGLMSMIDPPRAAVPDAVGKCRSAGIKVIMVTGDHPITAKAIAKGVGIISEGNETVEDIAARLNIPMSQVNPREAKACVVHGSDLKDMTSEQLDEILKNHTEIVFARTSPQQKLIIVEGCQRQGAIVAVTGDGVNDSPALKKADIGIAMGISGSDVSKQAADMILLDDNFASIVTGVEEGRLIFDNLKKSIAYTLTSNIPEITPFLLFIIANIPLPLGTVTILCIDLGTDMVPAISLAYEAAESDIMKRQPRNSQTDKLVNERLISMAYGQIGMIQALGGFFTYFVILAENGFLPSRLLGIRLDWDDRTMNDLEDSYGQEWTYEQRKVVEFTCHTAFFASIVVVQWADLIICKTRRNSVFQQGMKNKILIFGLLEETALAAFLSYCPGMGVALRMYPLKVTWWFCAFPYSLLIFIYDEVRKLILRRYPGGWVEKETYY

SEQ ID NO: 26 - protein, 1026 aa, homo sapiens
MGSGGSDSYRIATSQDKKDDKDSPKKNKGKERRDLDDLKKEVAMTEHKMSVEEVCRKYNTDCVQGLTHSKAQEILARDGPNALTPPPTTPEWVKFCRQLFGGFSILLWIGAILCFLAYGIQAGTEDDPSGDNLYLGIVLAAVVIITGCFSYYQEAKSSKIMESFKNMVPQQALVIREGEKMQVNAEEVVVGDLVEIKGGDRVPADLRIISAHGCKVDNSSLTGESEPQTRSPDCTHDNPLETRNITFFSTNCVEGTARGVVVATGDRTVMGRIATLASGLEVGKTPIAIEIEHFIQLITGVAVFLGVSFFILSLILGYTWLEAVIFLIGIIVANVPEGLLATVTVCLTLTAKRMARKNCLVKNLEAVETLGSTSTICSDKTGTLTQNRMTVAHMWFDNQIHEADTTEDQSGTSFDKSSHTWVALSHIAGLCNRAVFKGGQDNIPVLKRDVAGDASESALLKCIELSSGSVKLMRERNKKVAEIPFNSTNKYQLSIHETEDPNDNRYLLVMKGAPERILDRCSTILLQGKEQPLDEEMKEAFQNAYLELGGLGERVLGFCHYYLPEEQFPKGFAFDCDDVNFTTDNLCFVGLMSMIDPPRAAVPDAVGKCRSAGIKVIMVTGDHPITAKAIAKGVGIISEGNETVEDIAARLNIPVSQVNPRDAKACVIHGTDLKDFTSEQIDEILQNHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLLFIMANIPLPLGTITILCIDLGTDMVPAISLAYEAAESDIMKRQPRNPRTDKLVNERLISMAYGQIGMIQALGGFFSYFVILAENGFLPGNLVGIRLNWDDRTVNDLEDSYGQQWTYEQRKVVEFTCHTAFFVSIVVVQWADLIICKTRRNSVFQQGMKNKILIFGLFEETALAAFLSYCPGMDVALRMYPLKPSWWFCAFPYSFLIFVYDEIRKLILRRNPGGWVEKETYY

SEQ ID NO: 27 - protein, 1029 aa, Homo sapiens
MGLWGKKGTVAPHDQSPRRRPKKGLIKKKMVKREKQKRNMEELKKEVVMDDHKLTLEELSTKYSVDLTKGHSHQRAKEILTRGGPNTVTPPPTTPEWVKFCKQLFGGFSLLLWTGAILCFVAYSIQIYFNEEPTKDNLYLSIVLSVVVIVTGCFSYYQEAKSSKIMESFKNMVPQQALVIRGGEKMQINVQEVVLGDLVEIKGGDRVPADLRLISAQGCKVDNSSLTGESEPQSRSPDFTHENPLETRNICFFSTNCVEGTARGIVIATGDSTVMGRIASLTSGLAVGQTPIAAEIEHFIHLITVVAVFLGVTFFALSLLLGYGWLEAIIFLIGIIVANVPEGLLATVTVCLTLTAKRMARKNCLVKNLEAVETLGSTSTICSDKTGTLTQNRMTVAHMWFDMTVYEADTTEEQTGKTFTKSSDTWFMLARIAGLCNRADFKANQEILPIAKRATTGDASESALLKFIEQSYSSVAEMREKNPKVAEIPFNSTNKYQMSIHLREDSSQTHVLMMKGAPERILEFCSTFLLNGQEYSMNDEMKEAFQNAYLELGGLGERVLGFCFLNLPSSFSKGFPFNTDEINFPMDNLCFVGLISMIDPPRAAVPDAVSKCRSAGIKVIMVTGDHPITAKAIAKGVGIISEGTETAEEVAARLKIPISKVDASAAKAIVVHGAELKDIQSKQLDQILQNHPEIVFARTSPQQKLIIVEGCQRLGAVVAVTGDGVNDSPALKKADIGIAMGISGSDVSKQAADMILLDDNFASIVTGVEEGRLIFDNLKKSIMYTLTSNIPEITPFLMFIILGIPLPLGTITILCIDLGTDMVPAISLAYESAESDIMKRLPRNPKTDNLVNHRLIGMAYGQIGMIQALAGFFTYFVILAENGFRPVDLLGIRLHWEDKYLNDLEDSYGQQWTYEQRKVVEFTCQTAFFVTIVVVQWADLIISKTRRNSLFQQGMRNKVLIFGILEETLLAAFLSYTPGMDVALRMYPLKITWWLCAIPYSILIFVYDEIRKLLIRQHPDGWVERETYY

SEQ ID NO: 28 - protein, 279 aa, homo sapiens
MTKNEKKSLNQSLAEWKLFIYNPTTGEFLGRTAKSWGLILLFYLVFYGFLAALFSFTMWVMLQTLNDEVPKYRDQIPSPGLMVFPKPVTALEYTFSRSDPTSYAGYIEDLKKFLKPYTLEEQKNLTVCPDGALFEQKGPVYVACQFPISLLQACSGMNDPDFGYSQGNPCILVKMNRIIGLKPEGVPRIDCVSKNEDIPNVAVYPHNGMIDLKYFPYYGKKLHVGYLQPLVAVQVSFAPNNTGKEVTVECKIDGSANLKSQDDRDKFLGRVMFKITARA

SEQ ID NO: 29 - protein, 1258 aa, Homo sapiens
MGDMANNSVAYSGVKNSLKEANHDGDFGITLAELRALMELRSTDALRKIQESYGDVYGICTKLKTSPNEGLSGNPADLERREAVFGKNFIPPKKPKTFLQLVWEALQDVTLIILEIAAIVSLGLSFYQPPEGDNALCGEVSVGEEEGEGETGWIEGAAILLSVVCVVLVTAFNDWSKEKQFRGLQSRIEQEQKFTVIRGGQVIQIPVADITVGDIAQVKYGDLLPADGILIQGNDLKIDESSLTGESDHVKKSLDKDPLLLSGTHVMEGSGRMVVTAVGVNSQTGIIFTLLGAGGEEEEKKDEKKKEKKNKKQDGAIENRNKAKAQDGAAMEMQPLKSEEGGDGDEKDKKKANLPKKEKSVLQGKLTKLAVQIGKAGLLMSAITVIILVLYFVIDTFWVQKRPWLAECTPIYIQYFVKFFIIGVTVLVVAVPEGLPLAVTISLAYSVKKMMKDNNLVRHLDACETMGNATAICSDKTGTLTMNRMTVVQAYINEKHYKKVPEPEAIPPNILSYLVTGISVNCAYTSKILPPEKEGGLPRHVGNKTECALLGLLLDLKRDYQDVRNEIPEEALYKVYTFNSVRKSMSTVLKNSDGSYRIFSKGASEIILKKCFKILSANGEAKVFRPRDRDDIVKTVIEPMASEGLRTICLAFRDFPAGEPEPEWDNENDIVTGLTCIAVVGIEDPVRPEVPDAIKKCQRAGITVRMVTGDNINTARAIATKCGILHPGEDFLCLEGKDFNRRIRNEKGEIEQERIDKIWPKLRVLARSSPTDKHTLVKGIIDSTVSDQRQVVAVTGDGTNDGPALKKADVGFAMGIAGTDVAKEASDIILTDDNFTSIVKAVMWGRNVYDSISKFLQFQLTVNVVAVIVAFTGACITQDSPLKAVQMLWVNLIMDTLASLALATEPPTESLLLRKPYGRNKPLISRTMMKNILGHAFYQLVVVFTLLFAGEKFFDIDSGRNAPLHAPPSEHYTIVFNTFVLMQLFNEINARKIHGERNVFEGIFNNAIFCTIVLGTFVVQIIIVQFGGKPFSCSELSIEQWLWSIFLGMGTLLWGQLISTIPTSRLKFLKEAGHGTQKEEIPEEELAEDVEEIDHAERELRRGQILWFRGLNRIQTQMDVVNAFQSGSSIQGALRRQPSIASQHHDVTNISTPTHIRVVNAFRSSLYEGLEKPESRSSIHNFMTHPEFRIEDSEPHIPLIDDTDAEDDAPTKRNSSPPPSPNKNNNAVDSGIHLTIEMNKSATSSSPGSPLHSLETSL

SEQ ID NO: 30 - protein, 1272 aa, Homo sapiens
MGDMTNSDFYSKNQRNESSHGGEFGCTMEELRSLMELRGTEAVVKIKETYGDTEAICRRLKTSPVEGLPGTAPDLEKRKQIFGQNFIPPKKPKTFLQLVWEALQDVTLIILEIAAIISLGLSFYHPPGEGNEGCATAQGGAEDEGEAEAGWIEGAAILLSVICVVLVTAFNDWSKEKQFRGLQSRIEQEQKFTVVRAGQVVQIPVAEIVVGDIAQVKYGDLLPADGLFIQGNDLKIDESSLTGESDQVRKSVDKDPMLLSGTHVMEGSGRMLVTAVGVNSQTGIIFTLLGAGGEEEEKKDKKGVKKGDGLQLPAADGAAASNAADSANASLVNGKMQDGNVDASQSKAKQQDGAAAMEMQPLKSAEGGDADDRKKASMHKKEKSVLQGKLTKLAVQIGKAGLVMSAITVIILVLYFTVDTFVVNKKPWLPECTPVYVQYFVKFFIIGVTVLVVAVPEGLPLAVTISLAYSVKKMMKDNNLVRHLDACETMGNATAICSDKTGTLTTNRMTVVQAYVGDVHYKEIPDPSSINTKTMELLINAIAINSAYTTKILPPEKEGALPRQVGNKTECGLLGFVLDLKQDYEPVRSQMPEEKLYKVYTFNSVRKSMSTVIKLPDESFRMYSKGASEIVLKKCCKILNGAGEPRVFRPRDRDEMVKKVIEPMACDGLRTICVAYRDFPSSPEPDWDNENDILNELTCICVVGIEDPVRPEVPEAIRKCQRAGITVRMVTGDNINTARAIAIKCGIIHPGEDFLCLEGKEFNRRIRNEKGEIEQERIDKIWPKLRVLARSSPTDKHTLVKGIIDSTHTEQRQVVAVTGDGTNDGPALKKADVGFAMGIAGTDVAKEASDIILTDDNFSSIVKAVMWGRNVYDSISKFLQFQLTVNVVAVIVAFTGACITQDSPLKAVQMLWVNLIMDTFASLALATEPPTETLLLRKPYGRNKPLISRTMMKNILGHAVYQLALIFTLLFVGEKMFQIDSGRNAPLHSPPSEHYTIIFNTFVMMQLFNEINARKIHGERNVFDGIFRNPIFCTIVLGTFAIQIVIVQFGGKPFSCSPLQLDQWMWCIFIGLGELVWGQVIATIPTSRLKFLKEAGRLTQKEEIPEEELNEDVEEIDHAERELRRGQILWFRGLNRIQTQIEVVNTFKSGASFQGALRRQSSVTSQSQDIRVVKAFRSSLYEGLEKPESRTSIHNFMAHPEFRIEDSQPHIPLIDDTDLEEDAALKQNSSPPSSLNKNNSAIDSGINLTTDTSKSATSSSPGSPIHSLETSL

SEQ ID NO: 31 - protein, 1241 aa, Homo sapiens
MTNPSDRVLPANSMAESREGDFGCTVMELRKLMELRSRDALTQINVHYGGVQNLCSRLKTSPVEGLSGNPADLEKRRQVFGHNVIPPKKPKTFLELVWEALQDVTLIILEIAAIISLVLSFYRPAGEENELCGQVATTPEDENEAQAGWIEGAAILFSVIIVVLVTAFNDWSKEKQFRGLQCRIEQEQKFSIIRNGQLIQLPVAEIVVGDIAQVKYGDLLPADGILIQGNDLKIDESSLTGESDHVKKSLDKDPMLLSGTHVMEGSGRMVVTAVGVNSQTGIILTLLGVNEDDEGEKKKKGKKQGVPENRNKAKTQDGVALEIQPLNSQEGIDNEEKDKKAVKVPKKEKSVLQGKLTRLAVQIGKAGLLMSALTVFILILYFVIDNFVINRRPWLPECTPIYIQYFVKFFIIGITVLVVAVPEGLPLAVTISLAYSVKKMMKDNNLVRHLDACETMGNATAICSDKTGTLTMNRMTVVQAYIGGIHYRQIPSPDVFLPKVLDLIVNGISINSAYTSKILPPEKEGGLPRQVGNKTECALLGFVTDLKQDYQAVRNEVPEEKLYKVYTFNSVRKSMSTVIRNPNGGFRMYSKGASEIILRKCNRILDRKGEAVPFKNKDRDDMVRTVIEPMACDGLRTICIAYRDFDDTEPSWDNENEILTELTCIAVVGIEDPVRPEVPDAIAKCKQAGITVRMVTGDNINTARAIATKCGILTPGDDFLCLEGKEFNRLIRNEKGEVEQEKLDKIWPKLRVLARSSPTDKHTLVKGIIDSTVGEHRQVVAVTGDGTNDGPALKKADVGFAMGIAGTDVAKEASDIILTDDNFTSIVKAVMWGRNVYDSISKFLQFQLTVNVVAVIVAFTGACITQDSPLKAVQMLWVNLIMDTFASLALATEPPTESLLKRRPYGRNKPLISRTMMKNILGHAFYQLIVIFILVFAGEKFFDIDSGRKAPLHSPPSQHYTIVFNTFVLMQLFNEINSRKIHGEKNVFSGIYRNIIFCSVVLGTFICQIFIVEFGGKPFSCTSLSLSQWLWCLFIGIGELLWGQFISAIPTRSLKFLKEAGHGTTKEEITKDAEGLDEIDHAEMELRRGQILWFRGLNRIQTQIDVINTFQTGASFKGVLRRQNMGQHLDVKLVPSSSYIKVVKAFHSSLHESIQKPYNQKSIHSFMTHPEFAIEEELPRTPLLDEEEEENPDKASKFGTRVLLLDGEVTPYANTNNNAVDCNQVQLPQSDSSLQSLETSV

SEQ ID NO: 32 - protein, 1241 aa, homo sapiens; identical to SEQ ID NO: 31

SEQ ID NO: 33 - protein, 193 aa, homo sapiens
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 34 - protein, 1021 aa, homo sapiens
MAGISYVASFFLLLTKLSIGQREVTVQKGPLFRAEGYPVSIGCNVTGHQGPSEQHFQWSVYLPTNPTQEVQIISTKDAAFSYAVYTQRVRSGDVYVERVQGNSVLLHISKLQMKDAGEYECHTPNTDEKYYGSYSAKTNLIVIPDTLSATMSSQTLGKEEGEPLALTCEASKATAQHTHLSVTWYLTQDGGGSQATEIISLSKDFILVPGPLYTERFAASDVQLNKLGPTTFRLSIERLQSSDQGQLFCEATEWIQDPDETWMFITKKQTDQTTLRIQPAVKDFQVNITADSLFAEGKPLELVCLVVSSGRDPQLQGIWFFNGTEIAHIDAGGVLGLKNDYKERASQGELQVSKLGPKAFSLKIFSLGPEDEGAYRCVVAEVMKTRTGSWQVLQRKQSPDSHVHLRKPAARSVVMSTKNKQQVVWEGETLAFLCKAGGAESPLSVSWWHIPRDQTQPEFVAGMGQDGIVQLGASYGVPSYHGNTRLEKMDWATFQLEITFTAITDSGTYECRVSEKSRNQARDLSWTQKISVTVKSLESSLQVSLMSRQPQVMLTNTFDLSCVVRAGYSDLKVPLTVTWQFQPASSHIFHQLIRITHNGTIEWGNFLSRFQKKTKVSQSLFRSQLLVHDATEEETGVYQCEVEVYDRNSLYNNRPPRASAISHPLRIAVTLPESKLKVNSRSQVQELSINSNTDIECSILSRSNGNLQLAIIWYFSPVSTNASWLKILEMDQTNVIKTGDEFHTPQRKQKFHTEKVSQDLFQLHILNVEDSDRGKYHCAVEEWLLSTNGTWHKLGEKKSGLTELKLKPTGSKVRVSKVYWTENVTEHREVAIRCSLESVGSSATLYSVMWYWNRENSGSKLLVHLQHDGLLEYGEEGLRRHLHCYRSSSTDFVLKLHQVEMEDAGMYWCRVAEWQLHGHPSKWINQASDESQRMVLTVLPSEPTLPSRICSSAPLLYFLFICPFVLLLLLLISLLCLYWKARKLSTLRSNTRKEKALWVDLKEAGGVTTNRREDEEEDEGN

SEQ ID NO: 35 - protein, 20 aa, homo sapiens
MAGISYVASFFLLLTKLSIG

SEQ ID NO: 36 - DNA, 20 bp, Artificial Sequence
  Synthetic  Oligonucleotide
cgttggcagtccgccttaac

SEQ ID NO: 37 - DNA, 20 bp, Artificial Sequence
  Synthetic  oligonucleotide
catagtcactgacgttgcag

SEQ ID NO: 38 - DNA, 20 bp, Artificial Sequence
  Synthetic  oligonucleotide
ttgtggagcttgcaagcacc

SEQ ID NO: 39 - DNA, 20 bp, Artificial Sequence
  Synthetic  oligonucleotide
gttctttatgtggagctcca

SEQ ID NO: 40 - DNA, 20 bp, Artificial Sequence
  Synthetic  oligonucleotide
tatcccttgctgatcggcgt

SEQ ID NO: 41 - DNA, 20 bp, Artificial Sequence
  Synthetic  oligonucleotide
gctgcagtacccgatgagac

SEQ ID NO: 42 - protein, 38 aa, Artificial Sequence
  Synthetic Polypeptide
EHSAGGGGSDYKDDDDKGGGGSLSNPIEIDFQTSGPIF

SEQ ID NO: 43 - protein, 34 aa, Artificial Sequence
  Synthetic Polypeptide
EHSAGGGGSDYKDDDDKGGGGSIEIDFQTSGPIF

SEQ ID NO: 44 - protein, 30 aa, Artificial Sequence
  Synthetic Polypeptide
EHSAGGGGSDYKDDDDKGGGGSFQTSGPIF

SEQ ID NO: 45 - protein, 26 aa, Artificial Sequence
  Synthetic Polypeptide
EHSAGGGGSDYKDDDDKGGGGSGPIF

SEQ ID NO: 46 - protein, 42 aa, Artificial Sequence
  Synthetic Polypeptide
FITVKMDTLDPRSFLLRNPNDKYEPFWEDEEKNESGSDKTHT

SEQ ID NO: 47 - protein, 332 aa, Homo sapiens
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 48 - protein, 195 aa, Homo sapiens
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 49 - protein, 227 aa, Homo sapiens
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 50 - protein, 30 aa, Artificial Sequence
  Description of Artificial Sequence: Synthetic peptide
MGGKLSKKKKGYNVNDEKAKEKDKKAEGAA

SEQ ID NO: 51 - protein, 27 aa, Artificial Sequence
  Description of Artificial Sequence: Synthetic peptide
MGGKLSKKKKGYNVNDEKAKEKDKKAE

SEQ ID NO: 52 - protein, 24 aa, Artificial Sequence
  Description of Artificial Sequence: Synthetic peptide
MGGKLSKKKKGYNVNDEKAKEKDK

SEQ ID NO: 53 - protein, 21 aa, Artificial Sequence
  Description of Artificial Sequence: Synthetic peptide
MGGKLSKKKKGYNVNDEKAKE

SEQ ID NO: 54 - protein, 18 aa, Artificial Sequence
  Description of Artificial Sequence: Synthetic peptide
MGGKLSKKKKGYNVNDEK

SEQ ID NO: 55 - protein, 15 aa, Artificial Sequence
  Description of Artificial Sequence: Synthetic peptide
MGGKLSKKKKGYNVN

SEQ ID NO: 56 - protein, 12 aa, Artificial Sequence
  Description of Artificial Sequence: Synthetic peptide
MGGKLSKKKKGY

SEQ ID NO: 57 - protein, 11 aa, Artificial Sequence
  Description of Artificial Sequence: Synthetic peptide
MGGKLSKKKKG

SEQ ID NO: 58 - protein, 10 aa, Artificial Sequence
  Description of Artificial Sequence: Synthetic peptide
MGGKLSKKKK

SEQ ID NO: 59 - protein, 9 aa, Artificial Sequence
  Description of Artificial Sequence: Synthetic peptide
MGGKLSKKK

SEQ ID NO: 60 - protein, 8 aa, Artificial Sequence
  Description of Artificial Sequence: Synthetic peptide
MGGKLSKK

SEQ ID NO: 61 - protein, 7 aa, Artificial Sequence
  Description of Artificial Sequence: Synthetic peptide
MGGKLSK

SEQ ID NO: 62 - protein, 8 aa, Artificial Sequence
  Description of Artificial Sequence: Synthetic peptide
MGGKLAKK

SEQ ID NO: 63 - protein, 8 aa, Artificial Sequence
  Description of Artificial Sequence: Synthetic peptide
MGGKFSKK

SEQ ID NO: 64 - protein, 8 aa, Artificial Sequence
  Description of Artificial Sequence: Synthetic peptide
MGGKFAKK

SEQ ID NO: 65 - protein, 8 aa, Artificial Sequence
  Description of Artificial Sequence: Synthetic peptide
MGGKSSKK

SEQ ID NO: 66 - protein, 8 aa, Artificial Sequence
  Description of Artificial Sequence: Synthetic peptide
MGGKSAKK

SEQ ID NO: 67 - protein, 8 aa, Artificial Sequence
  Description of Artificial Sequence: Synthetic peptide
MGGKQSKK

SEQ ID NO: 68 - protein, 8 aa, Artificial Sequence
  Description of Artificial Sequence: Synthetic peptide
MGGKQAKK

SEQ ID NO: 69 - protein, 8 aa, Artificial Sequence
  Description of Artificial Sequence: Synthetic peptide
MGGQLSKK

SEQ ID NO: 70 - protein, 8 aa, Artificial Sequence
  Description of Artificial Sequence: Synthetic peptide
MGGQLAKK

SEQ ID NO: 71 - protein, 8 aa, Artificial Sequence
  Description of Artificial Sequence: Synthetic peptide
MGGQFSKK

SEQ ID NO: 72 - protein, 8 aa, Artificial Sequence
  Description of Artificial Sequence: Synthetic peptide
MGGQFAKK

SEQ ID NO: 73 - protein, 8 aa, Artificial Sequence
  Description of Artificial Sequence: Synthetic peptide
MGGQSSKK

SEQ ID NO: 74 - protein, 8 aa, Artificial Sequence
  Description of Artificial Sequence: Synthetic peptide
MGGQSAKK

SEQ ID NO: 75 - protein, 8 aa, Artificial Sequence
  Description of Artificial Sequence: Synthetic peptide
MGGQQSKK

SEQ ID NO: 76 - protein, 8 aa, Artificial Sequence
  Description of Artificial Sequence: Synthetic peptide
MGGQQAKK

SEQ ID NO: 77 - protein, 8 aa, Artificial Sequence
  Description of Artificial Sequence: Synthetic peptide
MGAKLSKK

SEQ ID NO: 78 - protein, 8 aa, Artificial Sequence
  Description of Artificial Sequence: Synthetic peptide
MGAKLAKK

SEQ ID NO: 79 - protein, 8 aa, Artificial Sequence
  Description of Artificial Sequence: Synthetic peptide
MGAKFSKK

SEQ ID NO: 80 - protein, 8 aa, Artificial Sequence
  Description of Artificial Sequence: Synthetic peptide
MGAKFAKK

SEQ ID NO: 81 - protein, 8 aa, Artificial Sequence
  Description of Artificial Sequence: Synthetic peptide
MGAKSSKK

SEQ ID NO: 82 - protein, 8 aa, Artificial Sequence
  Description of Artificial Sequence: Synthetic peptide
MGAKSAKK

SEQ ID NO: 83 - protein, 8 aa, Artificial Sequence
  Description of Artificial Sequence: Synthetic peptide
MGAKQSKK

SEQ ID NO: 84 - protein, 8 aa, Artificial Sequence
  Description of Artificial Sequence: Synthetic peptide
MGAKQAKK

SEQ ID NO: 85 - protein, 8 aa, Artificial Sequence
  Description of Artificial Sequence: Synthetic peptide
MGAQLSKK

SEQ ID NO: 86 - protein, 8 aa, Artificial Sequence
  Description of Artificial Sequence: Synthetic peptide
MGAQLAKK

SEQ ID NO: 87 - protein, 8 aa, Artificial Sequence
  Description of Artificial Sequence: Synthetic peptide
MGAQFSKK

SEQ ID NO: 88 - protein, 8 aa, Artificial Sequence
  Description of Artificial Sequence: Synthetic peptide
MGAQFAKK

SEQ ID NO: 89 - protein, 8 aa, Artificial Sequence
  Description of Artificial Sequence: Synthetic peptide
MGAQSSKK

SEQ ID NO: 90 - protein, 8 aa, Artificial Sequence
  Description of Artificial Sequence: Synthetic peptide
MGAQSAKK

SEQ ID NO: 91 - protein, 8 aa, Artificial Sequence
  Description of Artificial Sequence: Synthetic peptide
MGAQQSKK

SEQ ID NO: 92 - protein, 8 aa, Artificial Sequence
  Description of Artificial Sequence: Synthetic peptide
MGAQQAKK

SEQ ID NO: 93 - protein, 8 aa, Artificial Sequence
  Description of Artificial Sequence: Synthetic peptide
MGSKLSKK

SEQ ID NO: 94 - protein, 8 aa, Artificial Sequence
  Description of Artificial Sequence: Synthetic peptide
MGSKLAKK

SEQ ID NO: 95 - protein, 8 aa, Artificial Sequence
  Description of Artificial Sequence: Synthetic peptide
MGSKFSKK

SEQ ID NO: 96 - protein, 8 aa, Artificial Sequence
  Description of Artificial Sequence: Synthetic peptide
MGSKFAKK

SEQ ID NO: 97 - protein, 8 aa, Artificial Sequence
  Description of Artificial Sequence: Synthetic peptide
MGSKSSKK

SEQ ID NO: 98 - protein, 8 aa, Artificial Sequence
  Description of Artificial Sequence: Synthetic peptide
MGSKSAKK

SEQ ID NO: 99 - protein, 8 aa, Artificial Sequence
  Description of Artificial Sequence: Synthetic peptide
MGSKQSKK

SEQ ID NO: 100 - protein, 8 aa, Artificial Sequence
  Description of Artificial Sequence: Synthetic peptide
MGSKQAKK

SEQ ID NO: 101 - protein, 8 aa, Artificial Sequence
  Description of Artificial Sequence: Synthetic peptide
MGSQLSKK

SEQ ID NO: 102 - protein, 8 aa, Artificial Sequence
  Description of Artificial Sequence: Synthetic peptide
MGSQLAKK

SEQ ID NO: 103 - protein, 8 aa, Artificial Sequence
  Description of Artificial Sequence: Synthetic peptide
MGSQFSKK

SEQ ID NO: 104 - protein, 8 aa, Artificial Sequence
  Description of Artificial Sequence: Synthetic peptide
MGSQFAKK

SEQ ID NO: 105 - protein, 8 aa, Artificial Sequence
  Description of Artificial Sequence: Synthetic peptide
MGSQSSKK

SEQ ID NO: 106 - protein, 8 aa, Artificial Sequence
  Description of Artificial Sequence: Synthetic peptide
MGSQSAKK

SEQ ID NO: 107 - protein, 8 aa, Artificial Sequence
  Description of Artificial Sequence: Synthetic peptide
MGSQQSKK

SEQ ID NO: 108 - protein, 8 aa, Artificial Sequence
  Description of Artificial Sequence: Synthetic peptide
MGSQQAKK

SEQ ID NO: 109 - protein, 7 aa, Artificial Sequence
  Description of Artificial Sequence: Synthetic peptide
MGGKLAK

SEQ ID NO: 110 - protein, 7 aa, Artificial Sequence
  Description of Artificial Sequence: Synthetic peptide
MGGKFSK

SEQ ID NO: 111 - protein, 7 aa, Artificial Sequence
  Description of Artificial Sequence: Synthetic peptide
MGGKFAK

SEQ ID NO: 112 - protein, 7 aa, Artificial Sequence
  Description of Artificial Sequence: Synthetic peptide
MGGKSSK

SEQ ID NO: 113 - protein, 7 aa, Artificial Sequence
  Description of Artificial Sequence: Synthetic peptide
MGGKSAK

SEQ ID NO: 114 - protein, 7 aa, Artificial Sequence
  Description of Artificial Sequence: Synthetic peptide
MGGKQSK

SEQ ID NO: 115 - protein, 7 aa, Artificial Sequence
  Description of Artificial Sequence: Synthetic peptide
MGGKQAK

SEQ ID NO: 116 - protein, 7 aa, Artificial Sequence
  Description of Artificial Sequence: Synthetic peptide
MGGQLSK

SEQ ID NO: 117 - protein, 7 aa, Artificial Sequence
  Description of Artificial Sequence: Synthetic peptide
MGGQLAK

SEQ ID NO: 118 - protein, 7 aa, Artificial Sequence
  Description of Artificial Sequence: Synthetic peptide
MGGQFSK

SEQ ID NO: 119 - protein, 7 aa, Artificial Sequence
  Description of Artificial Sequence: Synthetic peptide
MGGQFAK

SEQ ID NO: 120 - protein, 7 aa, Artificial Sequence
  Description of Artificial Sequence: Synthetic peptide
MGGQSSK

SEQ ID NO: 121 - protein, 7 aa, Artificial Sequence
  Description of Artificial Sequence: Synthetic peptide
MGGQSAK

SEQ ID NO: 122 - protein, 7 aa, Artificial Sequence
  Description of Artificial Sequence: Synthetic peptide
MGGQQSK

SEQ ID NO: 123 - protein, 7 aa, Artificial Sequence
  Description of Artificial Sequence: Synthetic peptide
MGGQQAK

SEQ ID NO: 124 - protein, 7 aa, Artificial Sequence
  Description of Artificial Sequence: Synthetic peptide
MGAKLSK

SEQ ID NO: 125 - protein, 7 aa, Artificial Sequence
  Description of Artificial Sequence: Synthetic peptide
MGAKLAK

SEQ ID NO: 126 - protein, 7 aa, Artificial Sequence
  Description of Artificial Sequence: Synthetic peptide
MGAKFSK

SEQ ID NO: 127 - protein, 7 aa, Artificial Sequence
  Description of Artificial Sequence: Synthetic peptide
MGAKFAK

SEQ ID NO: 128 - protein, 7 aa, Artificial Sequence
  Description of Artificial Sequence: Synthetic peptide
MGAKSSK

SEQ ID NO: 129 - protein, 7 aa, Artificial Sequence
  Description of Artificial Sequence: Synthetic peptide
MGAKSAK

SEQ ID NO: 130 - protein, 7 aa, Artificial Sequence
  Description of Artificial Sequence: Synthetic peptide
MGAKQSK

SEQ ID NO: 131 - protein, 7 aa, Artificial Sequence
  Description of Artificial Sequence: Synthetic peptide
MGAKQAK

SEQ ID NO: 132 - protein, 7 aa, Artificial Sequence
  Description of Artificial Sequence: Synthetic peptide
MGAQLSK

SEQ ID NO: 133 - protein, 7 aa, Artificial Sequence
  Description of Artificial Sequence: Synthetic peptide
MGAQLAK

SEQ ID NO: 134 - protein, 7 aa, Artificial Sequence
  Description of Artificial Sequence: Synthetic peptide
MGAQFSK

SEQ ID NO: 135 - protein, 7 aa, Artificial Sequence
  Description of Artificial Sequence: Synthetic peptide
MGAQFAK

SEQ ID NO: 136 - protein, 7 aa, Artificial Sequence
  Description of Artificial Sequence: Synthetic peptide
MGAQSSK

SEQ ID NO: 137 - protein, 7 aa, Artificial Sequence
  Description of Artificial Sequence: Synthetic peptide
MGAQSAK

SEQ ID NO: 138 - protein, 7 aa, Artificial Sequence
  Description of Artificial Sequence: Synthetic peptide
MGAQQSK

SEQ ID NO: 139 - protein, 7 aa, Artificial Sequence
  Description of Artificial Sequence: Synthetic peptide
MGAQQAK

SEQ ID NO: 140 - protein, 7 aa, Artificial Sequence
  Description of Artificial Sequence: Synthetic peptide
MGSKLSK

SEQ ID NO: 141 - protein, 7 aa, Artificial Sequence
  Description of Artificial Sequence: Synthetic peptide
MGSKLAK

SEQ ID NO: 142 - protein, 7 aa, Artificial Sequence
  Description of Artificial Sequence: Synthetic peptide
MGSKFSK

SEQ ID NO: 143 - protein, 7 aa, Artificial Sequence
  Description of Artificial Sequence: Synthetic peptide
MGSKFAK

SEQ ID NO: 144 - protein, 7 aa, Artificial Sequence
  Description of Artificial Sequence: Synthetic peptide
MGSKSSK

SEQ ID NO: 145 - protein, 7 aa, Artificial Sequence
  Description of Artificial Sequence: Synthetic peptide
MGSKSAK

SEQ ID NO: 146 - protein, 7 aa, Artificial Sequence
  Description of Artificial Sequence: Synthetic peptide
MGSKQSK

SEQ ID NO: 147 - protein, 7 aa, Artificial Sequence
  Description of Artificial Sequence: Synthetic peptide
MGSKQAK

SEQ ID NO: 148 - protein, 7 aa, Artificial Sequence
  Description of Artificial Sequence: Synthetic peptide
MGSQLSK

SEQ ID NO: 149 - protein, 7 aa, Artificial Sequence
  Description of Artificial Sequence: Synthetic peptide
MGSQLAK

SEQ ID NO: 150 - protein, 7 aa, Artificial Sequence
  Description of Artificial Sequence: Synthetic peptide
MGSQFSK

SEQ ID NO: 151 - protein, 7 aa, Artificial Sequence
  Description of Artificial Sequence: Synthetic peptide
MGSQFAK

SEQ ID NO: 152 - protein, 7 aa, Artificial Sequence
  Description of Artificial Sequence: Synthetic peptide
MGSQSSK

SEQ ID NO: 153 - protein, 7 aa, Artificial Sequence
  Description of Artificial Sequence: Synthetic peptide
MGSQSAK

SEQ ID NO: 154 - protein, 7 aa, Artificial Sequence
  Description of Artificial Sequence: Synthetic peptide
MGSQQSK

SEQ ID NO: 155 - protein, 7 aa, Artificial Sequence
  Description of Artificial Sequence: Synthetic peptide
MGSQQAK

SEQ ID NO: 156 - protein, 9 aa, Artificial Sequence
  Description of Artificial Sequence: Synthetic peptide
MGAKLSKKK

SEQ ID NO: 157 - protein, 167 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 158 - protein, 167 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIY

SEQ ID NO: 159 - protein, 296 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGGSGGSGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 160 - protein, 296 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIYGSGGSGGSGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIY

SEQ ID NO: 161 - RNA, 680 nt, Artificial Sequence
  Description of Artificial Sequence: Synthetic polynucleotide
augaagcccaccgagaacaacgaagacuucaacaucguggccguggccagcaacuucgcgaccacggaucucgaugcugaccgcgggaaguugcccggcaagaagcugccgcuggaggugcucaaagaguuggaagccaaugcccggaaagcuggcugcaccaggggcugucugaucugccugucccacaucaagugcacgcccaagaugaagaaguucaucccaggacgcugccacaccuacgaaggcgacaaagaguccgcacagggcggcauaggcgaggcgaucgucgacauuccugagauuccuggguucaaggacuuggagcccuuggagcaguucaucgcacaggucgaucuguguguggacugcacaacuggcugccucaaagggcuugccaacgugcaguguucugaccugcucaagaaguggcugccgcaacgcugugcgaccuuugccagcaagauccagggccagguggacaagaucaagggggccgguggugacuaaggauccaucgauaagcuucaucgaaacaugaggaucacccauaucugcagucgacaucgaaacaugaggaucacccaugucugcagucgacaucgaaacaugaggaucacccaugucugcagucgacaucgaaacaugaggaucacccaugucugcagucgacaucgaaa

SEQ ID NO: 162 - protein, 9 aa, Artificial Sequence
  Description of Artificial Sequence: Synthetic peptide
  MOD_RES (3)..(3): Any Naturally Occuring Amino Acid
MGXKLSKKK

SEQ ID NO: 163 - protein, 9 aa, Artificial Sequence
  Description of Artificial Sequence: Synthetic peptide
  MOD_RES (3)..(3): See specification as filed for detailed description of substitutions and preferred embodiments
MGXKLSKKK

SEQ ID NO: 164 - protein, 8 aa, Artificial Sequence
  Description of Artificial Sequence: Synthetic peptide
  MOD_RES (3)..(3): Gly, Ala, or Ser
  MOD_RES (4)..(4): Lys or Gln
  MOD_RES (5)..(5): Leu, Phe, Ser, or Gln
  MOD_RES (6)..(6): Ser or Ala
  MOD_RES (6)..(6): Ser or Ala, See specification as filed for detailed description of substitutions and preferred embodiments
MGXXXXKK

SEQ ID NO: 165 - DNA, 20 bp, Artificial Sequence
  Description of Artificial Sequence: Synthetic primer
tggaggtgctcaaagagttg

SEQ ID NO: 166 - DNA, 17 bp, Artificial Sequence
  Description of Artificial Sequence: Synthetic primer
ttgggcgtgcacttgat

SEQ ID NO: 167 - DNA, 13 bp, Artificial Sequence
  Description of Artificial Sequence: Synthetic probe
gggcattggcttc

SEQ ID NO: 168 - protein, 60 aa, Artificial Sequence
  Description of Artificial Sequence: Synthetic polypeptide
MGGKLSKKKKGYNVNDEKAKEKDKKAEGAASAGGGGSDYKDDDDKGGGGSVSKGEELFTG

SEQ ID NO: 169 - protein, 60 aa, Artificial Sequence
  Description of Artificial Sequence: Synthetic polypeptide
MAGKLSKKKKGYNVNDEKAKEKDKKAEGAASAGGGGSDYKDDDDKGGGGSVSKGEELFTG

SEQ ID NO: 170 - protein, 60 aa, Artificial Sequence
  Description of Artificial Sequence: Synthetic polypeptide
MGAKLSKKKKGYNVNDEKAKEKDKKAEGAASAGGGGSDYKDDDDKGGGGSVSKGEELFTG

SEQ ID NO: 171 - protein, 60 aa, Artificial Sequence
  Description of Artificial Sequence: Synthetic polypeptide
MAAKLSKKKKGYNVNDEKAKEKDKKAEGAASAGGGGSDYKDDDDKGGGGSVSKGEELFTG

SEQ ID NO: 172 - protein, 57 aa, Artificial Sequence
  Description of Artificial Sequence: Synthetic polypeptide
MGGKLSKKKKGYNVNDEKAKEKDKKAESAGGGGSDYKDDDDKGGGGSVSKGEELFTG

SEQ ID NO: 173 - protein, 54 aa, Artificial Sequence
  Description of Artificial Sequence: Synthetic polypeptide
MGGKLSKKKKGYNVNDEKAKEKDKSAGGGGSDYKDDDDKGGGGSVSKGEELFTG

SEQ ID NO: 174 - protein, 51 aa, Artificial Sequence
  Description of Artificial Sequence: Synthetic polypeptide
MGGKLSKKKKGYNVNDEKAKESAGGGGSDYKDDDDKGGGGSVSKGEELFTG

SEQ ID NO: 175 - protein, 48 aa, Artificial Sequence
  Description of Artificial Sequence: Synthetic polypeptide
MGGKLSKKKKGYNVNDEKSAGGGGSDYKDDDDKGGGGSVSKGEELFTG

SEQ ID NO: 176 - protein, 45 aa, Artificial Sequence
  Description of Artificial Sequence: Synthetic polypeptide
MGGKLSKKKKGYNVNSAGGGGSDYKDDDDKGGGGSVSKGEELFTG

SEQ ID NO: 177 - protein, 42 aa, Artificial Sequence
  Description of Artificial Sequence: Synthetic polypeptide
MGGKLSKKKKGYSAGGGGSDYKDDDDKGGGGSVSKGEELFTG

SEQ ID NO: 178 - protein, 39 aa, Artificial Sequence
  Description of Artificial Sequence: Synthetic polypeptide
MGGKLSKKKSAGGGGSDYKDDDDKGGGGSVSKGEELFTG

SEQ ID NO: 179 - protein, 36 aa, Artificial Sequence
  Description of Artificial Sequence: Synthetic polypeptide
MGGKLSSAGGGGSDYKDDDDKGGGGSVSKGEELFTG

SEQ ID NO: 180 - protein, 33 aa, Artificial Sequence
  Description of Artificial Sequence: Synthetic polypeptide
MGGSAGGGGSDYKDDDDKGGGGSVSKGEELFTG

SEQ ID NO: 181 - protein, 54 aa, Artificial Sequence
  Description of Artificial Sequence: Synthetic polypeptide
MGGKLSKKKKGYNVNDEKAKEKDKKAEGAASAGGGGSDYKDDDDKGGGGSVSKG

SEQ ID NO: 182 - protein, 36 aa, Artificial Sequence
  Description of Artificial Sequence: Synthetic polypeptide
MGGKLSKKKKGYSAGGGGSDYKDDDDKGGGGSVSKG

SEQ ID NO: 183 - protein, 35 aa, Artificial Sequence
  Description of Artificial Sequence: Synthetic polypeptide
MGGKLSKKKKGSAGGGGSDYKDDDDKGGGGSVSKG

SEQ ID NO: 184 - protein, 34 aa, Artificial Sequence
  Description of Artificial Sequence: Synthetic polypeptide
MGGKLSKKKKSAGGGGSDYKDDDDKGGGGSVSKG

SEQ ID NO: 185 - protein, 33 aa, Artificial Sequence
  Description of Artificial Sequence: Synthetic polypeptide
MGGKLSKKKSAGGGGSDYKDDDDKGGGGSVSKG

SEQ ID NO: 186 - protein, 32 aa, Artificial Sequence
  Description of Artificial Sequence: Synthetic polypeptide
MGGKLSKKSAGGGGSDYKDDDDKGGGGSVSKG

SEQ ID NO: 187 - protein, 31 aa, Artificial Sequence
  Description of Artificial Sequence: Synthetic polypeptide
MGGKLSKSAGGGGSDYKDDDDKGGGGSVSKG

SEQ ID NO: 188 - protein, 30 aa, Artificial Sequence
  Description of Artificial Sequence: Synthetic polypeptide
MGGKLSSAGGGGSDYKDDDDKGGGGSVSKG

SEQ ID NO: 189 - protein, 30 aa, Artificial Sequence
  Description of Artificial Sequence: Synthetic polypeptide
MGGKLDKKKKGYNVNDEKAKEKDKKAEGAA

SEQ ID NO: 190 - protein, 30 aa, Artificial Sequence
  Description of Artificial Sequence: Synthetic polypeptide
MGGKLAKKKKGYNVNDEKAKEKDKKAEGAA

SEQ ID NO: 191 - protein, 30 aa, Artificial Sequence
  Description of Artificial Sequence: Synthetic polypeptide
MGGKQSKKKKGYNVNDEKAKEKDKKAEGAA

SEQ ID NO: 192 - protein, 30 aa, Artificial Sequence
  Description of Artificial Sequence: Synthetic polypeptide
MGAKKKKKRFSFKKSFKLSGFSFKKNKKEA

SEQ ID NO: 193 - protein, 30 aa, Artificial Sequence
  Description of Artificial Sequence: Synthetic polypeptide
MAAKKKKKRFSFKKSFKLSGFSFKKNKKEA

SEQ ID NO: 194 - protein, 30 aa, Artificial Sequence
  Description of Artificial Sequence: Synthetic polypeptide
MGAKKSKKRFSFKKSFKLSGFSFKKNKKEA

SEQ ID NO: 195 - protein, 30 aa, Artificial Sequence
  Description of Artificial Sequence: Synthetic polypeptide
MGAKKAKKRFSFKKPFKLSGFSFKKNKKEA

SEQ ID NO: 196 - protein, 153 aa, Artificial Sequence
  Description of Artificial Sequence: Synthetic polypeptide
MGGKLSKKKKSAGGSGGSTSGSGDYKDDDDKGSGFEMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 197 - protein, 4 aa, Artificial Sequence
  Peptide Linker
GGGG

SEQ ID NO: 198 - protein, 7 aa, Artificial Sequence
  Peptide Linker
SGGSGGS

SEQ ID NO: 199 - protein, 15 aa, Artificial Sequence
  Peptide Linker
GGSGGSGGSGGSGGG

SEQ ID NO: 200 - protein, 16 aa, Artificial Sequence
  Peptide Linker
GGSGGSGGGGSGGGGS

SEQ ID NO: 201 - protein, 18 aa, Artificial Sequence
  Peptide Linker
GGSGGSGGSGGSGGSGGS

SEQ ID NO: 202 - protein, 15 aa, Artificial Sequence
  Peptide Linker
GGGGSGGGGSGGGGS

SEQ ID NO: 203 - protein, 400 aa, Artificial Sequence
  Peptide Linker
  MISC_FEATURE (1)..(400): This sequence may encompass 1 - 100 “GGGS” repeating units.
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 204 - protein, 800 aa, Artificial Sequence
  Peptide Linker
  MISC_FEATURE (1)..(300): This sequence may encompass 1-100 “GGS” repeating units
  MISC_FEATURE (301)..(800): This sequence may encompass 1-100 “GGGGS” repeating units.
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 205 - protein, 4 aa, Artificial Sequence
  Effector Domain Sequence
KKKK

SEQ ID NO: 206 - protein, 5 aa, Artificial Sequence
  Effector Domain Sequence
KKKKK

SEQ ID NO: 207 - protein, 4 aa, Artificial Sequence
  Effector Domain Sequence
RRRR

SEQ ID NO: 208 - protein, 5 aa, Artificial Sequence
  Effector Domain Sequence
RRRRR

SEQ ID NO: 209 - protein, 4 aa, Artificial Sequence
  Effector Domain Sequence
  misc_feature (1)..(4): Xaa can be Lys or Arg
XXXX

SEQ ID NO: 210 - protein, 5 aa, Artificial Sequence
  Effector Domain Sequence
  misc_feature (1)..(5): Xaa can be Lys or Arg
XXXXX

SEQ ID NO: 211 - protein, 7 aa, Artificial Sequence
  N Terminus Domain Sequence
GGKLSKK

SEQ ID NO: 212 - protein, 7 aa, Artificial Sequence
  N Terminus Domain Sequence
GAKLSKK

SEQ ID NO: 213 - protein, 7 aa, Artificial Sequence
  N Terminus Domain Sequence
GGKQSKK

SEQ ID NO: 214 - protein, 7 aa, Artificial Sequence
  N Terminus Domain Sequence
GGKLAKK

SEQ ID NO: 215 - protein, 6 aa, Artificial Sequence
  N Terminus Domain Sequence
GGKLSK

SEQ ID NO: 216 - protein, 6 aa, Artificial Sequence
  N Terminus Domain Sequence
GAKLSK

SEQ ID NO: 217 - protein, 6 aa, Artificial Sequence
  N Terminus Domain Sequence
GGKQSK

SEQ ID NO: 218 - protein, 6 aa, Artificial Sequence
  N Terminus Domain Sequence
GGKLAK

SEQ ID NO: 219 - protein, 4 aa, Artificial Sequence
  N Terminus Domain Sequence
KKKG

SEQ ID NO: 220 - protein, 5 aa, Artificial Sequence
  N Terminus Domain Sequence
KKKGY

SEQ ID NO: 221 - protein, 6 aa, Artificial Sequence
  N Terminus Domain Sequence
KKKGYN

SEQ ID NO: 222 - protein, 7 aa, Artificial Sequence
  N Terminus Domain Sequence
KKKGYNV

SEQ ID NO: 223 - protein, 8 aa, Artificial Sequence
  N Terminus Domain Sequence
KKKGYNVN

SEQ ID NO: 224 - protein, 6 aa, Artificial Sequence
  N Terminus Domain Sequence
KKKGYS

SEQ ID NO: 225 - protein, 6 aa, Artificial Sequence
  N Terminus Domain Sequence
KKKGYG

SEQ ID NO: 226 - protein, 7 aa, Artificial Sequence
  N Terminus Domain Sequence
KKKGYGG

SEQ ID NO: 227 - protein, 5 aa, Artificial Sequence
  N Terminus Domain Sequence
KKKGS

SEQ ID NO: 228 - protein, 6 aa, Artificial Sequence
  N Terminus Domain Sequence
KKKGSG

SEQ ID NO: 229 - protein, 7 aa, Artificial Sequence
  N Terminus Domain Sequence
KKKGSGS

SEQ ID NO: 230 - protein, 4 aa, Artificial Sequence
  N Terminus Domain Sequence
KKKS

SEQ ID NO: 231 - protein, 5 aa, Artificial Sequence
  N Terminus Domain Sequence
KKKSG

SEQ ID NO: 232 - protein, 6 aa, Artificial Sequence
  N Terminus Domain Sequence
KKKSGG

SEQ ID NO: 233 - protein, 7 aa, Artificial Sequence
  N Terminus Domain Sequence
KKKSGGS

SEQ ID NO: 234 - protein, 8 aa, Artificial Sequence
  N Terminus Domain Sequence
KKKSGGSG

SEQ ID NO: 235 - protein, 8 aa, Artificial Sequence
  N Terminus Domain Sequence
KKSGGSGG

SEQ ID NO: 236 - protein, 10 aa, Artificial Sequence
  N Terminus Domain Sequence
KKKSGGSGGS

SEQ ID NO: 237 - protein, 8 aa, Artificial Sequence
  N Terminus Domain Sequence
KRFSFKKS

SEQ ID NO: 238 - protein, 8 aa, Artificial Sequence
  N Terminus Domain Sequence
GGKLSKKK

SEQ ID NO: 239 - protein, 8 aa, Artificial Sequence
  N Terminus Domain Sequence
GGKLSKKS

SEQ ID NO: 240 - protein, 8 aa, Artificial Sequence
  N Terminus Domain Sequence
GAKLSKKK

SEQ ID NO: 241 - protein, 8 aa, Artificial Sequence
  N Terminus Domain Sequence
GAKLSKKS

SEQ ID NO: 242 - protein, 8 aa, Artificial Sequence
  N Terminus Domain Sequence
GGKQSKKK

SEQ ID NO: 243 - protein, 8 aa, Artificial Sequence
  N Terminus Domain Sequence
GGKQSKKS

SEQ ID NO: 244 - protein, 8 aa, Artificial Sequence
  N Terminus Domain Sequence
GGKLAKKK

SEQ ID NO: 245 - protein, 8 aa, Artificial Sequence
  N Terminus Domain Sequence
GGKLAKKS

SEQ ID NO: 246 - protein, 14 aa, Artificial Sequence
  Scaffold Y Sequence
GGKLSKKKKGYNVN

SEQ ID NO: 247 - protein, 14 aa, Artificial Sequence
  Scaffold Y Sequence
GAKLSKKKKGYNVN

SEQ ID NO: 248 - protein, 14 aa, Artificial Sequence
  Scaffold Y Sequence
GGKQSKKKKGYNVN

SEQ ID NO: 249 - protein, 14 aa, Artificial Sequence
  Scaffold Y Sequence
GGKLAKKKKGYNVN

SEQ ID NO: 250 - protein, 14 aa, Artificial Sequence
  Scaffold Y Sequence
GGKLSKKKKGYSGG

SEQ ID NO: 251 - protein, 14 aa, Artificial Sequence
  Scaffold Y Sequence
GGKLSKKKKGSGGS

SEQ ID NO: 252 - protein, 14 aa, Artificial Sequence
  Scaffold Y Sequence
GGKLSKKKKSGGSG

SEQ ID NO: 253 - protein, 14 aa, Artificial Sequence
  Scaffold Y Sequence
GGKLSKKKSGGSGG

SEQ ID NO: 254 - protein, 14 aa, Artificial Sequence
  Scaffold Y Sequence
GGKLSKKSGGSGGS

SEQ ID NO: 255 - protein, 14 aa, Artificial Sequence
  Scaffold Y Sequence
GGKLSKSGGSGGSV

SEQ ID NO: 256 - protein, 14 aa, Artificial Sequence
  Scaffold Y Sequence
GAKKSKKRFSFKKS

SEQ ID NO: 257 - protein, 29 aa, Artificial Sequence
  Scaffold Y Sequence
GGKLSKKKKGYNVNDEKAKEKDKKAEGAA

SEQ ID NO: 258 - protein, 28 aa, Artificial Sequence
  Scaffold Y Sequence
GGKLSKKKKGYNVNDEKAKEKDKKAEGA

SEQ ID NO: 259 - protein, 27 aa, Artificial Sequence
  Scaffold Y Sequence
GGKLSKKKKGYNVNDEKAKEKDKKAEG

SEQ ID NO: 260 - protein, 26 aa, Artificial Sequence
  Scaffold Y Sequence
GGKLSKKKKGYNVNDEKAKEKDKKAE

SEQ ID NO: 261 - protein, 25 aa, Artificial Sequence
  Scaffold Y Sequence
GGKLSKKKKGYNVNDEKAKEKDKKA

SEQ ID NO: 262 - protein, 24 aa, Artificial Sequence
  Scaffold Y Sequence
GGKLSKKKKGYNVNDEKAKEKDKK

SEQ ID NO: 263 - protein, 23 aa, Artificial Sequence
  Scaffold Y Sequence
GGKLSKKKKGYNVNDEKAKEKDK

SEQ ID NO: 264 - protein, 22 aa, Artificial Sequence
  Scaffold Y Sequence
GGKLSKKKKGYNVNDEKAKEKD

SEQ ID NO: 265 - protein, 21 aa, Artificial Sequence
  Scaffold Y Sequence
GGKLSKKKKGYNVNDEKAKEK

SEQ ID NO: 266 - protein, 20 aa, Artificial Sequence
  Scaffold Y Sequence
GGKLSKKKKGYNVNDEKAKE

SEQ ID NO: 267 - protein, 19 aa, Artificial Sequence
  Scaffold Y Sequence
GGKLSKKKKGYNVNDEKAK

SEQ ID NO: 268 - protein, 18 aa, Artificial Sequence
  Scaffold Y Sequence
GGKLSKKKKGYNVNDEKA

SEQ ID NO: 269 - protein, 17 aa, Artificial Sequence
  Scaffold Y Sequence
GGKLSKKKKGYNVNDEK

SEQ ID NO: 270 - protein, 16 aa, Artificial Sequence
  Scaffold Y Sequence
GGKLSKKKKGYNVNDE

SEQ ID NO: 271 - protein, 15 aa, Artificial Sequence
  Scaffold Y Sequence
GGKLSKKKKGYNVND

SEQ ID NO: 272 - protein, 13 aa, Artificial Sequence
  Scaffold Y Sequence
GGKLSKKKKGYNV

SEQ ID NO: 273 - protein, 12 aa, Artificial Sequence
  Scaffold Y Sequence
GGKLSKKKKGYN

SEQ ID NO: 274 - protein, 11 aa, Artificial Sequence
  Scaffold Y Sequence
GGKLSKKKKGY

SEQ ID NO: 275 - protein, 10 aa, Artificial Sequence
  Scaffold Y Sequence
GGKLSKKKKG

SEQ ID NO: 276 - protein, 9 aa, Artificial Sequence
  Scaffold Y Sequence
GGKLSKKKK

SEQ ID NO: 277 - protein, 28 aa, Artificial Sequence
  Scaffold Y Sequence
GAKKSKKRFSFKKSFKLSGFSFKKNKKE

SEQ ID NO: 278 - protein, 27 aa, Artificial Sequence
  Scaffold Y Sequence
GAKKSKKRFSFKKSFKLSGFSFKKNKK

SEQ ID NO: 279 - protein, 26 aa, Artificial Sequence
  Scaffold Y Sequence
GAKKSKKRFSFKKSFKLSGFSFKKNK

SEQ ID NO: 280 - protein, 25 aa, Artificial Sequence
  Scaffold Y Sequence
GAKKSKKRFSFKKSFKLSGFSFKKN

SEQ ID NO: 281 - protein, 24 aa, Artificial Sequence
  Scaffold Y Sequence
GAKKSKKRFSFKKSFKLSGFSFKK

SEQ ID NO: 282 - protein, 23 aa, Artificial Sequence
  Scaffold Y Sequence
GAKKSKKRFSFKKSFKLSGFSFK

SEQ ID NO: 283 - protein, 22 aa, Artificial Sequence
  Scaffold Y Sequence
GAKKSKKRFSFKKSFKLSGFSF

SEQ ID NO: 284 - protein, 21 aa, Artificial Sequence
  Scaffold Y Sequence
GAKKSKKRFSFKKSFKLSGFS

SEQ ID NO: 285 - protein, 20 aa, Artificial Sequence
  Scaffold Y Sequence
GAKKSKKRFSFKKSFKLSGF

SEQ ID NO: 286 - protein, 19 aa, Artificial Sequence
  Scaffold Y Sequence
GAKKSKKRFSFKKSFKLSG

SEQ ID NO: 287 - protein, 18 aa, Artificial Sequence
  Scaffold Y Sequence
GAKKSKKRFSFKKSFKLS

SEQ ID NO: 288 - protein, 17 aa, Artificial Sequence
  Scaffold Y Sequence
GAKKSKKRFSFKKSFKL

SEQ ID NO: 289 - protein, 16 aa, Artificial Sequence
  Scaffold Y Sequence
GAKKSKKRFSFKKSFK

SEQ ID NO: 290 - protein, 15 aa, Artificial Sequence
  Scaffold Y Sequence
GAKKSKKRFSFKKSF

SEQ ID NO: 291 - skipped

SEQ ID NO: 292 - protein, 13 aa, Artificial Sequence
  Scaffold Y Sequence
GAKKSKKRFSFKK

SEQ ID NO: 293 - protein, 12 aa, Artificial Sequence
  Scaffold Y Sequence
GAKKSKKRFSFK

SEQ ID NO: 294 - protein, 11 aa, Artificial Sequence
  Scaffold Y Sequence
GAKKSKKRFSF

SEQ ID NO: 295 - protein, 10 aa, Artificial Sequence
  Scaffold Y Sequence
GAKKSKKRFS

SEQ ID NO: 296 - protein, 9 aa, Artificial Sequence
  Scaffold Y Sequence
GAKKSKKRF

SEQ ID NO: 297 - protein, 8 aa, Artificial Sequence
  Scaffold Y Sequence
GAKKSKKR

SEQ ID NO: 298 - protein, 7 aa, Artificial Sequence
  Scaffold Y Sequence
GAKKSKK

SEQ ID NO: 299 - protein, 29 aa, Artificial Sequence
  Scaffold Y Sequence
GAKKSKKRFSFKKSFKLSGFSFKKNKKEA

SEQ ID NO: 300 - protein, 29 aa, Artificial Sequence
  Scaffold Y Sequence
GAKKAKKRFSFKKSFKLSGFSFKKNKKEA

SEQ ID NO: 301 - protein, 28 aa, Artificial Sequence
  Scaffold Y Sequence
GAQESKKKKKKRFSFKKSFKLSGFSFKK

SEQ ID NO: 302 - protein, 27 aa, Artificial Sequence
  Scaffold Y Sequence
GAQESKKKKKKRFSFKKSFKLSGFSFK

SEQ ID NO: 303 - protein, 26 aa, Artificial Sequence
  Scaffold Y Sequence
GAQESKKKKKKRFSFKKSFKLSGFSF

SEQ ID NO: 304 - protein, 25 aa, Artificial Sequence
  Scaffold Y Sequence
GAQESKKKKKKRFSFKKSFKLSGFS

SEQ ID NO: 305 - protein, 24 aa, Artificial Sequence
  Scaffold Y Sequence
GAQESKKKKKKRFSFKKSFKLSGF

SEQ ID NO: 306 - protein, 23 aa, Artificial Sequence
  Scaffold Y Sequence
GAQESKKKKKKRFSFKKSFKLSG

SEQ ID NO: 307 - protein, 22 aa, Artificial Sequence
  Scaffold Y Sequence
GAQESKKKKKKRFSFKKSFKLS

SEQ ID NO: 308 - protein, 21 aa, Artificial Sequence
  Scaffold Y Sequence
GAQESKKKKKKRFSFKKSFKL

SEQ ID NO: 309 - protein, 20 aa, Artificial Sequence
  Scaffold Y Sequence
GAQESKKKKKKRFSFKKSFK

SEQ ID NO: 310 - protein, 19 aa, Artificial Sequence
  Scaffold Y Sequence
GAQESKKKKKKRFSFKKSF

SEQ ID NO: 311 - protein, 18 aa, Artificial Sequence
  Scaffold Y Sequence
GAQESKKKKKKRFSFKKS

SEQ ID NO: 312 - protein, 17 aa, Artificial Sequence
  Scaffold Y Sequence
GAQESKKKKKKRFSFKK

SEQ ID NO: 313 - protein, 16 aa, Artificial Sequence
  Scaffold Y Sequence
GAQESKKKKKKRFSFK

SEQ ID NO: 314 - protein, 15 aa, Artificial Sequence
  Scaffold Y Sequence
GAQESKKKKKKRFSF

SEQ ID NO: 315 - protein, 14 aa, Artificial Sequence
  Scaffold Y Sequence
GAQESKKKKKKRFS

SEQ ID NO: 316 - protein, 13 aa, Artificial Sequence
  Scaffold Y Sequence
GAQESKKKKKKRF

SEQ ID NO: 317 - protein, 12 aa, Artificial Sequence
  Scaffold Y Sequence
GAQESKKKKKKR

SEQ ID NO: 318 - protein, 11 aa, Artificial Sequence
  Scaffold Y Sequence
GAQESKKKKKK

SEQ ID NO: 319 - protein, 10 aa, Artificial Sequence
  Scaffold Y Sequence
GAQESKKKKK

SEQ ID NO: 320 - protein, 9 aa, Artificial Sequence
  Scaffold Y Sequence
GAQESKKKK

SEQ ID NO: 321 - protein, 8 aa, Artificial Sequence
  Scaffold Y Sequence
GAQESKKK

SEQ ID NO: 322 - protein, 7 aa, Artificial Sequence
  Scaffold Y Sequence
GAQESKK

SEQ ID NO: 323 - protein, 30 aa, Artificial Sequence
  Scaffold Y Sequence
GSQSSKKKKKKFSFKKPFKLSGLSFKRNRK

SEQ ID NO: 324 - protein, 29 aa, Artificial Sequence
  Scaffold Y Sequence
GSQSSKKKKKKFSFKKPFKLSGLSFKRNR

SEQ ID NO: 325 - protein, 28 aa, Artificial Sequence
  Scaffold Y Sequence
GSQSSKKKKKKFSFKKPFKLSGLSFKRN

SEQ ID NO: 326 - protein, 27 aa, Artificial Sequence
  Scaffold Y Sequence
GSQSSKKKKKKFSFKKPFKLSGLSFKR

SEQ ID NO: 327 - protein, 26 aa, Artificial Sequence
  Scaffold Y Sequence
GSQSSKKKKKKFSFKKPFKLSGLSFK

SEQ ID NO: 328 - protein, 25 aa, Artificial Sequence
  Scaffold Y Sequence
GSQSSKKKKKKFSFKKPFKLSGLSF

SEQ ID NO: 329 - protein, 24 aa, Artificial Sequence
  Scaffold Y Sequence
GSQSSKKKKKKFSFKKPFKLSGLS

SEQ ID NO: 330 - protein, 23 aa, Artificial Sequence
  Scaffold Y Sequence
GSQSSKKKKKKFSFKKPFKLSGL

SEQ ID NO: 331 - protein, 22 aa, Artificial Sequence
  Scaffold Y Sequence
GSQSSKKKKKKFSFKKPFKLSG

SEQ ID NO: 332 - protein, 21 aa, Artificial Sequence
  Scaffold Y Sequence
GSQSSKKKKKKFSFKKPFKLS

SEQ ID NO: 333 - protein, 20 aa, Artificial Sequence
  Scaffold Y Sequence
GSQSSKKKKKKFSFKKPFKL

SEQ ID NO: 334 - protein, 19 aa, Artificial Sequence
  Scaffold Y Sequence
GSQSSKKKKKKFSFKKPFK

SEQ ID NO: 335 - protein, 18 aa, Artificial Sequence
  Scaffold Y Sequence
GSQSSKKKKKKFSFKKPF

SEQ ID NO: 336 - protein, 17 aa, Artificial Sequence
  Scaffold Y Sequence
GSQSSKKKKKKFSFKKP

SEQ ID NO: 337 - protein, 16 aa, Artificial Sequence
  Scaffold Y Sequence
GSQSSKKKKKKFSFKK

SEQ ID NO: 338 - protein, 15 aa, Artificial Sequence
  Scaffold Y Sequence
GSQSSKKKKKKFSFK

SEQ ID NO: 339 - protein, 14 aa, Artificial Sequence
  Scaffold Y Sequence
GSQSSKKKKKKFSF

SEQ ID NO: 340 - protein, 13 aa, Artificial Sequence
  Scaffold Y Sequence
GSQSSKKKKKKFS

SEQ ID NO: 341 - protein, 12 aa, Artificial Sequence
  Scaffold Y Sequence
GSQSSKKKKKKF

SEQ ID NO: 342 - protein, 11 aa, Artificial Sequence
  Scaffold Y Sequence
GSQSSKKKKKK

SEQ ID NO: 343 - protein, 10 aa, Artificial Sequence
  Scaffold Y Sequence
GSQSSKKKKK

SEQ ID NO: 344 - protein, 9 aa, Artificial Sequence
  Scaffold Y Sequence
GSQSSKKKK

SEQ ID NO: 345 - protein, 8 aa, Artificial Sequence
  Scaffold Y Sequence
GSQSSKKK

SEQ ID NO: 346 - protein, 7 aa, Artificial Sequence
  Scaffold Y Sequence
GSQSSKK

SEQ ID NO: 347 - protein, 28 aa, Artificial Sequence
  Scaffold Y Sequence
GAKKAKKRFSFKKSFKLSGFSFKKNKKE

SEQ ID NO: 348 - protein, 27 aa, Artificial Sequence
  Scaffold Y Sequence
GAKKAKKRFSFKKSFKLSGFSFKKNKK

SEQ ID NO: 349 - protein, 26 aa, Artificial Sequence
  Scaffold Y Sequence
GAKKAKKRFSFKKSFKLSGFSFKKNK

SEQ ID NO: 350 - protein, 25 aa, Artificial Sequence
  Scaffold Y Sequence
GAKKAKKRFSFKKSFKLSGFSFKKN

SEQ ID NO: 351 - protein, 24 aa, Artificial Sequence
  Scaffold Y Sequence
GAKKAKKRFSFKKSFKLSGFSFKK

SEQ ID NO: 352 - protein, 23 aa, Artificial Sequence
  Scaffold Y Sequence
GAKKAKKRFSFKKSFKLSGFSFK

SEQ ID NO: 353 - protein, 22 aa, Artificial Sequence
  Scaffold Y Sequence
GAKKAKKRFSFKKSFKLSGFSF

SEQ ID NO: 354 - protein, 21 aa, Artificial Sequence
  Scaffold Y Sequence
GAKKAKKRFSFKKSFKLSGFS

SEQ ID NO: 355 - protein, 20 aa, Artificial Sequence
  Scaffold Y Sequence
GAKKAKKRFSFKKSFKLSGF

SEQ ID NO: 356 - protein, 19 aa, Artificial Sequence
  Scaffold Y Sequence
GAKKAKKRFSFKKSFKLSG

SEQ ID NO: 357 - protein, 18 aa, Artificial Sequence
  Scaffold Y Sequence
GAKKAKKRFSFKKSFKLS

SEQ ID NO: 358 - protein, 17 aa, Artificial Sequence
  Scaffold Y Sequence
GAKKAKKRFSFKKSFKL

SEQ ID NO: 359 - protein, 16 aa, Artificial Sequence
  Scaffold Y Sequence
GAKKAKKRFSFKKSFK

SEQ ID NO: 360 - protein, 15 aa, Artificial Sequence
  Scaffold Y Sequence
GAKKAKKRFSFKKSF

SEQ ID NO: 361 - protein, 14 aa, Artificial Sequence
  Scaffold Y Sequence
GAKKAKKRFSFKKS

SEQ ID NO: 362 - protein, 13 aa, Artificial Sequence
  Scaffold Y Sequence
GAKKAKKRFSFKK

SEQ ID NO: 363 - protein, 12 aa, Artificial Sequence
  Scaffold Y Sequence
GAKKAKKRFSFK

SEQ ID NO: 364 - protein, 11 aa, Artificial Sequence
  Scaffold Y Sequence
GAKKAKKRFSF

SEQ ID NO: 365 - protein, 10 aa, Artificial Sequence
  Scaffold Y Sequence
GAKKAKKRFS

SEQ ID NO: 366 - protein, 9 aa, Artificial Sequence
  Scaffold Y Sequence
GAKKAKKRF

SEQ ID NO: 367 - protein, 8 aa, Artificial Sequence
  Scaffold Y Sequence
GAKKAKKR

SEQ ID NO: 368 - protein, 7 aa, Artificial Sequence
  Scaffold Y Sequence
GAKKAKK

SEQ ID NO: 369 - protein, 94 aa, Artificial Sequence
  ESAT6 Protein Sequence
MTEQQWNFAGIEAAASAIQGNVTSIHSLDEGKQSLTKLAAAWGGSGSEAYQGVQQKWDATATELNNALQNLARTISEAGQAMASTEGNVTGMFA

SEQ ID NO: 370 - protein, 96 aa, Artificial Sequence
  TB10.4 Protein Sequence
MSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 371 - protein, 8 aa, Artificial Sequence
  Scaffold Y Sequence
  misc_feature (2)..(2): Xaa can be any naturally occurring amino acid
GXKLSKKK

SEQ ID NO: 372 - skipped

SEQ ID NO: 373 - protein, 400 aa, Artificial Sequence
  Linker
  MISC_FEATURE (1)..(400): This sequence may encompass 1-100 “GGGG” repeating units.
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 374 - protein, 10100 aa, Artificial Sequence
  Linker
  misc_Feature (1)..(10100): This sequence may encompass 1-100 “G100-S” repeating units, which then may repeat 1-100 times.
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGS

SEQ ID NO: 375 - protein, 250 aa, Artificial Sequence
  Linker
  MISC_FEATURE (1)..(250): This sequence may encompass 1-50 “G-S” repeating units, where G may repeat 1 to 4 times and S may be present or absent.
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 376 - protein, 100 aa, Artificial Sequence
  Linker
  misc_feature (1)..(100): This sequence may encompass 1-100 “G” repeating units
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 377 - protein, 200 aa, Artificial Sequence
  Synthetic
  MISC_FEATURE (1)..(200): This sequence may encompass 1-100 “GA” repeating units
GAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGA

SEQ ID NO: 378 - protein, 300 aa, Artificial Sequence
  Linker
  misc_feature (1)..(300): This sequence may encompass 1-100 “GGS” repeating units
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 379 - protein, 42 aa, Artificial Sequence [sequence omitted]

SEQ ID NO: 380 - protein, 6 aa, Artificial Sequence
  human amyloid-B protein fragment
AEFRHD

SEQ ID NO: 381 - protein, 40 aa, Artificial Sequence
  PolyGA
  misc_feature (1)..(40): This sequence may encompass 10-20 “GA” repeating units
GAGAGAGAGAGAGAGAGAGAGAGAGAGAGAGAGAGAGAGA

SEQ ID NO: 382 - protein, 13 aa, Artificial Sequence
  PADRE peptide
AKFVAAWTLKAAA

SEQ ID NO: 383 - protein, 14 aa, Artificial Sequence
  Tetanus fragment
QYIKANSKFIGITE

SEQ ID NO: 384 - protein, 15 aa, Artificial Sequence
  Diphtheria toxin fragment
QSIALSSLMVAQAIP

SEQ ID NO: 385 - protein, 119 aa, artificial sequence
  brain derived neurotrophic factor protein
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 386 - protein, 120 aa, artificial sequence
  nerve growth factor protein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 387 - protein, 119 aa, artificial sequence [sequence omitted]

SEQ ID NO: 388 - protein, 130 aa, artificial sequence
  neurotrophin-4 protein
GVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 389 - protein, 29 aa, artificial sequence
  RVG peptide
YTIWMPENPRPGTPCDIFTNSRGKRASNG

SEQ ID NO: 390 - protein, 14 aa, artificial sequence
  cyclized TAxI peptide
SACQSQSQMRCGGG

SEQ ID NO: 391 - protein, 7 aa, artificial sequence
  TAxI transport peptide #1
QSQSQMR

SEQ ID NO: 392 - protein, 7 aa, artificial sequence
  TAxI transport peptide #2
ASGAQAR

SEQ ID NO: 393 - protein, 14 aa, artificial sequence
  TAxI transport peptide #3
TSTAPHLRLRLTSR

SEQ ID NO: 394 - protein, 7 aa, artificial sequence
  nuclear localizing signal #1
PPKKRKV

SEQ ID NO: 395 - protein, 6 aa, artificial sequence
  nuclear localizing signal #2
PKKRKV

SEQ ID NO: 396 - protein, 19 aa, artificial sequence
  Angiopep-2
TFFYGGSRGKRNNFKTEEY

SEQ ID NO: 397 - protein, 39 aa, artificial sequence
  ApoB (3371 3409)
SSVIDALQYKLEGTTRLTRKRGLKLATALSLSNKFVEGS

SEQ ID NO: 398 - protein, 18 aa, artificial sequence
  ApoE (159 167)2
LRKLRKRLLLRKLRKRLL

SEQ ID NO: 399 - protein, 8 aa, artificial sequence
  Peptide-22
CMPRLRGC

SEQ ID NO: 400 - protein, 12 aa, artificial sequence
  THR
THRPPMWSPVWP

SEQ ID NO: 401 - protein, 12 aa, artificial sequence
  THR retro-enantio
PWVPSWMPPRHT

SEQ ID NO: 402 - protein, 9 aa, artificial sequence
  CRT
CRTIGPSVC

SEQ ID NO: 403 - protein, 30 aa, artificial sequence
  Leptin30
YQQILTSMPSRNVIQISNDLENLRDLLHVL

SEQ ID NO: 404 - protein, 29 aa, artificial sequence
  RVG29
YTIWMPENPRPGTPCDIFTNSRGKRASNG

SEQ ID NO: 405 - protein, 16 aa, artificial sequence
  CDX
GREIRTGRAERWSEKF

SEQ ID NO: 406 - protein, 18 aa, artificial sequence
  Apamin
CNCKAPETALCARRCQQH

SEQ ID NO: 407 - protein, 8 aa, artificial sequence
  MiniAp-4
KAPETALD

SEQ ID NO: 408 - protein, 2 aa, artificial sequence
  GSH
CG

SEQ ID NO: 409 - protein, 13 aa, artificial sequence
  G23
HLNILSTLWKYRC

SEQ ID NO: 410 - protein, 6 aa, artificial sequence
  g7
GFGFLS

SEQ ID NO: 411 - protein, 12 aa, artificial sequence
  TGN
TGNYKALHPHNG

SEQ ID NO: 412 - protein, 11 aa, artificial sequence
  TAT (47 57)
YGRKKRRQRRR

SEQ ID NO: 413 - protein, 18 aa, artificial sequence
  SynB1
RGGRLSYSRRRFSTSTGR

SEQ ID NO: 414 - protein, 21 aa, artificial sequence
  Diketopiperazines
GNYTCEVTELTREGETIIELK

SEQ ID NO: 415 - protein, 731 aa, artificial sequence; identical to SEQ ID NO: 2
  PhPro

SEQ ID NO: 416 - protein, 29 aa, artificial sequence
  Linker
GGSSGSGSGSGGGGSGGGGTGTSSSGTGT

SEQ ID NO: 417 - protein, 1255 aa, artificial sequence
  Spike Protein
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 418 - protein, 76 aa, artificial sequence
  Envelope Protein
MYSFVSEETGTLIVNSVLLFLAFVVFLLVTLAILTALRLCAYCCNIVNVSLVKPTVYVYSRVKNLNSSEGVPDLLV

SEQ ID NO: 419 - protein, 221 aa, artificial sequence
  Membrane Protein
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 420 - protein, 9 aa, artificial sequence
  T Cell Epitope
LLLNCLWSV

SEQ ID NO: 421 - protein, 9 aa, artificial sequence
  T Cell Epitope
FKDGIYFAA

SEQ ID NO: 422 - protein, 9 aa, artificial sequence
  T Cell Epitope
LITGRLAAL

SEQ ID NO: 423 - protein, 9 aa, artificial sequence
  T Cell Epitope
LITGRLQSL

SEQ ID NO: 424 - protein, 9 aa, artificial sequence
  T Cell Epitope
ISVVLIFVV

SEQ ID NO: 425 - protein, 9 aa, artificial sequence
  T Cell Epitope
FIAGLIAIV

SEQ ID NO: 426 - protein, 9 aa, artificial sequence
  T Cell Epitope
GILGFVFTL

SEQ ID NO: 427 - protein, 9 aa, artificial sequence
  T Cell Epitope
VVFLHVTYV

SEQ ID NO: 428 - protein, 9 aa, artificial sequence
  T Cell Epitope
RLQSLQTYV

SEQ ID NO: 429 - protein, 9 aa, artificial sequence
  T Cell Epitope
VLNDILSRL

SEQ ID NO: 430 - protein, 9 aa, artificial sequence
  T Cell Epitope
VLYENQKQI

SEQ ID NO: 431 - protein, 9 aa, artificial sequence
  T Cell Epitope
NLNESLIDL

SEQ ID NO: 432 - protein, 9 aa, artificial sequence
  T Cell Epitope
LLLQYGSFC

SEQ ID NO: 433 - protein, 9 aa, artificial sequence
  T Cell Epitope
KLPDDFMGC

SEQ ID NO: 434 - protein, 9 aa, artificial sequence
  T Cell Epitope
ILPDPLKPT

SEQ ID NO: 435 - protein, 9 aa, artificial sequence
  T Cell Epitope
MIFDNAFNC

SEQ ID NO: 436 - protein, 9 aa, artificial sequence
  T Cell Epitope
FIFLLFLTL

SEQ ID NO: 437 - protein, 9 aa, artificial sequence
  T Cell Epitope
VLYQDVNCT

SEQ ID NO: 438 - protein, 9 aa, artificial sequence
  T Cell Epitope
IITTDNTFV

SEQ ID NO: 439 - protein, 9 aa, artificial sequence
  T Cell Epitope
RLNEVAKNL

SEQ ID NO: 440 - protein, 9 aa, artificial sequence
  T Cell Epitope
LLTDDMIAA

SEQ ID NO: 441 - protein, 9 aa, artificial sequence
  T Cell Epitope
LLQYGSFCT

SEQ ID NO: 442 - protein, 9 aa, artificial sequence
  T Cell Epitope
ILLCCMTSC

SEQ ID NO: 443 - protein, 9 aa, artificial sequence
  T Cell Epitope
LLFNKVTLA

SEQ ID NO: 444 - protein, 9 aa, artificial sequence
  T Cell Epitope
TMNNKSQSV

SEQ ID NO: 445 - protein, 9 aa, artificial sequence
  T Cell Epitope
ALNTLVKQL

SEQ ID NO: 446 - protein, 9 aa, artificial sequence
  T Cell Epitope
ELCDNPFFA

SEQ ID NO: 447 - protein, 9 aa, artificial sequence
  T Cell Epitope
FPRGQGVPI

SEQ ID NO: 448 - protein, 9 aa, artificial sequence
  T Cell Epitope
RLNQLESKV

SEQ ID NO: 449 - protein, 8 aa, artificial sequence
  T Cell Epitope
ILLNKHID

SEQ ID NO: 450 - protein, 18 aa, artificial sequence
  T Cell Epitope
AFFGMSRIGMEVTPSGTW

SEQ ID NO: 451 - protein, 10 aa, artificial sequence
  T Cell Epitope
MEVTPSGTWL

SEQ ID NO: 452 - protein, 9 aa, artificial sequence
  T Cell Epitope
GMSRIGMEV

SEQ ID NO: 453 - protein, 9 aa, artificial sequence
  T Cell Epitope
ILLNKHIDA

SEQ ID NO: 454 - protein, 9 aa, artificial sequence
  T Cell Epitope
ALNTPKDHI

SEQ ID NO: 455 - protein, 17 aa, artificial sequence
  T Cell Epitope
IRQGTDYKHWPQIAQFA

SEQ ID NO: 456 - protein, 17 aa, artificial sequence
  T Cell Epitope
KHWPQIAQFAPSASAFF

SEQ ID NO: 457 - protein, 9 aa, artificial sequence
  T Cell Epitope
LALLLLDRL

SEQ ID NO: 458 - protein, 9 aa, artificial sequence
  T Cell Epitope
LLLDRLNQL

SEQ ID NO: 459 - protein, 18 aa, artificial sequence
  T Cell Epitope
LLNKHIDAYKTFPPTEPK

SEQ ID NO: 460 - protein, 9 aa, artificial sequence
  T Cell Epitope
LQLPQGTTL

SEQ ID NO: 461 - protein, 15 aa, artificial sequence
  T Cell Epitope
AQFAPSASAFFGMSR

SEQ ID NO: 462 - protein, 18 aa, artificial sequence
  T Cell Epitope
AQFAPSASAFFGMSRIGM

SEQ ID NO: 463 - protein, 15 aa, artificial sequence
  T Cell Epitope
RRPQGLPNNTASWFT

SEQ ID NO: 464 - protein, 16 aa, artificial sequence
  T Cell Epitope
YKTFPPTEPKKDKKKK

SEQ ID NO: 465 - protein, 16 aa, artificial sequence
  T Cell Epitope
GAALQIPFAMQMAYRF

SEQ ID NO: 466 - protein, 16 aa, artificial sequence
  T Cell Epitope
MAYRFNGIGVTQNVLY

SEQ ID NO: 467 - protein, 18 aa, artificial sequence
  T Cell Epitope
QLIRAAEIRASANLAATK

SEQ ID NO: 468 - protein, 9 aa, artificial sequence
  T Cell Epitope
LLLLDRLNQ

SEQ ID NO: 469 - protein, 10 aa, artificial sequence
  T Cell Epitope
LLLLDRLNQL

SEQ ID NO: 470 - protein, 17 aa, artificial sequence
  T Cell Epitope
QALNTLVKQLSSNFGAI

SEQ ID NO: 471 - protein, 9 aa, artificial sequence
  T Cell Epitope
AQFAPSASA

SEQ ID NO: 472 - protein, 9 aa, artificial sequence
  T Cell Epitope
VLNDILSRL

SEQ ID NO: 473 - protein, 9 aa, artificial sequence
  T Cell Epitope
TTLPKGFYA

SEQ ID NO: 474 - protein, 10 aa, artificial sequence
  T Cell Epitope
GLIAIVMVTI

SEQ ID NO: 475 - protein, 8 aa, artificial sequence
  T Cell Epitope
LQYGSFCT

SEQ ID NO: 476 - protein, 9 aa, artificial sequence
  T Cell Epitope
RLDKVEAEV

SEQ ID NO: 477 - protein, 10 aa, artificial sequence
  T Cell Epitope
VLQLPQGTTL

SEQ ID NO: 478 - protein, 10 aa, artificial sequence
  T Cell Epitope
GYQPYRVVVL

SEQ ID NO: 479 - protein, 9 aa, artificial sequence
  T Cell Epitope
PYRVVVLSF

SEQ ID NO: 480 - protein, 9 aa, artificial sequence
  T Cell Epitope
LSPRWYFYY

SEQ ID NO: 481 - protein, 10 aa, artificial sequence
  T Cell Epitope
DSFKEELDKY

SEQ ID NO: 482 - protein, 10 aa, artificial sequence
  T Cell Epitope
LIDLQELGKY

SEQ ID NO: 483 - protein, 9 aa, artificial sequence
  T Cell Epitope
GTTLPKGFY

SEQ ID NO: 484 - protein, 10 aa, artificial sequence
  T Cell Epitope
VTPSGTWLTY

SEQ ID NO: 485 - protein, 9 aa, artificial sequence
  T Cell Epitope
GSFCTQLNR

SEQ ID NO: 486 - protein, 9 aa, artificial sequence
  T Cell Epitope
GVVFLHVTY

SEQ ID NO: 487 - protein, 9 aa, artificial sequence
  T Cell Epitope
AQALNTLVK

SEQ ID NO: 488 - protein, 9 aa, artificial sequence
  T Cell Epitope
MTSCCSCLK

SEQ ID NO: 489 - protein, 9 aa, artificial sequence
  T Cell Epitope
ASANLAATK

SEQ ID NO: 490 - protein, 10 aa, artificial sequence
  T Cell Epitope
SLIDLQELGK

SEQ ID NO: 491 - protein, 9 aa, artificial sequence
  T Cell Epitope
SVLNDILSR

SEQ ID NO: 492 - protein, 10 aa, artificial sequence
  T Cell Epitope
TQNVLYENQK

SEQ ID NO: 493 - protein, 10 aa, artificial sequence
  T Cell Epitope
CMTSCCSCLK

SEQ ID NO: 494 - protein, 10 aa, artificial sequence
  T Cell Epitope
VQIDRLITGR

SEQ ID NO: 495 - protein, 9 aa, artificial sequence
  T Cell Epitope
KTFPPTEPK

SEQ ID NO: 496 - protein, 10 aa, artificial sequence
  T Cell Epitope
KTFPPTEPKK

SEQ ID NO: 497 - protein, 9 aa, artificial sequence
  T Cell Epitope
ASAFFGMSR

SEQ ID NO: 498 - protein, 10 aa, artificial sequence
  T Cell Epitope
ATEGALNTPK

SEQ ID NO: 499 - protein, 10 aa, artificial sequence
  T Cell Epitope
QLPQGTTLPK

SEQ ID NO: 500 - protein, 9 aa, artificial sequence
  T Cell Epitope
QQQGQTVTK

SEQ ID NO: 501 - protein, 10 aa, artificial sequence
  T Cell Epitope
QQQQGQTVTK

SEQ ID NO: 502 - protein, 10 aa, artificial sequence
  T Cell Epitope
SASAFFGMSR

SEQ ID NO: 503 - protein, 10 aa, artificial sequence
  T Cell Epitope
SQASSRSSSR

SEQ ID NO: 504 - protein, 9 aa, artificial sequence
  T Cell Epitope
TPSGTWLTY

SEQ ID NO: 505 - protein, 10 aa, artificial sequence
  T Cell Epitope
FPNITNLCPF

SEQ ID NO: 506 - protein, 10 aa, artificial sequence
  T Cell Epitope
APHGVVFLHV

SEQ ID NO: 507 - protein, 10 aa, artificial sequence
  T Cell Epitope
APSASAFFGM

SEQ ID NO: 508 - protein, 15 aa, artificial sequence
  T Cell Epitope
GAALQIPFAMQMAYR

SEQ ID NO: 509 - protein, 15 aa, artificial sequence
  T Cell Epitope
GWTFGAGAALQIPFA

SEQ ID NO: 510 - protein, 15 aa, artificial sequence
  T Cell Epitope
IDRLITGRLQSLQTY

SEQ ID NO: 511 - protein, 15 aa, artificial sequence
  T Cell Epitope
ISGINASVVNIQKEI

SEQ ID NO: 512 - protein, 15 aa, artificial sequence
  T Cell Epitope
LDKYFKNHTSPDVDL

SEQ ID NO: 513 - protein, 15 aa, artificial sequence
  T Cell Epitope
LGDISGINASVVNIQ

SEQ ID NO: 514 - protein, 15 aa, artificial sequence
  T Cell Epitope
LGFIAGLIAIVMVTI

SEQ ID NO: 515 - protein, 15 aa, artificial sequence
  T Cell Epitope
LNTLVKQLSSNFGAI

SEQ ID NO: 516 - protein, 15 aa, artificial sequence
  T Cell Epitope
LQDVVNQNAQALNTL

SEQ ID NO: 517 - protein, 15 aa, artificial sequence
  T Cell Epitope
LQSLQTYVTQQLIRA

SEQ ID NO: 518 - protein, 15 aa, artificial sequence
  T Cell Epitope
LQTYVTQQLIRAAEI

SEQ ID NO: 519 - protein, 15 aa, artificial sequence
  T Cell Epitope
AQKFNGLTVLPPLLT

SEQ ID NO: 520 - protein, 15 aa, artificial sequence
  T Cell Epitope
PCSFGGVSVITPGTN

SEQ ID NO: 521 - protein, 15 aa, artificial sequence
  T Cell Epitope
QIPFAMQMAYRFNGI

SEQ ID NO: 522 - protein, 15 aa, artificial sequence
  T Cell Epitope
QQLIRAAEIRASANL

SEQ ID NO: 523 - protein, 15 aa, artificial sequence
  T Cell Epitope
QTYVTQQLIRAAEIR

SEQ ID NO: 524 - protein, 15 aa, artificial sequence
  T Cell Epitope
AYRFNGIGVTQNVLY

SEQ ID NO: 525 - protein, 15 aa, artificial sequence
  T Cell Epitope
SSNFGAISSVLNDIL

SEQ ID NO: 526 - protein, 15 aa, artificial sequence
  T Cell Epitope
TGRLQSLQTYVTQQL

SEQ ID NO: 527 - protein, 15 aa, artificial sequence
  T Cell Epitope
WLGFIAGLIAIVMVT

SEQ ID NO: 528 - protein, 15 aa, artificial sequence
  T Cell Epitope
CVNFNFNGLTGTGVL

SEQ ID NO: 529 - protein, 15 aa, artificial sequence
  T Cell Epitope
DKYFKNHTSPDVDLG

SEQ ID NO: 530 - protein, 15 aa, artificial sequence
  T Cell Epitope
IDAYKTFPPTEPKKD

SEQ ID NO: 531 - protein, 15 aa, artificial sequence
  T Cell Epitope
MSRIGMEVTPSGTWL

SEQ ID NO: 532 - protein, 15 aa, artificial sequence
  T Cell Epitope
NKHIDAYKTFPPTEP

SEQ ID NO: 533 - protein, 15 aa, artificial sequence
  T Cell Epitope
VLQLPQGTTLPKGFY

SEQ ID NO: 534 - protein, 9 aa, artificial sequence
  T Cell Epitope
LQIPFAMQM

SEQ ID NO: 535 - protein, 9 aa, artificial sequence
  T Cell Epitope
RVDFCGKGY

SEQ ID NO: 536 - protein, 10 aa, artificial sequence
  T Cell Epitope
YEQYIKWPWY

SEQ ID NO: 537 - protein, 9 aa, artificial sequence
  T Cell Epitope
GRLQSLQTY

SEQ ID NO: 538 - protein, 9 aa, artificial sequence
  T Cell Epitope
VRFPNITNL

SEQ ID NO: 539 - protein, 17 aa, artificial sequence
  T Cell Epitope
DVVNQNAQALNTLVKQL

SEQ ID NO: 540 - protein, 17 aa, artificial sequence
  T Cell Epitope
EAEVQIDRLITGRLQSL

SEQ ID NO: 541 - protein, 28 aa, artificial sequence
  T Cell Epitope
EIDRLNEVAKNLNESLIDLQELGKYEQY

SEQ ID NO: 542 - protein, 17 aa, artificial sequence
  T Cell Epitope
EVAKNLNESLIDLQELG

SEQ ID NO: 543 - protein, 17 aa, artificial sequence
  T Cell Epitope
GAALQIPFAMQMAYRFN

SEQ ID NO: 544 - protein, 8 aa, artificial sequence
  T Cell Epitope
GAGICASY

SEQ ID NO: 545 - protein, 17 aa, artificial sequence
  T Cell Epitope
AISSVLNDILSRLDKVE

SEQ ID NO: 546 - protein, 8 aa, artificial sequence
  T Cell Epitope
GSFCTQLN

SEQ ID NO: 547 - protein, 17 aa, artificial sequence
  T Cell Epitope
ILSRLDKVEAEVQIDRL

SEQ ID NO: 548 - protein, 8 aa, artificial sequence
  T Cell Epitope
KGIYQTSN

SEQ ID NO: 549 - protein, 8 aa, artificial sequence
  T Cell Epitope
AMQMAYRF

SEQ ID NO: 550 - protein, 17 aa, artificial sequence
  T Cell Epitope
KNHTSPDVDLGDISGIN

SEQ ID NO: 551 - protein, 17 aa, artificial sequence
  T Cell Epitope
MAYRFNGIGVTQNVLYE

SEQ ID NO: 552 - protein, 17 aa, artificial sequence
  T Cell Epitope
AATKMSECVLGQSKRVD

SEQ ID NO: 553 - protein, 17 aa, artificial sequence
  T Cell Epitope
PFAMQMAYRFNGIGVTQ

SEQ ID NO: 554 - protein, 17 aa, artificial sequence
  T Cell Epitope
QLIRAAEIRASANLAAT

SEQ ID NO: 555 - protein, 6 aa, artificial sequence
  T Cell Epitope
QQFGRD

SEQ ID NO: 556 - protein, 17 aa, artificial sequence
  T Cell Epitope
RASANLAATKMSECVLG

SEQ ID NO: 557 - protein, 17 aa, artificial sequence
  T Cell Epitope
RLITGRLQSLQTYVTQQ

SEQ ID NO: 558 - protein, 17 aa, artificial sequence
  T Cell Epitope
SLQTYVTQQLIRAAEIR

SEQ ID NO: 559 - protein, 17 aa, artificial sequence
  T Cell Epitope
DLGDISGINASVVNIQK

SEQ ID NO: 560 - protein, 17 aa, artificial sequence
  T Cell Epitope
FFGMSRIGMEVTPSGTW

SEQ ID NO: 561 - protein, 18 aa, artificial sequence
  T Cell Epitope
GLPNNTASWFTALTQHGK

SEQ ID NO: 562 - protein, 6 aa, artificial sequence
  T Cell Epitope
GTTLPK

SEQ ID NO: 563 - protein, 20 aa, artificial sequence
  T Cell Epitope
KHIDAYKTFPPTEPKKDKKK

SEQ ID NO: 564 - protein, 19 aa, artificial sequence
  T Cell Epitope
YNVTQAFGRRGPEQTQGNF

SEQ ID NO: 565 - protein, 15 aa, artificial sequence
  T Cell Epitope
KTFPPTEPKKDKKKK

SEQ ID NO: 566 - protein, 6 aa, artificial sequence
  T Cell Epitope
LLPAAD

SEQ ID NO: 567 - protein, 17 aa, artificial sequence
  T Cell Epitope
LNKHIDAYKTFPPTEPK

SEQ ID NO: 568 - protein, 10 aa, artificial sequence
  T Cell Epitope
LPQGTTLPKG

SEQ ID NO: 569 - protein, 8 aa, artificial sequence
  T Cell Epitope
LPQRQKKQ

SEQ ID NO: 570 - protein, 18 aa, artificial sequence
  T Cell Epitope
PKGFYAEGSRGGSQASSR

SEQ ID NO: 571 - protein, 17 aa, artificial sequence
  T Cell Epitope
QFAPSASAFFGMSRIGM

SEQ ID NO: 572 - protein, 8 aa, artificial sequence
  T Cell Epitope
QGTDYKHW

SEQ ID NO: 573 - protein, 15 aa, artificial sequence
  T Cell Epitope
QLPQGTTLPKGFYAE

SEQ ID NO: 574 - protein, 18 aa, artificial sequence
  T Cell Epitope
QLPQGTTLPKGFYAEGSR

SEQ ID NO: 575 - protein, 22 aa, artificial sequence
  T Cell Epitope
QLPQGTTLPKGFYAEGSRGGSQ

SEQ ID NO: 576 - protein, 8 aa, artificial sequence
  T Cell Epitope
TFPPTEPK

SEQ ID NO: 577 - protein, 8 aa, artificial sequence
  T Cell Epitope
SQASSRSS

SEQ ID NO: 578 - protein, 16 aa, artificial sequence
  T Cell Epitope
SRGGSQASSRSSSRSR

SEQ ID NO: 579 - protein, 5 aa, artificial sequence
  Spacer
CPGPG

SEQ ID NO: 580 - protein, 6 aa, artificial sequence
  Spacer
GSGSGS

SEQ ID NO: 581 - protein, 206 aa, artificial sequence
  RBD Protein
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNL

(19) United States
(12) Patent Application Publication
(10) Pub. No.: US 2025/0114445 A1
(43) Pub. Date: Apr. 10, 2025

(54) EXTRACELLULAR VESICLES FOR THERAPY

(71) Applicant: LONZA SALES AG, BASEL (CH)

(72) Inventors: Sriram SATHYANARAYANAN, Lexington, MA (US); Tim SOOS, Boston, MA (US); Ke XU, Belmont, MA (US); Aaron NOYES, Cambridge, MA (US); Kevin P. DOOLEY, Cambridge, MA (US); Eric ZHANG, Cambridge, MA (US); Christine MCCOY, Cambridge, MA (US); Jonathan FINN, Cambridge, MA (US)

(73) Assignee: LONZA SALES AG, BASEL (CH)

(21) Appl. No.: 17/906,849

(22) PCT Filed: Mar. 22, 2021
(86) PCT No.: PCT/US2021/023485

§ 371 Date: Sep. 20, 2022

Related U.S. Application Data
(63) Continuation of application No. PCT/US2020/024023, filed on Mar. 20, 2020.
(60) Provisional application No. 63/142,418, filed on Jan. 27, 2021.
(60) Provisional application No. 63/017,514, filed on Apr. 29, 2020.
(60) Provisional application No. 63/006,585, filed on Apr. 7, 2020.
(60) Provisional application No. 63/003,171, filed on Mar. 31, 2020.
(60) Provisional application No. 63/001,088, filed on Mar. 27, 2020.

(51) Int. Cl. A61K 39/215; A61K 39/00; A61P 37/04

(52) U.S. Cl. CPC A61K 39/215; A61P 37/04; A61K 2039/55511; A61K 2039/70

(57) ABSTRACT

The present disclosure relates to extracellular vesicles comprising one or more antigens from a coronavirus (e.g., SARS-CoV-1 or SARS-CoV-2) and optionally an adjuvant. Also provided herein are methods for producing the EVs and methods for using the EVs to treat and/or prevent diseases or disorders, e.g., infectious diseases.

### CROSS-REFERENCE TO RELATED APPLICATION

This PCT application claims the priority benefit of International Application No. PCT/US2020/024023, filed on Mar. 20, 2020; U.S. Provisional Application No. 63/001,088, filed on Mar. 27, 2020; 63/003,171, filed on Mar. 31, 2020; 63/006,585, filed on Apr. 7, 2020; 63/017,514, filed on Apr. 29, 2020; and 63/142,418, filed on Jan. 27, 2021; each of which is herein incorporated by reference in its entirety.

### REFERENCE TO SEQUENCE LISTING SUBMITTED ELECTRONICALLY VIA EFS-WEB

The content of the electronically submitted sequence listing (Name: 0132-0285US1_Seqlisting_ST25.txt, Size: 462,938 bytes; and Date of Creation: Oct. 31, 2023) submitted in this application is incorporated herein by reference in its entirety.

### FIELD OF DISCLOSURE

The present disclosure relates to modified extracellular vesicles, e.g., exosomes (e.g., comprises one or more payloads, e.g., an antigen and adjuvant/immune modulator) that is useful as a vaccine that can be used to treat and/or prevent a range of medical disorders, including, but not limited to, infectious diseases. The present disclosure also relates to methods of producing such EVs and uses thereof.

### BACKGROUND OF DISCLOSURE

EVs are important mediators of intercellular communication. They are also important biomarkers in the diagnosis and prognosis of many diseases, such as cancer. As drug delivery vehicles, EVs offer many advantages over traditional drug delivery methods (e.g., peptide immunization, DNA vaccines) as a new treatment modality in many therapeutic areas. However, despite its advantages, many EVs have had limited clinical efficacy. For example, dendritic-cell derived exosomes (DEX) were investigated in a Phase II clinical trial as maintenance immunotherapy after first line chemotherapy in patients with inoperable non-small cell lung cancer (NSCLC). However, the trial was terminated because the primary endpoint (at least 50% of patients with progression-free survival (PFS) at 4 months after chemotherapy cessation) was not reached. Besse, B., et al., Oncoimmunology 5 (4):e1071008 (2015).

Accordingly, new and more effective engineered-EVs are necessary to better enable therapeutic use and other applications of EV-based technologies.

### Summary of Disclosure

Provided herein is an isolated extracellular vesicle (EV) comprising at least one antigen derived from a coronavirus. In some aspects, the coronavirus is a severe acute respiratory syndrome (SARS) coronavirus. In some aspects, the antigen is a universal SARS coronavirus antigen.

In some aspects, an EV disclosed herein comprises at least 2, 3, 4, 5, 6, 7, 8, 9, 10 or more antigens, wherein the first antigen is derived from a SARS-CoV-1 or SARS-CoV-2 (COVID-19) virus. In some aspects, wherein the second antigen is derived from a SARS-CoV-1 or SARS-CoV-2 (COVID-19) virus. In some aspects, the second antigen is not derived from a SARS-CoV-1 or SARS-CoV-2 (COVID-19) virus. In certain aspects, the first and second antigens are the same. In some aspects, the first and second antigens are different.

In some aspects, an antigen derived from COVID-19 virus that can be expressed in an EV disclosed herein is derived from a spike(S) protein. In some aspects, the antigen comprises a receptor-binding domain (RBD) of the S protein. In certain aspects, the antigen comprises at least 5, 6, 7, 8, 9, 10, 11, 12, 13, 14, 15, 16, 17, 18, 19, or 20 amino acids of the S protein.

In some aspects, an antigen derived from COVID-19 virus expressed in an EV of the present disclosure is derived from an envelope (E) protein. In certain aspects, the antigen comprises at least 5, 6, 7, 8, 9, 10, 11, 12, 13, 14, 15, 16, 17, 18, 19, or 20 amino acids of the E protein.

In some aspects, an antigen derived from COVID-19 virus that can be expressed in an EV of the present disclosure is derived from a membrane (M) protein. In certain aspects, the antigen comprises at least 5, 6, 7, 8, 9, 10, 11, 12, 13, 14, 15, 16, 17, 18, 19, or 20 amino acids of the M protein.

In some aspects, an EV comprises a second antigen, wherein the second antigen is derived from a spike(S) protein of a COVID-19 virus. In some aspects, the second antigen comprises a receptor-binding domain (RBD) of the S protein. In certain aspects, the second antigen comprises at least 5, 6, 7, 8, 9, 10, 11, 12, 13, 14, 15, 16, 17, 18, 19, or 20 amino acids of the S protein.

In some aspects, an EV comprises a second antigen, wherein the second antigen is derived from an envelope (E) protein of a COVID-19 virus. In certain aspects, the second antigen comprises at least 5, 6, 7, 8, 9, 10, 11, 12, 13, 14, 15, 16, 17, 18, 19, or 20 amino acids of the E protein.

In some aspects, an EV comprises a second antigen, wherein the second antigen derived from COVID-19 virus is derived from a membrane (M) protein. In certain aspects, the second antigen comprises at least 5, 6, 7, 8, 9, 10, 11, 12, 13, 14, 15, 16, 17, 18, 19, or 20 amino acids of the M protein.

In some aspects, an EV of the present disclosure comprises a first antigen and a second antigen, wherein the first antigen comprises a receptor-binding domain (RBD) of the S protein and the second antigen comprises a T-antigen.

In some aspects, an EV of the present disclosure further comprises at least one adjuvant.

In some aspects, an EV disclosed herein induces a cellular immune response, a humoral immune response, or both cellular and humoral immune responses. In certain aspects, the induction of the cellular immune response, the humoral immune response, or both cellular and humor immune responses is increased by at least about 5%, at least about 10%, at least about 20%, at least about 30%, at least about 40%, at least about 50%, at least about 60%, at least about 70%, at least about 80%, at least about 90%, at least about 100% or more, compared to (i) a corresponding EV that does not comprise the adjuvant or the antigen or (ii) the adjuvant or the antigen without the EV.

In some aspects, an EV disclosed herein induces a CD4+ T cell response, a CD8+ T cell response, or both CD4+ and CD8+ T cell responses. In some aspects, an EV disclosed herein induces a CD8+ T cell response. In some aspects, an EV expands a tissue resident memory T-cell response.

In some aspects, an EV of the present disclosure further comprises a first scaffold moiety. In certain aspects, the first antigen is linked to the first scaffold moiety. In some aspects, the second antigen is linked to the first scaffold moiety. In some aspects, an EV comprising a first scaffold moiety disclosed herein further comprises a second scaffold moiety. In certain aspects, the first antigen is linked to the first scaffold moiety, and the second antigen is linked to the second scaffold moiety. In some aspects, the first scaffold moiety and the second scaffold moiety are the same. In some aspects, the first scaffold moiety and the second scaffold moiety are different.

In some aspects, the first scaffold moiety is a Scaffold X. In some aspects, the first scaffold moiety is a Scaffold Y. In some aspects, the second scaffold moiety is a Scaffold Y. In some aspects, the second scaffold moiety is a Scaffold X.

In some aspects, a Scaffold X that can be expressed in an EV disclosed herein is capable of: (i) anchoring the first antigen on the luminal surface of the EV; (ii) anchoring the first antigen on the exterior surface of the EV; (iii) anchoring the second antigen on the luminal surface of the EV; (iv) anchoring the second antigen on the exterior surface of the EV; or (v) combinations thereof. In some aspects, the Scaffold X is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), and any combination thereof.

In some aspects, a Scaffold Y that can be expressed in an EV disclosed herein is capable of: (i) anchoring the first antigen on the luminal surface of the EV; (ii) anchoring the second antigen on the luminal surface of the EV; or (iii) both. In certain aspects, the Scaffold Y is selected from the group consisting of myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), and any combination thereof.

In some aspects, the first antigen is linked to a first scaffold moiety on the luminal surface of the EV, and the second antigen is linked to a second scaffold moiety on the luminal surface of the EV. In certain aspects, a) each of the first scaffold moiety and the second scaffold moiety is Scaffold Y; b) the first scaffold moiety is Scaffold Y, and the second scaffold moiety is Scaffold X; c) the first scaffold moiety is Scaffold X, and the second scaffold moiety is Scaffold Y; or d) each of the first scaffold moiety and the second scaffold moiety is Scaffold X.

In some aspects, the first antigen is linked to a first scaffold moiety on the luminal surface of the EV, and the second antigen is in the lumen of the EV. In some aspects, the first antigen is in the lumen of the EV, and the second antigen is linked to a first scaffold moiety on the luminal surface of the EV. In some aspects, the first antigen is linked to a first scaffold moiety on the luminal surface of the EV and the second antigen is linked to a second scaffold moiety on the exterior surface of the EV. In some aspects, the first scaffold moiety is Scaffold Y, and the second scaffold moiety is Scaffold X; or b) each of the first scaffold moiety and the second scaffold moiety is Scaffold X.

In some aspects, the first antigen is linked to a first scaffold moiety on the exterior surface of the EV, and the second antigen is linked to a second scaffold moiety on the luminal surface of the EV. In certain aspects, a) first scaffold moiety is Scaffold X, and the second scaffold moiety is Scaffold Y; or b) each of the first scaffold moiety and the second scaffold moiety is Scaffold X.

In some aspects, the first antigen is in the lumen of the EV and the second antigen is in the lumen of the EV.

In some aspects, the antigen is linked to a first scaffold moiety on the exterior surface of the EV and the adjuvant is linked to a second scaffold moiety on the exterior surface of the EV. In some of such aspects, the first scaffold and the second scaffold moiety are Scaffold X.

In some aspects, the first antigen is linked to a first scaffold moiety on the exterior surface of the EV and the second antigen is in the lumen of the EV. In some of such aspects, the first scaffold moiety is Scaffold X.

In some aspects, the first antigen is in the lumen of the EV and the second antigen is linked to a first scaffold moiety on the exterior surface of the EV. In some of such aspects, the first scaffold moiety is Scaffold X.

In some aspects, the first antigen is linked to a first scaffold moiety on the surface of the EV and the second antigen is linked to the first scaffold moiety on the luminal surface of the EV. In some aspects, the first antigen is linked to a first scaffold moiety on the luminal surface of the EV and the second antigen is linked to the first scaffold moiety on the exterior surface of the EV. In some of such aspects, the first scaffold moiety is Scaffold X.

In some aspects, an EV disclosed herein comprises a second antigen, wherein (i) the second antigen is linked to the first scaffold moiety by a linker, an affinity ligand, or both, (ii) the second antigen is linked to the second scaffold moiety by a linker, an affinity ligand, or both, (iii) the second antigen is linked to the first scaffold moiety by a linker, an affinity ligand, or both, (iv) the second antigen is linked to the second scaffold moiety by a linker, an affinity ligand, or both, or (v) combinations thereof. In certain aspects, the linker and/or the affinity ligand is a polypeptide. In certain aspects, the linker is a non-polypeptide moiety.

In some aspects, the first scaffold moiety or the second scaffold moiety of an EV disclosed herein is PTGFRN protein, In some aspects, the first scaffold moiety or the second scaffold moiety comprises an amino acid sequence as set forth in SEQ ID NO: 33. In some aspects, the first scaffold moiety or the second scaffold moiety comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO: 1.

In some aspects, the first scaffold moiety or the second scaffold moiety of an EV disclosed herein is BASP1 protein. In some aspects, the first scaffold moiety or the second scaffold moiety comprises a peptide of (G)(π)(X)(Φ/π)(π)(+)(+), wherein each parenthetical position represents an amino acid, and wherein a is any amino acid selected from the group consisting of Pro, Gly, Ala, and Ser, X is any amino acid, Φ is any amino acid selected from the group consisting of Val, Ile, Leu, Phe, Trp, Tyr, and Met, and (+) is any amino acid selected from the group consisting of Lys, Arg, and His; and wherein position five is not (+) and position six is neither (+) nor (Asp or Glu). In some aspects, the first scaffold moiety or the second scaffold moiety comprises an amino acid sequence set forth in any one of SEQ ID NOs: 50-155. In some aspects, the first scaffold moiety or the second scaffold moiety comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO: 3.

Also provided herein is an isolated extracellular vesicle comprising (i) a first antigen derived from a SARS-CoV-1 or SARS-CoV-2 (COVID-19) virus and (ii) a second antigen derived from a SARS-CoV-1 or SARS-CoV-2 (COVID-19) virus, wherein: (a) the first antigen is linked to a first Scaffold Y on the luminal surface, and the second antigen is linked to a second Scaffold Y on the luminal surface of the EV; (b) the first antigen is linked to a Scaffold Y on the luminal surface, and the second antigen is in the lumen of the EV; (c) the first antigen is in the lumen of the EV, and the second antigen is linked to a Scaffold Y on the luminal surface of the EV; (d) the first antigen is linked to a Scaffold Y on the luminal surface of the EV, and the second antigen is linked to a Scaffold X on the exterior surface of the EV; (c) the first antigen is in the lumen of the EV, and the second antigen is linked to a Scaffold X on the exterior surface of the EV; (f) the first antigen is linked to a Scaffold Y on the luminal surface of the EV, and the second antigen is linked to a Scaffold X on the luminal surface of the EV; (g) the first antigen is in the lumen of the EV, and the second antigen is linked to a Scaffold X on the luminal surface of the EV; (h) the first antigen is linked to a Scaffold X on the luminal surface of the EV, and the second antigen is linked to the Scaffold X on the exterior surface of the EV; (i) the first antigen is linked to a first Scaffold X on the exterior surface of the EV, and the second antigen is linked to a second Scaffold X on the exterior surface of the EV; (j) the first antigen is linked to a Scaffold X on the exterior surface of the EV, and the second antigen is linked to a Scaffold Y on the luminal surface of the EV; (k) the first antigen is linked to a Scaffold X on the exterior surface of the EV, and the second antigen is in the lumen of the EV; (1) the first antigen is linked to a Scaffold X on the exterior surface of the EV, and the second antigen is linked to the Scaffold X on the luminal surface of the EV; (m) the first antigen is linked to a first Scaffold X on the luminal surface of the EV, and the second antigen is linked to a second Scaffold X on the luminal surface of the EV; (n) the first antigen is linked to a Scaffold X on the luminal surface of the EV, and the second antigen is linked to a Scaffold Y on the luminal surface of the EV; (o) the first antigen is linked to a Scaffold X on the luminal surface of the EV, and the second antigen is in the lumen of the EV; (p) the first antigen is linked to a first Scaffold X on the exterior surface of the EV, and the second antigen is linked to a second Scaffold X on the luminal surface of the EV; (q) the first antigen is linked to a first Scaffold X on the luminal surface of the EV, and the second antigen is linked to a second Scaffold X on the exterior surface of the EV; (r) the first antigen is in the lumen of the EV, and the second antigen is in the lumen of the EV; (s) the first antigen is linked directly to the luminal surface of the EV, and the second antigen is linked directly to the luminal surface of the EV; (t) the first antigen is linked directly to the luminal surface of the EV, and the second antigen is in the lumen of the EV; (u) the first antigen is linked directly to the luminal surface of the EV, and the second antigen is linked to a Scaffold Y on the luminal surface of the EV; (v) the first antigen is linked directly to the luminal surface of the EV, and the second antigen is linked to a Scaffold X on the luminal surface of the EV; (w) the first antigen is linked directly to the luminal surface of the EV, and the second antigen is linked directly to the exterior surface of the EV; (x) the first antigen is linked directly to the luminal surface of the EV, and the second antigen is linked to a Scaffold X on the exterior surface of the EV; (y) the first antigen is linked to a Scaffold Y on the luminal surface of the EV, and the second antigen is linked directly to the luminal surface of the EV; (z) the first antigen is linked to a Scaffold Y on the luminal surface of the EV, and the second antigen is linked directly to the exterior surface of the EV; (aa) the first antigen is linked to a Scaffold X on the luminal surface of the EV, and the second antigen is linked directly to the luminal surface of the EV; (bb) the first antigen is linked to a Scaffold X on the luminal surface of the EV, and the second antigen is linked directly to the exterior surface of the EV; (cc) the first antigen is in the lumen of the EV, and the second antigen is linked directly to the luminal surface of the EV; or (dd) the first antigen is in the lumen of the EV, and the second antigen is linked directly to the exterior of the EV.

In some aspects, an EV disclosed herein further comprises an immune modulator. In certain aspects, the immune modulator is linked directly to the luminal surface or exterior surface of the EV. In some aspects, the immune modulator is linked to a Scaffold X on the exterior surface of the EV or on the luminal surface of the EV. In some aspects, the immune modulator is linked to a Scaffold Y on the luminal surface of the EV.

In some aspects, an EV disclosed herein comprises an adjuvant, wherein the adjuvant is linked directly to the luminal surface or exterior surface of the EV. In certain aspects, the adjuvant is linked to a Scaffold X on the exterior surface of the EV or in the lumen of the EV. In some aspects, the adjuvant is linked to a Scaffold Y on the luminal surface of the EV. In some aspects, the adjuvant is in the lumen of the EV.

In some aspects, the adjuvant is a Stimulator of Interferon Genes (STING) agonist, a toll-like receptor (TLR) agonist, an inflammatory mediator, RIG-I agonists, alpha-gal-cer (NKT agonist), heat shock proteins (e.g., HSP65 and HSP70), C-type lectin agonists (e.g., beta glucan (Dectin 1), chitin, and curdlan), or any combination thereof.

In some aspects, the adjuvant is a STING agonist. In some aspects, the STING agonist comprises a cyclic dinucleotide STING agonist or a non-cyclic dinucleotide STING agonist.

In some aspects, the adjuvant is a TLR agonist. In some aspects, the TLR agonist comprises a TLR2 agonist (e.g., lipoteichoic acid, atypical LPS, MALP-2 and MALP-404, OspA, porin, LerV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly (I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), or any combination thereof.

In some aspects, an EV disclosed herein is an exosome.

In some aspects, an EV of the present disclosure further comprises a targeting moiety. In some aspects, the targeting moiety specifically binds to a marker for a dendritic cell. In certain aspects, the marker is present only on the dendritic cell. In some aspects, the dendritic cell comprises a plasmacytoid dendritic cell (pDC), a myeloid/conventional dendritic cell 1 (cDC1), a myeloid/conventional dendritic cell 2 (cDC2), inflammatory monocyte derived dendritic cells, Langerhans cells, dermal dendritic cells, lysozyme-expressing dendritic cells (LysoDCs), Kupffer cells, or any combination thereof. In some aspects, the dendritic cell is cDC1. In some aspects, the marker comprises a C-type lectin domain family 9 member A (Clec9a) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, Clec10a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), Langerin, CD206, CD11b, CD11c, CD123, CD304, XCR1, AXL, Siglec 6, CD209, SIRPA, CX3CR1, GPR182, CD14, CD16, CD32, CD34, CD38, CD10, or any combination thereof. In certain aspects, the marker is Clec9a protein.

In some aspects, the targeting moiety specifically binds to a marker for a T cell. In certain aspects, the marker comprises a CD3 molecule.

In some aspects, the targeting moiety is linked directly to the exterior surface of the EV. In certain aspects, the targeting moiety is linked to a Scaffold X on the exterior surface of the EV. In some aspects, the targeting moiety is linked directly to the exterior surface of the EV by a linker, an affinity ligand, or both. In some aspects, the targeting moiety is linked to the Scaffold X by a linker. In certain aspects, the linker and/or the affinity ligand is a polypeptide.

Provided herein is a pharmaceutical composition comprising an EV described herein and a pharmaceutically acceptable carrier.

Provided herein is a cell that produces an EV of the present disclosure. Present disclosure further provides a cell comprising one or more vectors, wherein the vectors comprises a nucleic acid sequence encoding: (i) an antigen (e.g., those described herein), (ii) adjuvant (e.g., those described herein), (iii) immune modulator, (iv) targeting moiety (e.g., those described herein), or (v) combinations thereof.

Provided herein is a kit comprising an EV described herein and instructions for use. Also provided herein is an EV-drug conjugate comprising any of the EVs described herein.

Provided herein is a method of making EVs comprising culturing a cell disclosed herein under a suitable condition and obtaining the EVs.

Provided herein is a method of inducing an immune response in a subject in need thereof comprising administering any of the EVs disclosed herein to the subject.

Provided herein is a method of preventing or treating a disease in a subject in need thereof, comprising administering any of the EVs disclosed herein to the subject, wherein the disease is associated with the antigen. In certain aspects, the disease is an infectio.

In some aspects, an EV disclosed herein is administered parenterally, orally, intravenously, intramuscularly, intranasally, subcutaneously, or intraperitoneally.

In some aspects, methods disclosed herein (e.g., inducing an immune response or preventing or treating a disease) comprises administering an additional therapeutic agent to the subject.

In some aspects, the present disclosure is directed to a method of vaccinating a subject in need thereof, comprising (i) administering a priming dose which comprises an extracellular vesicle comprising an adjuvant and an antigen to the subject and (ii) administering a boosting dose which comprises an extracellular vesicle comprising the antigen to the subject.

In some aspects, the antigen is derived from a coronavirus.

In some aspects, the priming dose is administered subcutaneously. In some aspects, the boost dose is administered intranasally.

In some aspects, the adjuvant is a STING agonist. In some aspects, the antigen is linked to a scaffold moiety. In some aspects, the scaffold moiety is Scaffold X.

In some aspects, the EV in the boosting dose does not contain any adjuvant.

In some aspects, the antigen, adjuvant, immune modulator, and/or targeting moiety is linked to a surface of the EV by an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof.

Present disclosure further provides a method of preparing an extracellular vesicle (EV) for a vaccine, comprising loading an antigen to an EV that has been isolated from a producer cell. Also disclosed herein is a method of manufacturing a vaccine for a disease or disorder, comprising loading an antigen to an extracellular vesicle (EV) that has been isolated from a producer cell.

In some aspects, the EV further comprises an adjuvant. In certain aspects, the EV comprises the adjuvant prior to the loading of the antigen to the EV.

In some aspects, the method of preparing an EV for a vaccine and/or the method of manufacturing a vaccine for a disease or disorder further comprises loading the adjuvant. In certain aspects, the adjuvant is loaded before or after the loading of the antigen. In certain aspects, the adjuvant is loaded together with the antigen.

In some aspects, the antigen of an EV useful for the method of preparing an EV for a vaccine and/or the method of manufacturing a vaccine for a disease or disorder described herein is linked to the exterior surface and/or the luminal surface of the EV by an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof.

In some aspects, the antigen is derived from and/or comprises a virus, a bacterium, a parasite, a fungus, a protozoa, a tumor, an allergen, a self-antigen, or any combination thereof. In certain aspects, the antigen is derived from a virus causing a pandemic. In certain aspects, the antigen is derived from a coronavirus, an influenza virus, an Ebola virus, a Chikungunya virus (CHIKV), a Crimean-Congo hemorrhagic fever (CCGF) virus, a Hendra virus, a Lassa virus, a Marburg virus, a monkeypox virus, a Nipah virus, a Hendra virus, a Rift Valley fever (RVF) virus, a Variola virus, a yellow fever virus, a Zika virus, a measles virus, a human immunodeficiency virus (HIV), a hepatitis C virus (HCV), a dengue fever virus (DENV), a parvovirus (e.g., B19 virus), a norvovirus, a respiratory syncytial virus (RSV), a lentivirus, an adenovirus, a flavivirus, a filovirus, a rhinovirus, a human papillomavirus (HPV), or any combination thereof. In some aspects, the antigen is derived from Vibrio cholera, Yersinia pestis bacteria, Mycobacterium tuberculosis (MTB), streptococcus bacteria (e.g., Streptococcus pyogenes, Streptococcus agalactiae, Streptococcus pneumoniae), staphylococcal bacteria (e.g., Staphylococcus aureus), shigella bacteria, Escherichia coli, salmonella, chlamydia bacteria (e.g., Chlamydia trachomatis), Pseudomonas aeruginosa, Klebsiella pneumoniae, Haemophilus influenza, Clostridia difficile, Plasmodium, Leishmania, Schistosoma, Trypanosoma, Brucella, Cryptosporidium, Entamoeba, Neisseria meningitis, Bacillus subtilis, Haemophilus influenzae, Neisseria gonorrhoeae, Borrelia burgdorferi, Corynebacterium diphtheriae, Moraxella catarrhalis, Campylobacter jejuni, clostridium tetanus, Clostridium perfringens, Treponema pallidum or any combination thereof.

In some aspects, the loading of the antigen to the EV occurs at least about 1 day, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 2 years, at least about 3 years, at least about 4 years, at least about 5 years, at least 6 years, at least 7 years, at least 8 years, at least 9 years, at least 10 years, at least 15 years, at least 20 years, at least 25 years or more after isolating the EV from the producer cell.

In some aspects, the time required for manufacturing the vaccine (“manufacturing time) is reduced compared to a reference manufacturing time (e.g., manufacturing time of a method wherein the loading of the antigen occurs by introducing the antigen into the producer cell, or manufacturing time of a method for producing a vaccine that does not comprise an EV, such as a traditional peptide vaccine). In certain aspects, the manufacturing time is reduced by at least about 10%, at least about 20%, at least about 30%, at least about 40%, at least about 50%, at least about 60%, at least about 70%, at least about 80%, or at least about 90% or more, compared to the reference manufacturing time. In some aspects, the manufacturing time is less than about 12 months, less than about 11 months, less than about 10 months, less than about 9 months, less than about 8 months, less than about 7 months, less than about 6 months, less than about 5 months, less than about 4 months, less than about 3 months, less than about 2 months, or less than about 1 month. In some aspects, the manufacturing time is less than about 6 months.

In some aspects, an EV useful for the method of preparing an EV for a vaccine and/or the method of manufacturing a vaccine for a disease or disorder described herein further comprises a targeting moiety. In some aspects, the targeting moiety prior to the loading of the antigen to the EV.

In some aspects, the method of preparing an EV for a vaccine and/or the method of manufacturing a vaccine for a disease or disorder further comprises loading the targeting moiety. In certain aspects, the targeting moiety are loaded before or after the loading of the antigen. In certain aspects, the targeting moiety is loaded together with the antigen.

In some aspects, after the loading of the antigen, the EV is capable of inducing a T-cell immune response, a B-cell immune response, or both T-cell and B-cell immune responses.

Also provided herein is an EV prepared by any of the methods described herein. Provided herein is a kit comprising such an EV, and instructions for use.

Also provided herein is a vaccine comprising any of the EVs described herein, wherein the antigen is capable of eliciting an immune response in a subject that receives an administration of the vaccine. In certain aspects, the vaccine is regionalized or individualized.

Provided herein is a method of treating a disease or disorder in a subject in need thereof, comprising administering to the subject any of the EVs or vaccines described herein. In certain aspects, the disease or disorder comprises an infectious disease.

### BRIEF DESCRIPTION OF FIGURES

FIG. 1 shows an exemplary EV comprising one or more antigens, one or more adjuvants, one or more molecules for targeting moiety, or any combination thereof.

FIG. 2 shows an exemplary EV comprising one or more SARS-CoV2 antigens (e.g., spike proteins, which are linked to a Scaffold X on the exterior surface; and T cell epitopes, which are linked to a Scaffold X on the luminal surface) and one or more adjuvants (e.g., STING agonist loaded into the lumen).

FIG. 3 provides an illustration of the down (closed) and up (open) conformation of the coronavirus spike protein. As shown, in the up conformation, the receptor-binding domain (RBD) of the spike protein is exposed to the surface.

FIG. 4 provides an illustration of exemplary ways in which a coronavirus spike antigen can be expressed in an EV disclosed herein. As described herein, in some aspects, the full-length trimeric spike protein can be directly linked to the surface (e.g., exterior surface) of an EV (see diagram “I”). In some aspects, a monomeric subunit of the full-length trimeric spike protein can be directly linked to the surface (e.g., exterior surface) of the EV (see diagram “II”). In some aspects, a monomeric subunit of the full-length trimeric spike protein can be expressed on the surface (e.g., exterior surface) of the EV linked to a Scaffold X (see diagram “III”). In some aspects, a receptor-binding domain of a spike protein (i.e., subunit) can be expressed on the surface (e.g., exterior surface) of the EV linked to a Scaffold X (see diagram “IV”).

FIG. 5 provides an illustration of how different subunits of a coronavirus spike protein can be expressed separately on the surface (e.g., exterior surface) of an EV linked to a scaffold moiety (e.g., Scaffold X).

FIG. 6 provides an illustration of exemplary methods in which a spike protein antigen and a T-antigen can be expressed together in an EV. As shown, in the 1^{st} step, the spike protein antigen can be linked to a Scaffold X and then expressed on the surface of the EV. Then, the EV can be further modified to express the T-antigen on the luminal surface of the EV. On the left of the illustration, the T-antigen is linked to the C-terminus of Scaffold X, allowing other payloads (e.g., adjuvants and/or immune modulators) to be linked to Scaffold Y. On the right, the T-antigen is linked to a Scaffold Y.

FIG. 7 provides an illustration of exemplary scaffold X fusion proteins comprising an affinity ligand described in the present disclosure. As shown, the affinity ligand can be synthesized as a fusion to a coronavirus spike protein antigen (e.g., a monomeric subunit of the full-length trimeric spike protein (see diagram “1”) or the RBD of the spike protein (see diagram “IT”). Then, the affinity ligand can be used to link or conjugate the coronavirus spike protein antigen to a moiety on EVs, e.g., a Scaffold X moiety.

FIG. 8 provides a table describing the administration schedule and experimental design of an exemplary “prime-pull” administration strategy described herein. The different treatment groups were as follows: (1) 1^{st} dose: PBS subcutaneously; 2^{nd} dose: PBS intranasally; (2) 1^{st} dose: PrX-OVA-STING subcutaneously; 2^{nd} dose: PrX-OVA-STING intranasally; (3) 1^{st} dose: PrX-OVA-STING subcutaneously; 2^{nd} dose: PrX-OVA intranasally; (4) 1^{st} dose: PrX-OVA-STING intranasally; 2^{nd} dose: PrX OVA subcutaneously; (5) 1^{st} dose: PrX-OVA-STING intranasally; 2^{nd} dose: PrX-OVA-STING intranasally; (6) 1^{st} dose: PrX-OVA-STING subcutaneously; 2^{nd} dose: PrX-OVA-STING subcutaneously; (7) 1^{st} dose: PrX-OVA-STING intradermally; 2^{nd} dose: PrX-OVA intranasally; (8) 1^{st} dose: PrX-OVA-STING intradermally; 2^{nd} dose: PrX-OVA-STING intradermally; and (9) 1^{st} dose: recombinant OVA+soluble STING agonist subcutaneously; 2^{st} dose: recombinant OVA+soluble STING agonist intranasally.

FIGS. 9A and 9B provide the number of OVA-specific CD8+ effector memory T cells in the lungs of animals treated with EVs using the “prime-pull” administration strategy (as described in FIG. 8). The OVA-specific CD8+ effector memory T cells were quantified using flow cytometry (CD44^{hi} and CD62^{lo}). The results are shown as both percentage of total cells in the lung (FIG. 9A) and total number of OVA-specific CD8+ effector memory T cells (FIG. 9B).

FIGS. 10A, 10B, and 10C provide the number of total OVA-specific T cells (as measured using IFN-γ ELISPOT) in the lungs of animals treated with EVs using the “prime-pull” administration strategy (as described in FIG. 8). FIG. 10A provides the number of OVA-specific CD4+ T cells. FIG. 10B provides the number of OVA-specific CD8+ T cells. FIG. 10C provides comparison of non-specific T cell inflammatory activation in the lungs of animals from the different groups (as described in FIG. 8).

FIGS. 11A and 11B provide the total number T cells in the lungs of animals treated with EVs using the “prime-pull” administration strategy (as described in FIG. 8). In FIG. 11A, the number of T cells was quantified using flow cytometry by gating on cells with the following phenotypic expression: Ly6G−, IA/IE-CD24−, CD11c−, and CD11b−. In FIG. 11B, the number of T cells in the lung was quantified using IFN-γ ELISPOT by stimulating the lung cells with PMA/ionomycin, which activates T cells in an antigen-independent manner.

FIGS. 12A and 12B provide the number of OVA-specific CD4+ and CD8+ T cells, respectively, in the spleen of animals treated with EVs using the “prime-pull” administration strategy (as described in FIG. 8). In FIGS. 12A and 12B, the number of antigen-specific T cells was quantified using IFN-γ ELISPOT.

FIGS. 13A and 13B provide the number of OVA-specific CD8+ effector memory T cells in the spleen of animals treated with EVs using the “prime-pull” administration strategy (as described in FIG. 8). The OVA-specific CD8+ effector memory T cells were quantified using flow cytometry (CD44^{hi} and CD62^{lo}). The results are shown as both percentage of total cells in the lung (FIG. 13A) and total number of OVA-specific CD8+ effector memory T cells (FIG. 13B).

FIGS. 14A, 14B, 14C, and 14D provide comparison of anti-RBD antibody responses in animals vaccinated with EVs comprising the RBD region of coronavirus spike protein. Animals from each of the groups received two doses of the EV construct: priming dose at day 0 and a boosting dose at day 14. FIG. 14A shows the different treatment groups and the route of administration for each of the doses (either subcutaneous (SQ) or intranasal (IN)). For all the EVs tested, the RBD was fused to the N-terminus of PTGFRN (full-length or truncated) and associated with the exterior surface of the EV. The different EVs included: (i) “exoRBD(s)”=RBD fused to truncated PTGFRN; (ii) “STING exoRBD(s)”=RBD fused to truncated PTGFRN+STING agonist in the lumen of the EV; (iii) “STING”=soluble STING agonist (i.e., not associated with an EV); (iv) “exoRBD (1)”=RBD fused to full-length PTGFRN; (v) “STING exoRBD (1)”=RBD fused to full-length PTGFRN+STING agonist in the lumen of the EV; or (vi) “PTGFRN”=comprising PTGFRN alone (i.e., not fused to RBD)+STING agonist in the lumen of the EV. FIG. 14B show the amount of anti-RBD antibodies detected in the sera of animals from the different treatment groups at day 28 post initial vaccination. A commercially available anti-RBD antibody was used as a positive control (“Ab”) and a well containing RBD only was used as a negative control (“RBD”). FIGS. 14C and 14D show the neutralization data. FIG. 14C provides the results for groups 1-5. FIG. 14D provides the results for groups 6-10. Each of the curves for the different groups shown in FIGS. 14C and 14D represent an individual animal.

FIGS. 15A, 15B, 15C, and 15D provide the effect of different B cell co-stimulators on anti-RBD antibody responses in animals vaccinated with EVs comprising the RBD of coronavirus spike protein. Animals from each of the groups were vaccinated subcutaneously twice: priming dose at day 0 and a boosting dose at day 14 with different vaccine compositions described in FIG. 15A. The different vaccination regimens included: (i) “exoRBD+STING”=RBD fused to PTGFRN co-loaded with STING in the lumen; (ii) “exoRBD+STING/PrX-Itgb1+STING”=RBD fused to PTGFRN co-loaded with STING in the lumen and co-administered with PTGFRN exosomes with a T helper peptide epitope to Itgb1 attached to the surface and co-loaded with STING in the lumen; (iii) “exoRBD+STING/PrX-OVA+STING”=RBD fused to PTGFRN co-loaded with STING in the lumen and co-administered with exosomes expressing OVA (Ovalbumin) fused to PTGFRN co-loaded with STING; (iv) “exoRBD+STING/Anti-CD40 agonist (IP)”=RBD fused to PTGFRN co-loaded with STING in the lumen and soluble anti-CD40 agonist antibody administered intraperitoneally; (v) “PrX Spike+STING”=coronavirus spike protein attached to the surface of PTGFRN exosomes and co-loaded with STING in the lumen; (vi) “rRBD+STING”=recombinant RBD protein+soluble STING; (vii) “rSpike+STING”=recombinant Spike protein+STING; or (viii) PBS alone. FIG. 15B shows the amount of anti-RBD antibodies detected in the sera of animals from the different groups after subcutaneous dosing at day 35 post initial vaccination. FIGS. 15C and 15D show the neutralization data after subcutaneous administration. FIG. 15C provides the results for groups 1-5. FIG. 15D provides the results for groups 6-10. FIGS. Each of the curves for the different groups shown in FIGS. 15C and 15D represent an individual animal.

FIGS. 16A, 16B, 16C, 16D, 16E, 16F, and 16G show the effect of alum and CpG adjuvants on the ability of EVs described herein to induce antigen-specific antibodies in vivo. FIG. 16A provides a table showing the administration schedule and experimental design. FIGS. 16B, 16C, and 16D provide comparison of anti-OVA IgG levels in the sera of animals from the different treatment groups at days 14, 28, and 42 post initial treatment, respectively. FIGS. 16E, 16F, and 16G provide comparison of anti-OVA IgM levels in the sera of animals from the different treatment groups at days 14, 28, and 42 post initial treatment, respectively

FIGS. 17A, 17B, 17C, 17D, 17E, and 17F show the use of plug-and-play system described herein to modify EVs to comprise a coronavirus antigen. FIG. 17A provides a schematic illustrating the interaction between ALFA-tagged RBD protein and ALFA nanobody (NbALFA) fused to Scaffold X (e.g., PTGFRN) on the exterior surface of the EV (“Exo-ALFA-RBD”). FIG. 17B shows the purification of RBD-ALFAtag-His protein by immobilized metal affinity chromatography. FIG. 17C shows SDS-PAGE demonstrating the purity and molecular weight of the purified RBD-ALFAtag-His protein. FIGS. 17D and 17E show the loading of RBD-ALFAtag protein onto NbALFA EVs as measured by SDS-PAGE and Western blot, respectively. FIG. 17F provides a quantitative comparison of the results shown in FIGS. 17D and 17E.

FIGS. 18A and 18B provide comparison of anti-RBD antibody levels in animals vaccinated with recombinant RBD protein or with RBD protein presented on the exterior surface of an EV. The RBD protein was either fused directly to the N-terminus of PTGFRN (FIG. 18A) or conjugated using the ALFA plug-and-play system described herein (FIG. 18B). In FIG. 18A, the animals were vaccinated with one of the following: (i) “exoRBD”=RBD fused directly to PTGFRN; (ii) “rRBD+STING”=recombinant RBD protein+soluble STING agonist; (iii) “rRBD+Exo”=recombinant RBD protein+EV alone (i.e., RBD not associated with the EV); and (iv) PBS alone. In FIG. 18B, the animals were vaccinated with one of the following: (i) “NbALFA Exo+RBD-ALFAtag”=RBD conjugated to PTGFRN using the ALFA plug-and-play system; (ii) “rRBD”=recombinant RBD protein alone; (iii) “rRBD”=recombinant RBD protein+soluble STING agonist; (iv) “Exo+rRBD”=recombinant RBD protein+EV alone (i.e., RBD not associated with the EV); and (v) PBS alone. The amount of RBD administered is given over each bar.

FIGS. 19A, 19B, and 19C provide schematic of the different ways in which the RBD and T cell epitopes of coronavirus can be displayed in the EVs described herein. In FIG. 19A, the RBD protein is fused to the N-terminus of the full-length PTGFRN (see diagrams I, II, and III) or a PTGFRN fragment (see diagrams IV and V). The T cell epitopes of coronavirus is displayed on the luminal surface of the EV as either concatemer protein antigen (see diagram I) or concatemer peptide antigens (see diagrams II-V). In FIG. 19B, the RBD protein is fused to the N-terminus of the full-length PTGFRN, and a single peptide comprising a single pan SARS T cell epitope (e.g., derived from the spike(S) protein, nucleocapsid (N) protein, or membrane (M) protein) is displayed on the luminal surface fused to the C-terminus of the PTGFRN. In FIG. 19C, the RBD protein is fused to the N-terminus of the full-length PTGFRN, and the S2 (left image) and ORF3a (right image) proteins of coronavirus are displayed on the luminal surface (e.g., fused to BASP-1 moiety or the C-terminus of PTGFRN).

FIGS. 20A and 20B show the expression of the T cell epitopes included in the EVs described in FIG. 19A (i.e., diagrams I, II, III, IV, and V) as measured by Western blot and HiBiT assay.

FIGS. 21A and 21B show that the EVs described herein can express multiple copies of coronavirus antigen. FIG. 21A provides schematic of RBD protein fused to the N-terminus of full-length PTGFRN (diagram I) or whole spike protein fused to the N-terminus of a PTGFRN fragment (diagrams II and III) on the exterior surface of the EV. In diagrams I and III, HiBiT was conjugated to the C-terminus of PTGFRN. FIG. 21B shows the expression level of the constructs in the EVs as measured using HiBiT Assay.

FIGS. 22A, 22B, and 22C show that EVs described herein can be modified to comprise T cell epitopes of coronavirus either on the exterior or on the luminal surface of the EVs. FIG. 22A (1-4) provides a schematic of RBD protein fused to the N-terminus of full-length PTGFRN (1) and the exterior surface expression of a concatemer consisting of 8 T cell epitope peptides (8-mer) fused to the N-terminus of full-length PTGFRN (2) or the luminal expression of the 8-mer concatemer by fusion to the C terminus of full-length PTGFRN (3); or by fusion to BASP-1 (4). FIG. 22A (5-10) provides a schematic of the 8-mer concatemer described in (2-4) above expressed as two 4-mer concatemer peptides (Group 1 and 2) each fused to the N-terminus of full-length PTGFRN for surface expression (5 and 8) or expressed in the lumen by fusion to the C terminus of full-length PTGFRN (6 and 9); or by fusion to BASP-1 (5 and 10). For each, FLAG tag and HiBiT was conjugated to either the C-terminus of PTGFRN (1, 2 and 5) or the c terminus of the 8-mer (3 and 4) or 4-mers (6, 7, 9 and 10). FIGS. 22B and C shows the expression level of the constructs in the EVs as measured using HiBiT Assay and a western blot for the HiBit tag respectively.

FIGS. 23A, 23B, 23C, and 23D show the anti-tumor effects of EVs described herein in an E.G7-OVA tumor model. FIG. 23A provides the administration schedule and experimental design. FIG. 23B provides the tumor volume in the animals from the different treatment groups at various time points post treatment. FIG. 23C provides the survival rate of the animals from the different treatment groups. FIG. 23D provides the tumor growth rate in the animals.

FIG. 24 shows a schematic illustrating the overall process involved in preparing the EV-based vaccines of the present disclosure.

FIGS. 25A, 25B, 25C, and 25D show exosomes that have been modified to comprise an acceptor domains fused to the N-terminus of a PTGFRN protein. The acceptor domains included: (1) SpyCatcher (diamond), (2) CfaC (square), and (3) ALFANb (triangle). FIG. 25A shows the cell growth of stable HEK293 cells lines engineered to overexpress each acceptor domain fused to PTGFRN during exosome production. FIG. 25B shows the viability of the stable cell pool over the same time period as shown in FIG. 25A. FIG. 25C shows the successful purification of exosomes comprising the acceptor domain-PTGFRN fusion protein using iodixanol density gradient ultracentrifugation. FIG. 25D provides SDS-PAGE analysis showing the transgene expression of each acceptor domain compared to un-engineered exosomes (WT).

FIG. 26 provides a workflow for assessing the loading of NanoLuc fused to a donor domain onto exosomes expressing the cognate acceptor domain fused to PTGFRN. Unbound NanoLuc is removed by ultracentrifugation (UC) and the resulting exosomes are analyzed by a variety of assays.

FIG. 27A provides a workflow for labeling exosomes overexpressing ALFA nanobody (ALFANb) with NanoLuc fused to the ALFAtag. FIG. 27B provides SDS-PAGE analysis post-loading and UC cleanup showing association of NanoLuc-ALFAtag with ALFANb expressing exosomes.

FIG. 28A provides standard curves of NanoLuc fused to ALFA tag or poly-histidine tags. FIG. 28B provides a quantitative analysis of the loading of (i) NanoLuc fused to ALFA tag or (ii) NanoLuc fused to poly-histidine tag on the modified exosomes. The quantitative value was determined using the standard curves from FIG. 28A. The fold-change over the background histidine-tag expression is noted

FIG. 29A provides a workflow for assessing the stability of the ALFA-NanoLuc interaction over time. FIG. 29B provides a quantitative analysis of the stability of the ALFA-NanoLuc expression compared to NanoLuc fused to a poly-histidine tag.

FIG. 30A provides a workflow for labeling exosomes overexpressing CfaC with NanoLuc fused to the CfaN. FIG. 30B provides SDS-PAGE analysis post-loading and UC cleanup showing association of NanoLuc-CfaN with CfaC expressing exosomes.

FIG. 31A provides standard curves of NanoLuc fused to CfaN or a polyhistidine tag. FIG. 31B provides a quantitative analysis of the loading of (i) NanoLuc fused to CfaN or (ii) NanoLuc fused to a poly-histidine tag on the modified exosomes. The quantitative value was determined using the standard curves from FIG. 31A. The fold-change over the background histidine-tag expression is noted.

FIG. 32A provides the workflow for labeling exosomes overexpressing SpyCatcher with NanoLuc fused to the SpyTag. FIG. 32B provides SDS-PAGE analysis post-loading and UC cleanup showing association of NanoLuc-SpyTag with SpyCatcher expressing exosomes.

FIG. 33A provides standard curves of NanoLuc fused to Spy Tag or a poly-histidine tag. FIG. 33B provides a quantitative analysis of the loading of (i) NanoLuc fused to SpyTag or (ii) NanoLuc fused to poly-histidine tag on the modified exosomes. The quantitative value was determined using the standard curves from FIG. 33A. The fold-change over the background histidine-tag expression is noted.

FIG. 34 provides a comparison of the loading of NanoLuc fused to one of the following donor domains in the modified exosomes: (i) ALFAtag, (ii) CfaN, and (iii) SpyTag.

FIG. 35 shows a schematic illustrating surface conjugation of a moiety of interest to a scaffold protein associated with an exosome membrane.

FIG. 36 shows a table listing mechanistic characteristics of small molecules and oligonucleotides conjugated to exosomes. pAB-pNP: p-Aminobenzyl alcholog p-Nitrophenyl carbonate; DBCO: dibenzylcyclooctyne.

FIG. 37 shows a table listing lipid-linker-reactive groups as intermediates. The reactive group on the linker and its respective functional group on the moiety of interest is listed.

FIGS. 38A, 38B, 38C, and 38D show examples of MCC-950, a small molecule, conjugation. FIG. 38A shows MCC-950-Val-Cit-Maleimide conjugation. FIG. 38B shows MCC-950-Val-Cit-Methacrylate conjugation. FIG. 38C shows MCC-950-Val-Cit-Pyridinyl Disulfide conjugation. FIG. 38D shows a schematic illustrating the conjugation site of a sulfhydryl group on an exosome bound scaffold protein.

FIGS. 39A and 39B show examples of oligonucleotide/protein conjugation. FIG. 39A shows an oligo-maleimide conjugation and a schematic illustrating its conjugation site of a sulfhydryl group on an exosome bound scaffold protein. FIG. 39B shows an oligo-NHS ester conjugation and a schematic illustrating its conjugation site on a primary amine group on an exosome bound scaffold protein.

FIG. 40 shows a table listing the function group on the scaffold protein of exosomes, activation thereof, and functional group on the protein/peptide to be conjugated, and the degradability of the linkage. Due to the same functional groups, options for protein to protein/peptide conjugation is limited to click chemistry. DBCO: dibenzylcyclooctyne.

FIG. 41 shows an example of protein/peptide conjugation (Schiff base enabled).

FIG. 42 shows an example of protein/peptide conjugation (click chemistry).

FIGS. 43A and 43B provide schematic of EVs comprising the ALFA plug and play system described herein. As shown in FIG. 43A, ALFA nanobody (NbALFA) is fused to Scaffold X (e.g., PTGFRN) on the exterior surface of the EV (“NbALFA EV”). The crystal structure shows the stable, non-covalent interaction of the NbALFA with the ALFAtag. FIG. 43B shows the loading of moieties of interest (MOI) onto the exterior surface of the NbALFA EVs. As further described herein, the moieties of interest are fused to an ALFAtag and then, mixed with the NbALFA EVs, resulting in the stable association of the ALFAtagged moieties of interest to the exterior surface of the NbALFA EVs. The EVs can be loaded with the same moiety of interest (see top drawing) or a mixture of different moiety of interest (see bottom drawing).

FIG. 44 provides SDS-PAGE (top) and Western blot (bottom) results demonstrating that NbALFA EVs can be simultaneously loaded with multiple moieties of interest. Wild-type EVs or NbALFA EVs were mixed with 10 μg of NLuc-ALFAtag or molar equivalent of mouse IL-12 fused to ALFAtag (“mIL12-ALFAtag”).

### DETAILED DESCRIPTION OF DISCLOSURE

The present disclosure is directed to an engineered EV that delivers one or more antigens, e.g., derived from a coronavirus, e.g., SARS-CoV-1 virus and/or SARS-CoV-2 virus. The EV platform allows luminal expression of one or more antigens and surface expression of one or more antigens designed to create a modular vaccination system. Various adjuvants can be incorporated into the EVs to enhance the immune response against a broad array of antigens. The engineered EVs can comprise one or more payloads and can improve at least one property (e.g., such as those disclosed herein) of the EV, and uses thereof. In some aspects, the one or more payloads comprise an antigen, an adjuvant, and/or an immune modulator. In certain aspects, the EV comprises one or more additional moieties (e.g., targeting moiety). In some aspects, the one or more payloads (e.g., antigen, adjuvant, and/or the immune modulator) and/or the one or more additional moieties (e.g., targeting moiety) can be attached (or linked) to one or more scaffold moieties on the surface of EVs or on the luminal surface of EVs. Non-limiting examples of the various aspects are shown in the present disclosure.

### Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In certain aspects, an extracellular vehicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane), Extracellular vesicles can be derived from a living or dead organism, explanted tissues or organs, prokaryotic or eukaryotic cells, and/or cultured cells. In some aspects, the extracellular vesicles are produced by cells that express one or more transgene products.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome or multi-vesicular body with the plasma membrane. In certain aspects, an exosome comprises a scaffold moiety. As described infra, exosome can be derived from a producer cell, and isolated from the producer cell based on its size, density, biochemical parameters, or a combination thereof. In some aspects, the EVs of the present disclosure are produced by cells that express one or more transgene products. Unless indicated otherwise, the terms “extracellular vesicle” and “exosomes” can be used interchangeably.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In certain aspects, a nanovesicle comprises a scaffold moiety. Nanovesicles, once derived from a producer cell, can be isolated from the producer cell based on its size, density, biochemical parameters, or a combination thereof.

As used herein, the term “associated with,” when used in the context of an EV of the present disclosure, refers to a physical interaction with the EV. For instance, when a moiety of interest (e.g., antigen, adjuvant, immune modulator, and/or targeting moiety) is associated with an EV, the moiety of interest is linked to the exterior surface of the EV, linked to the luminal surface of the EV, and/or in the lumen of the EV. As described herein, the moiety of interest can be directly linked to the exterior and/or luminal surface of the EV. In some aspects, the moiety of interest is linked to the exterior and/or luminal surface via one or more scaffold/anchoring moieties, such as those described herein. Accordingly, when a moiety of interest is not associated with an EV, in some aspects, the moiety of interest is in a soluble form.

As used herein the term “surface-engineered EVs” (e.g., Scaffold X-engineered EVs) refers to an EV with the membrane or the surface of the EV modified in its composition so that the surface of the engineered EV is different from that of the EV prior to the modification or of the naturally occurring EV. The engineering can be on the surface of the EV or in the membrane of the EV so that the surface of the EV is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV comprises an exogenous protein (i.e., a protein that the EV does not naturally express) or a fragment or variant thereof that can be exposed to the surface of the EV or can be an anchoring point (attachment) for a moiety exposed on the surface of the EV. In other aspects, a surface-engineered EV comprises a higher expression (e.g., higher number) of a natural exosome protein (e.g., Scaffold X) or a fragment or variant thereof that can be exposed to the surface of the EV or can be an anchoring point (attachment) for a moiety exposed on the surface of the EV.

As used herein the term “lumen-engineered EVs” (e.g., Scaffold Y-engineered EVs) refers to an EV with the membrane or the lumen of the EV modified in its composition so that the lumen of the engineered EV is different from that of the EV prior to the modification or of the naturally occurring EV. The engineering can be directly in the lumen or in the membrane of the EV so that the lumen of the EV is changed. For example, the membrane is modified in its composition of a protein, a lipid, a small molecule, a carbohydrate, etc. so that the lumen of the EV is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises an exogenous protein (i.e., a protein that the EV does not naturally express) or a fragment or variant thereof that can be exposed in the lumen of the EV or can be an anchoring point (attachment) for a moiety exposed on the inner layer of the EV. In other aspects, a lumen-engineered EV comprises a higher expression of a natural exosome protein (e.g., Scaffold X or Scaffold Y) or a fragment or variant thereof that can be exposed to the lumen of the exosome or can be an anchoring point (attachment) for a moiety exposed in the lumen of the exosome.

The term “modified,” when used in the context of EVs described herein, refers to an alteration or engineering of an EV and/or its producer cell, such that the modified EV is different from a naturally-occurring EV. In some aspects, a modified EV described herein comprises a membrane that differs in composition of a protein, a lipid, a small molecular, a carbohydrate, etc. compared to the membrane of a naturally-occurring EV (e.g., membrane comprises higher density or number of natural exosome proteins and/or membrane comprises proteins that are not naturally found in exosomes (e.g., antigen, adjuvant, and/or immune modulator). In certain aspects, such modifications to the membrane changes the exterior surface of the EV (e.g., surface-engineered EVs described herein). In certain aspects, such modifications to the membrane changes the lumen of the EV (e.g., lumen-engineered EVs described herein).

As used herein, the term “scaffold moiety” refers to a molecule that can be used to anchor a payload or any other compound of interest (e.g., antigen, adjuvant, and/or immune modulator) to the EV either on the luminal surface or on the exterior surface of the EV. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV. In some aspects, a scaffold moiety comprises a lipid, carbohydrate, or protein that does not naturally exist in the EV. In certain aspects, a scaffold moiety is Scaffold X. In some aspects, a scaffold moiety is Scaffold Y. In further aspects, a scaffold moiety comprises both Scaffold X and Scaffold Y. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin, LAMP2, and LAMP2B.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV). In some aspects, a Scaffold X can anchor a moiety (e.g., antigen, adjuvant, and/or immune modulator) to the external surface or the luminal surface of the exosome.

As used herein, the term “Scaffold Y” refers to exosome proteins that were newly identified within the lumen of exosomes. See, e.g., International Appl. No. PCT/US2018/061679, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety to the luminal surface of the exosome). In some aspects, a Scaffold Y can anchor a moiety (e.g., antigen, adjuvant, and/or immune modulator) to the luminal surface of the EV.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV. Similarly, in certain aspects, a functional fragment of a Scaffold Y protein retains the ability to anchor a moiety on the luminal surface of the EV. Whether a fragment is a functional fragment can be assessed by any art known methods to determine the protein content of EVs including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, antigen, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is b12seq, part of the BLAST suite of programs available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at worldwideweb.tcoffee.org, and alternatively available, e.g., from the EBI. It will also be appreciated that the final alignment used to calculate percent sequence identity can be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268:2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein, when a molecule described herein (e.g., antigen, adjuvant, immune modulator, targeting moiety, affinity ligand, and/or scaffold moiety) is “expressed” in an EV, it means that the molecule is present in or on the EV. As described herein, an EV can express a molecule of interest on its exterior surface, on its luminal surface, in the lumen, or combinations thereof. In some aspects, a molecule can be exogenously introduced into a producer cell or directly into an EV, such that the EV expresses the molecule of interest. In some aspects, a molecule of interest can be produced separately from an EV and then conjugated or linked to a moiety present in the EV, such that the EV expresses the molecule. For instance, in some aspects, an antigen (e.g., derived from a spike S protein of a coronavirus, e.g., receptor-binding domain of a spike S protein) can be fused to an affinity ligand disclosed herein. Then, the antigen-affinity ligand fusion can be linked or conjugated to a scaffold moiety expressed on the surface of an EV via the affinity ligand. Additional disclosure relating to different methods of expressing a molecule of interest in or on an EV is described elsewhere in the present disclosure.

As used herein, the term “linked to,” “fused,” or “conjugated to” are used interchangeably and refer to a covalent or non-covalent bond formed between a first moiety and a second moiety, e.g., Scaffold X and an antigen (or adjuvant or immune modulator), respectively, e.g., a scaffold moiety expressed in or on the extracellular vesicle and an antigen, e.g., Scaffold X (e.g., a PTGFRN protein), respectively, in the luminal surface of or on the external surface of the extracellular vesicle. In some aspects, a payload disclosed herein (e.g., antigen, adjuvant, and/or immune modulator) and/or a targeting moiety can be directly linked to the exterior surface and/or the luminal surface of an EV. As used herein, the term “directly linked,” “directly fused,” or “directly conjugated to” refer to the process of linking (fusing or conjugating) a moiety (e.g., a payload and/or targeting moiety) to the surface of an EV without the use of a scaffold moiety disclosed herein.

As used herein, the term “fusion protein” refers to two or more proteins that are linked or conjugated to each other. For instance, in some aspects, a fusion protein that can be expressed in an EV disclosed herein comprises (i) a payload (e.g., antigen, adjuvant, and/or immune modulator) and (ii) a scaffold moiety (e.g., Scaffold X and/or Scaffold Y). In some aspects, the payload (e.g., antigen, adjuvant, and/or immune modulator) is linked or conjugated to the scaffold moiety via an affinity ligand (e.g., those described herein). In some aspects, a fusion protein that can be expressed in an EV useful for the present disclosure comprises (i) a targeting moiety and (ii) a scaffold moiety (e.g., Scaffold X and/or Scaffold Y). In some aspects, the targeting moiety is linked or conjugated to the scaffold moiety via an affinity ligand (e.g., those described herein). As described herein, in some aspects, EVs of the present disclosure can express multiple fusion proteins, wherein a first fusion protein comprises (i) a payload (e.g., antigen, adjuvant, and/or immune modulator) and (ii) a scaffold moiety (e.g., Scaffold X and/or Scaffold Y), and wherein a second fusion protein comprises (i) a targeting moiety and (ii) a scaffold moiety (e.g., Scaffold X and/or Scaffold Y).

The term “encapsulated”, or grammatically different forms of the term (e.g., encapsulation, or encapsulating) refers to a status or process of having a first moiety (e.g., antigen, adjuvant, or immune modulator) inside a second moiety (e.g., an EV) without chemically or physically linking the two moieties. In some aspects, the term “encapsulated” can be used interchangeably with the terms “in the lumen of” and “loaded”. Non-limiting examples of encapsulating (or loading) a first moiety (e.g., payload, e.g., antigen, adjuvant, or immune modulator) into a second moiety (e.g., EVs) are disclosed elsewhere herein.

As used herein, the term “producer cell” refers to a cell used for generating an EV. A producer cell can be a cell cultured in vitro, or a cell in vivo. A producer cell includes, but not limited to, a cell known to be effective in generating EVs e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof. In some aspects, a producer cell is not a naturally-existing antigen-presenting cell (i.e., has been modified). In some aspects, a producer cell is not a naturally-existing dendritic cell, a B cell, a mast cell, a macrophage, a neutrophil, Kupffer-Browicz cell, cell derived from any of these cells, or any combination thereof. Additional disclosures relating to such producer cells are provided elsewhere in the present disclosure. In some aspects, the EVs useful in the present disclosure do not carry an antigen on MHC class I or class II molecule (i.e., antigen is not presented on MHC class I or class II molecule) exposed on the surface of the EV but instead can carry an antigen in the lumen of the EV or on the surface of the EV by attachment to Scaffold X and/or Scaffold Y.

As used herein, an “MHC class I molecule” refers to a protein product of a wild-type or variant HLA class I gene encoding an MHC class I molecule. Accordingly, “HLA class I molecule” and “MHC class I molecule” are used interchangeably herein.

MHC class I molecules are one of two primary classes of major histocompatibility complex (MHC) molecules (the other being MHC class II) and are found on the cell surface of all nucleated cells in the bodies of jawed vertebrates. They also occur on platelets, but not on red blood cells. Their function is to display peptide fragments of proteins from within the cell to cytotoxic T cells; this will trigger an immediate response from the immune system against a particular non-self antigen displayed with the help of an MHC class I protein. Because MHC class I molecules present peptides derived from cytosolic proteins, the pathway of MHC class I presentation is often called cytosolic or endogenous pathway.

In humans, the HLAs corresponding to MHC class I are HLA-A, HLA-B, and HLA-C. The MHC Class I molecule comprises two protein chains: the alpha chain and the β2-microglobulin (B2m) chain. Human B2m is encoded by the B2M gene. Class I MHC molecules bind peptides generated mainly from degradation of cytosolic proteins by the proteasome. The MHC I:peptide complex is then inserted via endoplasmic reticulum into the external plasma membrane of the cell. The epitope peptide is bound on extracellular parts of the class I MHC molecule. Thus, the function of the class I MHC is to display intracellular proteins to cytotoxic T cells (CTLs). However, class I MHC can also present peptides generated from exogenous proteins, in a process known as cross-presentation.

A normal cell will display peptides from normal cellular protein turnover on its class I MHC, and CTLs will not be activated in response to them due to central and peripheral tolerance mechanisms. When a cell expresses foreign proteins, such as after viral infection, a fraction of the class I MHC will display these peptides on the cell surface. Consequently, CTLs specific for the MHC:peptide complex will recognize and kill presenting cells. Alternatively, class I MHC itself can serve as an inhibitory ligand for natural killer cells (NKs). Reduction in the normal levels of surface class I MHC, a mechanism employed by some viruses to evade CTL responses, activates NK cell killing.

As used herein, an “MHC class II molecule” refers to a protein product of a wild-type or variant HLA class II gene encoding an MHC class II molecule. Accordingly, “HLA class II molecule” and “MHC class II molecule” are used interchangeably herein.

MHC class II molecules are a class of major histocompatibility complex (MHC) molecules normally found only on professional antigen-presenting cells such as dendritic cells, mononuclear phagocytes, some endothelial cells, thymic epithelial cells, and B cells. These cells are important in initiating immune responses. The antigens presented by class II peptides are derived from extracellular proteins (not cytosolic as in MHC class I).

Like MHC class I molecules, class II molecules are also heterodimers, but in this case consist of two homogenous peptides, an α and β chain, both of which are encoded in the MHC. The subdesignation α1, α2, etc. refers to separate domains within the HLA gene; each domain is usually encoded by a different exon within the gene, and some genes have further domains that encode leader sequences, transmembrane sequences, etc. These molecules have both extracellular regions as well as a transmembrane sequence and a cytoplasmic tail. The α1 and β1 regions of the chains come together to make a membrane-distal peptide-binding domain, while the α2 and β2 regions, the remaining extracellular parts of the chains, form a membrane-proximal immunoglobulin-like domain. The antigen binding groove, where the antigen or peptide binds, is made up of two α-helixes walls and β-sheet. Because the antigen-binding groove of MHC class II molecules is open at both ends while the corresponding groove on class I molecules is closed at each end, the antigens presented by MHC class II molecules are longer, generally between 15 and 24 amino acid residues long. Loading of a MHC class II molecule occurs by phagocytosis; extracellular proteins are endocytosed, digested in lysosomes, and the resulting epitopic peptide fragments are loaded onto MHC class II molecules prior to their migration to the cell surface. In humans, the MHC class II protein complex is encoded by the human leukocyte antigen gene complex (HLA). HLAs corresponding to MHC class II are HLA-DP, HLA-DM, HLA-DOA, HLA-DOB, HLA-DQ, and HLA-DR. Mutations in the HLA gene complex can lead to bare lymphocyte syndrome (BLS), which is a type of MHC class II deficiency.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “immune modulator” refers to an agent (i.e., payload) that acts on a target (e.g., a target cell) that is contacted with the extracellular vesicle, and regulates the immune system. Non-limiting examples of immune modulator that can be introduced into an EV and/or a producer cell include agents such as, modulators of checkpoint inhibitors, ligands of checkpoint inhibitors, cytokines, derivatives thereof, or any combination thereof. The immune modulator can also include an agonist, an antagonist, an antibody, an antigen-binding fragment, a polynucleotide, such as siRNA, antisense oligonucleotide, a phosphorodiamidate morpholino oligomer (PMO), a peptide-conjugated phosphorodiamidate morpholino oligomer (PPMO), miRNA, lncRNA, mRNA DNA, or a small molecule.

As used herein, the term a “bio-distribution modifying agent,” which refers to an agent (i.e., payload) that can modify the distribution of extracellular vesicles (e.g., exosomes, nanovesicles) in vivo or in vitro (e.g., in a mixed culture of cells of different varieties). In some aspects, the term “targeting moiety” can be used interchangeably with the term bio-distribution modifying agent. In some aspects, the targeting moiety alters the tropism of the EV (“tropism moiety”). As used herein, the term “tropism moiety” refers to a targeting moiety that when expressed on an EV alters and/or enhances the natural movement of the EV. For example, in some aspects, a tropism moiety can promote the EV to be taken up by a particular cell, tissue, or organ. Non-limiting examples of tropism moieties that can be used with the present disclosure include those that can bind to a marker expressed specifically on a dendritic cell (e.g., Clec9A or DEC205) or T cells (e.g., CD3). Unless indicated otherwise, the term “targeting moiety,” as used herein, encompasses tropism moieties. The bio-distribution agent can be a biological molecule, such as a protein, a peptide, a lipid, or a carbohydrate, or a synthetic molecule. For example, the bio-distribution modifying agent can be an affinity ligand (e.g., antibody, VHH domain, phage display peptide, fibronectin domain, camelid, VNAR), a synthetic polymer (e.g., PEG), a natural ligand/molecule (e.g., CD40L, albumin, CD47, CD24, CD55, CD59), a recombinant protein (e.g., XTEN), but not limited thereto.

In certain aspects, the bio-distribution modifying agent, and/or targeting moiety, is displayed on the surface of EVs. The bio-distribution modifying agent can be displayed on the EV surface by being fused to a scaffold protein (e.g., Scaffold X) (e.g., as a genetically encoded fusion molecule). In some aspects, the bio-distribution modifying agent can be displayed on the EV surface by chemical reaction attaching the bio-distribution modifying agent to an EV surface molecule. A non-limiting example is PEGylation. In some aspects, EVs disclosed herein can further comprise a bio-distribution modifying agent, in addition to an antigen, adjuvant, or immune modulator. Non-limiting examples of bio-distribution modifying agent or targeting moiety that can be used with the present disclosure include a C-type lectin domain family 9 member A (Clec9a) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), CD3, or any combination thereof. In certain aspects, the targeting moiety is Clec9a protein. In some aspects, the targeting moiety is a CD3 molecule.

As used herein, the term “C-type lectin domain family 9 member A” (Clec9a) protein refers to a group V C-type lectin-like receptor (CTLR) that functions as an activation receptor and is expressed on myeloid lineage cells (e.g., DCs). Huysamen et al., J Biol Chem 283 (24): 16693-701 (2008); U.S. Pat. No. 9,988,431 B2, each of which is herein incorporated by reference in its entirety. Synonyms of Clec9a are known and include CD370, DNGR-1, 5B5, HEEE9341, and C-type lectin domain containing 9A. In some aspects, Clec9a protein is expressed on human cDC1 cells. In some aspects, Clec9a protein is expressed on mouse cDC1 and pDC cells. Unless indicated otherwise, Clec9a, as used herein, can refer to Clec9a from one or more species (e.g., humans, non-human primates, dogs, cats, guinea pigs, rabbits, rats, mice, horses, cattle, and bears).

As used herein, the term “CD3” or “cluster of differentiation 3” refers to the protein complex associated with the T cell receptor (TCR). The CD3 molecule is made up of four distinct chains (CD3γ, CD3δ, and two CD3ε chains). These chains associate with the T-cell receptor (TCR) and the ζ-chain to generate an activation signal in T lymphocytes. The TCR, ζ-chain, and CD3 molecules together constitute the TCR complex. CD3 molecules are expressed on all T cells, including both CD4+ T cells and CD8+ T cells. Unless indicated otherwise, CD3, as used herein, can refer to CD3 from one or more species (e.g., humans, non-human primates, dogs, cats, guinea pigs, rabbits, rats, mice, horses, cattle, and bears).

As used herein, the term “payload” refers to an agent that acts on a target (e.g., a target cell) that is contacted with the EV. In some aspects, unless indicated otherwise, the term payload can be used interchangeably with the term “biologically active molecules.” Non-limiting examples of payload that can be included on the EV are an antigen, an adjuvant, and/or an immune modulator. Payloads that can be introduced into an EV and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, siRNA, antisense oligonucleotide, a phosphorodiamidate morpholino oligomer (PMO), a peptide-conjugated phosphorodiamidate morpholino oligomer (PPMO), or combinations thereof), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an antigen. As used herein, the term “antigen” refers to any agent that when introduced into a subject elicits an immune response (cellular or humoral) to itself.

As used herein, the term “affinity ligand” refers to a molecule that can selectively and preferentially bind to a specific marker, e.g., expressed on a target cell or on EVs, e.g., a scaffold moiety, e.g., PTGFRN on EVs. As described herein, in some aspects, an affinity ligand comprises a peptide (e.g., linear peptide) or protein that can increase the binding of a molecule of interest (e.g., antigen, adjuvant, immune modulator, and/or targeting moiety) to a moiety on the surface of EVs, e.g., a scaffold moiety disclosed herein. Non-limiting examples of affinity ligands that can be used with the present disclosure include an antibody, phage display peptide, fibronectin domain, camelid, VNAR, VHH domain, and combinations thereof. As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. Further description of affinity ligands that can be used with an EV are provided elsewhere in the present disclosure (see, e.g., section II.I).

As used herein, the term “acceptor domain” refers to a protein sequence that forms a stable interaction (either covalent or non-covalent) with a cognate protein “donor domain.” As demonstrated herein, in some aspects, acceptor domains can be displayed on the surface of EVs via fusion to a scaffold moiety (e.g., PTGFRN). In some aspects, by mixing the acceptor EVs (i.e., EVs that comprise the acceptor domain) with a soluble donor (comprising the donor domain and a target molecule of interest), it can be possible to display the target molecule of interest on the surface of EVs.

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects, the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substantially free” means that the sample comprising EVs comprise less than about 10% of macromolecules by mass/volume (m/v) percentage concentration. Some fractions can contain less than about 0.001%, less than about 0.01%, less than about 0.05%, less than about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, less than about 5%, less than about 6%, less than about 7%, less than about 8%, less than about 9%, or less than about 10% (m/v) of macromolecules.

As used herein, the term “macromolecule” means nucleic acids, contaminant proteins, lipids, carbohydrates, metabolites, or a combination thereof.

As used herein, the term “conventional exosome protein” means a protein previously known to be enriched in exosomes, including but is not limited to CD9, CD63, CD81, PDGFR, GPI anchor proteins, lactadherin LAMP2, and LAMP2B, a fragment thereof, or a peptide that binds thereto.

“Administering,” as used herein, means to give a composition comprising an EV disclosed herein to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. EVs can be administered as part of a pharmaceutical composition comprising at least one excipient.

An “immune response,” as used herein, refers to a biological response within a vertebrate against foreign agents or abnormal, e.g., corona virus, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly an immune response can comprise a humoral immune response (e.g., mediated by B-cells), cellular immune response (e.g., mediated by T cells), or both humoral and cellular immune responses. In some aspects, an immune response is an “inhibitory” immune response. An “inhibitory” immune response is an immune response that blocks or diminishes the effects of a stimulus (e.g., antigen). In certain aspects, the inhibitory immune response comprises the production of inhibitory antibodies against the stimulus. In some aspects, an immune response is a “stimulatory” immune response. A “stimulatory” immune response is an immune response that results in the generation of effectors cells (e.g., cytotoxic T lymphocytes) that can destroy and clear a target antigen of coronaviruses.

As used herein, the term “cellular immune response” can be used interchangeably with the term “cell-mediated immune response” and refers to an immune response that does not predominantly involve antibodies. Instead, a cellular immune response involves the activation of different immune cells (e.g., phagocytes and antigen-specific cytotoxic T-lymphocytes) that produce various effector molecules (e.g., cytokines, perforin, granzymes) upon activation (e.g., via antigen stimulation). As used herein, the term “humoral immune response” refers to an immune response predominantly mediated by macromolecules found in extracellular fluids, such as secreted antibodies, complement proteins, and certain antimicrobial peptides. The term “antibody-mediated immune response” refers to an aspect of a humoral immune response that is mediated by antibodies.

As used herein, the term “immune cells” refers to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell expresses CD3. In certain aspects, the CD3-expressing immune cells are T cells (e.g., CD4+ T cells or CD8+ T cells). In some aspects, an immune cell that can be targeted with a targeting moiety disclosed herein (e.g., anti-CD3) comprises a naïve CD4+ T cell. In some aspects, an immune cell comprises a memory CD4+ T cell. In some aspects, an immune cell comprises an effector CD4+ T cell. In some aspects, an immune cell comprises a naïve CD8+ T cell. In some aspects, an immune cell comprises a memory CD8+ T cell. In some aspects, an immune cell comprises an effector CD8+ T cell. In some aspects, an immune cell is a dendritic cell. In certain aspects, a dendritic cell comprises a plasmacytoid dendritic cell (pDC), a conventional dendritic cell 1 (cDC1), a conventional dendritic cell 2 (cDC2), inflammatory monocyte derived dendritic cells, Langerhans cells, dermal dendritic cells, lysozyme-expressing dendritic cells (LysoDCs), Kupffer cells, or any combination thereof. Accordingly, in certain aspects, an immune cell that an EV disclosed herein can specifically target includes a conventional dendritic cell 1 (cDC1) and/or plasmacytoid dendritic cells (pDC).

As used herein, the term “T cell” or “T-cell” refers to a type of lymphocyte that matures in the thymus. T cells play an important role in cell-mediated immunity and are distinguished from other lymphocytes, such as B cells, by the presence of a T-cell receptor on the cell surface. T-cells include all types of immune cells expressing CD3, including T-helper cells (CD4+ cells), cytotoxic T-cells (CD8+ cells), natural killer T-cells, T-regulatory cells (Treg), and gamma-delta T cells.

A “naïve” T cell refers to a mature T cell that remains immunologically undifferentiated (i.e., not activated). Following positive and negative selection in the thymus, T cells emerge as either CD4+ or CD8+naïve T cells. In their naïve state, T cells express L-selectin (CD62L+), IL-7 receptor-α (IL-7R-α), and CD132, but they do not express CD25, CD44, CD69, or CD45RO. As used herein, “immature” can also refers to a T cell which exhibits a phenotype characteristic of either a naïve T cell or an immature T cell, such as a TSCM cell or a TCM cell. For example, an immature T cell can express one or more of L-selectin (CD62L+), IL-7Rα, CD132, CCR7, CD45RA, CD45RO, CD27, CD28, CD95, CXCR3, and LFA-1. Naïve or immature T cells can be contrasted with terminal differentiated effector T cells, such as T_{EM} cells and T_{EFF} cells.

As used herein, the term “effector” T cells or “T_{EFF}” cells refers to a T cell that can mediate the removal of a pathogen or cell without requiring further differentiation. Thus, effector T cells are distinguished from naive T cells and memory T cells, and these cells often have to differentiate and proliferate before becoming effector cells.

As used herein, the term “memory” T cells refer to a subset of T cells that have previously encountered and responded to their cognate antigen. In some aspects, the term is synonymous with “antigen-experienced” T cells. In some aspects, memory T cells can be effector memory T cells or central memory T cells. In some aspects, the memory T cells are tissue-resident memory T cells. As used herein, the term “tissue-resident memory T cells” or “TRM cells” refers to a lineage of T cells that occupies tissues (e.g., skin, lung, gastrointestinal tract) without recirculating. TRM cells are transcriptionally, phenotypically and functionally distinct from central memory and effector memory T cells which recirculate between blood, the T cell zones of secondary lymphoid organs, lymph and nonlymphoid tissues. One of the roles of TRM cells is to provide immune protection against infection in extralymphoid tissues.

As used herein, the term “dendritic cells” or “DCs” refers to a class of bone-marrow-derived immune cells that are capable of processing extracellular and intracellular proteins and to present antigens in the context of MHC molecules to prime naïve T cells. In some aspects, dendritic cells can be divided into further subtypes, such as conventional dendritic cell 1 (cDC1), conventional dendritic cell 2 (cDC2), plasmacytoid dendritic cell (pDC), inflammatory monocyte derived dendritic cells, Langerhans cells, dermal dendritic cells, lysozyme-expressing dendritic cells (LysoDCs), Kupffer cells, and combinations thereof. In certain aspects, the different DC subsets can be distinguished based on their phenotypic expression. For example, in some aspects, human cDC1 cells are CD1c; and CD1414. In some aspects, human cDC2 cells are CD1c⁺ and CD141⁻. In some aspects, human pDC cells are CD123⁺. In some aspects, mouse cDC1 cells are XCR1⁺, Clec9a⁺, and Sirpa⁻. In some aspects, mouse cDC2 cells are CD8⁺, CD11b⁺, Sirpa⁺, XCR1⁻, and CD1c, b⁺. In some aspects, mouse pDC cells are CD137⁺, XCR1⁻, and Sirpa⁻. Other phenotypic markers for distinguishing the different DC subsets are known in the art. See, e.g., Collin et al., Immunology 154 (1): 3-20 (2018). In some aspects, the different DC subsets can be distinguished based on their functional properties. For example, in certain aspects, pDCs produce large amounts of IFN-α, while cDC1s and cDC2s produce inflammatory cytokines, such as IL-12, IL-6, and TNF-α. Other methods of distinguishing the different DC subsets are known in the art. See, e.g., U.S. Pat. Nos. 8,426,565 B2 and 9,988,431, each of which is herein incorporated by reference in its entirety.

The term “immunoconjugate,” as used herein, refers to a compound comprising a binding molecule (e.g., an antibody) and one or more moieties, e.g., therapeutic or diagnostic moieties, chemically conjugated to the binding molecule. In general, an immunoconjugate is defined by a generic formula: A-(L-M) n, wherein A is a binding molecule (e.g., an antibody), L is an optional linker, and M is a heterologous moiety which can be for example a therapeutic agent, a detectable label, etc., and n is an integer. In some aspects, multiple heterologous moieties can be chemically conjugated to the different attachment points in the same binding molecule (e.g., an antibody). In other aspects, multiple heterologous moieties can be concatenated and attached to an attachment point in the binding molecule (e.g., an antibody). In some aspects, multiple heterologous moieties (being the same or different) can be conjugated to the binding molecule (e.g., an antibody).

Immunoconjugates can also be defined by the generic formula in reverse order. In some aspects, the immunoconjugate is an “antibody-Drug Conjugate” (“ADC”). In the context of the present disclosure, the term “immunoconjugate” is not limited to chemically or enzymatically conjugates molecules. The term “immunoconjugate” as used in the present disclosure also includes genetic fusions. In some aspects of the present disclosure, the biologically active molecule is an immunoconjugate. The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule (i.e., a payload) is an antibody-drug conjugate.

“Treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

“Prevent” or “preventing,” as used herein, refers to decreasing or reducing the occurrence or severity of a particular outcome. In some aspects, preventing an outcome is achieved through prophylactic treatment.

### II. Extracellular Vesicles

Disclosed herein are EVs capable of regulating the immune system of a subject. As described herein, EVs described herein differ from other platforms (e.g., protein immunization) for regulating an immune system of a subject in that the EVs comprise one or more of the following properties: (i) flexibility of moiety (e.g., antigen) display, (ii) diverse adjuvant and immunomodulatory combinations, (iii) enhanced cell-specific tropism, (iv) enhanced clearance inhibition, or (v) any combination thereof.

In some aspects, EVs of the present disclosure provide flexibility of moiety display. For instance, the moieties of interest (e.g., antigen) (i) can be directly linked to a surface of the EV (e.g., exterior surface and/or luminal surface), (ii) can be linked to a scaffold moiety (e.g., Scaffold X and/or Scaffold Y) and then expressed on a surface of the EV (e.g., exterior surface and/or luminal surface), (iii) can be expressed in the lumen of the EV, or (iv) combinations thereof. Such ability to rapidly engineer EVs is particularly useful in developing EV-based vaccines for treating the diseases and disorders described herein. For instance, a single EV engineered to express certain payloads and/or targeting moieties can be used in treating a wide range of diseases or disorders by simply “plugging” a moiety (e.g., antigen of interest) into the EVs (or rapidly attaching a moiety (e.g., antigen of interest) as a “clip-on” attachment to the EVs). Methods of producing such modular or “plug and play” EVs are provided elsewhere in the present disclosure.

In some aspects, EVs of the present disclosure allow for the diverse combinations of different moieties of interest (e.g., antigens, adjuvants, immunomodulators, and/or targeting moieties). In certain aspects, the EVs allow for the combination of a wide range of adjuvants and immunomodulators. Non-limiting examples of adjuvants and immunomodulators that can be combined in a single EV include small molecule agonists (e.g., STING), small molecule antagonists, co-stimulatory proteins, anti-sense and bacterial adjuvant oligonucleotides. Additional disclosure relating to the different moieties that can be combined together are provided elsewhere in the present disclosure.

In some aspects, EVs described herein can be engineered to exhibit enhanced cell-specific tropism. For instance, the EVs can be engineered to express on their exterior surface a targeting moiety (e.g., antibodies and/or proteins) that can specifically bind to a marker on a specific cell. In some aspects, EVs described herein can be engineered to induce certain types of immune responses (e.g., T cell, B cell, and/or Treg/tolerogenic immune responses). Additional disclosure relating to such properties are provided elsewhere in the present disclosure.

The EVs useful in the present disclosure have been engineered to produce multiple (e.g., at least two) exogenous biologically active molecules (e.g., an antigen, an antibody or an antigen-binding fragment thereof, an adjuvant, and/or an immune modulator), and/or other moieties (e.g., a targeting moiety) together in a single EV. In some aspects, an EV comprises three exogenous biologically active molecules. In other aspects, an EV comprises four exogenous biologically active molecules. In further aspects, an EV comprises five or more exogenous biologically active molecules. In some aspects, an EV comprises 6, 7, 8, 9, 10, 11, 12, 13, 14, 15, 16, 17, 18, 19, 20 or more exogenous biologically active molecules.

In some aspects, an EV comprises two or more exogenous biologically active molecules, e.g., (i) one or more antigens and (ii) one or more adjuvants. In other aspects, an EV comprises two or more exogenous biologically active molecules, e.g., (1) one or more antigens and (ii) one or more immune modulators. In further aspects, an EV comprises two or more exogenous biologically active molecules, e.g., (i) one or more antigens, (ii) one or more immune modulators, and (iii) one or more adjuvants. In certain aspects, an EV can further comprise one or more additional moieties, e.g., targeting moieties. In some aspects, an antigen is not expressed (or presented) on major histocompatibility complex I and/or II molecules. In other aspects, while an antigen in the EV is not expressed or presented as part of the MHC class I or II complex, the EV can still contain MHC class I/II molecules on the surface of the EV. Accordingly, in certain aspects, EVs disclosed herein do not directly interact with T-cell receptors (TCRs) of T cells to induce an immune response against the antigen. Similarly, in certain aspects, EVs of the present disclosure do not transfer the antigen directly to the surface of the target cell (e.g., dendritic cell) through cross-dressing. Cross-dressing is a mechanism commonly used by EVs derived from dendritic cells (DEX) to induce T cell activation. See Pitt, J. M., et al., J Clin Invest 126 (4): 1224-32 (2016). In other aspects, the EVs of the present disclosure are engulfed by antigen presenting cells and can be expressed on the surface of the antigen presenting cells as MHC class I and/or MHC class II complex.

In certain aspects, an EV disclosed herein can also comprise additional moieties, such as a targeting moiety. In some aspects, an antigen is expressed or presented on major histocompatibility complex I and/or II molecules. In other aspects, while an antigen in the EV is expressed or presented as part of the MHC class I or II complex, the EV can contain MHC class I/II molecules on the surface of the EV. Accordingly, in certain aspects, EVs disclosed herein directly interact with T-cell receptors (TCRs) of T cells to induce an immune response against the antigen. Similarly, in certain aspects, EVs of the present disclosure transfer the antigen directly to the surface of the target cell (e.g., dendritic cell) through cross-dressing. “Cross-dressing” is a mechanism commonly used by EVs derived from dendritic cells (DEX) to induce T cell activation. See Pitt, J. M., et al., J Clin Invest 126 (4): 1224-32 (2016). In other aspects, the EVs of the present disclosure are engulfed by antigen presenting cells and can be expressed on the surface of the antigen presenting cells as MHC class I and/or MHC class II complex.

As described supra, EVs described herein are extracellular vesicles with a diameter between about 20-300 nm. In certain aspects, an EV of the present disclosure has a diameter between about 20-290 nm, between about 20-280 nm, between about 20-270 nm, between about 20-260 nm, between about 20-250 nm, between about 20-240 nm, between about 20-230 nm, between about 20-220 nm, between about 20-210 nm, between about 20-200 nm, between about 20-190 nm, between about 20-180 nm, between about 20-170 nm, between about 20-160 nm, between about 20-150 nm, between about 20-140 nm, between about 20-130 nm, between about 20-120 nm, between about 20-110 nm, between about 20-100 nm, between about 20-90 nm, between about 20-80 nm, between about 20-70 nm, between about 20-60 nm, between about 20-50 nm, between about 20-40 nm, or between about 20-30 nm. The size of the EV described herein can be measured according to methods described, infra.

In some aspects, an EV of the present disclosure comprises a bi-lipid membrane (“EV membrane”), comprising an interior surface and an exterior surface. In certain aspects, the interior surface faces the inner core (i.e., lumen) of the EV. In certain aspects, the exterior surface can be in contact with the endosome, the multivesicular bodies, or the membrane/cytoplasm of a producer cell or a target cell

In some aspects, the EV membrane comprises lipids and fatty acids. In some aspects, the EV membrane comprises phospholipids, glycolipids, fatty acids, sphingolipids, phosphoglycerides, sterols, cholesterols, and phosphatidylserines.

In some aspects, the EV membrane comprises an inner leaflet and an outer leaflet. The composition of the inner and outer leaflet can be determined by transbilayer distribution assays known in the art, see, e.g., Kuypers et al., Biohim Biophys Acta 1985 819:170. In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine.

In some aspects, the EV membrane comprises one or more polysaccharide, such as glycan.

In some aspects, the EV membrane further comprises one or more scaffold moieties, which are capable of anchoring, e.g., an antigen and/or an adjuvant and/or an immune modulator, to the EV (e.g., either on the luminal surface or on the exterior surface). In certain aspects, scaffold moieties are polypeptides (“exosome proteins”). In other aspects, scaffold moieties are non-polypeptide moieties. In some aspects, exosome proteins include various membrane proteins, such as transmembrane proteins, integral proteins and peripheral proteins, enriched on the exosome membranes. They can include various CD proteins, transporters, integrins, lectins, and cadherins. In certain aspects, a scaffold moiety comprises Scaffold X. In other aspects, a scaffold moiety comprises Scaffold Y. In further aspects, a scaffold moiety comprises both a Scaffold X and a Scaffold Y.

In some aspects, an EV disclosed herein is capable of delivering a payload (e.g., an antigen, an adjuvant, and/or an immune modulator) to a target. The payload is an agent that acts on a target (e.g., a target cell) that is contacted with the EV. Contacting can occur in vitro or in a subject. Non-limiting examples of payloads that can be introduced into an EV include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, siRNA, antisense oligonucleotide, a phosphorodiamidate morpholino oligomer (PMO), or a peptide-conjugated phosphorodiamidate morpholino oligomer (PPMO)), amino acids (e.g., amino acids comprising a detectable moiety or a toxin that disrupt translation), polypeptides (e.g., enzymes), lipids, carbohydrates, and small molecules (e.g., small molecule drugs and toxins).

As demonstrated herein, in some aspects, EVs of the present disclosure are capable of inducing effector and memory T cells. In certain aspects, the memory T cells are tissue-resident memory T cells. Such EVs could be particularly useful as vaccines for certain infectious diseases, such as those described herein, e.g., coronavirus, e.g., SARS-CoV-1 and/or SARS-CoV-2 (COVID-19) virus.

In some aspects, EVs disclosed herein are inherently capable of inducing the activation of a signaling pathway involved in an immune response. In certain aspects, the signaling pathway involved in an immune response comprises toll-like receptors (TLRs), retinoid acid-inducible gene I (RIG-I)-like receptors (RLRs), stimulator of interferon genes (STING) pathway, or combinations thereof. In some aspects, the activation of such signaling pathway can result in the production of a type I interferon. For example, in certain aspects, the bi-lipid membrane of an EV disclosed herein comprises one or more lipids that share one of the following features: (i) unsaturated lipid tail, (ii) dihydroimidazole linker, (iii) cyclic amine head groups, and (iv) combinations thereof. Lipids with such features have been shown to activate the TLR/RLR-independent STING pathway. See Miao et al., Nature Biotechnology 37:1174-1185 (October 2019), which is herein incorporated by reference in its entirety.

### II.A Antigen

In some aspects, an antigen that can be added to a base EV to produce the EV-based vaccines disclosed herein comprises any antigen known in the art, which is capable of eliciting a beneficial immune response in a subject. As used herein, a “beneficial immune response” is an immune response that is capable of treating (e.g., reducing and/or alleviating one or more symptoms) and/or preventing a disease or disorder, such as those described herein.

In some aspects, an antigen comprises a peptide. In certain aspects, the peptide comprises a natural peptide (e.g., such as that derived from a naturally-existing organism, e.g., virus). In some aspects, the peptide comprises a synthetic peptide. In some aspects, the peptide comprises both natural and synthetic peptides.

In some aspects, the peptide is less than about 150 amino acids in length, less than about 140 amino acids in length, less than about 130 amino acids in length, less than about 120 amino acids in length, less than about 110 amino acids in length, less than about 100 amino acids in length, less than about 90 amino acids in length, less than about 80 amino acids in length, less than about 70 amino acids in length, less than about 60 amino acids in length, less than about 50 amino acids in length, less than about 40 amino acids in length, less than about 30 amino acids in length, less than about 20 amino acids in length, or less than about 10 amino acids in length. In certain aspects, the peptide is less than about 100 amino acids in length. In some aspects, the peptide is less than about 80 amino acids in length.

In some aspects, an antigen useful for the present disclosure is a polynucleotide, e.g., an mRNA. In some aspects, an antigen useful for the disclosure is a synthetic mRNA encoding an epitope.

As described herein, in some aspects, an antigen can be linked to the exterior surface and/or luminal surface of EVs by various methods, including, but not limited to, anchoring moieties, affinity agents, chemical conjugation, or combinations thereof. To improve the attachment of the antigens to a surface of the EVs using such methods, the antigens described herein can be further modified. In certain aspects, an antigen comprises a peptide, which has been modified to contain a N-terminal lysine.

In some aspects, such a modification allows for the attachment of the antigen to a surface of the EV with chemical conjugation. For example, to enable click chemistry conjugation, an azide or strained alkyne (e.g., difluorinated cyclooctyne (DIFO)) would have to be attached (or linked) to the EV and/or to the antigen. In certain aspects, DIFO conjugates to the to the primary amine side chain on the N-terminal lysine of the antigen, which in turn can interact with the azide that can be attached to a surface of the EV. In some aspects, the azide can be attached to the antigen (via the primary amine side chain on the N-terminal lysine), and the strained alkyne can be attached to a surface of the EV.

In some aspects, modifying the antigen to comprise a N-terminal lysine can also be useful in linking the antigens to a surface of the EVs using anchoring moieties. In such aspects, the anchoring moiety (i.e., cholesterol, fatty acid, and/or vitamin E) can attach to the N-terminal lysine via the primary amine side chain. Once attached, the antigens can be readily inserted into the membrane of the EVs via the anchoring moieties.

As will be apparent to those skilled in the arts, the above described approaches to linking an antigen to the exterior surface and/or luminal surface of the EVs can also be performed by modifying one or more proteins on the EVs to contain unnatural amino acids with side chains to allow for the binding of molecules such as the azide, strained alkyne (e.g., difluorinated cyclooctyne (DIFO), or combinations thereof. Additional disclosure regarding such approaches to linking an antigen to a surface of the EVs are provided elsewhere in the present disclosure. While the above disclosures are provided in the context of antigens, it will be readily apparent to those skilled in the arts that similar approaches can be used to link other moieties of interest (e.g., adjuvant, targeting moiety, and/or scaffold moiety) to a surface of the EVs.

As is apparent from the present disclosure, an antigen useful for the present disclosure can comprise various structure and/or length. In some aspects, the differences in the structure and/or length can affect the potency of the EV-based vaccines described herein. In some aspects, the antigen comprises a linear epitope of a protein from which it is derived (e.g., T and/or B cell antigen of a coronavirus), a conformational epitope, or both.

Non-limiting examples of possible antigen structure are provided below. As described herein, any single aspect of the exemplary structures can be modified. For instance, the length of any of the components noted below can be modified (lengthened or shortened). Additionally, any of the components may be removed (e.g., remove a spacer) or additional components can be added (e.g., add more spacers).

In some aspects, the antigen comprises a single antigen. In some aspects, the antigen comprises a T cell antigen, which comprises a CD8+ T cell epitope, CD4+ T cell epitope, or both. For example, in certain aspects, an antigen has the following structure: (first flanking region)-(T cell antigen)-(second flanking region). In some aspects, the T cell antigen comprises a CD8+ T cell epitope and is 9 amino acids in length (e.g., T cell antigen of a coronavirus), and each of the first and second flanking regions is 5 amino acids in length, such that the entire antigen is 19 amino acids in length. In some aspects, the T cell antigen comprises a CD4+ T cell epitope and is 25 amino acids in length (e.g., T cell antigen of a coronavirus), and each of the first and second flanking regions is 5 amino acids in length, such that the entire antigen is 35 amino acids in length. As described herein, in some aspects, the antigen comprises a N-terminal lysine.

In some aspects, the antigen comprises a B cell antigen. In certain aspects, such antigen has the following structure: (B cell antigen)-(spacer)-(T helper peptide). In certain aspects, the B cell antigen (e.g., S2 antigen of a coronavirus) is 31 amino acids in length, the spacer is 3 amino acids in length, and the T helper peptide (e.g., PADRE) is 13 amino acids in length, such that the entire antigen is 47 amino acids in length. Non-limiting examples of spacers that can be used comprise one the following amino acid sequences: CPGPG (SEQ ID NO: 579), AAY, GSGSGS (SEQ ID NO: 580), or combinations thereof. As described herein, in some aspects, the antigen comprises a N-terminal lysine.

In some aspects, the antigen comprises a concatemer of multiple epitopes of an antigen. For instance, in certain aspects, an antigen has the following structure: (first flanking region)-(first T cell antigen)-(first spacer)-(second T cell antigen)-(second spacer)-(third T cell antigen)-(second flanking region). In some aspects, the first and second flanking regions are 5 amino acids in length; the first, second, and third T cell antigens are 9 amino acids in length; and the first and second spacers are 3 amino acids in length, such that the entire antigen is 43 amino acids in length. As described herein, in some aspects, the antigen comprises a N-terminal lysine.

In some aspects, the potency of the EV-based vaccines of the present disclosure can be regulated by modifying the structure and/or overall length (e.g., the lengths of the flanking region, spacers, and/or T and B cell antigens).

As described elsewhere in the present disclosure, the EV-based vaccines described herein can be used to treat a wide range of diseases and disorders, e.g., by simply adding an antigen of interest to the base EV. In some aspects, the antigen is derived from and/or comprises a virus, a bacterium, a parasite, a fungus, a protozoa, a tumor, an allergen, a self-antigen, or any combination thereof.

In some aspects, the antigen is derived from a virus. In some aspects, the antigen is derived from a virus causing a pandemic. As used herein, the term “pandemic” refers to the rapid spread of a certain disease, involving a wide area, and a large proportion of the population, which can form a worldwide epidemic across provincial, national, or even continental borders in a short period of time. In certain aspects, an antigen that can be added to an EV to produce an EV-based vaccine described herein is derived from a virus selected from a coronavirus, an influenza virus, an Ebola virus, a Chikungunya virus (CHIKV), a Crimean-Congo hemorrhagic fever (CCGF) virus, a Hendra virus, a Lassa virus, a Marburg virus, a monkeypox virus, a Nipah virus, a Hendra virus, a Rift Valley fever (RVF) virus, a Variola virus, a yellow fever virus, a Zika virus, a measles virus, a human immunodeficiency virus (HIV), a hepatitis C virus (HCV), a dengue fever virus (DENV), a parvovirus (e.g., B19 virus), a norvovirus, a respiratory syncytial virus (RSV), a lentivirus, an adenovirus, a flavivirus, a filovirus, a rhinovirus, a human papillomavirus (HPV), or any combination thereof.

In some aspects, the antigen is derived from a non-viral pathogen (e.g., a bacterium, parasite, fungus, protozoa, or combinations thereof). Non-limiting examples of such pathogens include: Vibrio cholera, Yersinia pestis bacteria, Mycobacterium tuberculosis (MTB), streptococcus (e.g., Streptococcus pyogenes, Streptococcus agalactiae, Streptococcus pneumoniae), staphylococcal bacteria (e.g., Staphylococcus aureus), shigella, Escherichia coli, salmonella, chlamydia (e.g., Chlamydia trachomatis), Pseudomonas aeruginosa, Klebsiella pneumoniae, Haemophilus influenza, Clostridia difficile, Plasmodium, Leishmania, Schistosoma, Trypanosoma, Brucella, Cryptosporidium, Entamoeba, Neisseria meningitis, Bacillus subtilis, Haemophilus influenzae, Neisseria gonorrhoeae, Borrelia burgdorferi, Corynebacterium diphteriae, Moraxella catarrhalis, Campylobacter jejuni, clostridium tetanus, Clostridium perfringens, Treponema pallidum, or any combination thereof.

Additional examples of antigens that can be expressed in an EV of the present disclosure (e.g., using the plug-and-play methods described herein) are provided in WO2020191361A2, which is incorporated herein by reference in its entirety.

In some aspects, the payload is an antigen derived from a coronavirus (also referred to herein as “coronavirus antigen”), which is capable of inducing an immune response in a subject. In some aspects, the coronavirus is an alphacoronavirus, a betacoronavirus, a gammacoronavirus, a deltacoronavirus, or combinations thereof. Exemplary description of such coronaviruses are provided in, e.g., Krichel et al., Sci Adv 7 (10): eabf1004 (March 2021), which is incorporated herein by reference in its entirety. In some aspects, the coronavirus comprises SARS-CoV-1 and/or SARS-CoV-2 (COVID-19). In some aspects, the coronavirus comprises Middle East respiratory syndrome-related coronavirus (MERS-CoV; also known as EMC/2012). Unless indicated otherwise, an antigen that can be expressed in an EV disclosed herein can be derived from any species of coronavirus. For instance, in certain aspects, an EV described herein comprises an antigen derived from a bat coronavirus (BtCoV), such as that described in Tsuda et al., Arch Virol 157 (12): 2349-55 (December 2012), which is incorporated herein by reference in its entirety. In some aspects, the coronavirus comprises zoonotic coronaviruses (i.e., jumped from an animal to human). See, e.g., Cohen et al., Science 371 (6530): 735-741 (February 2021), which is incorporated herein by reference in its entirety. While certain aspects of the present disclosure relates to SARS-CoV-1 and/or SARS-CoV-2 (COVID-19), it will be apparent to those skilled in the arts that, in some aspects, such disclosures can equally apply to other types of coronaviruses (e.g., MERS-CoV). Additionally, it will be apparent to those skilled in the arts that any relevant disclosure relating to a coronavirus described herein can equally apply to any other antigens described herein.

In some aspects, an EV disclosed herein comprises a single antigen. In some aspects, an EV disclosed herein comprises multiple antigens. In certain aspects, each of the multiple antigens is different. For instance, in some aspects, the multiple antigens can be derived from the same species of coronavirus (e.g., multiple different proteins of COVID-19). In some aspects, the multiple antigens can be derived from different species of coronavirus (e.g., one or more proteins from SARS-CoV-1, SARS-CoV-2, and MERS-CoV). In some aspects, an EV disclosed herein comprises at least 2, 3, 4, 5, 6, 7, 8, 9, 10 or more different antigens. As disclosed herein, an antigen can be linked to a surface of an EV using a scaffold moiety (e.g., Scaffold X and/or Scaffold Y). In certain aspects, an antigen can be directly linked (i.e., without the use of a scaffold moiety) to a surface of an EV. In some aspects, an antigen can be in the lumen of the EV. As is apparent from the present disclosure, in some aspects, an EV comprising an antigen (e.g., coronavirus antigen) can be used in combination with one or more additional antigens which are in a soluble form (i.e., not associated with an EV). For instance, in certain aspects, an EV-based vaccine described herein comprises (i) an EV comprising the RBD protein of a coronavirus (“exoRBD”), and (ii) a soluble peptide antigen comprising a coronavirus T cell epitope. As described herein, in some aspects, the exoRBD can further comprise an adjuvant (e.g., STING agonist).

In some aspects, an EV comprises the one or more antigens in combination with one or more additional payloads described herein (e.g., adjuvant and/or immune modulator). In some aspects, an EV can comprise one or more additional moieties (e.g., targeting moiety). For instance, in certain aspects, an EV disclosed herein can comprise (i) one or more additional antigens, (ii) one or more additional payloads (e.g., adjuvant and/or immune modulator), and (iii) one or more targeting moieties.

In some aspects, the antigen useful for the present disclosure is a universal antigen that is capable of inducing an immune response against any SARS coronavirus, e.g., SARS-CoV-1 and SARS-CoV-2 (COVID-19) virus. Therefore, in some aspects, the antigen useful for the present disclosure comprises an amino acid sequence that has at least about 90%, at least about 95%, at least about 96%, at least about 97%, at least about 98%, at least about 99%, or about 100% identical to at least five consecutive amino acids from a protein of a SARS coronavirus. In certain aspects, the universal antigen is derived from proteins of multiple coronaviruses (e.g., SARS-CoV-1, SARS-CoV-2, and MERS), such that an EV comprising the universal antigen is capable of inducing an immune response against many different types of coronaviruses (i.e., cross-reactive immune response) when the EV is administered to a subject. As described herein, in some aspects, the universal antigen is derived from the RBD protein of different coronaviruses (e.g., RBD from SARS-CoV-1, SARS-CoV-2, and MERS).

The coronavirus antigen that is useful for the present disclosure can be derived from any coding region of a coronavirus genome. For instance, in some aspects, the coronavirus antigen is derived from one or more of the open reading frames (ORFs), which code for accessory proteins, such as those that are not essential for virus replication but appear to have a role in pathogenesis. Non-limiting examples of such ORFs include ORF1a, ORF1b, ORF3a, ORF3b, ORF7b, ORF9b, ORF9c, ORF10, or combinations thereof. See, e.g., Michel et al., Virol J 17 (1): 131 (August 2020); and Finkel et al., Nature 589:125-130 (September 2020); each of which is incorporated herein by reference in its entirety. In certain aspects, the coronavirus antigen is derived from one or more of the coronavirus structural proteins: spike protein, membrane protein, envelope protein, and nucleocapsid protein.

In some aspects, the EVs of the present disclosure comprises at least two antigens, wherein the first antigen is capable of inducing a humoral immune response, e.g., a B cell response and the second antigen is capable of inducing a cellular immune response, e.g., a T cell (e.g., CD8+ cell) response. In some aspects, the humoral immune response inducing antigen is on the exterior surface of the EVs. In some aspects, the cellular immune response inducing antigen is in the lumen (on the luminal surface) of the EVs. In some aspects, the humoral immune response inducing antigen is in the lumen (on the luminal surface) of the EVs. In some aspects, the cellular immune response inducing antigen is on the exterior surface of the EVs.

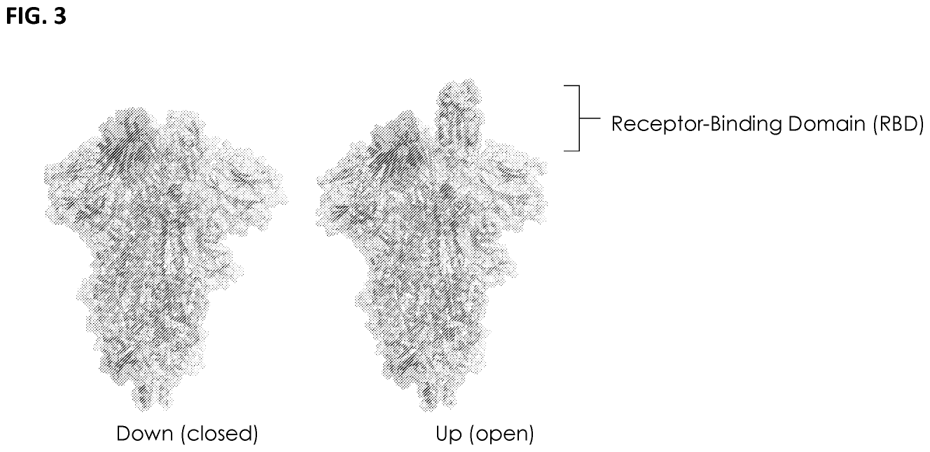

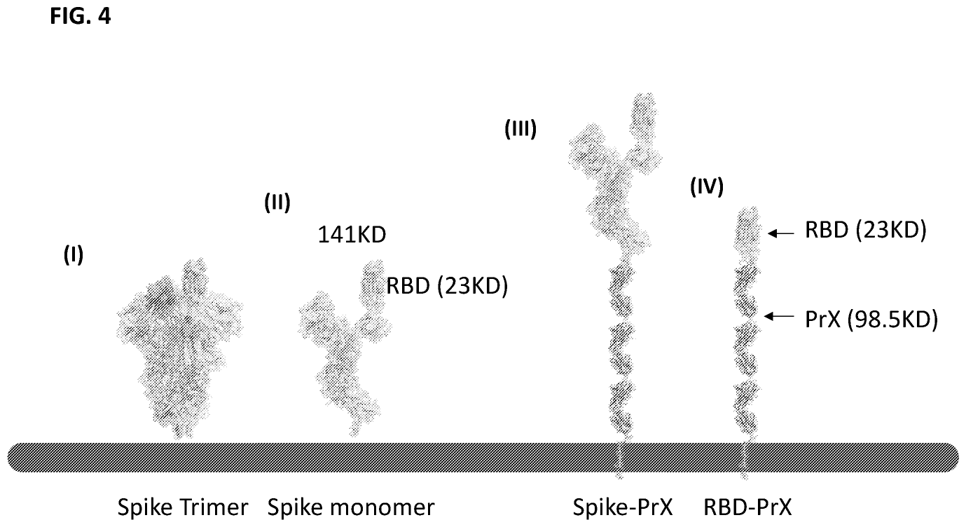

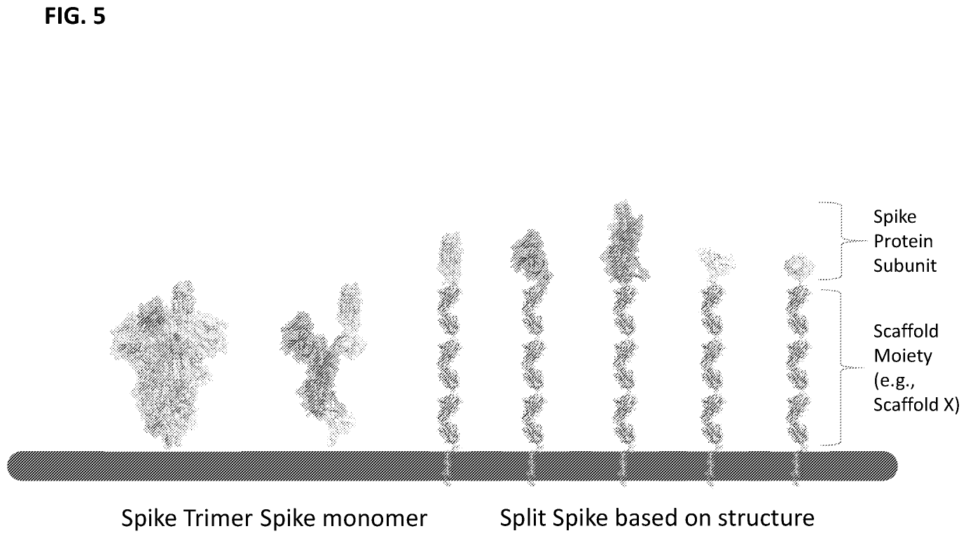

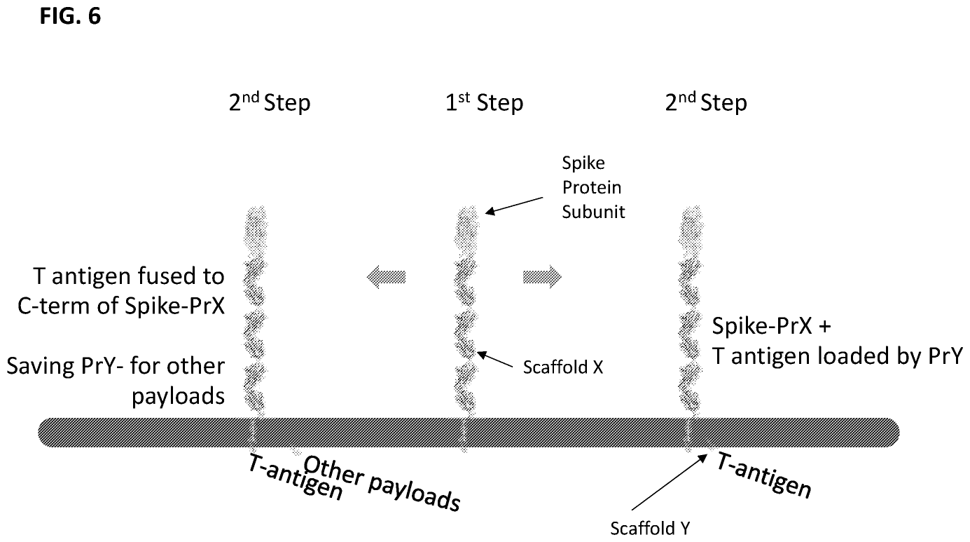

In some aspects, the antigen useful for the present disclosure comprises a receptor binding motif (also known as the “receptor-binding domain” (RBD)) of a spike protein derived from a SARS-CoV-1 and/or SARS-CoV-2 (COVID-19) virus. As used herein, the term “receptor-binding domain” or “RBD” includes all known RBDs of a coronavirus, such as that set forth in SEQ ID NO: 581, and any variants thereof. In some aspects, the antigen comprises at least five amino acids of the extracellular domain of an S protein from a SARS-CoV-1 and/or SARS-CoV-2 (COVID-19) virus. A spike protein of a coronavirus disclosed herein comprises a trimeric class I fusion protein, which can be in the down (closed) or up (open) conformation. As shown in FIG. 3, the receptor-binding domain of a coronavirus spike protein is exposed when the spike protein is in the up (open) conformation. As described herein, in some aspects, an EV comprises the spike protein of a coronavirus (or a fragment thereof) as the antigen. In such aspects, the spike protein can be in the trimeric configuration. In some aspects, the spike protein can be displayed in the EV as a monomeric subunit. Additional disclosures relating to the spike protein of a coronavirus disclosed herein, as well as the different subunits (e.g., RBD), are provided in Du, L., et al., Nature Reviews Microbiology 7:226-236 (2009), which is herein incorporated by reference in its entirety. In certain aspects, the RBD is a monomer. In some aspects, the RBD is a dimer, such as that described in Dai et al., Cell 182 (3): 722-733 (August 2020), which is herein incorporated by reference in its entirety.

In some aspects, the antigen useful for the present disclosure comprises at least five amino acids from spike protein S1, S2, and/or S2′ from a SARS-CoV-1 and/or SARS-CoV-2 (COVID-19) virus. In some aspects, the antigen useful for the present disclosure comprises at least five amino acids from spike protein S1 from a SARS-CoV-1 and/or SARS-CoV-2 (COVID-19) virus. In some aspects, the antigen useful for the present disclosure comprises at least five amino acids from spike protein S2 from a SARS-CoV-1 and/or SARS-CoV-2 (COVID-19) virus. In some aspects, the antigen comprises a highly conserved region of a coronavirus spike S2 protein that lacks glycosylation (“glycan hole”). See, e.g., Yuan et al., Nat Commun 8:15092 (April 2017), which is incorporated herein by reference in its entirety. In some aspects, the antigen useful for the present disclosure comprises at least five amino acids from spike protein S2′ from a SARS-CoV-1 and/or SARS-CoV-2 (COVID-19) virus. In some aspects, the antigen useful for the present disclosure comprises at least five amino acids from spike protein S2′ from a SARS-CoV-1 and/or SARS-CoV-2 (COVID-19) virus. In certain aspects, the antigen comprises a linear epitope derived from the S2 protein. In some aspects, the antigen comprises a conformation epitope of the S2 protein, wherein the conformational epitope is capable of eliciting an immune response and/or is capable of folding into a fold naturally found in the S2 protein.

In some aspects, the antigen comprises an amino acid epitope derived from a coronavirus, e.g., a coronavirus, e.g., SARS-CoV-1 and/or SARS-CoV-2 (COVID-19) virus. In some aspects, the EV comprises at least 2, 3, 4, 5, 6, 7, 8, 9, 10 or more antigens, wherein the first antigen is derived from a coronavirus, e.g., SARS-CoV-1 and/or SARS-CoV-2 (COVID-19) virus. In some aspects, the second antigen is also derived from a coronavirus, e.g., a SARS-CoV-1 and/or SARS-CoV-2 (COVID-19) virus. In some aspects, the second antigen is not derived from a coronavirus, e.g., a SARS-CoV-1 and/or SARS-CoV-2 (COVID-19) virus. In some aspects, the first and second antigens are derived from a coronavirus, e.g., a SARS-CoV-1 and/or SARS-CoV-2 (COVID-19) virus. In some aspects, the first and second antigens are the same. In some aspects, the first and second antigens are the same. In other aspects, the first antigen is derived from a coronavirus, e.g., a SARS-CoV-1 and/or SARS-CoV-2 (COVID-19) virus, and the second antigen is not derived from a coronavirus, e.g., a SARS-CoV-1 and/or SARS-CoV-2 (COVID-19) virus.

In some aspects, the antigen derived from a coronavirus, e.g., a SARS-CoV-1 and/or SARS-CoV-2 (COVID-19) virus, is derived from a spike(S) protein, including any variants thereof. In certain aspects, the antigen comprises the entire S protein. In some aspects, the antigen comprises a fragment of the S protein. In some aspects, the antigen comprises at least 5, 6, 7, 8, 9, 10, 11, 12, 13, 14, 15, 16, 17, 18, 19, or 20 amino acids of the S protein.

In some aspects, the antigen derived from a coronavirus, e.g., a SARS-CoV-1 and/or SARS-CoV-2 (COVID-19) virus, is derived from an envelope (E) protein, including any variants thereof. In certain aspects, the antigen comprises the entire E protein. In some aspects, the antigen comprises a fragment of the E protein. In some aspects, the antigen comprises at least 5, 6, 7, 8, 9, 10, 11, 12, 13, 14, 15, 16, 17, 18, 19, or 20 amino acids of the E protein.

In some aspects, the antigen derived from a coronavirus, e.g., a SARS-CoV-1 and/or SARS-CoV-2 (COVID-19) virus, is derived from a membrane (M) protein, including any variants thereof. In certain aspects, the antigen comprises the entire M protein. In some aspects, the antigen comprises a fragment of the M protein. In some aspects, the antigen comprises at least 5, 6, 7, 8, 9, 10, 11, 12, 13, 14, 15, 16, 17, 18, 19, or 20 amino acids of the M protein.

In some aspects, a first antigen is derived from an S protein of a coronavirus, e.g., a SARS-CoV-1 and/or SARS-CoV-2 (COVID-19) virus, e.g., a COVID-19 virus, and the second antigen is derived from an antigen from a coronavirus, e.g., an S protein of a coronavirus, e.g., a SARS-CoV-1 and/or SARS-CoV-2 (COVID-19) virus. In some aspects, a first antigen is derived from an S protein of a coronavirus, e.g., a SARS-CoV-1 and/or SARS-CoV-2 (COVID-19) virus, e.g., a COVID-19 virus, and the second antigen is derived from an E protein of a coronavirus, e.g., a SARS-CoV-1 and/or SARS-CoV-2 (COVID-19) virus. In some aspects, a first antigen is derived from an S protein of a coronavirus, e.g., a SARS-CoV-1 and/or SARS-CoV-2 (COVID-19) virus, and the second antigen is derived from an M protein of a coronavirus, e.g., a SARS-CoV-1 and/or SARS-CoV-2 (COVID-19) virus.

In some aspects, a first antigen is derived from an E protein of a coronavirus, e.g., a SARS-CoV-1 and/or SARS-CoV-2 (COVID-19) virus, and the second antigen is derived from an S protein of a coronavirus, e.g., a SARS-CoV-1 and/or SARS-CoV-2 (COVID-19) virus. In some aspects, a first antigen is derived from an E protein of a coronavirus, e.g., a SARS-CoV-1 and/or SARS-CoV-2 (COVID-19) virus, and the second antigen is derived from an E protein of a coronavirus, e.g., a SARS-CoV-1 and/or SARS-CoV-2 (COVID-19) virus. In some aspects, a first antigen is derived from an E protein of a coronavirus, e.g., a SARS-CoV-1 and/or SARS-CoV-2 (COVID-19) virus, and the second antigen is derived from an M protein of a coronavirus, e.g., a SARS-CoV-1 and/or SARS-CoV-2 (COVID-19) virus.

In some aspects, a first antigen is derived from an M protein of a coronavirus, e.g., a SARS-CoV-1 and/or SARS-CoV-2 (COVID-19) virus, and the second antigen is derived from an S protein of a coronavirus, e.g., a SARS-CoV-1 and/or SARS-CoV-2 (COVID-19) virus. In some aspects, a first antigen is derived from an M protein of a coronavirus, e.g., a SARS-CoV-1 and/or SARS-CoV-2 (COVID-19) virus, and the second antigen is derived from an E protein of a coronavirus, e.g., a SARS-CoV-1 and/or SARS-CoV-2 (COVID-19) virus. In some aspects, a first antigen is derived from an M protein of a coronavirus, e.g., a SARS-CoV-1 and/or SARS-CoV-2 (COVID-19) virus, and the second antigen is derived from an M protein of a coronavirus, e.g., a SARS-CoV-1 and/or SARS-CoV-2 (COVID-19) virus. Exemplary coronavirus sequences are disclosed below:

TABLE 1
SARS-CoV-1 Spike Protein (UniProtKB-P59594 (SPIKE_CVESA))
MFIFLLFLTL TSGSDLDRCT TFDDVQAPNY TQHTSSMRGV YYPDEIFRSD
TLYLTQDLFL PFYSNVTGFH TINHTFGNPV IPFKDGIYFA ATEKSNVVRG
WVFGSTMNNK SQSVIIINNS TNVVIRACNF ELCDNPFFAV SKPMGTQTHT
MIFDNAFNCT FEYISDAFSL DVSEKSGNFK HLREFVFKNK DGFLYVYKGY
QPIDVVRDLP SGFNTLKPIF KLPLGINITN FRAILTAFSP AQDIWGTSAA
AYFVGYLKPT TFMLKYDENG TITDAVDCSQ NPLAELKCSV KSFEIDKGIY
QTSNFRVVPS GDVVRFPNIT NLCPFGEVFN ATKFPSVYAW ERKKISNCVA
DYSVLYNSTF FSTFKCYGVS ATKLNDLCFS NVYADSFVVK GDDVRQIAPG
QTGVIADYNY KLPDDFMGCV LAWNTRNIDA TSTGNYNYKY RYLRHGKLRP
FERDISNVPF SPDGKPCTPP ALNCYWPLND YGFYTTTGIG YQPYRVVVLS
FELLNAPATV CGPKLSTDLI KNQCVNFNFN GLTGTGVLTP SSKRFQPFQQ
FGRDVSDFTD SVRDPKTSEI LDISPCSFGG VSVITPGTNA SSEVAVLYQD
VNCTDVSTAI HADQLTPAWR IYSTGNNVFQ TQAGCLIGAE HVDTSYECDI
PIGAGICASY HTVSLLRSTS QKSIVAYTMS LGADSSIAYS NNTIAIPTNF
SISITTEVMP VSMAKTSVDC NMYICGDSTE CANLLLQYGS FCTQLNRALS
GIAAEQDRNT REVFAQVKQM YKTPTLKYFG GFNFSQILPD PLKPTKRSFI
EDLLFNKVTL ADAGFMKQYG ECLGDINARD LICAQKFNGL TVLPPLLTDD
MIAAYTAALV SGTATAGWTF GAGAALQIPF AMQMAYRFNG IGVTQNVLYE
NQKQIANQFN KAISQIQESL TTTSTALGKL QDVVNQNAQA LNTLVKQLSS
NFGAISSVLN DILSRLDKVE AEVQIDRLIT GRLQSLQTYV TQQLIRAAEI
RASANLAATK MSECVLGQSK RVDFCGKGYH LMSFPQAAPH GVVFLHVTYV
PSQERNFTTA PAICHEGKAY FPREGVFVFN GTSWFITQRN FFSPQIITTD
NTFVSGNCDV VIGIINNTVY DPLQPELDSF KEELDKYFKN HTSPDVDLGD
ISGINASVVN IQKEIDRLNE VAKNLNESLI DLQELGKYEQ YIKWPWYVWL
GFIAGLIAIV MVTILLCCMT SCCSCLKGAC SCGSCCKFDE DDSEPVLKGV
KLHYT (SEQ ID NO: 417)
Extracellular domain: 14-1195
Transmembrane domain: 1196-1216
Cytoplasmic domain: 1217-1255

| | | |
|---|---|---|
| Signal peptide: | 1-13 | |
| Chain | 14-1255 | Spike glycoprotein |
| Chain | 14-667 | Spike protein S1 |
| Chain | 668-1255 | Spike protein S2 |
| Chain | 798-1255 | Spike protein S2′ |

SARS-CoV-1 Envelop Protein (UniProtKB-P59637 (VEMP_CVHSA))
MYSFVSEETG TLIVNSVLLF LAFVVFLLVT LAILTALRLC AYCCNIVNVS
LVKPTVYVYS RVKNLNSSEG VPDLLV (SEQ ID NO: 418)
SARS-CoV-1 Membrane Protein (UniProtKB-P59596 (VME1_CVESA))
MADNGTITVE ELKQLLEQWN LVIGFLFLAW IMLLQFAYSN RNRFLYIIKL
VFLWLLWPVT LACFVLAAVY RINWVTGGIA IAMACIVGLM WLSYFVASFR
LFARTRSMWS FNPETNILLN VPLRGTIVTR PLMESELVIG AVIIRGHLRM
AGHSLGRCDI KDLPKEITVA TSRTLSYYKL GASQRVGTDS GFAAYNRYRI
GNYKLNTDHA GSNDNIALLV Q (SEQ ID NO: 419)

In some aspects, an antigen (e.g., for inducing a humoral immune response) that is useful for the present disclosure is derived from a region of a coronavirus other than the receptor binding domain of a spike protein (S1 protein) and/or other conserved regions (e.g., within the M. and/or N protein). In certain aspects, an EV comprising such an antigen can reduce the risk of antibody dependent enhancement of viral infection.

As described herein, in some aspects, an antigen derived from a coronavirus is expressed in an EV linked to a scaffold moiety (e.g., Scaffold X and/or Scaffold Y). In some aspects, a coronavirus antigen disclosed herein can be linked directly to the surface (e.g., exterior surface) of an EV. As further described elsewhere in the present disclosure, in some aspects, a coronavirus antigen that can be expressed in an EV of the present disclosure comprises a spike protein derived from a coronavirus. In certain aspects, the coronavirus comprises SARS-CoV-1, SARS-CoV-2 (COVID-19), MERS-CoV, or combinations thereof.

In some aspects, a coronavirus spike protein (i.e., antigen) that can be expressed in an EV comprises the entire (i.e., full-length) trimeric protein (i.e., “spike trimer”). Accordingly, in certain aspects, the spike trimer is linked directly to the surface (e.g., exterior surface) of an EV.

In some aspects, a coronavirus spike protein (i.e., antigen) that can be expressed in an EV comprises a monomeric subunit of the trimeric spike protein (i.e., “spike monomer”). In some aspects, the spike monomer is linked directly to the surface (e.g., exterior surface) of an EV. In some aspects, the spike monomer is expressed on the surface (e.g., exterior surface) of an EV linked to a scaffold moiety disclosed herein (e.g., Scaffold X and/or Scaffold Y). In certain aspects, the spike monomer is expressed on the exterior surface of an EV linked to a Scaffold X.

In some aspects, a coronavirus spike protein (i.e., antigen) that can be expressed in an EV comprises one or more subunits of a full-length spike protein (i.e., “exo-split-Spike”). The structure of the coronavirus spike protein, along with its different subunits, is known in the art. See, e.g., Fang Li, Annu Rev Virol 3 (1): 237-261 (September 2016). In some aspects, any of the subunits of a coronavirus spike protein can be expressed in an EV of the present disclosure. As described herein, in some aspects, the one or more subunits of a spike protein comprises a receptor-binding domain (RBD) of the spike protein. In some aspects, the RBD is linked directly to the surface (e.g., exterior surface) of an EV. In some aspects, the RBD is expressed on the surface (e.g., exterior surface) of an EV linked to a scaffold moiety disclosed herein (e.g., Scaffold X and/or Scaffold Y). In certain aspects, the RBD of a coronavirus spike protein is expressed on the exterior surface of an EV linked to a Scaffold X. As will be apparent to those skilled in the art,

As described herein, in some aspects, an EV described herein can express multiple (e.g., two or more) coronavirus antigens (e.g., disclosed herein). In certain aspects, an EV disclosed herein can express a spike protein (e.g., full-length protein or subunit thereof) and a coronavirus antigen comprising a T cell epitope (“T-antigen”) (see, e.g., FIG. 2). In some of these aspects, the spike protein antigen (e.g., receptor-binding domain) can be expressed on exterior surface of the EV while the T-antigen is expressed on the luminal surface of the EV.

Accordingly, in some aspects, an EV disclosed herein comprises: (i) a spike protein antigen (e.g., receptor-binding domain) and (ii) a T-antigen, wherein the spike protein antigen is linked to a Scaffold X (e.g., at the N-terminus) on the exterior surface of the EV, and the T-antigen is linked to a Scaffold X (e.g., at the C-terminus) on the luminal surface of the EV. In some aspects, an EV comprises: (i) a spike protein antigen (e.g., receptor-binding domain) and (ii) a T-antigen, wherein the spike protein antigen is linked to a Scaffold X (e.g., at the N-terminus) on the exterior surface of the EV, and the T-antigen is linked to a Scaffold Y on the luminal surface of the EV. In some aspects, an EV comprises: (i) a spike protein antigen (e.g., receptor-binding domain) and (ii) a T-antigen, wherein the spike protein antigen is linked to a Scaffold X (e.g., at the N-terminus) on the exterior surface of the EV, and the T-antigen is linked directly to the luminal surface of the EV. In some aspects, an EV comprises: (i) a spike protein antigen (e.g., receptor-binding domain) and (ii) a T-antigen, wherein the spike protein antigen is linked directly to the exterior surface of the EV, and the T-antigen is linked to a Scaffold X (e.g., at the C-terminus) on the luminal surface of the EV. In some aspects, an EV comprises (i) a spike protein antigen (e.g., receptor-binding domain) and (ii) a T-antigen, wherein the spike protein antigen is linked directly to the exterior surface of the EV, and the T-antigen is linked to a Scaffold Y on the luminal surface of the EV. In some aspects, an EV comprises (i) a spike protein antigen (e.g., receptor-binding domain) and (ii) a T-antigen, wherein the spike protein antigen is linked directly to the exterior surface of the EV, and the T-antigen is linked directly to the luminal surface of the EV.

In some aspects, an antigen that can be added to an EV to produce the EV-based vaccines described herein a CD8+ T cell epitope of a coronavirus T cell antigen, such as those described in Wang et al., J Virol 78 (11): 5612-8 (June 2004); Wang et al., Blood 104 (1): 200-6 (July 2004); Tsao et al., Biochem Biophys Res Commun 344 (1): 63-71 (May 2006); Lv et al., BMC Immunol 10:61 (December 2009); and Ahmed et al., Viruses 12 (3): 254 (March 2020); Grifoni et al., Cell Host & Microbe 27:671-680 (April 2020); each of which is incorporated herein by reference in its entirety. See also Table 2 (below).

TABLE 2
Exemplary Antigens Comprising T Cell Epitope of Coronavirus
Antigen Sequence
LLLNCLWSV (HCoV-229e, 77-85) (SEQ ID NO: 420)
FKDGIYFAA (SARS-CoV, 83-91) (SEQ ID NO: 421)
LITGRLAAL (HCoV-229e, 881-889) (SEQ ID NO: 422)
LITGRLQSL (SARS-CoV, 978-986) (SEQ ID NO: 423)
ISVVLIFVV (HCoV-229e, 1121-1129) (SEQ ID NO: 424)
FIAGLIAIV (SARS-CoV, 1203-1211) (SEQ ID NO: 425)
GILGFVFTL (influenza virus, 58-66) (SEQ ID NO: 426)
VVFLHVTYV (SEQ ID NO: 427)
RLQSLQTYV (SEQ ID NO: 428)
VLNDILSRL (SEQ ID NO: 429)
VLYENQKQI (SEQ ID NO: 430)
NLNESLIDL (SEQ ID NO: 431)
LLLQYGSFC (SEQ ID NO: 432)
KLPDDFMGC (SEQ ID NO: 433)
ILPDPLKPT (SEQ ID NO: 434)
MIFDNAFNC (SEQ ID NO: 435)
FIFLLFLTL (SEQ ID NO: 436)
VLYQDVNCT (SEQ ID NO: 437)
ITTDNTFV (SEQ ID NO: 438)
RLNEVAKNL (SEQ ID NO: 439)
LLTDDMIAA (SEQ ID NO: 440)
LLQYGSFCT (SEQ ID NO: 441)
ILLCCMTSC (SEQ ID NO: 442)
LLFNKVTLA (SEQ ID NO: 443)
TMNNKSQSV (SEQ ID NO: 444)
ALNTLVKQL (SEQ ID NO: 445)
ELCQNPFFA (SEQ ID NO: 446)
RLNQLESKV (SEQ ID NO: 448)
ILLNKHID (SEQ ID NO: 449)
AFFGMSRIGMEVTPSGTW (SEQ ID NO: 450)
MEVTPSGTWL (SEQ ID NO: 451)
GMSRIGMEV (SEQ ID NO: 452)
ILLNKHIDA (SEQ ID NO: 453)
ALNTPKDHI (SEQ ID NO: 454)
IRQGTDYKHWPQIAQFA (SEQ ID NO: 455)
KHWPQIAQFAPSASAFF (SEQ ID NO: 456)
LALLLLDRL (SEQ ID NO: 457)
LLLDRLNQL (SEQ ID NO: 458)
LLNKHIDAYKTFPPTEPK (SEQ ID NO: 459)
LQLPQGTTL (SEQ ID NO: 460)
AQFAPSASAFFGMSR (SEQ ID NO: 461)
AQFAPSASAFFGMSRIGM (SEQ ID NO: 462)
RRPQGLPNNTASWFT (SEQ ID NO: 463)
YKTFPPTEPKKDKKKK (SEQ ID NO: 464)
GAALQIPFAMQMAYRF (SEQ ID NO: 465)
MAYRENGIGVTQNVLY (SEQ ID NO: 466)
QLIRAAEIRASANLAATK (SEQ ID NO: 467)
QALNTLVKQLSSNFGAI (SEQ ID NO: 470)
VLNDILSRL (SEQ ID NO: 472)
GLIAIVMVTI (SEQ ID NO: 474),
LQYGSFCT (SEQ ID NO: 475),
RLDKVEAEV (SEQ ID NO: 476),
FPRGQGVPI (SEQ ID NO: 447),
LLLLDRINQ (SEQ ID NO: 468),
LLLLDRINQL (SEQ ID NO: 469),
AQFAPSASA (SEQ ID NO: 471),
TTLPKGFYA (SEQ ID NO: 473),
VLQLPQGTTL (SEQ ID NO: 477)
GYQPYRVVVL (SEQ ID NO: 478),
PYRVVVLSF (SEQ ID NO: 479),
LSPRWYFYY (SEQ ID NO: 480)
DSFKEELDKY (SEQ ID NO: 481),
LIDLQELGKY (SEQ ID NO: 482),
GTTLPKGFY (SEQ ID NO: 483),
VTPSGTWLTY (SEQ ID NO: 484)
GSFCTQLNR (SEQ ID NO: 485),
GVVFLHVTY (SEQ ID NO: 486),
AQALNTLVK (SEQ ID NO: 487),
MTSCCSCLK (SEQ ID NO: 488),
ASANLAATK (SEQ ID NO: 489),
SLIDLQELGK (SEQ ID NO: 490),
SVLNDILSR (SEQ ID NO: 491),
TQNVLYENQK (SEQ ID NO: 492),
CMTSCCSCLK (SEQ ID NO: 493),
VQIDRLITGR (SEQ ID NO: 494),
KTFPPTEPK (SEQ ID NO: 495),
KTFPPTEPKK (SEQ ID NO: 496),
ASAFFGMSR (SEQ ID NO: 497),
ATEGALNTPK (SEQ ID NO: 498),
QLPQGTTLPK (SEQ ID NO: 499),
QQQGQTVTK (SEQ ID NO: 500),
QQQQGQTVTK (SEQ ID NO: 501),
SASAFFGMSR (SEQ ID NO: 502),
SQASSRSSSR (SEQ ID NO: 503),
TPSGTWLTY (SEQ ID NO: 504)
FPNITNLCPF (SEQ ID NO: 505),
APHGVVFLHV (SEQ ID NO: 506),
APSASAFFGM (SEQ ID NO: 507)
GAALQIPFAMQMAYR (SEQ ID NO: 508),
GWTFGAGAALQIPFA (SEQ ID NO: 509),
IDRLITGRLQSLQTY (SEQ ID NO: 510),
ISGINASVVNIQKEI (SEQ ID NO: 511),
LDKYFKNHTSPDVDL (SEQ ID NO: 512),
LGDISGINASVVNIQ (SEQ ID NO: 513),
LGFIAGLIAIVMVTI (SEQ ID NO: 514),
LNTLVKQLSSNFGAI (SEQ ID NO: 515),
LQDVVNQNAQALNTL (SEQ ID NO: 516),
LQSLQTYVTQQLIRA (SEQ ID NO: 517),
LQTYVTQQLIRAAEI (SEQ ID NO: 518),
AQKFNGLTVLPPLLT (SEQ ID NO: 519),
PCSFGGVSVITPGTN (SEQ ID NO: 520),
QIPFAMQMAYRENGI (SEQ ID NO: 521),
QQLIRAAEIRASANL (SEQ ID NO: 522),
QTYVTQQLIRAAEIR (SEQ ID NO: 523),
AYRENGIGVTQNVLY (SEQ ID NO: 524),
SSNFGAISSVLNDIL (SEQ ID NO: 525),
TGRLQSLQTYVTQQL (SEQ ID NO: 526),
WLGFIAGLIAIVMVT (SEQ ID NO: 527),
CVNFNFNGLTGTGVL (SEQ ID NO: 528),
DKYFKNHTSPDVDLG (SEQ ID NO: 529),
IDAYKTFPPTEPKKD (SEQ ID NO: 530),
MSRIGMEVTPSGTWL (SEQ ID NO: 531),
NKHIDAYKTFPPTEP (SEQ ID NO: 532),
VLQLPQGTTLPKGFY (SEQ ID NO: 533)
LQIPFAMQM (SEQ ID NO: 534),
RVDFCGKGY (SEQ ID NO: 535)
YEQYIKWPWY (SEQ ID NO: 536)
GRLQSLQTY (SEQ ID NO: 537),
VRFPNITNL (SEQ ID NO: 538)
DVVNQNAQALNTLVKQL (SEQ ID NO: 539)
EAEVQIDRLITGRLQSL (SEQ ID NO: 540)
EIDRLNEVAKNLNESLIDLQELGKYEQY (SEQ ID NO: 541)
EVAKNLNESLIDLQELG (SEQ ID NO: 542)
GAALQIPFAMQMAYRFN (SEQ ID NO: 543)
GAGICASY (SEQ ID NO: 544)
AISSVLNDILSRLDKVE (SEQ ID NO: 545)
GSFCTQLN (SEQ ID NO: 546)
ILSRLDKVEAEVQIDRL (SEQ ID NO: 547)
KGIYQTSN (SEQ ID NO: 548)
AMQMAYRF (SEQ ID NO: 549)
KNHTSPDVDLGDISGIN (SEQ ID NO: 550)
MAYRENGIGVTQNVLYE (SEQ ID NO: 551)
AATKMSECVLGQSKRVD (SEQ ID NO: 552)
PFAMQMAYRENGIGVTQ (SEQ ID NO: 553)
QLIRAAEIRASANLAAT (SEQ ID NO: 554)
QQFGRD (SEQ ID NO: 555)
RASANLAATKMSECVLG (SEQ ID NO: 556)
RLITGRLQSLQTYVTQQ (SEQ ID NO: 557)
SLQTYVTQQLIRAAEIR (SEQ ID NO: 558)
DLGDISGINASVVNIQK (SEQ ID NO: 559)
FFGMSRIGMEVTPSGTW (SEQ ID NO: 560)
GLPNNTASWFTALTQHGK (SEQ ID NO: 561)
GTTLPK (SEQ ID NO: 562)
KHIDAYKTFPPTEPKKDKKK (SEQ ID NO: 563)
YNVTQAFGRRGPEQTQGNF (SEQ ID NO: 564)
KTFPPTEPKKDKKKK (SEQ ID NO: 565)
LLPAAD (SEQ ID NO: 566)
LNKHIDAYKTFPPTEPK (SEQ ID NO: 567)
LPQGTTLPKG (SEQ ID NO: 568)
LPQRQKKQ (SEQ ID NO: 569)
PKGFYAEGSRGGSQASSR (SEQ ID NO: 570)
QFAPSASAFFGMSRIGM (SEQ ID NO: 571)
QGTDYKHW (SEQ ID NO: 572)
QLPQGTTLPKGFYAE (SEQ ID NO: 573)
QLPQGTTLPKGFYAEGSR (SEQ ID NO: 574)
QLPQGTTLPKGFYAEGSRGGSQ (SEQ ID NO: 575)
TFPPTEPK (SEQ ID NO: 576)
SQASSRSS (SEQ ID NO: 577)
SRGGSQASSRSSSRSR (SEQ ID NO: 578)

As described herein, in some aspects, an EV comprising a spike protein antigen and a T-antigen described above can further express one or more additional moieties disclosed herein (e.g., adjuvant, immune modulator, and/or targeting moiety). Additional disclosure relating to such EVs are provided elsewhere in the present disclosure.

In some aspects, an antigen, e.g., a first antigen and/or a second antigen, is expressed on the exterior surface or in the lumen (e.g., on the luminal surface) of the EV. In some aspects, a first and/or second antigen is expressed on the exterior surface or in the luminal surface of the EVs directly connected to the lipid bilayer. In such aspects, the first antigen and/or the second can be linked to a scaffold moiety (e.g., Scaffold X and/or Scaffold Y).

In some aspects, an EVs described herein comprises a first scaffold moiety. In certain aspects, the first antigen is linked to the first scaffold moiety. In other aspects, the second antigen is linked to the first scaffold moiety. In further aspects, both the first antigen and the second antigen are linked to the first scaffold moiety. In some aspects, an EVs further comprises a second scaffold moiety. In certain aspects, the first antigen is linked to the first scaffold moiety, and the second antigen is linked to the second scaffold moiety. In some aspects, the first scaffold moiety and the second scaffold moiety are the same (e.g., both Scaffold X or both Scaffold Y). In other aspects, the first scaffold moiety and the second scaffold moiety are different (e.g., first scaffold moiety is Scaffold X and the second scaffold moiety is Scaffold Y; or first scaffold moiety is Scaffold Y and the second scaffold moiety is Scaffold X).

Non-limiting examples of Scaffold X include: prostaglandin F2 receptor negative regulator (PTGFRN); basigin (BSG); immunoglobulin superfamily member 2 (IGSF2); immunoglobulin superfamily member 3 (IGSF3); immunoglobulin superfamily member 8 (IGSF8); integrin beta-1 (ITGB1), integrin alpha-4 (ITGA4); 4F2 cell-surface antigen heavy chain (SLC3A2); and a class of ATP transporter proteins (ATP1A1, ATP1A2, ATP1A3, ATP1A4, ATP1B3, ATP2B1, ATP2B2, ATP2B3, ATP2B). In certain aspects, Scaffold X is a whole protein. In other aspects, Scaffold X is a protein fragment (e.g., functional fragment).

In other aspects, the scaffold moiety useful for the present disclose, a first scaffold moiety, a second scaffold moiety, and/or a third scaffold moiety, includes a conventional exosome protein, including, but not limiting, tetraspanin molecules (e.g., CD63, CD81, CD9 and others), lysosome-associated membrane protein 2 (LAMP2 and LAMP2B), platelet-derived growth factor receptor (PDGFR), GPI anchor proteins, lactadherin and fragments thereof, peptides that have affinity to any of these proteins or fragments thereof, or any combination thereof.

Non-limiting examples of Scaffold Y include: the myristoylated alanine rich Protein Kinase C substrate (MARCKS) protein; myristoylated alanine rich Protein Kinase C substrate like 1 (MARCKSL1) protein; and brain acid soluble protein 1 (BASP1) protein. In some aspects, Scaffold Y is a whole protein. In certain aspects, Scaffold Y is a protein fragment (e.g., functional fragment).

In some aspects, the first antigen is linked to a first scaffold moiety on the luminal surface of the EVs and the second antigen is in the lumen of the EV. As used herein, when a molecule (e.g., first antigen or second antigen) is described as “in the lumen” of the EV it means that the molecule is not linked to a scaffold moiety described herein. In some aspects, the first antigen is in the lumen of the EV and the second antigen is linked to a first scaffold moiety on the luminal surface of the EV. In such aspects, the first scaffold moiety can be Scaffold X or Scaffold Y.

In some aspects, the first antigen is linked to a first scaffold moiety on the luminal surface of the EV and the second antigen is linked to a second scaffold moiety on the exterior surface of the EV. In some aspects, the second antigen is linked to a first scaffold moiety on the luminal surface of the EV and the first antigen is linked to a second scaffold moiety on the exterior surface of the EV. In these aspects, the first scaffold moiety can be Scaffold Y, and the second scaffold moiety can be Scaffold X. In other aspects, each of the first scaffold moiety and the second scaffold moiety can be Scaffold X.

In some aspects, the first antigen is linked to a first scaffold moiety on the exterior surface of the EVs and the second antigen is linked to a second scaffold moiety on the luminal surface of the EV. In other aspects, the second antigen is linked to a first scaffold moiety on the exterior surface of the EVs and the first antigen is linked to a second scaffold moiety on the luminal surface of the EV. In such aspects, the first scaffold moiety is Scaffold X, and the second scaffold moiety is Scaffold Y; or each of the first scaffold moiety and the second scaffold moiety is Scaffold X.

In some aspects, the first antigen is in the lumen of the EVs and the second antigen is in the lumen of the EV.

In some aspects, the first antigen is linked to a first scaffold moiety on the exterior surface of the EVs and the second antigen is linked to a second scaffold moiety on the exterior surface of the EV. In other aspects, the second antigen is linked to a first scaffold moiety on the exterior surface of the EVs and the first antigen is linked to a second scaffold moiety on the exterior surface of the EV. In some aspects, the first scaffold moiety and the second scaffold moiety are Scaffold X.

In some aspects, the first antigen is linked to a first scaffold moiety on the exterior surface of the EVs and the second antigen is in the lumen of the EV. In some aspects, the first antigen is in the lumen of the EVs and the second antigen is linked to a first scaffold moiety on the exterior surface of the EV. In such aspects, the first scaffold moiety can be Scaffold X.

In some aspects, the first antigen is linked to a first scaffold moiety on the exterior surface of the EV and the second antigen is linked to the first scaffold moiety on the luminal surface of the EV. In other aspects, the first antigen is linked to a first scaffold moiety on the luminal surface of the EV and the second antigen is linked to the first scaffold moiety on the exterior surface of the EV. In these aspects, the first scaffold moiety can be Scaffold X.

Non-limiting examples of specific aspects include EVs comprising (i) a first antigen and (ii) a second antigen, wherein:
(a) the first antigen is linked to a first Scaffold Y on the luminal surface of the EV and the second antigen is linked to a second Scaffold Y on the luminal surface of the EV;
(b) the first antigen is linked to a Scaffold Y on the luminal surface of the EV and the second antigen is in the lumen of the EV not linked to any scaffold moiety;
(c) the first antigen is in the lumen of the EV not linked to any scaffold moiety, and the second antigen is linked to a Scaffold Y on the luminal surface of the EV;
(d) the first antigen is linked to a Scaffold Y on the luminal surface of the EV and the second antigen is linked to a Scaffold X on the exterior surface of the EV;
(e) the first antigen is in the lumen of the EV not linked to any scaffold moiety, and the second antigen is linked to a Scaffold X on the exterior surface of the EV;
(f) the first antigen is linked to a Scaffold Y on the luminal surface of the EV and the second antigen is linked to a Scaffold X on the luminal surface of the EV;
(g) the first antigen is in the lumen of the EV not linked to any scaffold moiety, and the second antigen is linked to a Scaffold X on the luminal surface of the EV;
(h) the first antigen is linked to a Scaffold X on the luminal surface of the EV and the second antigen is linked to the Scaffold X on the exterior surface of the EV;
(i) the first antigen is linked to a first Scaffold X on the exterior surface of the EV and the second antigen is linked to a second Scaffold X on the exterior surface of the EV;
(j) the first antigen is linked to a Scaffold X on the exterior surface of the EV and the second antigen is linked to a Scaffold Y on the luminal surface of the EV;
(k) the first antigen is linked to a Scaffold X on the exterior surface of the EV and the second antigen is in the lumen of the EV not linked to any scaffold moiety;
(l) the first antigen is linked to a Scaffold X on the exterior surface of the EV and the second antigen is linked to the Scaffold X on the luminal surface of the EV;
(m) the first antigen is linked to a first Scaffold X on the luminal surface of the EV and the second antigen is linked to a second Scaffold X on the luminal surface of the EV;
(n) the first antigen is linked to a Scaffold X on the luminal surface of the EV and the second antigen is linked to a Scaffold Y on the luminal surface of the EV;
(o) the first antigen is linked to a Scaffold X on the luminal surface of the EV and the second antigen is in the lumen of the EV not linked to any scaffold moiety;
(p) the first antigen is linked to a first Scaffold X on the exterior surface of the EV and the second antigen is linked to a second Scaffold X on the luminal surface of the EV;
(q) the first antigen is linked to a first Scaffold X on the luminal surface of the EV and the second antigen is linked to a second Scaffold X on the exterior surface of the EV;
(r) the first antigen is in the lumen of the EV not linked to any scaffold moiety, and the second antigen is in the lumen of the EV not linked to any scaffold moiety;
(s) the first antigen is linked directly to the luminal surface of the EV and the second antigen is linked directly to the luminal surface of the EV;
(t) the first antigen is linked directly to the luminal surface of the EV and the second antigen is in the lumen of the EV;
(u) the first antigen is linked directly to the luminal surface of the EV and the second antigen is linked to a Scaffold Y on the luminal surface of the EV;
(v) the first antigen is linked directly to the luminal surface of the EV and the second antigen is linked to a Scaffold X on the luminal surface of the EV;
(w) the first antigen is linked directly to the luminal surface of the EV and the second antigen is linked directly to the exterior of the EV;
(x) the first antigen is linked directly to the luminal surface of the EV and the second antigen is linked to a Scaffold X on the exterior of the EV;
(y) the antigen is linked to a Scaffold Y on the luminal surface of the EV and the second antigen is linked directly to the luminal surface of the EV;
(z) the first antigen is linked to a Scaffold Y on the luminal surface of the EV and the second antigen is linked directly to the exterior of the EV;
(aa) the first antigen is linked to a Scaffold X on the luminal surface of the EV and the second antigen is linked directly to the luminal surface of the EV;
(bb) the first antigen is linked to a Scaffold X on the luminal surface of the EV and the second antigen is linked directly to the exterior of the EV,
(cc) the first antigen is in the lumen of the EV and the second antigen is linked directly to the luminal surface of the EV; or
(dd) the first antigen is in the lumen of the EV and the second antigen is linked directly to the exterior of the EV.

In some aspects, an EV of the present disclosure comprises (i) a first antigen and (ii) a second antigen, wherein the first antigen is linked to a first Scaffold Y on the luminal surface of the EV and the second antigen is linked to a second Scaffold Y on the luminal surface of the EV. In some aspects, an EV of the present disclosure comprises (i) a first antigen and (ii) a second antigen, wherein the first antigen is linked to a Scaffold Y on the luminal surface of the EV and the second antigen is in the lumen of the EV not linked to any scaffold moiety. In some aspects, an EV of the present disclosure comprises (i) a first antigen and (ii) a second antigen, wherein the first antigen is in the lumen of the EV not linked to any scaffold moiety, and the second antigen is linked to a Scaffold Y on the luminal surface of the EV. In some aspects, an EV of the present disclosure comprises (i) a first antigen and (ii) a second antigen, wherein the first antigen is linked to a Scaffold Y on the luminal surface of the EV and the second antigen is linked to a Scaffold X on the exterior surface of the EV. In some aspects, an EV of the present disclosure comprises (i) a first antigen and (ii) a second antigen, wherein the first antigen is in the lumen of the EV not linked to any scaffold moiety, and the second antigen is linked to a Scaffold X on the exterior surface of the EV. In some aspects, an EV of the present disclosure comprises (i) a first antigen and (ii) a second antigen, wherein the first antigen is linked to a Scaffold Y on the luminal surface of the EV and the second antigen is linked to a Scaffold X on the luminal surface of the EV. In some aspects, an EV of the present disclosure comprises (i) a first antigen and (ii) a second antigen, wherein the first antigen is in the lumen of the EV not linked to any scaffold moiety, and the second antigen is linked to a Scaffold X on the luminal surface of the EV. In some aspects, an EV of the present disclosure comprises (i) a first antigen and (ii) a second antigen, wherein the first antigen is linked to a Scaffold X on the luminal surface of the EV and the second antigen is linked to the Scaffold X on the exterior surface of the EV. In some aspects, an EV of the present disclosure comprises (i) a first antigen and (ii) a second antigen, wherein the first antigen is linked to a first Scaffold X on the exterior surface of the EV and the second antigen is linked to a second Scaffold X on the exterior surface of the EV. In some aspects, an EV of the present disclosure comprises (i) a first antigen and (ii) a second antigen, wherein the first antigen is linked to a Scaffold X on the exterior surface of the EV and the second antigen is linked to a Scaffold Y on the luminal surface of the EV. In some aspects, an EV of the present disclosure comprises (i) a first antigen and (ii) a second antigen, wherein the first antigen is linked to a Scaffold X on the exterior surface of the EV and the second antigen is in the lumen of the EV not linked to any scaffold moiety. In some aspects, an EV of the present disclosure comprises (i) a first antigen and (ii) a second antigen, wherein the first antigen is linked to a Scaffold X on the exterior surface of the EV and the second antigen is linked to the Scaffold X on the luminal surface of the EV. In some aspects, an EV of the present disclosure comprises (i) a first antigen and (ii) a second antigen, wherein the first antigen is linked to a first Scaffold X on the luminal surface of the EV and the second antigen is linked to a second Scaffold X on the luminal surface of the EV. In some aspects, an EV of the present disclosure comprises (i) a first antigen and (ii) a second antigen, wherein the first antigen is linked to a Scaffold X on the luminal surface of the EV and the second antigen is linked to a Scaffold Y on the luminal surface of the EV. In some aspects, an EV of the present disclosure comprises (i) a first antigen and (ii) a second antigen, wherein the first antigen is linked to a Scaffold X on the luminal surface of the EV and the second antigen is in the lumen of the EV not linked to any scaffold moiety. In some aspects, an EV of the present disclosure comprises (i) a first antigen and (ii) a second antigen, wherein the first antigen is linked to a first Scaffold X on the exterior surface of the EV and the second antigen is linked to a second Scaffold X on the luminal surface of the EV. In some aspects, an EV of the present disclosure comprises (i) a first antigen and (ii) a second antigen, wherein the first antigen is linked to a first Scaffold X on the luminal surface of the EV and the second antigen is linked to a second Scaffold X on the exterior surface of the EV. In some aspects, an EV of the present disclosure comprises (i) a first antigen and (ii) a second antigen, wherein the first antigen is in the lumen of the EV not linked to any scaffold moiety, and the second antigen is in the lumen of the EV not linked to any scaffold moiety. In some aspects, an EV comprises (i) a first antigen and (ii) a second antigen, wherein the first antigen is linked directly to the luminal surface of the EV, and the second antigen is linked directly to the luminal surface of the EV. In some aspects, an EV comprises (i) a first antigen and (ii) a second antigen, wherein the first antigen is linked directly to the luminal surface of the EV, and the second antigen is in the lumen of the EV. In some aspects, an EV comprises (i) a first antigen and (ii) a second antigen, wherein the first antigen is linked directly to the luminal surface of the EV, and the second antigen is linked to a Scaffold Y on the luminal surface of the EV. In some aspects, an EV comprises (i) a first antigen and (ii) a second antigen, wherein the first antigen is linked directly to the luminal surface of the EV, and the second antigen is linked to a Scaffold X on the luminal surface of the EV. In some aspects, an EV comprises (i) a first antigen and (ii) a second antigen, wherein the first antigen is linked directly to the luminal surface of the EV, and the second antigen is linked directly to the exterior of the EV. In some aspects, an EV comprises (i) a first antigen and (ii) a second antigen, wherein the first antigen is linked directly to the luminal surface of the EV, and the second antigen is linked to a Scaffold X on the exterior of the EV. In some aspects, an EV comprises (i) a first antigen and (ii) a second antigen, wherein the first antigen is linked to a Scaffold Y on the luminal surface of the EV, and the second antigen is linked directly to the luminal surface of the EV. In some aspects, an EV comprises (i) a first antigen and (ii) a second antigen, wherein the first antigen is linked to a Scaffold Y on the luminal surface of the EV, and the second antigen is linked directly to the exterior of the EV. In some aspects, an EV comprises (i) a first antigen and (ii) a second antigen, wherein the first antigen is linked to a Scaffold X on the luminal surface of the EV, and the second antigen is linked directly to the luminal surface of the EV. In some aspects, an EV comprises (i) a first antigen and (ii) a second antigen, wherein the first antigen is linked to a Scaffold X on the luminal surface of the EV, and the second antigen is linked directly to the exterior of the EV. In some aspects, an EV comprises (i) a first antigen and (ii) a second antigen, wherein the first antigen is in the lumen of the EV, and the second antigen is linked directly to the luminal surface of the EV. In some aspects, an EV comprises (i) a first antigen and (ii) a second antigen, wherein the first antigen is in the lumen of the EV, and the second antigen is linked directly to the exterior of the EV.

### II.B Adjuvants

As described supra, EVs of the present disclosure can comprise an adjuvant (e.g., in combination with an antigen and/or other payloads disclosed herein). In some aspects, an EV disclosed herein comprises multiple adjuvants. In certain aspects, each of the multiple adjuvants is different. In some aspects, an EV disclosed herein comprises at least 2, 3, 4, 5, 6, 7, 8, 9, 10 or more different adjuvants. As disclosed herein, an adjuvant can be linked to a surface of an EV using a scaffold moiety (e.g., Scaffold X and/or Scaffold Y). In certain aspects, an adjuvant can be directly linked (i.e., without the use of a scaffold moiety) to a surface of an EV. In some aspects, an adjuvant can be in the lumen of the EV.

In some aspects, an EV comprises the one or more adjuvants in combination with one or more additional payloads (e.g., antigen, and/or immune modulator). For example, in certain aspects, an EV described herein comprises an antigen and an adjuvant, wherein the adjuvant is present in the EV prior to the addition of the antigen. In such aspects, the adjuvant can be introduced into a producer cell when producing the EV (e.g., base EV). In some aspects, the adjuvant can be added to the EVs after being isolated from the producer cells. In such aspects, the adjuvant can be added to the isolated EVs before adding the antigen. In some aspects, the adjuvant is added to the EV after adding the antigen. In some aspects, the adjuvant is added to the EV together with the antigen. In some aspects, an EV can comprise one or more additional moieties (e.g., targeting moieties). For instance, in certain aspects, an EV disclosed herein can comprise (i) one or more additional adjuvants, (ii) one or more additional payloads (e.g., antigen and/or immune modulator), and (iii) one or more targeting moieties. As described herein, in some aspects, any of the adjuvants, additional payloads, and/or targeting moieties can be present in the EV prior to the addition of any of the other moieties of the EV (e.g., the adjuvant, immune modulator, and/or targeting moiety can be present in the EV prior to the addition of the antigen). In certain aspects, the adjuvants, additional payloads, and/or targeting moieties can be added to the EV concurrently.

As used herein, the term “adjuvant” refers to any substance that enhances the therapeutic effect of the payload (e.g., increasing an immune response to the antigen). Accordingly, EVs described herein comprising an adjuvant are capable of increasing an immune response, e.g., to an antigen, by at least about 5%, at least about 10%, at least about 20%, at least about 30%, at least about 40%, at least about 50%, at least about 60%, at least about 70%, at least about 80%, at least about 90%, at least about 100%, at least about 250%, at least about 500%, at least about 750%, at least about 1,000% or more or more, compared to a reference (e.g., corresponding EV without the adjuvant or a non-EV delivery vehicle comprising an antigen alone or in combination with the adjuvant). In some aspects, incorporating an adjuvant disclosed herein to an EV can increase an immune response, e.g., to an antigen, by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to a reference (e.g., corresponding EV comprising the antigen alone or a non-EV delivery vehicle comprising an antigen alone or in combination with the adjuvant).

In some aspects, EVs described herein (e.g., those that can be used as vaccines) comprises a single adjuvant. In some aspects, an EV disclosed herein comprises multiple adjuvants. In such aspects, some or all of the multiple adjuvants: (i) can be introduced into the producer cells, such that the base EVs are produced comprising the adjuvants; (ii) can be added to the EVs are they are isolated from the producer cells but prior to the addition of the antigen; (iii) can be added to the EVs after the addition of the antigen; (iv) can be added to the EVs together with the antigen; or (v) any combination thereof.

In some aspects, each of the multiple adjuvants is different. In some aspects, an EV disclosed herein comprises at least 2, 3, 4, 5, 6, 7, 8, 9, 10 or more different adjuvants. As disclosed herein, an adjuvant can be linked to a surface of an EV using a scaffold moiety (e.g., Scaffold X and/or Scaffold Y). In certain aspects, an adjuvant can be directly linked (i.e., without the use of a scaffold moiety) to a surface of an EV. In some aspects, an adjuvant can be in the lumen of the EV.

Non-limiting examples of adjuvants that can be used with the present disclosure include: Stimulator of Interferon Genes (STING) agonist, a toll-like receptor (TLR) agonist, an inflammatory mediator, RIG-I agonists, alpha-gal-cer (NKT agonist), heat shock proteins (e.g., HSP65 and HSP70), C-type lectin agonists (e.g., beta glucan (Dectin 1), chitin, and curdlan), and combinations thereof. Additional examples of adjuvants that can be used with the EVs described herein are provided throughout the present disclosure.

In some aspects, the adjuvant is a TLR9 agonist. In some aspects, the TLR9 agonist comprises a CpG oligonucleotide. As used herein, the term “CpG oligonucleotide” (CpG ODN) refers to a short synthetic single-stranded nucleic acid molecules that contain unmethylated CpG dinucleotides in particular sequence contexts (CpG motifs). There are three major classes of CpG ODNs: Class A (Type D), Class B (Type K), and Class C. In some aspects, the adjuvant is a CpG-A ODN. “CpG-A” ODNs are characterized by a phosphodiester (PO) central CpG-containing palindromic motif and a phosphorothioated (PS)-modified 3′ poly-G string. They induce high IFN-α production from pDCs but are weak stimulators of TLR9-dependent NF-κB signaling and pro-inflammatory cytokine (e.g. IL-6) production. In some aspects, the adjuvant is a CpG-B ODN. “CpG-B” ODNs contain a full PS backbone with one or more CpG dinucleotides. They strongly activate B cells and TLR9-dependent NF-κB signaling but weakly stimulate IFN-α secretion. In some aspects, the adjuvant is a CpG-C ODN. “CpG-C” ODNs combine features of both classes A and B. They contain a complete PS backbone and a CpG-containing palindromic motif. C-Class CpG ODNs induce strong IFN-α production from pDC as well as B cell stimulation.

In some aspects, the adjuvant is a TLR4 agonist. In certain aspects, the TLR4 agonist comprises monophosphoryl lipid A (MPLA), e.g., a derivative of lipid A from Salmonella minnesota R595 lipopolysaccharide (LPS or endotoxin).

In some aspects, incorporating an adjuvant (e.g., such as those disclosed herein) to an EV can broaden an immune response induced by the EV. As used herein, to “broaden an immune response” refers to enhancing the diversity of an immune response. In some aspects, the diversity of an immune response can be enhanced through epitope spreading (i.e., inducing and/or increasing an immune response (cellular and/or humoral immune response) against a greater number/variety of epitopes on an antigen). In some aspects, the diversity of an immune response can be enhanced through the production of different and/or multiple antibody isotypes (e.g., IgG, IgA, IgD, IgM, and/or IgE).

In some aspects, an adjuvant (e.g., such as those disclosed herein) can also help regulate the type of immune response induced by the EV. For example, in some aspects, incorporating an adjuvant to an EV can help drive an immune response towards a more Th1 phenotype. As used herein, a “Th1” immune response is generally characterized by the production of IFN-γ, which can activate the bactericidal activities of innate cells (e.g., macrophages), help induce B cells to make opsonizing (marking for phagocytosis) and complement-fixing antibodies, and/or lead to cell-mediated immunity (i.e., not mediated by antibodies). In general, Th1 responses are more effective against intracellular pathogens (viruses and bacteria that are inside host cells).

In some aspects, incorporating an adjuvant to an EV can help drive an immune response towards a more Th2 phenotype. As used herein, a “Th2” immune response can be characterized by the release of certain cytokines, such as IL-5 (induces eosinophils in the clearance of parasites) and IL-4 (facilitates B cell isotype switching). In general, Th2 responses are more effective against extracellular bacteria, parasites including helminths and toxins.

In some aspects, incorporating an adjuvant to an EV can help drive an immune response towards a more Th17 phenotype. As used herein, a “Th17” immune response is mediated by Th17 cells. As used herein, “Th17 cells” refer to a subset of CD4+ T cells characterized by the production of pro-inflammatory cytokines, such as IL-17A, IL-17F, IL-21, IL-22, and granulocyte-macrophage colony-stimulating factor (GM-CSF). Th17 cells are generally thought to play an important role in host defense against infection, by recruiting neutrophils and macrophages to infected tissues.

In some aspects, incorporating an adjuvant to an EV can help drive an immune response towards a more cellular immune response (e.g., T-cell mediated). In some aspects, incorporating an adjuvant to an EV can help drive an immune response towards a more humoral immune response (e.g., antibody-mediated).

In some aspects, an adjuvant induces the activation of a cytosolic pattern recognition receptor. In some aspects, such adjuvants are viral nucleic acid mimetics. Not to be bound by any one theory, EVs comprising such adjuvants are capable of preferentially inducing Th1 (e.g., IFN) and/or antibody-mediated immune responses. Non-limiting examples of cytosolic pattern recognition receptor includes: stimulator of interferon genes (STING), retinoic acid-inducible gene 1 (RIG-1), Melanoma Differentiation-Associated protein 5 (MDA5), Nucleotide-binding oligomerization domain, Leucine rich Repeat and Pyrin domain containing (NLRP), inflammasomes, or combinations thereof. In certain aspects, an adjuvant is a STING agonist. Stimulator of Interferon Genes (STING) is a cytosolic sensor of cyclic dinucleotides that is typically produced by bacteria. Upon activation, it leads to the production of type I interferons (e.g., IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin)) and initiates an immune response. In certain aspects, the STING agonist comprises a cyclic dinucleotide STING agonist or a non-cyclic dinucleotide STING agonist. As described herein, in some aspects, the STING agonist is loaded in the lumen of the EV. In some aspects, such EVs are referred to herein as “exoSTING.” Non-limiting examples of exoSTING are provided in International Publication No. WO 2019183578A1, which is herein incorporated by reference in its entirety. Further disclosures of useful STING agonists are also provided throughout the present disclosure.

Cyclic purine dinucleotides such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, are known to stimulate or enhance an immune or inflammation response in a patient. The CDNs can have 2′2′, 2′3′, 2′5′, 3′3′, or 3′5′ bonds linking the cyclic dinucleotides, or any combination thereof.

Cyclic purine dinucleotides can be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides can be modified analogues. Any suitable modification known in the art can be used, including, but not limited to, phosphorothioate, biphosphorothioate, fluorinate, and difluorinate modifications.

Non cyclic dinucleotide agonists can also be used, such as 5,6-Dimethylxanthenone-4-acetic acid (DMXAA), or any other non-cyclic dinucleotide agonist known in the art.

Non-limiting examples of STING agonists that can be used with the present disclosure include: DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMP, CAIM(PS)2, 3′3′-cAIMP, 3′3′-cAIMPdFSH, 2′2′-cGAMP, 2′3′-cGAM(PS)2, 3′3′-cGAMP, and combinations thereof. Non-limiting examples of the STING agonists can be found at U.S. Pat. No. 9,695,212, WO 2014/189805 A1, WO 2014/179335 A1, WO 2018/100558 A1, U.S. Pat. No. 10,011,630 B2, WO 2017/027646 A1, WO 2017/161349 A1, and WO 2016/096174 A1, each of which is incorporated by reference in its entirety.

In some aspects, the STING agonist useful for the present disclosure comprises the compound or a pharmaceutically acceptable salt thereof. See WO 2016/096174 A1, which is incorporated herein by reference in its entirety.

In some aspects, the STING agonist useful for the present disclosure comprises a compound described in WO 2014/093936, WO 2014/189805, WO 2015/077354, Cell reports 11, 1018-1030 (2015), WO 2013/185052, Sci. Transl. Med. 283,283ra52 (2015), WO 2014/189806, WO 2015/185565, WO 2014/179760, WO 2014/179335, WO 2015/017652, WO 2016/096577, WO 2016/120305, WO 2016/145102, WO 2017/027646, WO 2017/075477, WO 2017/027645, WO 2018/100558, WO 2017/175147, WO 2017/175156, each of which is incorporated herein by reference in its entirety.

In some aspects, the STING agonist useful for the present disclosure is CL606, CL611, CL602, CL655, CL604, CL609, CL614, CL656, CL647, CL626, CL629, CL603, CL632, CL633, CL659, or a pharmaceutically acceptable salt thereof. In some aspects, the STING agonist useful for the present disclosure is CL606 or a pharmaceutically acceptable salt thereof. In some aspects, the STING agonist useful for the present disclosure is CL611 or a pharmaceutically acceptable salt thereof. In some aspects, the STING agonist useful for the present disclosure is CL602 or a pharmaceutically acceptable salt thereof. In some aspects, the STING agonist useful for the present disclosure is CL655 or a pharmaceutically acceptable salt thereof. In some aspects, the STING agonist useful for the present disclosure is CL604 or a pharmaceutically acceptable salt thereof. In some aspects, the STING agonist useful for the present disclosure is CL609 or a pharmaceutically acceptable salt thereof. In some aspects, the STING agonist useful for the present disclosure is CL614 or a pharmaceutically acceptable salt thereof. In some aspects, the STING agonist useful for the present disclosure is CL656 or a pharmaceutically acceptable salt thereof. In some aspects, the STING agonist useful for the present disclosure is CL647 or a pharmaceutically acceptable salt thereof. In some aspects, the STING agonist useful for the present disclosure is CL626 or a pharmaceutically acceptable salt thereof. In some aspects, the STING agonist useful for the present disclosure is CL629 or a pharmaceutically acceptable salt thereof. In some aspects, the STING agonist useful for the present disclosure is CL603 or a pharmaceutically acceptable salt thereof. In some aspects, the STING agonist useful for the present disclosure is CL632 or a pharmaceutically acceptable salt thereof. In some aspects, the STING agonist useful for the present disclosure is CL633 or a pharmaceutically acceptable salt thereof. In some aspects, the STING agonist useful for the present disclosure is CL659 or a pharmaceutically acceptable salt thereof.

In some aspects, the EV comprises a cyclic dinucleotide STING agonist and/or a non-cyclic dinucleotide STING agonist. In some aspects, when several cyclic dinucleotide STING agonist are present on an EV disclosed herein, such STING agonists can be the same or they can be different. In some aspects, when several non-cyclic dinucleotide STING agonist are present, such STING agonists can be the same or they can be different. In some aspects, an EV composition of the present disclosure can comprise two or more populations of EVs wherein each population of EVs comprises a different STING agonist or combination thereof.

The STING agonists can also be modified to increase encapsulation (i.e., loading) of the agonist in an extracellular vesicle or EV (e.g., cither unbound in the lumen). In some aspects, the STING agonists are linked to a scaffold moiety, e.g., Scaffold Y. In certain aspects, the modification allows better expression of the STING agonist on the exterior surface of the EV (e.g., linked to a scaffold moiety disclosed herein, e.g., Scaffold X). This modification can include the addition of a lipid binding tag by treating the agonist with a chemical or enzyme, or by physically or chemically altering the polarity or charge of the STING agonist. The STING agonist can be modified by a single treatment, or by a combination of treatments, e.g., adding a lipid binding tag only, or adding a lipid binding tag and altering the polarity. The previous example is meant to be a non-limiting illustrative instance. It is contemplated that any combination of modifications can be practiced. The modification can increase encapsulation (i.e., loading) of the agonist in the EV by between about 2-fold and about 10,000 fold, between about 10-fold and about 1,000 fold, or between about 100-fold and about 500-fold compared to encapsulation (i.e., loading) of an unmodified agonist. The modification can increase encapsulation (i.e., loading) of the agonist in the EV by at least about 2-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about, 2000-fold, at least about 3,000-fold, at least about 4,000-fold, at least about 5,000-fold, at least about 6,000-fold, at least about 7,000-fold, at least about 8,000-fold, at least about 9,000-fold, or at least about 10,000-fold compared to encapsulation (i.e., loading) of an unmodified agonist.

In some aspects, STING agonists can be modified to allow for better expression of the agonists on the surface of the EV (e.g., exterior and/or luminal surface of the EV (e.g., linked to a scaffold moiety disclosed herein, e.g., Scaffold X and/or Scaffold Y)). Any of the modifications described above can be used. The modification can increase expression of the agonist in the EV, e.g., on the surface and/or luminal surface of the exosome, by about between 2-fold and 10,000-fold, about between 10-fold and 1,000-fold, or about between 100-fold and 500-fold compared to corresponding expression of an unmodified agonist. The modification can increase expression of the agonist on the exterior surface of the EV by at least about 2-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, or at least about 10,000-fold compared to expression of an unmodified agonist. The modification can increase expression of the agonist on the luminal surface of the EV by at least about 2-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, or at least about 10,000-fold compared to expression of an unmodified agonist.

The concentration of the STING agonist associated with the EV can be about 0.01 μM to about 1000 μM. The concentration of the associated STING agonist can be between about 0.01-0.05 μM, between about 0.05-0.1 μM, between about 0.1-0.5 μM, between about 0.5-1 μM, between about 1-5 μM, between about 5-10 μM, between about 10-15 μM, between about 15-20 μM, between about 20-25 μM, between about 25-30 μM, between about 30-35 μM, between about 35-40 μM, between about 45-50 μM, between about 55-60 μM, between about 65-70 μM, between about 70-75 μM, between about 75-80 μM, between about 80-85 μM, between about 85-90 μM, between about 90-95 μM, between about 95-100 μM, between about 100-150 μM, between about 150-200 μM, between about 200-250 μM, between about 250-300 μM, between about 300-350 μM, between about 250-400 μM, between about 400-450 μM, between about 450-500 μM, between about 500-550 μM, between about 550-600 μM, between about 600-650 μM, between about 650-700 μM, between about 700-750 μM, between about 750-800 μM, between about 800-850 μM, between about 805-90 μM, between about 900-950 μM, or between about 950-1000 μM. The concentration of the associated STING agonist can be equal to or greater than about 0.01 μM, about 0.1 μM, about 0.5 μM, about 1 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 950 μM, or about 1,000 μM.

In some aspects, an adjuvant is a TLR agonist. Non-limiting examples of TLR agonists include: TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly (I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), and combinations thereof. Non-limiting examples of TLR agonists can be found at WO2008115319A2, US20130202707A1, US20120219615A1, US20100029585A1, WO2009030996A1, WO2009088401A2, and WO2011044246A1, each of which are incorporated by reference in its entirety. Not to be bound by any one theory, in some aspects, EVs comprising a TLR agonist as an adjuvant is capable of preferentially inducing a Th1 and/or antibody-mediated immune response.

In some aspects, an adjuvant is an inflammatory mediator.

In some aspects, an adjuvant comprises an aluminum-containing adjuvant (also referred to herein as “alum”). In some aspects, an adjuvant comprises an aluminum salt. In certain aspects, the aluminum salt is aluminum hydroxide. Not to be bound by any one theory, in some aspects, an EV comprising an aluminum salt as an adjuvant is capable of mediating damage-associated molecular pattern (DAMP) (e.g., NLRP3) activation of antigen-presenting cells (APCs). In certain aspects, such EVs are capable of preferentially inducing a Th2-cell and/or antibody-mediated immune response. In some aspects, the aluminum-containing adjuvant can be used in combination with one or more additional adjuvants, such as CpG.

In some aspects, an adjuvant that can be used with the EVs of the present disclosure comprises emulsions (water-in-oil). In certain aspects, the emulsions include MF59 and AS03. Not to be bound by any one theory, in some aspects, an EV comprising an emulsion as an adjuvant is capable of enhancing APC antigen uptake. In certain aspects, such EVs are capable of inducing robust neutralizing antibodies. In some aspects, such EVs are useful for inducing both Th1 and Th2-mediated immune responses. In some aspects, any suitable adjuvants known in the art can be used with the present disclosure (e.g., AS04 and AS01).

In some aspects, one or more antigens are expressed on the exterior surface or in the lumen (e.g., on the luminal surface) of the EV. In some aspects, an adjuvant is expressed on the exterior surface or in the luminal surface of the EVs directly connected to the lipid bilayer. In such aspects, the antigen, e.g., a first antigen and/or a second antigen, and/or the adjuvant can be linked to a scaffold moiety (e.g., Scaffold X and/or Scaffold Y).

In some aspects, an EVs described herein comprises a first scaffold moiety. In certain aspects, the antigen is linked to the first scaffold moiety. In other aspects, the adjuvant is linked to the first scaffold moiety. In further aspects, both the antigen and the adjuvant are linked to the first scaffold moiety. In some aspects, an EVs further comprises a second scaffold moiety. In certain aspects, the antigen is linked to the first scaffold moiety, and the adjuvant is linked to the second scaffold moiety. In some aspects, the first scaffold moiety and the second scaffold moiety are the same (e.g., both Scaffold X or both Scaffold Y). In other aspects, the first scaffold moiety and the second scaffold moiety are different (e.g., first scaffold moiety is Scaffold X and the second scaffold moiety is Scaffold Y; or first scaffold moiety is Scaffold Y and the second scaffold moiety is Scaffold X).

Non-limiting examples of Scaffold X include: prostaglandin F2 receptor negative regulator (PTGFRN); basigin (BSG); immunoglobulin superfamily member 2 (IGSF2); immunoglobulin superfamily member 3 (IGSF3); immunoglobulin superfamily member 8 (IGSF8); integrin beta-1 (ITGB1); integrin alpha-4 (ITGA4); 4F2 cell-surface antigen heavy chain (SLC3A2); and a class of ATP transporter proteins (ATP1A1, ATP1A2, ATP1A3, ATP1A4, ATP1B3, ATP2B1, ATP2B2, ATP2B3, ATP2B). In certain aspects, Scaffold X is a whole protein. In other aspects, Scaffold X is a protein fragment (e.g., functional fragment).

In other aspects, the scaffold moiety useful for the present disclose, a first scaffold moiety, a second scaffold moiety, and/or a third scaffold moiety, includes a conventional exosome protein, including, but not limiting, tetraspanin molecules (e.g., CD63, CD81, CD9 and others), lysosome-associated membrane protein 2 (LAMP2 and LAMP2B), platelet-derived growth factor receptor (PDGFR), GPI anchor proteins, lactadherin and fragments thereof, peptides that have affinity to any of these proteins or fragments thereof, or any combination thereof.

Non-limiting examples of Scaffold Y include: the myristoylated alanine rich Protein Kinase C substrate (MARCKS) protein; myristoylated alanine rich Protein Kinase C substrate like 1 (MARCKSL1) protein; and brain acid soluble protein 1 (BASP1) protein. In some aspects, Scaffold Y is a whole protein. In certain aspects, Scaffold Y is a protein fragment (e.g., functional fragment).

In some aspects, the antigen, e.g., derived from a coronavirus, e.g., a SARS-CoV-1 and/or SARS-CoV-2 (COVID-19) virus, is linked to a first scaffold moiety on the luminal surface of the EVs and the adjuvant is in the lumen of the EV. In some aspects, the antigen, e.g., derived from a coronavirus, e.g., a SARS-CoV-1 and/or SARS-CoV-2 (COVID-19) virus, is in the lumen of the EV and the adjuvant is linked to a first scaffold moiety on the luminal surface of the EV. In such aspects, the first scaffold moiety can be Scaffold X or Scaffold Y.

In some aspects, the antigen, e.g., derived from a coronavirus, e.g., a SARS-CoV-1 and/or SARS-CoV-2 (COVID-19) virus, is linked to a first scaffold moiety on the luminal surface of the EV and the adjuvant is linked to a second scaffold moiety on the exterior surface of the EV. In some aspects, the adjuvant is linked to a first scaffold moiety on the luminal surface of the EV and the antigen, e.g., derived from a coronavirus, e.g., a SARS-CoV-1 and/or SARS-CoV-2 (COVID-19) virus, is linked to a second scaffold moiety on the exterior surface of the EV. In these aspects, the first scaffold moiety can be Scaffold Y, and the second scaffold moiety can be Scaffold X. In other aspects, each of the first scaffold moiety and the second scaffold moiety can be Scaffold X.

In some aspects, the antigen, e.g., derived from a coronavirus, e.g., a SARS-CoV-1 and/or SARS-CoV-2 (COVID-19) virus, is linked to a first scaffold moiety on the exterior surface of the EVs and the adjuvant is linked to a second scaffold moiety on the luminal surface of the EV. In other aspects, the adjuvant is linked to a first scaffold moiety on the exterior surface of the EVs and the antigen, e.g., derived from a coronavirus, e.g., a SARS-CoV-1 and/or SARS-CoV-2 (COVID-19) virus, is linked to a second scaffold moiety on the luminal surface of the EV. In such aspects, the first scaffold moiety is Scaffold X, and the second scaffold moiety is Scaffold Y; or each of the first scaffold moiety and the second scaffold moiety is Scaffold X.

In some aspects, the antigen, e.g., derived from a coronavirus, e.g., a SARS-CoV-1 and/or SARS-CoV-2 (COVID-19) virus, is in the lumen of the EVs and the adjuvant is in the lumen of the EV.

In some aspects, the antigen, e.g., derived from a coronavirus, e.g., a SARS-CoV-1 and/or SARS-CoV-2 (COVID-19) virus, is linked to a first scaffold moiety on the exterior surface of the EVs and the adjuvant is linked to a second scaffold moiety on the exterior surface of the EV. In other aspects, the adjuvant is linked to a first scaffold moiety on the exterior surface of the EVs and the antigen, e.g., derived from a coronavirus, e.g., a SARS-CoV-1 and/or SARS-CoV-2 (COVID-19) virus, is linked to a second scaffold moiety on the exterior surface of the EV. In some aspects, the first scaffold moiety and the second scaffold moiety are Scaffold X.

In some aspects, the antigen, e.g., derived from a coronavirus, e.g., a SARS-CoV-1 and/or SARS-CoV-2 (COVID-19) virus, is linked to a first scaffold moiety on the exterior surface of the EVs and the adjuvant is in the lumen of the EV. In some aspects, the antigen, e.g., derived from a coronavirus, e.g., a SARS-CoV-1 and/or SARS-CoV-2 (COVID-19) virus, is in the lumen of the EVs and the adjuvant is linked to a first scaffold moiety on the exterior surface of the EV. In such aspects, the first scaffold moiety can be Scaffold X.

In some aspects, the antigen, e.g., derived from a coronavirus, e.g., a SARS-CoV-1 and/or SARS-CoV-2 (COVID-19) virus, is linked to a first scaffold moiety on the exterior surface of the EV and the adjuvant is linked to the first scaffold moiety on the luminal surface of the EV. In other aspects, the antigen, e.g., derived from a coronavirus, e.g., a SARS-CoV-1 and/or SARS-CoV-2 (COVID-19) virus, is linked to a first scaffold moiety on the luminal surface of the EV and the adjuvant is linked to the first scaffold moiety on the exterior surface of the EV. In these aspects, the first scaffold moiety can be Scaffold X.

Non-limiting examples of specific aspects, include EVs comprising (i) an antigen, e.g., derived from a coronavirus, e.g., a SARS-CoV-1 and/or SARS-CoV-2 (COVID-19) virus, and (ii) an adjuvant, wherein;
(a) the antigen is linked to a first Scaffold Y on the luminal surface of the EV and the adjuvant is linked to a second Scaffold Y on the luminal surface of the EV;
(b) the antigen is linked to a Scaffold Y on the luminal surface of the EV and the adjuvant is in the lumen of the EV not linked to any scaffold moiety;
(c) the antigen is in the lumen of the EV not linked to any scaffold moiety, and the adjuvant is linked to a Scaffold Y on the luminal surface of the EV;
(d) the antigen is linked to a Scaffold Y on the luminal surface of the EV and the adjuvant is linked to a Scaffold X on the exterior surface of the EV;
(e) the antigen is in the lumen of the EV not linked to any scaffold moiety, and the adjuvant is linked to a Scaffold X on the exterior surface of the EV;
(f) the antigen is linked to a Scaffold Y on the luminal surface of the EV and the adjuvant is linked to a Scaffold X on the luminal surface of the EV;
(g) the antigen is in the lumen of the EV not linked to any scaffold moiety, and the adjuvant is linked to a Scaffold X on the luminal surface of the EV;
(h) the antigen is linked to a Scaffold X on the luminal surface of the EV and the adjuvant is linked to the Scaffold X on the exterior surface of the EV;
(i) the antigen is linked to a first Scaffold X on the exterior surface of the EV and the adjuvant is linked to a second Scaffold X on the exterior surface of the EV;
(j) the antigen is linked to a Scaffold X on the exterior surface of the EV and the adjuvant is linked to a Scaffold Y on the luminal surface of the EV;
(k) the antigen is linked to a Scaffold X on the exterior surface of the EV and the adjuvant is in the lumen of the EV not linked to any scaffold moiety;
(l) the antigen is linked to a Scaffold X on the exterior surface of the EV and the adjuvant is linked to the Scaffold X on the luminal surface of the EV;
(m) the antigen is linked to a first Scaffold X on the luminal surface of the EV and the adjuvant is linked to a second Scaffold X on the luminal surface of the EV;
(n) the antigen is linked to a Scaffold X on the luminal surface of the EV and the adjuvant is linked to a Scaffold Y on the luminal surface of the EV;
(o) the antigen is linked to a Scaffold X on the luminal surface of the EV and the adjuvant is in the lumen of the EV not linked to any scaffold moiety;
(p) the antigen is linked to a first Scaffold X on the exterior surface of the EV and the adjuvant is linked to a second Scaffold X on the luminal surface of the EV;
(q) the antigen is linked to a first Scaffold X on the luminal surface of the EV and the adjuvant is linked to a second Scaffold X on the exterior surface of the EV;
(r) the antigen is in the lumen of the EV not linked to any scaffold moiety, and the adjuvant is in the lumen of the EV not linked to any scaffold moiety;
(s) the antigen is linked directly to the luminal surface of the EV and the adjuvant is linked directly to the luminal surface of the EV;
(t) the antigen is linked directly to the luminal surface of the EV and the adjuvant is in the lumen of the EV;
(u) the antigen is linked directly to the luminal surface of the EV and the adjuvant is linked to a Scaffold Y on the luminal surface of the EV;
(v) the antigen is linked directly to the luminal surface of the EV and the adjuvant is linked to a Scaffold X on the luminal surface of the EV;
(w) the antigen is linked directly to the luminal surface of the EV and the adjuvant is linked directly to the exterior of the EV;
(x) the antigen is linked directly to the luminal surface of the EV and the adjuvant is linked to a Scaffold X on the exterior of the EV;
(y) the antigen is linked to a Scaffold Y on the luminal surface of the EV and the adjuvant is linked directly to the luminal surface of the EV;
(z) the antigen is linked to a Scaffold Y on the luminal surface of the EV and the adjuvant is linked directly to the exterior of the EV;
(aa) the antigen is linked to a Scaffold X on the luminal surface of the EV and the adjuvant is linked directly to the luminal surface of the EV;
(bb) the antigen is linked to a Scaffold X on the luminal surface of the EV and the adjuvant is linked directly to the exterior of the EV;
(cc) the antigen is in the lumen of the EV and the adjuvant is linked directly to the luminal surface of the EV; or
(dd) the antigen is in the lumen of the EV and the adjuvant is linked directly to the exterior of the EV.

In some aspects, an EV of the present disclosure comprises (i) an antigen, e.g., derived from a coronavirus, e.g., a SARS-CoV-1 and/or SARS-CoV-2 (COVID-19) virus, and (ii) an adjuvant, wherein the antigen is linked to a first Scaffold Y on the luminal surface of the EV and the adjuvant is linked to a second Scaffold Y on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold Y on the luminal surface of the EV and the adjuvant is in the lumen of the EV not linked to any scaffold moiety. In some aspects, an EV of the present disclosure comprises (i) an antigen, e.g., derived from a coronavirus, e.g., a SARS-CoV-1 and/or SARS-CoV-2 (COVID-19) virus, and (ii) an adjuvant, wherein the antigen is in the lumen of the EV not linked to any scaffold moiety, and the adjuvant is linked to a Scaffold Y on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold Y on the luminal surface of the EV and the adjuvant is linked to a Scaffold X on the exterior surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an adjuvant, wherein the antigen is in the lumen of the EV not linked to any scaffold moiety, and the adjuvant is linked to a Scaffold X on the exterior surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold Y on the luminal surface of the EV and the adjuvant is linked to a Scaffold X on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an adjuvant, wherein the antigen is in the lumen of the EV not linked to any scaffold moiety, and the adjuvant is linked to a Scaffold X on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold X on the luminal surface of the EV and the adjuvant is linked to the Scaffold X on the exterior surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an adjuvant, wherein the antigen is linked to a first Scaffold X on the exterior surface of the EV, and the adjuvant is linked to a second Scaffold X on the exterior surface of the EV. In some aspects, an EV comprises an antigen, e.g., derived from a coronavirus, e.g. a SARS-CoV-1 and/or SARS-CoV-2 (COVID-19) virus, and (ii) an adjuvant, wherein the antigen is linked to a Scaffold X on the exterior surface of the EV and the adjuvant is linked to a Scaffold Y on the luminal surface of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold X on the exterior surface of the EV and the adjuvant is in the lumen of the EV not linked to any scaffold moiety. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold X on the exterior surface of the EV and the adjuvant is linked to the Scaffold X on the luminal surface of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked to a first Scaffold X on the luminal surface of the EV and the adjuvant is linked to a second Scaffold X on the luminal surface of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold X on the luminal surface of the EV and the adjuvant is linked to a Scaffold Y on the luminal surface of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold X on the luminal surface of the EV and the adjuvant is in the lumen of the EV not linked to any scaffold moiety. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked to a first Scaffold X on the exterior surface of the EV and the adjuvant is linked to a second Scaffold X on the luminal surface of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked to a first Scaffold X on the luminal surface of the EV and the adjuvant is linked to a second Scaffold X on the exterior surface of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is in the lumen of the EV not linked to any scaffold moiety and the adjuvant is in the lumen of the EV not linked to any scaffold moiety. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked directly to the luminal surface of the EV and the adjuvant is linked directly to the luminal surface of the EV. In some aspects, an EV comprises (1) an antigen, e.g., derived from a coronavirus, e.g., a SARS-CoV-1 and/or SARS-CoV-2 (COVID-19) virus, and (ii) an adjuvant, wherein the antigen is linked directly to the luminal surface of the EV and the adjuvant is in the lumen of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked directly to the luminal surface of the EV and the adjuvant is linked to a Scaffold Y on the luminal surface of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked directly to the luminal surface of the EV and the adjuvant is linked to a Scaffold X on the luminal surface of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked directly to the luminal surface of the EV and the adjuvant is linked directly to the exterior of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked directly to the luminal surface of the EV and the adjuvant is linked to a Scaffold X on the exterior of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold Y on the luminal surface of the EV and the adjuvant is linked directly to the luminal surface of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold Y on the luminal surface of the EV and the adjuvant is linked directly to the exterior of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold X on the luminal surface of the EV and the adjuvant is linked directly to the luminal surface of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is linked to a Scaffold X on the luminal surface of the E, and the adjuvant is linked directly to the exterior of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is in the lumen of the EV and the adjuvant is linked directly to the luminal surface of the EV. In some aspects, an EV comprises (i) an antigen, e.g., derived from a coronavirus, e.g., a SARS-CoV-1 and/or SARS-CoV-2 (COVID-19) virus, and (ii) an adjuvant, wherein the antigen is in the lumen of the EV and the adjuvant is linked directly to the exterior of the EV.

In some aspects, an adjuvant and/or antigen can be modified to increase encapsulation (i.e., loading) in an EV. This modification can include the addition of a lipid binding tag by treating the agonist (i.e., adjuvant and/or antigen) with a chemical or enzyme, or by physically or chemically altering the polarity or charge of the adjuvant and/or antigen. The adjuvant and/or antigen can be modified by a single treatment, or by a combination of treatments, e.g., adding a lipid binding tag only, or adding a lipid binding tag and altering the polarity. The previous example is meant to be a non-limiting illustrative instance. It is contemplated that any combination of modifications can be practiced. The modification can increase encapsulation (i.e., loading) of the adjuvant and/or antigen in the EV by between about 2-fold and about 10,000-fold, between about 10-fold and 1,000-fold, or between about 100-fold and about 500-fold compared to encapsulation (i.e., loading) of an unmodified agonist (i.e., adjuvant and/or antigen). The modification can increase encapsulation (i.e., loading) of the adjuvant and/or antigen in the EV by at least about 2-fold, about 5-fold, about 10-fold, about 20-fold, about 30-fold, about 40-fold, about 50-fold, about 60-fold, about 70-fold, about 80-fold, about 90-fold, about 100-fold, about 200-fold, about 300-fold, about 400-fold, about 500-fold, about 600-fold, about 700-fold, about 800-fold, about 900-fold, about 1,000-fold, about 2,000-fold, about 3,000-fold, about 4,000-fold, about 5,000-fold, about 6,000-fold, about 7,000-fold, about 8,000-fold, about 9,000-fold, or about 10,000-fold compared to encapsulation (i.e., loading) of an unmodified adjuvant and/or antigen.

In some aspects, an adjuvant and/or antigen can be modified to allow for better expression on the surface of the EV (e.g., exterior and/or luminal surface of the EV, e.g., linked to a scaffold moiety disclosed herein, e.g., Scaffold X and/or Scaffold Y). Any of the modifications described above can be used. The modification can increase expression of the agonist in the EV, e.g., on the surface and/or luminal surface of the exosome, by about between 2-fold and 10,000-fold, about between 10-fold and 1,000-fold, or about between 100-fold and 500-fold compared to corresponding expression of an unmodified agonist. The modification can increase expression of the agonist on the exterior surface of the EV by at least about 2-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, or at least about 10,000-fold compared to expression of an unmodified agonist. The modification can increase expression of the agonist on the luminal surface of the EV by at least about 2-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, or at least about 10,000-fold compared to expression of an unmodified agonist.

### II.C. Targeting Moiety (e.g., Tropism Moiety)

In some aspects, the EV is further modified to display an additional protein (or fragment thereof) that can help direct EV uptake (e.g., targeting moiety), activate, or block cellular pathways to enhance the combinatorial effects associated with the EV (e.g., effect of a payload loaded into an exosome, e.g., STING agonist). In certain aspects, the EV disclosed herein further comprises a targeting moiety that can modify the distribution of the EVs in vivo or in vitro. In some aspects, the targeting moiety can be a biological molecule, such as a protein, a peptide, a lipid, or a synthetic molecule. Not to be bound by any one theory, as will be apparent from the present disclosure, in some aspects, the addition of such moieties can further enhance the therapeutic effects of the EVs described herein, e.g., when administered to a subject. Moreover, as described herein, in some aspects, the targeting moiety can be present in the EV prior to the addition of other moieties described herein (e.g., an antigen). In such aspects, the targeting moiety can be introduced into a producer cell when producing the EV (e.g., base EV). In some aspects, the targeting moiety can be added to the EVs after being isolated from the producer cells. In such aspects, the targeting moiety can be added to the isolated EVs before adding the other moieties described herein (e.g., an antigen). In some aspects, the targeting moiety is added to the EV after adding the other moieties described herein (e.g., an antigen). In some aspects, the targeting moiety is added to the EV together with the other moieties described herein.

In some aspects, a targeting moiety of the present disclosure specifically binds to a marker for a particular type of cells. In some aspects, the cell is an immune cell, e.g., dendritic cell. In certain aspects, the marker is expressed only on dendritic cells. In some aspects, dendritic cells comprise a progenitor (Pre) dendritic cells, inflammatory mono dendritic cells, plasmacytoid dendritic cell (pDC), a myeloid/conventional dendritic cell 1 (cDC1), a myeloid/conventional dendritic cell 2 (cDC2), inflammatory monocyte derived dendritic cells, Langerhans cells, dermal dendritic cells, lysozyme-expressing dendritic cells (LysoDCs), Kupffer cells, nonclassical monocytes, or any combination thereof. Markers that are expressed on these dendritic cells are known in the art. See, e.g., Collin et al., Immunology 154 (1): 3-20 (2018). In some aspects, the targeting moiety is a protein, wherein the protein is an antibody or a fragment thereof that can specifically bind to a marker selected from DEC205, CLEC9A, CLEC6, DCIR, DC-SIGN, LOX-1, MARCO, Clec12a, Clec10a, DC-asialoglycoprotein receptor (DC-ASGPR), DC immunoreceptor 2 (DCIR2), Dectin-1, macrophage mannose receptor (MMR), BDCA-2 (CD303, Clec4c), Dectin-2, Bst-2 (CD317), Langerin, CD206, CD11b, CD11c, CD123, CD304, XCR1, AXL, Siglec 6, CD209, SIRPA, CX3CR1, GPR182, CD14, CD16, CD32, CD34, CD38, CD10, or any combination thereof. In some aspects, a marker useful for the present disclosure comprises a C-type lectin like domain. In certain aspects, a marker is Clec9a and the dendritic cell is cDC1.

In some aspects, a targeting moiety disclosed herein can bind to both human and mouse Clec9a, including any variants thereof. In some aspects, a targeting moiety of the present disclosure can bind to Clec9a from other species, including but not limited to chimpanzee, rhesus monkey, dog, cow, horse, or rat. Sequences for such Clec9a protein are known in the art. See, e.g., U.S. Pat. No. 8,426,565 B2, which is herein incorporated by reference in its entirety.

In some aspects, a targeting moiety of the present disclosure specifically binds to a marker for a T cell. In certain aspects, the T cell is a CD4+ T cell. In some aspects, the T cell is a CD8+ T cell.

In some aspects, a targeting moiety disclosed herein binds to human CD3 protein or a fragment thereof. Sequences for human CD3 protein are known in the art.

In some aspects, a targeting moiety disclosed herein can bind to both human and mouse CD3, including any variants thereof. In some aspects, a targeting moiety of the present disclosure can bind to CD3 from other species, including but not limited to chimpanzee, rhesus monkey, dog, cow, horse, or rat. Sequences for such CD3 protein are also known in the art.

In some aspects, a targeting moiety that is useful for the present disclosure specifically binds to a marker expressed on a B cell. Non-limiting examples of markers expressed on B cells include CD40, CD22, CD19, B220, IgM, MHCII, or combinations thereof.

In some aspects, a targeting moiety can increase the uptake of an EV by follicular DCs. As used herein, “follicular” DCs (FDCs) are non-migratory population of immune cells found in primary and secondary follicles of the B cell areas of lymphoid tissues (e.g., lymph nodes, spleen, and mucosa-associated lymphoid tissue (MALT)). FDCs differ from DCs in that they are not derived from bone-marrow hematopoietic stem cells but are of mesenchymal origin. FDCs present antigen to B cells within the germinal center and regulate B cell antibody affinity maturation and B cell memory responses. Non-limiting examples of such tropism moieties include IgG, IgG-antigen complex, IgG-Fc, S aureus D domain dimer, anti-CR1 antibody, anti-CR2 antibody, or combinations thereof.

In some aspects, a targeting moiety disclosed herein can allow for greater uptake of an EV by a cell expressing a marker specific for the targeting moiety (e.g., CD3: CD4+ T cell and/or CD8+ T cell; Clec9a: dendritic cells; CD40, CD22, or CD19: B cells). In some aspects, the uptake of an EV is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to a reference (e.g., corresponding EV without the targeting moiety or a non-EV delivery vehicle). In some aspects, a reference comprises an EV that does not express a targeting moiety disclosed herein.

In some aspects, the increased uptake of an EV disclosed herein can allow for greater immune response. Accordingly, in certain aspects, an EV expressing a targeting moiety disclosed herein can increase an immune response (e.g., against a coronavirus antigen loaded onto the exosome) by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to a reference (e.g., corresponding EV without the targeting moiety or a non-EV delivery vehicle). In some aspects, a reference comprises an EV that does not express a targeting moiety disclosed herein. In certain aspects, an immune response is mediated by T cells (e.g., CD8+ T cells or CD4+ T cells) and/or B cells.

As described supra, a targeting moiety disclosed herein can comprise a peptide, an antibody or an antigen binding fragment thereof, a chemical compound, or any combination thereof.

In some aspects, the targeting moiety is a peptide that can specifically bind to Clec9a. See, e.g., Yan et al., Oncotarget 7 (26): 40437-40450 (2016). For example, in certain aspects, the peptide comprises a soluble fragment of Clec9a. A non-limiting example of such a peptide is described in U.S. Pat. No. 9,988,431 B2, which is herein incorporated by reference in its entirety. In certain aspects, the peptide comprises a ligand (natural or synthetic) of Clec9a, such as those described in Ahrens et al., Immunity 36 (4): 635-45 (2012); and Zhang et al., Immunity 36 (4): 646-57 (2012). A non-limiting example of a peptide comprising a Clec9a ligand is described in International Publ. No. WO 2013/053008 A2, which is herein incorporated by reference in its entirety.

In some aspects, the targeting moiety is a peptide that can specifically bind to CD3. For example, in certain aspects, the peptide comprises a soluble fragment of CD3. In certain aspects, the peptide comprises a ligand (natural or synthetic) of CD3.

In some aspects, the targeting moiety is an antibody or an antigen binding fragment thereof. In certain aspects, a targeting moiety is a single-chain Fv antibody fragment. In certain aspects, a targeting moiety is a single-chain F(ab) antibody fragment. In certain aspects, a targeting moiety is a nanobody. In certain aspects, a targeting moiety is a monobody.

In some aspects, an EV disclosed herein comprises one or more (e.g., 2, 3, 4, 5, or more) targeting moieties. In certain aspects, the one or more targeting moieties are expressed in combination with other exogenous biologically active molecules disclosed herein (e.g., therapeutic molecule, adjuvant, or immune modulator). In some aspects, the one or more targeting moieties can be expressed on the exterior surface of the EV. Accordingly, in certain aspects, the one or more targeting moieties are linked to a scaffold moiety (e.g., Scaffold X) on the exterior surface of the EV. When the one or more targeting moieties are expressed in combination with other exogenous biologically active molecules (e.g., therapeutic molecule, adjuvant, or immune modulator), the other exogenous biologically active molecules can be expressed on the surface (e.g., exterior surface or luminal surface) or in the lumen of the EV.

The producer cell can be modified to comprise an additional exogenous sequence encoding for the additional protein or fragment thereof. Alternatively, the additional protein or fragment thereof can be covalently linked or conjugated to the EV via any appropriate linking chemistry known in the art. Non-limiting examples of appropriate linking chemistry include amine-reactive groups, carboxyl-reactive groups, sulfhydryl-reactive groups, aldehyde-reactive groups, photoreactive groups, ClickIT chemistry, biotin-streptavidin or other avidin conjugation, or any combination thereof.

### II.D Immune Modulator

In some aspects, an EV of the present disclosure can comprise an immune modulator (e.g., along with an antigen and/or other payloads disclosed herein). In some aspects, an EV disclosed herein comprises multiple immune modulators. In certain aspects, each of the multiple immune modulators is different. In some aspects, an EV disclosed herein comprises at least 2, 3, 4, 5, 6, 7, 8, 9, 10 or more different immune modulators.

In certain aspects, an EV comprises the one or more immune modulators in combination with one or more additional payloads (e.g., antigen and/or adjuvants). In some aspects, an EV can comprise one or more additional moieties (e.g., targeting moieties). For instance, in certain aspects, an EV disclosed described herein can comprise (i) one or more immune modulators, (ii) one or more additional payloads (e.g., antigen and/or adjuvant), and (iii) one or more targeting moieties. As described herein, in certain aspects, the immune modulator can be present in the EV prior to the addition of other moieties described herein (e.g., an antigen). In such aspects, the immune modulator can be introduced into a producer cell when producing the EV (e.g., base EV). In some aspects, the immune modulator can be added to the EVs after being isolated from the producer cells. In such aspects, the immune modulator can be added to the isolated EVs before adding the other moieties described herein (e.g., an antigen). In some aspects, the immune modulator is added to the EV after adding the other moieties described herein (e.g., an antigen). In some aspects, the immune modulator is added to the EV together with the other moieties described herein.

In some aspects, an immune modulator can be expressed on the surface (e.g., exterior surface or luminal surface) or in the lumen of the EV. Accordingly, in certain aspects, the immune modulator is linked to a scaffold moiety (e.g., Scaffold X) on the exterior surface of the EV or on the luminal surface of the EV. In other aspects, the immune modulator is linked to a scaffold moiety (e.g., Scaffold Y) on the luminal surface of the EV. In further aspects, the immune modulator is in the lumen of the exosome (i.e., not linked to either Scaffold X or Scaffold Y). In some aspects, an immune modulator can be directly linked (i.e., without the use of a scaffold moiety) to the exterior surface and/or luminal surface of an EV.

Non-limiting examples of such aspects, include EVs comprising (i) an antigen, e.g., derived from a coronavirus, e.g., a SARS-CoV-1 and/or SARS-CoV-2 (COVID-19) virus, and (ii) an immune modulator, wherein:
(a) the antigen is linked to a first Scaffold Y on the luminal surface of the EV and the immune modulator is linked to a second Scaffold Y on the luminal surface of the EV;
(b) the antigen is linked to a Scaffold Y on the luminal surface of the EV and the immune modulator is in the lumen of the EV not linked to any scaffold moiety;
(c) the antigen is in the lumen of the EV not linked to any scaffold moiety and the immune modulator is linked to a Scaffold Y on the luminal surface of the EV;
(d) the antigen is linked to a Scaffold Y on the luminal surface of the EV and the immune modulator is linked to a Scaffold X on the exterior surface the EV;
(e) the antigen is linked to a Scaffold X on the luminal surface of the EV and the immune modulator is linked to a Scaffold Y on the luminal surface of the EV;
(f) the antigen is linked to a Scaffold X on the luminal surface of the EV and the immune modulator is linked to a Scaffold Y on the luminal surface of the EV; or
(g) the antigen is linked to a Scaffold X on the exterior surface of the EV and the immune modulator is linked to a Scaffold Y on the luminal surface of the EV.

Non-limiting examples of specific aspects, include EVs comprising (i) an antigen, e.g., derived from a coronavirus, e.g., a SARS-CoV-1 and/or SARS-CoV-2 (COVID-19) virus, and (ii) an immune modulator, wherein:
(a) the antigen is linked to a first Scaffold Y on the luminal surface of the EV and the immune modulator is linked to a second Scaffold Y on the luminal surface of the EV;
(b) the antigen is linked to a Scaffold Y on the luminal surface of the EV and the immune modulator is in the lumen of the EV not linked to any scaffold moiety;
(c) the antigen is in the lumen of the EV not linked to any scaffold moiety, and the immune modulator is linked to a Scaffold Y on the luminal surface of the EV;
(d) the antigen is linked to a Scaffold Y on the luminal surface of the EV and the immune modulator is linked to a Scaffold X on the exterior surface of the EV;
(e) the antigen is in the lumen of the EV not linked to any scaffold moiety, and the immune modulator is linked to a Scaffold X on the exterior surface of the EV;
(f) the antigen is linked to a Scaffold Y on the luminal surface of the EV and the immune modulator is linked to a Scaffold X on the luminal surface of the EV;
(g) the antigen is in the lumen of the EV not linked to any scaffold moiety, and the immune modulator is linked to a Scaffold X on the luminal surface of the EV;
(h) the antigen is linked to a Scaffold X on the luminal surface of the EV and the immune modulator is linked to the Scaffold X on the exterior surface of the EV;
(i) the antigen is linked to a first Scaffold X on the exterior surface of the EV and the immune modulator is linked to a second Scaffold X on the exterior surface of the EV;
(j) the antigen is linked to a Scaffold X on the exterior surface of the EV and the immune modulator is linked to a Scaffold Y on the luminal surface of the EV;
(k) the antigen is linked to a Scaffold X on the exterior surface of the EV and the immune modulator is in the lumen of the EV not linked to any scaffold moiety;
(l) the antigen is linked to a Scaffold X on the exterior surface of the EV and the immune modulator is linked to the Scaffold X on the luminal surface of the EV;
(m) the antigen is linked to a first Scaffold X on the luminal surface of the EV and the immune modulator is linked to a second Scaffold X on the luminal surface of the EV;
(n) the antigen is linked to a Scaffold X on the luminal surface of the EV and the immune modulator is linked to a Scaffold Y on the luminal surface of the EV;
(o) the antigen is linked to a Scaffold X on the luminal surface of the EV and the immune modulator is in the lumen of the EV not linked to any scaffold moiety;
(p) the antigen is linked to a first Scaffold X on the exterior surface of the EV and the immune modulator is linked to a second Scaffold X on the luminal surface of the EV;
(q) the antigen is linked to a first Scaffold X on the luminal surface of the EV and the immune modulator is linked to a second Scaffold X on the exterior surface of the EV;
(r) the antigen is in the lumen of the EV not linked to any scaffold moiety, and the immune modulator is in the lumen of the EV not linked to any scaffold moiety;
(s) the antigen is linked directly to the luminal surface of the EV and the immune modulator is linked directly to the luminal surface of the EV;
(t) the antigen is linked directly to the luminal surface of the EV and the immune modulator is in the lumen of the EV;
(u) the antigen is linked directly to the luminal surface of the EV and the immune modulator is linked to a Scaffold Y on the luminal surface of the EV;
(v) the antigen is linked directly to the luminal surface of the EV and the immune modulator is linked to a Scaffold X on the luminal surface of the EV;
(w) the antigen is linked directly to the luminal surface of the EV and the immune modulator is linked directly to the exterior of the EV;
(x) the antigen is linked directly to the luminal surface of the EV and the immune modulator is linked to a Scaffold X on the exterior of the EV;
(y) the antigen is linked to a Scaffold Y on the luminal surface of the EV and the immune modulator is linked directly to the luminal surface of the EV;
(z) the antigen is linked to a Scaffold Y on the luminal surface of the EV and the immune modulator is linked directly to the exterior of the EV;
(aa) the antigen is linked to a Scaffold X on the luminal surface of the EV and the immune modulator is linked directly to the luminal surface of the EV;
(bb) the antigen is linked to a Scaffold X on the luminal surface of the EV and the immune modulator is linked directly to the exterior of the EV;
(cc) the antigen is in the lumen of the EV and the immune modulator is linked directly to the luminal surface of the EV; or
(dd) the antigen is in the lumen of the EV and the immune modulator is linked directly to the exterior of the EV.

In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a first Scaffold Y on the luminal surface of the EV and the immune modulator is linked to a second Scaffold Y on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold Y on the luminal surface of the EV and the immune modulator is in the lumen of the EV not linked to any scaffold moiety. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is in the lumen of the EV not linked to any scaffold moiety, and the immune modulator is linked to a Scaffold Y on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold Y on the luminal surface of the EV and the immune modulator is linked to a Scaffold X on the exterior surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is in the lumen of the EV not linked to any scaffold moiety, and the immune modulator is linked to a Scaffold X on the exterior surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold Y on the luminal surface of the EV and the immune modulator is linked to a Scaffold X on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is in the lumen of the EV not linked to any scaffold moiety, and the immune modulator is linked to a Scaffold X on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold X on the luminal surface of the EV and the immune modulator is linked to the Scaffold X on the exterior surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a first Scaffold X on the exterior surface of the EV and the immune modulator is linked to a second Scaffold X on the exterior surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold X on the exterior surface of the EV and the immune modulator is linked to a Scaffold Y on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold X on the exterior surface of the EV and the immune modulator is in the lumen of the EV not linked to any scaffold moiety. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold X on the exterior surface of the EV and the immune modulator is linked to the Scaffold X on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a first Scaffold X on the luminal surface of the EV and the immune modulator is linked to a second Scaffold X on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold X on the luminal surface of the EV and the immune modulator is linked to a Scaffold Y on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold X on the luminal surface of the EV and the immune modulator is in the lumen of the EV not linked to any scaffold moiety. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a first Scaffold X on the exterior surface of the EV and the immune modulator is linked to a second Scaffold X on the luminal surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is linked to a first Scaffold X on the luminal surface of the EV and the immune modulator is linked to a second Scaffold X on the exterior surface of the EV. In some aspects, an EV of the present disclosure comprises (i) an antigen, e.g., derived from a coronavirus, e.g., a SARS-CoV-1 and/or SARS-CoV-2 (COVID-19) virus, and (ii) an immune modulator, wherein the antigen is in the lumen of the EV not linked to any scaffold moiety, and the immune modulator is in the lumen of the EV not linked to any scaffold moiety. In some aspects, an EV comprises (i) an antigen, e.g., derived from a coronavirus, e.g., a SARS-CoV-1 and/or SARS-CoV-2 (COVID-19) virus, and (ii) an immune modulator, wherein the antigen is linked directly to the luminal surface of the EV, and the immune modulator is linked directly to the luminal surface of the EV. In some aspects, an EV comprises (i) an antigen, e.g., derived from a coronavirus, e.g., a SARS-CoV-1 and/or SARS-CoV-2 (COVID-19) virus, and (ii) an immune modulator, wherein the antigen is linked directly to the luminal surface of the EV, and the immune modulator is in the lumen of the EV. In some aspects, an EV comprises (i) an antigen, e.g., derived from a coronavirus, e.g., a SARS-CoV-1 and/or SARS-CoV-2 (COVID-19) virus, and (ii) an immune modulator, wherein the antigen is linked directly to the luminal surface of the EV, and the immune modulator is linked to a Scaffold Y on the luminal surface of the EV. In some aspects, an EV comprises (i) an antigen, e.g., derived from a coronavirus, e.g., a SARS-CoV-1 and/or SARS-CoV-2 (COVID-19) virus, and (ii) an immune modulator, wherein the antigen is linked directly to the luminal surface of the EV, and the immune modulator is linked to a Scaffold X on the luminal surface of the EV. In some aspects, an EV comprises (i) an antigen, e.g., derived from a coronavirus, e.g., a SARS-CoV-1 and/or SARS-CoV-2 (COVID-19) virus, and (ii) an immune modulator, wherein the antigen is linked directly to the luminal surface of the EV, and the immune modulator is linked directly to the exterior of the EV. In some aspects, an EV comprises (i) an antigen, e.g., derived from a coronavirus, e.g., a SARS-CoV-1 and/or SARS-CoV-2 (COVID-19) virus, and (ii) an immune modulator, wherein the antigen is linked directly to the luminal surface of the EV, and the immune modulator is linked to a Scaffold X on the exterior of the EV. In some aspects, an EV comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold Y on the luminal surface of the EV, and the immune modulator is linked directly to the luminal surface of the EV. In some aspects, an EV comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold Y on the luminal surface of the EV, and the immune modulator is linked directly to the exterior of the EV. In some aspects, an EV comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold X on the luminal surface of the EV, and the immune modulator is linked directly to the luminal surface of the EV. In some aspects, an EV comprises (i) an antigen, e.g., derived from a coronavirus, e.g., a SARS-CoV-1 and/or SARS-CoV-2 (COVID-19) virus, and (ii) an immune modulator, wherein the antigen is linked to a Scaffold X on the luminal surface of the EV, and the immune modulator is linked directly to the exterior of the EV. In some aspects, an EV comprises (i) an antigen, e.g., derived from a coronavirus, e.g., a SARS-CoV-1 and/or SARS-CoV-2 (COVID-19) virus, and (ii) an immune modulator, wherein antigen is in the lumen of the EV, and the immune modulator is linked directly to the luminal surface of the EV. In some aspects, an EV comprises (i) an antigen, e.g., derived from a coronavirus, e.g., a SARS-CoV-1 and/or SARS-CoV-2 (COVID-19) virus, and (ii) an immune modulator, wherein antigen is in the lumen of the EV, and the immune modulator is linked directly to the exterior of the EV.

In some aspects, an immune modulator can regulate innate immune response. In some aspects, an immune modulator can regulate adaptive immune response. In some aspects, the immune modulator regulates adaptive immune response by targeting cytotoxic T cells. In further aspects, the immune modulator regulates adaptive immune response by targeting B cells (e.g., resulting in the production of antigen-specific antibodies). In certain aspects, an immune modulator disclosed herein can modulate the distribution of an exosome to a cytotoxic T cell or a B cell (i.e., bio-distribution modifying agent).

In some aspects, an immune modulator useful for the present disclosure can specifically induce the activation of certain lymphocyte subsets. For instance, in some aspects, an immune modulator can specifically induce the activation of CD4+ T helper cells. CD4+ T helper cells are arguably the most important cells in adaptive immunity, as they are required for almost all adaptive immune responses. They not only help activate B cells to secrete antibodies and macrophages to destroy ingested microbes, but they also help activate cytotoxic T cells to kill infected target cells. Crott S., Nat Rev Immunol 15 (3): 185-189 (March 2015). In certain aspects, the immune modulator is a peptide that can specifically induce the activation of CD4+ helper T cells. In some aspects, such peptides are referred to herein as “CD4+ T helper peptide”. In some aspects, the CD4+ T helper peptides are derived from tetanus, measles, diphtheria toxins, or combinations thereof. The CD4+ T help peptides that are useful for the present disclosure can also comprise the PADRE peptide (AKFVAAWTLKAAA; SEQ ID NO: 386). In certain aspects, such peptides are referred to herein as “universal CD4+ T helper peptide,” as they are capable of inducing the activation of CD4+ helper T cells in an antigen-independent manner (i.e., non-specific activation). In some aspects, the CD4+ T cell epitope comprises the amino acid sequence QYIKANSKFIGITE (SEQ ID NO: 383) (amino acid residues 830-843 of tetanus). In some aspects, the CD4+ T cell epitope comprises the amino acid sequence QSIALSSLMVAQAIP (SEQ ID NO: 384) (amino acid residues 356-370 of diphtheria toxin).

In some aspects, an immune modulator comprises an inhibitor for a negative checkpoint regulator or an inhibitor for a binding partner of a negative checkpoint regulator. In certain aspects, the negative checkpoint regulator comprises cytotoxic T-lymphocyte-associated protein 4 (CTLA-4), programmed cell death protein 1 (PD-1), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, or any combination thereof.

In some aspects, the immune modulator is an inhibitor of cytotoxic T-lymphocyte-associate protein 4 (CTLA-4). In certain aspects, the CTLA-4 inhibitor is a monoclonal antibody of CTLA-4 (“anti-CTLA-4 antibody”). In certain aspects, the inhibitor is a fragment of a monoclonal antibody of CTLA-4. In certain aspects, the antibody fragment is a scFv, (scFv)₂, Fab, Fab′, and F(ab′)₂, F(ab1)₂, Fv, dAb, or Fd of a monoclonal antibody of CTLA-4. In certain aspects, the inhibitor is a nanobody, a bispecific antibody, or a multispecific antibody against CTLA-4. In some aspects, the anti-CTLA-4 antibody is ipilimumab. In other aspects, the anti-CTLA-4 antibody is tremelimumab.

In some aspects, the immune modulator is an inhibitor of programmed cell death protein 1 (PD-1). In some aspects, the immune modulator is an inhibitor of programmed death-ligand 1 (PD-L1). In some aspects, the immune modulator is an inhibitor of programmed death-ligand 2 (PD-L2). In certain aspects, the inhibitor of PD-1, PD-L1, or PD-L2 is a monoclonal antibody of PD-1 (“anti-PD-1 antibody”), PD-L1 (“anti-PD-L1 antibody”), or PD-L2 (“anti-PD-L2 antibody”). In some aspects, the inhibitor is a fragment of an anti-PD-1 antibody, anti-PD-L1 antibody, or anti-PD-L2 antibody. In certain aspects, the antibody fragment is a scFv, (scFv)₂, Fab, Fab′, and F(ab′)₂, F(ab1)₂, Fv, dAb, or Fd of a monoclonal antibody of PD-1, PD-L1, or PD-L2. In certain aspects, the inhibitor is a nanobody, a bispecific antibody, or a multispecific antibody against PD-1, PD-L1, or PD-L2. In some aspects, the anti-PD-1 antibody is nivolumab. In some aspects, the anti-PD-1 antibody is pembrolizumab. In some aspects, the anti-PD-1 antibody is pidilizumab. In some aspects, the anti-PD-L1 antibody is atezolizumab. In other aspects, the anti-PD-L1 antibody is avelumab.

In some aspects, the immune modulator is an inhibitor of lymphocyte-activated gene 3 (LAG3). In certain aspects, the inhibitor of LAG3 is a monoclonal antibody of LAG3 (“anti-LAG3 antibody”). In some aspects, the inhibitor is a fragment of an anti-LAG3 antibody, e.g., scFv, (scFv)₂, Fab, Fab′, and F(ab′)₂, F(ab1)₂, Fv, dAb, or Fd. In certain aspects, the inhibitor is a nanobody, a bispecific antibody, or a multispecific antibody against LAG3.

In some aspects, the immune modulator is an inhibitor of T-cell immunoglobulin mucin-containing protein 3 (TIM-3). In some aspects, the immune modulator is an inhibitor of B and T lymphocyte attenuator (BTLA). In some aspects, the immune modulator is an inhibitor of T cell immunoreceptor with Ig and ITIM domains (TIGIT). In some aspects, the immune modulator is an inhibitor of V-domain Ig suppressor of T cell activation (VISTA). In some aspects, the immune modulator is an inhibitor of adenosine A2a receptor (A2aR). In some aspects, the immune modulator is an inhibitor of killer cell immunoglobulin like receptor (KIR). In some aspects, the immune modulator is an inhibitor of indoleamine 2,3-dioxygenase (IDO). In some aspects, the immune modulator is an inhibitor of CD20, CD39, or CD73.

In some aspects, the immune modulator comprises an activator for a positive co-stimulatory molecule or an activator for a binding partner of a positive co-stimulatory molecule. In certain aspects, the positive co-stimulatory molecule comprises a TNF receptor superfamily member (e.g., CD120a, CD120b, CD18, OX40, CD40, Fas receptor, M68, CD27, CD30, 4-1BB, TRAILR1, TRAILR2, TRAILR3, TRAILR4, RANK, OCIF, TWEAK receptor, TACI, BAFF receptor, ATAR, CD271, CD269, AITR, TROY, CD358, TRAMP, and XEDAR). In some aspects, the activator for a positive co-stimulatory molecule is a TNF superfamily member (e.g., TNFα, TNF-C, OX40L, CD40L, FasL, LIGHT, TLIA, CD27L, Siva, CD153, 4-1BB ligand, TRAIL, RANKL, TWEAK, APRIL, BAFF, CAMLG, NGF, BDNF, NT-3, NT-4, GITR ligand, and EDA-2).

In some aspects, the immune modulator is an activator of TNF Receptor Superfamily Member 4 (OX40). In certain aspects, the activator of OX40 is an agonistic anti-OX40 antibody. In further aspects, the activator of OX40 is a OX40 ligand (OX40L).

In some aspects, the immune modulator is an activator of CD27. In certain aspects, the activator of CD27 is an agonistic anti-CD27 antibody. In other aspects, the activator of CD27 is a CD27 ligand (CD27L).

In some aspects, the immune modulator is an activator of CD40. In certain aspects, the activator of CD40 is an agonistic anti-CD40 antibody. In some aspects, the activator of CD40 is a CD40 ligand (CD40L). In certain aspects, the CD40L is a monomeric CD40L. In other aspects, the CD40L is a trimeric CD40L.

In some aspects, the immune modulator is an activator of glucocorticoid-induced TNFR-related protein (GITR). In certain aspects, the activator of GITR is an agonistic anti-GITR antibody. In other aspects, the activator of GITR is a natural ligand of GITR.

In some aspects, the immune modulator is an activator of 4-1BB. In specific aspects, the activator of 4-1BB is an agonistic anti-4-1BB antibody. In certain aspects, the activator of 4-1BB is a natural ligand of 4-1BB.

In some aspects, the immune modulator is a Fas receptor (Fas). In such aspects, the Fas receptor is displayed on the surface of the EV. In some aspects, the immune modulator is Fas ligand (FasL). In certain aspects, the Fas ligand is displayed on the surface of the EV. In some aspects, the immune modulator is an anti-Fas antibody or an anti-FasL antibody.

In some aspects, the immune modulator is an activator of a CD28-superfamily co-stimulatory molecule. In certain aspects, the CD28-superfamily co-stimulatory molecule is ICOS or CD28. In certain aspects, the immunomodulating component is ICOSL, CD80, or CD86.

In some aspects, the immune modulator is an activator of inducible T cell co-stimulator (ICOS). In certain aspects, the activator of ICOS is an agonistic anti-ICOS antibody. In other aspects, the activator of ICOS is a ICOS ligand (ICOSL).

In some aspects, the immune modulator is an activator of CD28. In some aspects, the activator of CD28 is an agonistic anti-CD28 antibody. In other aspects, the activator of CD28 is a natural ligand of CD28. In certain aspects, the ligand of CD28 is CD80.

In some aspects, the immune modulator comprises a cytokine or a binding partner of a cytokine. In some aspects, the cytokine is selected from (i) common gamma chain family of cytokines; (ii) IL-1 family of cytokines; (iii) hematopoietic cytokines; (iv) interferons (e.g., type I, type II, or type III); (v) TNF family of cytokines; (vi) IL-17 family of cytokines; (vii) damage-associated molecular patterns (DAMPs); (viii) tolerogenic cytokines; or (ix) combinations thereof. In certain aspects, the cytokine comprises IL-2, IL-4, IL-7, IL-10, IL-12, IL-15, IL-21, IFN-γ, IL-1α, IL-1β, IL-1ra, IL-18, IL-33, IL-36α, IL-36β, IL-36γ, IL-36ra, IL-37, IL-38, IL-3, IL-5, IL-6, IL-11, IL-13, IL-23, granulocyte-macrophage colony stimulating factor (GM-CSF), granulocyte-colony stimulating factor (G-CSF), leukemia inhibitory factor (LIF), stem cell factor (SCF), thrombopoietin (TPO), macrophage-colony stimulating factor (M-CSF), erythropoietien (EPO), Flt-3, IFN-α, IFN-β, IFN-γ, IL-19, IL-20, IL-22, IL-24, TNF-α, TNF-β, BAFF, APRIL, lymphotoxin beta (TNF-γ), IL-17A, IL-17B, IL-17C, IL-17D, IL-17E, IL-17F, IL-25, TSLP, IL-35, IL-27, TGF-β, or combinations thereof.

In some aspects, the immune modulator comprises a chemokine. In certain aspects, chemokine comprises a (i) CC chemokine (e.g., CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28); (ii) CXC chemokine (e.g., CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17); (iii) C chemokine (e.g., XCL1, XCL2); (iv) CX3C chemokine (e.g., CX3CL1); (v) or combinations thereof.

In some aspects, the immune modulator comprises an inhibitor of lysophosphatidic acid (LPA). LPA is a highly potent endogenous lipid mediator that protects and rescues cells from programmed cell death. LPA, through its high affinity LPA-1 receptor, is an important mediator of fibrogenesis.

In some aspects, the immune modulator that can be used with the present disclosure comprises a protein that supports intracellular interactions required for germinal center responses. In certain aspects, such a protein comprises a signaling lymphocyte activation molecule (SLAM) family member or a SLAM-associated protein (SAP). In some aspects, a SLAM family members comprises SLAM, CD48, CD229 (Ly9), Ly108, 2B4, CD84, NTB-A, CRACC, BLAME, CD2F-10, or combinations thereof. Non-limiting examples of other immune modulators that can play a role in germinal center response includes: ICOS-ICOSL, CD40-40L, CD28/B7, PD-1/L1, IL-4/IL4R, IL21/IL21R, TLR4, TLR7, TLR8, TLR9, CD180, CD22, and combinations thereof.

In some aspects, the immune modulator comprises a T-cell receptor (TCR) or a derivative thereof. In certain aspects, the immune modulator is a TCR α-chain or a derivative thereof. In other aspects, the immune modulator is a TCR β-chain or a derivative thereof. In further aspects, the immune modulator is a co-receptor of the T-cell or a derivative thereof.

In some aspects, the immune modulator comprises a chimeric antigen receptor (CAR) or a derivative thereof. In certain aspects, the CAR binds to one or more of the antigens disclosed herein (e.g., tumor antigen, e.g., alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen, MART-1 gp100, and TNF-related apoptosis-inducing ligand).

In some aspects, the immune modulator comprises an activator of a T-cell receptor or co-receptor. In certain aspects, the immunomodulating component is an activator of CD3. In certain aspects, the activator is a fragment of a monoclonal antibody of CD3. In certain aspects, the antibody fragment is a scFv, (scFv)₂, Fab, Fab′, and F(ab′)₂, F(ab1)₂, Fv, dAb, or Fd of a monoclonal antibody against CD3. In certain aspects, the activator is a nanobody, a bispecific antibody, or a multispecific antibody against CD3. In certain aspects, the immunomodulating component is an activator of CD28. In certain aspects, the activator is a fragment of a monoclonal antibody of CD28. In certain aspects, the antibody fragment is a scFv, (scFv)₂, Fab, Fab′, and F(ab′)₂, F(ab1)₂, Fv, dAb, or Fd of a monoclonal antibody of CD28. In certain aspects, the activator is a nanobody, a bispecific antibody, or a multispecific antibody against CD28.

In some aspects, the immune modulator comprises a tolerance inducing agent. In certain aspects, the tolerance inducing agent comprises a NF-κB inhibitor. Non-limiting examples of NF-κB inhibitors that can be used with the present disclosure includes: IKK complex inhibitors (e.g., TPCA-1, NF-κB Activation Inhibitor VI (BOT-64), BMS 345541, Amlexanox, SC-514 (GK 01140), IMD 0354, IKK-16), IκB degradation inhibitor (e.g., BAY 11-7082, MG-115, MG-132, Lactacystin, Epoxomicin, Parthenolide, Carfilzomib, MLN-4924 (Pevonedistat)), NF-κB nuclear translocation inhibitor (e.g., JSH-23, Rolipram), p65 acetylation inhibitor (e.g., Gallic acid, Anacardic acid), NF-κB-DNA binding inhibitor (e.g., GYY 4137, p-XSC, CV 3988, Prostaglandin E2 (PGE2)), NF-κB transactivation inhibitor (e.g., LY 294002, Wortmannin, Mesalamine), or combinations thereof. See also Gupta, S. C., et al., Biochim Biophys Acta 1799:775-787 (2010), which is herein incorporated by reference in its entirety. In some aspects, an immune modulator that can inhibit NF-κB activity and be used with the EVs disclosed herein comprises an antisense-oligonucleotide that specifically targets NF-κB. In further aspects, an immune modulator capable of inducing tolerance comprises a COX-2 inhibitor, mTOR inhibitor (e.g., rapamycin and derivatives, e.g., antisense oligonucleotides targeting mTor), prostaglandins, nonsteroidal anti-inflammatory agents (NSAIDS), antileukotriene, aryl hydrocarbon receptor (AbR) ligand, vitamin D, retinoic acid, steroids, Fas receptor/ligand, CD22 ligand, IL-10, IL-35, IL-27, metabolic regulator (e.g., glutamate), glycans (e.g., ES62, LewisX, LNFPIII), peroxisome proliferator-activated receptor (PPAR) agonists, immunoglobulin-like transcript (ILT) family of receptors (e.g., ILT3, ILT4, HLA-G, ILT-2), minocycline, TLR4 agonists, or combinations thereof.

In some aspects, the immune modulator is an agonist. In certain aspects, the agonist is an endogenous agonist, such as a hormone, or a neurotransmitter. In other aspects, the agonist is an exogenous agonist, such as a drug. In some aspects, the agonist is a physical agonist, which can create an agonist response without binding to the receptor. In some aspects, the agonist is a superagonist, which can produce a greater maximal response than the endogenous agonist. In certain aspects, the agonist is a full agonist with full efficacy at the receptor. In other aspects, the agonist is a partial agonist having only partial efficacy at the receptor relative to a full agonist. In some aspects, the agonist is an inverse agonist that can inhibit the constitutive activity of the receptor. In some aspects, the agonist is a co-agonist that works with other co-agonists to produce an effect on the receptor. In certain aspects, the agonist is an irreversible agonist that binds permanently to a receptor through formation of covalent bond. In certain aspects, the agonist is selective agonist for a specific type of receptor

In some aspects, the immune modulator is an antagonist. In specific aspects, the antagonist is a competitive antagonist, which reversibly binds to the receptor at the same binding site as the endogenous ligand or agonist without activating the receptor. Competitive antagonist can affect the amount of agonist necessary to achieve a maximal response. In other aspects, the antagonist is a non-competitive antagonist, which binds to an active site of the receptor or an allosteric site of the receptor. Non-competitive antagonist can reduce the magnitude of the maximum response that can be attained by any amount of agonist. In further aspects, the antagonist is an uncompetitive antagonist, which requires receptor activation by an agonist before its binding to a separate allosteric binding site.

In some aspects, the immune modulator comprises an antibody or an antigen-binding fragment. The immunomodulating component can be a full length protein or a fragment thereof. The antibody or antigen-binding fragment can be derived from natural sources, or partly or wholly synthetically produced. In some aspects, the antibody is a monoclonal antibody. In some of these aspects, the monoclonal antibody is an IgG antibody. In certain aspects, the monoclonal antibody is an IgG1, IgG2, IgG3, or IgG4. In some other aspects, the antibody is a polyclonal antibody. In certain aspects, the antigen-binding fragment is selected from Fab, Fab′, and F(ab′)₂, F(ab1)₂, Fv, dAb, and Fd fragments. In certain aspects, the antigen-binding fragment is an scFv or (scFv)₂ fragment. In certain other aspects, the antibody or antigen-binding fragment is a Nanobody® (single-domain antibody). In some aspects, the antibody or antigen-binding fragment is a bispecific or multispecific antibody.

In various aspects, the antibody or antigen-binding fragment is fully human. In some aspects, the antibody or antigen-binding fragment is humanized. In some aspects, the antibody or antigen-binding fragment is chimeric. In some of these aspects, the chimeric antibody has non-human V region domains and human C region domains. In some aspects, the antibody or antigen-binding fragment is non-human, such as murine or veterinary.

In certain aspects, the immunomodulating component is a polynucleotide. In some of these aspects, the polynucleotide includes, but is not limited to, an mRNA, a miRNA, an siRNA, an antisense oligonucleotide (e.g., antisense RNA or antisense DNA), a phosphorodiamidate morpholino oligomer (PMO), a peptide-conjugated phosphorodiamidate morpholino oligomer (PPMO), an shRNA, a lncRNA, a dsDNA, and combinations thereof. In some aspects, the polynucleotide is an RNA (e.g., an mRNA, a miRNA, an siRNA, an antisense oligonucleotide (e.g., antisense RNA), an shRNA, or an lncRNA). In some of these aspects, when the polynucleotide is an mRNA, it can be translated into a desired polypeptide. In some aspects, the polynucleotide is a microRNA (miRNA) or pre-miRNA molecule. In some of these aspects, the miRNA is delivered to the cytoplasm of the target cell, such that the miRNA molecule can silence a native mRNA in the target cell. In some aspects, the polynucleotide is a small interfering RNA (siRNA) or a short hairpin RNA (shRNA) capable of interfering with the expression of an oncogene or other dysregulating polypeptides. In some of these aspects, the siRNA is delivered to the cytoplasm of the target cell, such that the siRNA molecule can silence a native mRNA in the target cell. In some aspects, the polynucleotide is an antisense oligonucleotide (e.g., antisense RNA) that is complementary to an mRNA. In some aspects, the polynucleotide is a long non-coding RNA (lncRNA) capable of regulating gene expression and modulating diseases. In some aspects, the polynucleotide is a DNA that can be transcribed into an RNA. In some of these aspects, the transcribed RNA can be translated into a desired polypeptide.

In some aspects, the immunomodulating component is a protein, a peptide, a glycolipid, or a glycoprotein.

In various aspects, the EV composition comprises two or more above mentioned immunomodulating components, including mixtures, fusions, combinations and conjugates, of atoms, molecules, etc. In some aspects, the composition comprises one, two, three, four, five, six, seven, eight, nine, ten, eleven, or twelve different immunomodulating components associated with the membrane or enclosed within the enclosed volume of the extracellular vesicle. In certain aspects, the composition comprises a nucleic acid combined with a polypeptide. In certain aspects, the composition comprises two or more polypeptides conjugated to each other. In certain aspects, the composition comprises a protein conjugated to a biologically active molecule. In some of these aspects, the biologically active molecule is a prodrug.

In some aspects, any suitable method can be used to link an antigen or any other molecules of interest (e.g., adjuvant, immune modulator, and/or targeting moiety described herein) to an exterior surface and/or luminal surface of the EV. In certain aspects, the antigen or any other molecules of interest is linked to the exterior surface and/or the luminal surface of the EV by any suitable coupling strategies known in the art. In some aspects, the coupling strategy comprises: an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof. In some aspects, the anchoring moiety comprises a cholesterol, fatty acid (e.g., palmitate), tocopherol (e.g., vitamin E), alkyl chain, aromatic ring, or any combination thereof. In some aspects, the chemical conjugation comprises a maleimide moiety, copper-free, biorthogonal click chemistry (e.g., azide/strained alkyne (DIFO)), metal-catalyzed click chemistry (e.g., CUAAC, RUAAC), or any combination thereof. Additional description relating to the different approaches of linking an antigen or any other molecules of interest are provided elsewhere in the present disclosure. For instance, in some aspects, any of the coupling strategies described above can be used in combination with a scaffold moiety described herein (e.g., Scaffold X, e.g., PTGFRN).

### II.E Scaffold X-Engineered EVs

In some aspects, EVs of the present disclosure comprise a membrane modified in its composition. For example, their membrane compositions can be modified by changing the protein, lipid, or glycan content of the membrane.

In some aspects, the surface-engineered EVs are generated by chemical and/or physical methods, such as PEG-induced fusion and/or ultrasonic fusion. In other aspects, the surface-engineered EVs are generated by genetic engineering. EVs produced from a genetically-modified producer cell or a progeny of the genetically-modified cell can contain modified membrane compositions. In some aspects, surface-engineered EVs have scaffold moiety (e.g., Scaffold X) at a higher or lower density (e.g., higher number) or include a variant or a fragment of the scaffold moiety.

For example, surface (e.g., Scaffold X)-engineered EVs, can be produced from a cell (e.g., HEK293 cells) transformed with an exogenous sequence encoding a scaffold moiety (e.g., Scaffold X) or a variant or a fragment thereof. EVs including scaffold moiety expressed from the exogenous sequence can include modified membrane compositions.

Various modifications or fragments of the scaffold moiety can be used for the aspects of the present disclosure. For example, scaffold moiety modified to have enhanced affinity to a binding agent can be used for generating surface-engineered EV that can be purified using the binding agent. Scaffold moieties modified to be more effectively targeted to EVs and/or membranes can be used. Scaffold moieties modified to comprise a minimal fragment required for specific and effective targeting to exosome membranes can be also used.

Scaffold moieties can be engineered to be expressed as a fusion molecule, e.g., fusion molecule of Scaffold X to an antigen, an adjuvant, and/or an immune modulator. For example, the fusion molecule can comprise a scaffold moiety disclosed herein (e.g., Scaffold X, e.g., PTGFRN, BSG, IGSF2, IGSF3, IGSF8, ITGB1, ITGA4, SLC3A2, ATP transporter, or a fragment or a variant thereof) linked to an antigen, an adjuvant, and/or an immune modulator. In case of the fusion molecule, the antigen, adjuvant, and/or immune modulator can be a natural peptide, a recombinant peptide, a synthetic peptide, or any combination thereof. In some aspects, a fusion molecule disclosed herein further comprises an affinity ligand. For example, in certain aspects, an affinity ligand is fused to a molecule of interest (e.g., antigen, adjuvant, immune modulator, and/or targeting moiety), and then the molecule of interest is conjugated to a moiety on EVs, e.g., Scaffold X via the affinity ligand. In certain aspects, the affinity ligand increases the binding of the molecule of interest to a moiety on EVs, e.g., Scaffold X.

In some aspects, the surface (e.g., Scaffold X)-engineered EVs described herein demonstrate superior characteristics compared to EVs known in the art. For example, surface (e.g., Scaffold X)-engineered contain modified proteins more highly enriched on their surface than naturally occurring EVs or the EVs produced using conventional exosome proteins. Moreover, the surface (e.g., Scaffold X)-engineered EVs of the present disclosure can have greater, more specific, or more controlled biological activity compared to naturally occurring EVs or the EVs produced using conventional exosome proteins.

In some aspects, the Scaffold X comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at TABLE 7 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 100, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the fragments of PTGFRN polypeptide lack one or more functional or structural domains, such as IgV.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 33. In other aspects, the Scaffold X comprises the amino acid sequence of SEQ ID NO: 33, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 33 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 33.

In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 2, 3, 4, 5, 6, or 7. In other aspects, the Scaffold X comprises the amino acid sequence of SEQ ID NO: 2, 3, 4, 5, 6, or 7, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 2, 3, 4, 5, 6, or 7 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 2, 3, 4, 5, 6, or 7.

TABLE 7
Exemplary Scaffold X Protein Sequences

| Protein | Sequence |
|---|---|
| The | MGRLASRPLLLALLSLALCRGRVVRVPTATLVRVVGTELVIPCNVSDYDGPSEQNFDWSF |
| PTGFRN | SSLGSSFVELASTWEVGFPAQLYQERLQRGEILLRRTANDAVELHIKNVQPSDQGHYKCS |
| Protein | TPSTDATVQGNYEDTVQVKVLADSLHVGPSARPPPSLSLREGEPFELRCTAASASPLHTH |
| (SEQ ID | LALLWEVHRGPARRSVLALTHEGRFHPGLGYEQRYHSGDVRLDTVGSDAYRLSVSRALSA |
| NO: 1) | DQGSYRCIVSEWIAEQGNWQEIQEKAVEVATVVIQPSVLRAAVPKNVSVAEGKELDLTCN |
| | ITTDRADDVRPEVTWSFSRMPDSTLPGSRVLARLDRDSLVHSSPHVALSHVDARSYHLLV |
| | RDVSKENSGYYYCHVSLWAPGHNRSWHKVAEAVSSPAGVGVTWLEPDYQVYLNASKVPGF |
| | ADDPTELACRVVDTKSGEANVRFTVSWYYRMNRRSDNVVTSELLAVMDGDWTLKYGERSK |
| | QRAQDGDFIFSKEHTDTFNFRIQRTTEEDRGNYYCVVSAWTKQRNNSWVKSKDVFSKPVN |
| | IFWALEDSVLVVKARQPKPFFAAGNTFEMTCKVSSKNIKSPRYSVLIMAEKPVGDLSSPN |
| | ETKYIISLDQDSVVKLENWTDASRVDGVVLEKVQEDEFRYRMYQTQVSDAGLYRCMVTAW |
| | SPVRGSLWREAATSLSNPIEIDFQTSGPIFNASVHSDTPSVIRGDLIKLFCIITVEGAAL |
| | DPDDMAFDVSWFAVHSFGLDKAPVLLSSLDRKGIVTTSRRDWKSDLSLERVSVLEFLLQV |
| | HGSEDQDFGNYYCSVTPWVKSPTGSWQKEAEIHSKPVFITVKMDVLNAFKYPLLIGVGLS |
| | TVIGLLSCLIGYCSSHWCCKKEVQETRRERRRLMSMEMD |
| The | GPIFNASVHSDTPSVIRGDLIKLFCIITVEGAALDPDDMAFDVSWFAVHSFGLDKAPVLL |
| PTGFRN | SSLDRKGIVTTSRRDWKSDLSLERVSVLEFLLQVHGSEDQDFGNYYCSVTPWVKSPTGSW |
| protein | QKEAEIHSKPVFITVKMDVLNAFKYPLLIGVGLSTVIGLLSCLIGYCSSHWCCKKEVQET |
| Fragment | RRERRRLMSMEM |
| (SEQ ID | 687-878 of SEQ ID NO: 1 |
| NO: 33) | |

Non-limiting examples of other Scaffold X proteins can be found at U.S. Pat. No. 10,195,290B1, issued Feb. 5, 2019, which is incorporated by reference in its entireties.

In some aspects, the sequence encodes a fragment of the scaffold moiety lacking at least 5, 10, 50, 100, 200, 300, 400, 500, 600, 700, or 800 amino acids from the N-terminus of the native protein. In some aspects, the sequence encodes a fragment of the scaffold moiety lacking at least 5, 10, 50, 100, 200, 300, 400, 500, 600, 700, or 800 amino acids from the C-terminus of the native protein. In some aspects, the sequence encodes a fragment of the scaffold moiety lacking at least 5, 10, 50, 100, 200, 300, 400, 500, 600, 700, or 800 amino acids from both the N-terminus and C-terminus of the native protein. In some aspects, the sequence encodes a fragment of the scaffold moiety lacking one or more functional or structural domains of the native protein.

In some aspects, the scaffold moieties, e.g., Scaffold X, e.g., a PTGFRN protein, are linked to one or more heterologous proteins. The one or more heterologous proteins can be linked to the N-terminus of the scaffold moieties. The one or more heterologous proteins can be linked to the C-terminus of the scaffold moieties. In some aspects, the one or more heterologous proteins are linked to both the N-terminus and the C-terminus of the scaffold moieties. In some aspects, the heterologous protein is a mammalian protein. In some aspects, the heterologous protein is a human protein.

In some aspects, Scaffold X can be used to link any moiety to the luminal surface and on the exterior surface of the EV at the same time. For example, the PTGFRN polypeptide can be used to link one or more payloads disclosed herein (e.g., an antigen, an adjuvant, and/or an immune modulator) inside the lumen (e.g., on the luminal surface) in addition to the exterior surface of the EV. Therefore, in certain aspects, Scaffold X can be used for dual purposes, e.g., an antigen on the luminal surface and an adjuvant or immune modulator on the exterior surface of the EV an antigen on the exterior surface of the EV and the adjuvant or immune modulator on the luminal surface, an adjuvant on the luminal surface and an immune modulator on the exterior surface of the EV or an immune modulator on the luminal surface and an adjuvant on the exterior surface of the EV.

### II.F Scaffold Y-Engineered EVs

In some aspects, EVs of the present disclosure comprise an internal space (i.e., lumen) that is different from that of the naturally occurring EVs. For example, the EV can be changed such that the composition in the luminal surface of the EV has the protein, lipid, or glycan content different from that of the naturally occurring exosomes.

In some aspects, engineered EVs can be produced from a cell transformed with an exogenous sequence encoding a scaffold moiety (e.g., Scaffold Y) or a modification or a fragment of the scaffold moiety that changes the composition or content of the luminal surface of the EV. Various modifications or fragments of the exosome protein that can be expressed on the luminal surface of the EV can be used for the aspects of the present disclosure.

In some aspects, the exosome proteins that can change the luminal surface of the EVs include, but are not limited to, the myristoylated alanine rich Protein Kinase C substrate (MARCKS) protein, the myristoylated alanine rich Protein Kinase C substrate like 1 (MARCKSL1) protein, the brain acid soluble protein 1 (BASP1) protein, or any combination thereof.

TABLE 8
Exemplary Scaffold Y Protein Sequences

| Protein | Sequence |
|---|---|
| The BASP1 | MGGKLSKKKK GYNVNDEKAK EKDKKAEGAA TEEEGTPKES EPQAAAEPAE |
| protein | AKEGKEKPDQ DAEGKAEEKE GEKDAAAAKE EAPKAEPEKT EGAAEAKAEP |
| (SEQ ID NO: | PKAPEQEQAA PGPAAGGEAP KAAEAAAAPA ESAAPAAGEE PSKEEGEPKK |
| 49) | TEAPAAPAAQ ETKSDGAPAS DSKPGSSEAA PSSKETPAAT EAPSSTPKAQ |
| | GPAASAEEPK PVEAPAANSD QTVTVKE |

The mature BASP1 protein sequence is missing the first Met from SEQ ID NO: 49 and thus contains amino acids 2 to 227 of SEQ ID NO: 49. Similarly, the mature MARCKS and MARCKSL1 proteins also lack the first Met from SEQ ID NOs: 47 and 48, respectively. Accordingly, the mature MARCKS protein contains amino acids 2 to 332 of SEQ ID NO: 47. The mature MARCKSL1 protein contains amino acids 2 to 227 of SEQ ID NO: 48.

In other aspects, Scaffold Y useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 2 to 227 of SEQ ID NO: 49. In other aspects, the Scaffold Y comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to any one of SEQ ID NOs: 50-155. In other aspects, a Scaffold Y useful for the present disclosure comprises the amino acid sequence of SEQ ID NO: 49, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, a Scaffold Y useful for the present disclosure comprises the amino acid sequence of any one of SEQ ID NOs: 50-155 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NOs: 50-155.

In some aspects, the protein sequence of any of SEQ ID NOs: 47-155 is sufficient to be a Scaffold Y for the present disclosure (e.g., scaffold moiety linked to an antigen and/or an adjuvant and/or an immune modulator).

Non-limiting examples of scaffold proteins can be found at WO/2019/099942, published May 23, 2019 and WO/2020/101740, published May 22, 2020, which are incorporated by reference in their entireties.

In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached posttranslationally to internal serine/threonine or tyrosine residues via a hydroxyester linkage. Membrane anchors known in the art are presented in the following table:

| Modification | Modifying Group |
|---|---|
| S-Palmitoylation | 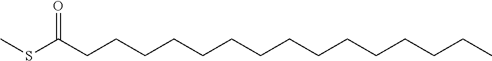 [SMILES: CCCCCCCCCCCCCCCC(=O)SC] |
| N-Palmitoylation | 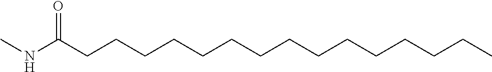 [SMILES: CCCCCCCCCCCCCCCC(=O)NC] |
| N-Myristoylation | 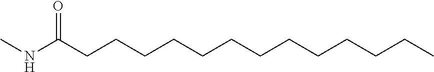 [SMILES: CCCCCCCCCCCCCC(=O)NC] |
| O-Acylation | 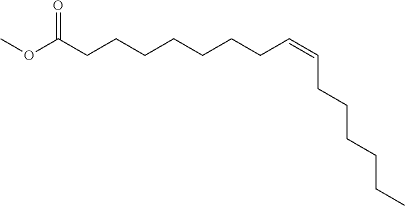 [SMILES: CCCCCC/C=C\CCCCCCCC(=O)OC] |
| Farnesylation | 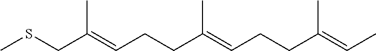 [SMILES: C/C=C(\C)CC/C=C(\C)CC/C=C(\C)CSC] |
| Geranylgeranylation | 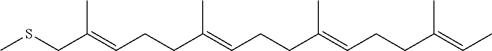 [SMILES: C/C=C(\C)CC/C=C(\C)CC/C=C(\C)CC/C=C(\C)CSC] |
| Cholesterol | 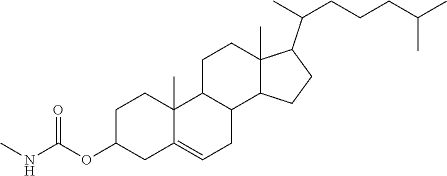 [SMILES: CNC(=O)OC1CCC2(C)C(=CCC3C2CCC2(C)C(C(C)CCCC(C)C)CCC32)C1] |

### II.G. Conjugated EVs

Unlike antibodies, EVs can accommodate large numbers of molecules attached to their surface, e.g., on the order of thousands to tens of thousands of molecules per EV. EV-drug conjugates thus represent a platform to deliver a high concentration of therapeutic compound to discrete cell types, while at the same time limiting overall systemic exposure to the compound, which in turn reduces off-target toxicity.

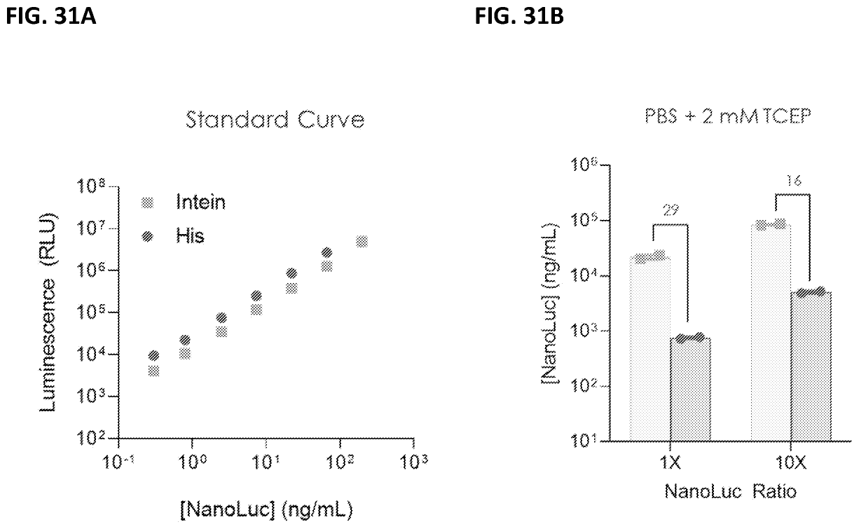

The present disclosure provide EVs that have been engineered by reacting a first molecular entity comprising a free thiol group with a second molecular entity comprising a maleimide group, wherein the maleimide moiety covalently links the first molecular entity with the second molecular entity via a maleimide moiety as presented in FIG. 31.

Non-limiting examples of biologically active molecules that can attached to an EV via a maleimide moiety include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, or siRNA), morpholino, amino acids (e.g., amino acids comprising a detectable moiety or a toxin that disrupt translation), polypeptides (e.g., enzymes), lipids, carbohydrates, small molecules (e.g., small molecule drugs and toxins), antigens (e.g., vaccine antigens), adjuvants (e.g., vaccine adjuvants), etc.

In some aspects, an EV of the present disclosure can comprise more than one type of biologically active molecule. In some aspects, biologically active molecules can be, e.g., small molecules such as cyclic dinucleotides, toxins such as auristatins (e.g., monoethyl auristatin E, MMAE), antibodies (e.g., naked antibodies or antibody-drug conjugates), STING agonists, tolerizing agents, antisense oligonucleotides, PROTACs, morpholinos, lysophosphatidic acid receptor antagonists (e.g., LPA1 antagonists) or any combinations thereof. In some aspects, an EV of the present disclosure can comprise, e.g., a vaccine antigen and optionally a vaccine adjuvant. In some aspects, an EV of the present disclosure can comprise a therapeutic payload (e.g., a STING or one payload disclosed below) and a targeting moiety and/or a tropism moiety.

### II.H Linkers

As described supra, extracellular vesicles (EVs) of the present disclosure can comprises one or more linkers that link a molecule of interest (e.g., antigen, adjuvant, or immune modulator) to the EVs (e.g., to the exterior surface or on the luminal surface). In some aspects, the molecule of interest (i.e., payload) (e.g., antigen, adjuvant, or immune modulator) is linked to the EVs directly or via a scaffold moiety (e.g., Scaffold X or Scaffold Y). For example, in certain aspects, a payload (e.g., an antigen, adjuvant, and/or immune modulator) is linked to the exterior surface of an exosome via Scaffold X. In further aspects, a payload (e.g., an antigen, adjuvant, and/or immune modulator) is linked to the luminal surface of an exosome via Scaffold X or Scaffold Y. In some aspects, a payload (e.g., an antigen, adjuvant, and/or immune modulator) is linked to the luminal surface of an exosome via Scaffold X. In some aspects, a payload (e.g., an antigen, adjuvant, and/or immune modulator) is linked to the luminal surface of an exosome via Scaffold Y. In some aspects, a payload (e.g., an antigen, adjuvant, and/or immune modulator) is linked to the luminal surface of an exosome via Scaffold X and Scaffold Y. In some aspects, a payload (e.g., an antigen, adjuvant, and/or immune modulator) is conjugated to Scaffold X via a linker (e.g., those described herein). In certain aspects, a payload (e.g., an antigen, adjuvant, and/or immune modulator) is conjugated to Scaffold X using more than one linker (i.e., “linker combination”). In some aspects, a payload (e.g., an antigen, adjuvant, and/or immune modulator) is conjugated to Scaffold Y via a linker (e.g., those described herein). In certain aspects, a payload (e.g., an antigen, adjuvant, and/or immune modulator) is conjugated to Scaffold Y using a linker combination. In some aspects, a linker combination comprises at least 2, at least 3, at least 4, at least 5, or at least 6 or more different linkers disclosed herein. In some aspects, linkers in a linker combination can be linked by an ester linkage (e.g., phosphodiester or phosphorothioate ester).

The linker can be any chemical moiety known in the art.

As used herein, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present, each of the linkers can be the same or different. Generally, linkers provide flexibility or prevent/ameliorate steric hindrances. Linkers are not typically cleaved; however in certain aspects, such cleavage can be desirable. Accordingly, in some aspects, a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker can comprise at least about 110, at least about 120, at least about 130, at least about 140, at least about 150, at least about 160, at least about 170, at least about 180, at least about 190, or at least about 200 amino acids.

In other aspects, the peptide linker can comprise at least about 200, at least about 300, at least about 400, at least about 500, at least about 600, at least about 700, at least about 800, at least about 900, or at least about 1,000 amino acids. The peptide linker can comprise between 1 and about 5 amino acids, between 1 and about 10 amino acids, between 1 and about 20 amino acids, between about 10 and about 50 amino acids, between about 50 and about 100 amino acids, between about 100 and about 200 amino acids, between about 200 and about 300 amino acids, between about 300 and about 400 amino acids, between about 400 and about 500 amino acids, between about 500 and about 600 amino acids, between about 600 and about 700 amino acids, between about 700 and about 800 amino acids, between about 800 and about 900 amino acids, or between about 900 and about 1000 amino acids.

As described herein, in some aspects, a linker useful for the present disclosure comprises a glycine/serine linker. In some aspects, the peptide linker is glycine/serine linker according to the formula [(Gly) n-Ser]m (SEQ ID NO: 374), where n is any integer from 1 to 100 and m is any integer from 1 to 100. In some aspects, the glycine/serine linker is according to the formula [(Gly)x-Sery]z (SEQ ID NO: 375), wherein x in an integer from 1 to 4, y is 0 or 1, and z is an integers from 1 to 50. In some aspects, the peptide linker comprises the sequence Gn (SEQ ID NO: 376), where n can be an integer from 1 to 100. In some aspects, the peptide linker can comprise the sequence (Gly Ala)n (SEQ ID NO: 377), wherein n is an integer between 1 and 100. In some aspects, the peptide linker can comprise the sequence (GlyGlySer)n (SEQ ID NO: 378), wherein n is an integer between 1 and 100. In certain aspects, the peptide linker comprises the sequence GGGG (SEQ ID NO: 197).

In some aspects, the peptide linker comprises the sequence (GGGS)n (SEQ ID NO: 203). In certain aspects, the peptide linker comprises the sequence (GGS)n (GGGGS)n (SEQ ID NO: 204). In such aspects, n can be an integer from 1 to 100. In some aspects, n can be an integer from one to 20, i.e., 1, 2, 3, 4, 5, 6, 7, 8, 9, 10, 11, 12, 13, 14, 15, 16, 17, 18, 19, or 20. In some aspects, n is an integer from 1 to 100.

Examples of linkers that are useful for the present disclosure include, but are not limited to, GGG, SGGSGGS (SEQ ID NO: 198), GGSGGSGGSGGSGGG (SEQ ID NO: 199), GGSGGSGGGGSGGGGS (SEQ ID NO: 200), GGSGGSGGSGGSGGSGGS (SEQ ID NO: 201), or GGGGSGGGGSGGGGS (SEQ ID NO: 202). In some aspects, the linker is a poly-G sequence (GGGG)n (SEQ ID NO: 373), where n can be an integer from 1-100.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect, the peptide linker can comprise non-naturally occurring polypeptides which are modified forms of naturally occurring polypeptides (e.g., comprising a mutation such as an addition, substitution or deletion).

In some aspects, the peptide linker can comprise non-naturally occurring amino acids. In yet other aspects, the peptide linker can comprise naturally occurring amino acids occurring in a linear sequence that does not occur in nature. In still other aspects, the peptide linker can comprise a naturally occurring polypeptide sequence.

In some aspects, the linker comprises a non-peptide linker. In other aspects, the linker consists of a non-peptide linker. In some aspects, the non-peptide linker can be, e.g., maleimido caproyl (MC), maleimido propanoyl (MP), methoxyl polyethyleneglycol (MPEG), succinimidyl 4-(N-maleimidomethyl)-cyclohexane-1-carboxylate (SMCC), m-maleimidobenzoyl-N-hydroxysuccinimide ester (MBS), succinimidyl 4-(p-maleimidophenyl) butyrate (SMPB), N-succinimidyl (4-iodoacetyl)aminobenzonate (SIAB), succinimidyl 6-[3-(2-pyridyldithio)-propionamide]hexanoate (LC-SPDP), 4-succinimidyloxycarbonyl-alpha-methyl-alpha-(2-pyridyldithio) toluene (SMPT), etc. (see, e.g., U.S. Pat. No. 7,375,078, which is herein incorporated by reference in its entirety).

Linkers can be susceptible to cleavage (“cleavable linker”) thereby facilitating release of the biologically active molecule (e.g., antigen, adjuvant, or immune modulator). Therefore, in some aspects, a linker that can be used with the present disclosure comprises a cleavable linker. Such cleavable linkers can be susceptible, for example, to acid-induced cleavage, photo-induced cleavage, peptidase-induced cleavage, esterase-induced cleavage, and disulfide bond cleavage, at conditions under which the biologically active molecule remains active. In some aspects, a cleavable linker comprises a spacer. In certain aspects, a spacer comprises PEG.

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the linker comprises a non-cleavable linker (i.e., resistant or substantially resistant to cleavage).

In some aspects, a linker combination disclosed herein comprises only cleavable linkers. In some aspects, a linker combination disclosed herein comprises only non-cleavable linkers. In some aspects, a linker combination disclosed herein comprises both cleavable and non-cleavable linkers. Additional disclosure relating to cleavable and non-cleavable linkers that can be used with the present disclosure are provided below.

### II.I Affinity Ligands

EVs disclosed herein can comprise one or more affinity ligands that link or conjugate a molecule of interest (e.g., antigen, adjuvant, immune modulator, and/or targeting moiety) to the EVs (e.g., to the exterior surface or on the luminal surface) or to a target cell. In some aspects, an affinity ligand disclosed herein has one or more of the following properties: (i) derived from a synthetic library, (ii) sub-nanomolar affinity for a scaffold moiety (e.g., Scaffold X) with emphasis on slow off rate, (iii) binds epitope on membrane-distal IgV domain of a scaffold moiety (e.g., Scaffold X), (iv) free of disulfide linkages, (v) free of N-linked glycosylation sites, (vi) less than 20 amino acids in length, (vii) monomeric, (viii) electroneutral at physiological pH, (ix) hydrophilic, (x) resistant to protease digestion, (xi) amenable to expression in prokaryotic and eukaryotic hosts, (xii) can accommodate N- or C-terminus fusion, (xiii) nonimmunogenic, (xiv) can contain a tag for purification and/or separation, e.g., of an EV, and (xv) combinations thereof. As described herein, in some aspects, an affinity ligand disclosed herein can specifically bind (e.g., with high affinity) to a moiety expressed on the surface of an EV. In certain aspects, an affinity ligand specifically binds to a scaffold moiety expressed on the surface of an EV. In some aspects, an affinity ligand specifically binds to any moiety expressed on the surface of an EV (e.g., cholesterol). In some aspects, an affinity ligand disclosed herein can specifically bind (e.g., with high affinity) to a moiety expressed on a target cell. Non-limiting examples of such affinity ligands are provided throughout the present disclosure.

As described above, an affinity ligand useful for the present disclosure can be engineered to express one or more tags. In some aspects, such tags can be useful in the purification and/or separation of an agent that is conjugated to the affinity ligand. For example, in some aspects, an EV comprises a scaffold moiety that is conjugated to an affinity ligand fusion, which comprises a molecule of interest (e.g., antigen, adjuvant, immune modulator, and/or targeting moiety) and a tag. In such aspects, the tag can be used to purify and/or separate the EV from a sample comprising the EV. In some aspects, a tag of an affinity ligand fusion described above is present between the affinity ligand and the molecule of interest. In some aspects, the tag of an affinity ligand fusion described above can be present at an end (e.g., N-terminus) of the molecule of interest, as long as the tag does not interfere with the activity of the molecule of interest. Any tags useful in the art for purifying and/or separating an agent from a sample can be used in the present disclosure. Non-limiting examples of such tags include polyhistidine tags, polyarginine tags, glutathione-S-transferase (GST), maltose binding protein (MBP), S-tag, influenza virus HA tag, thioredoxin, staphylococcal protein A tag, FLAG™ epitope, AviTag epitope (for subsequent biotinylation), c-myc epitope, and combinations thereof. See, e.g., U.S. Pat. No. 7,655,413, which is herein incorporated by reference in its entirety.

As described herein, in some aspects, a molecule of interest can be expressed on the surface of an EV via a scaffold moiety. In some of these aspects, the molecule of interest can be linked or conjugated to the scaffold moiety via an affinity ligand. For instance, as described herein, in certain aspects, an affinity ligand can be fused to a molecule of interest (e.g., antigen, adjuvant, immune modulator, and/or targeting moiety), and then the molecule of interest can be conjugated to a moiety expressed on the surface of an EV (e.g., scaffold moiety) via the affinity ligand. In some aspects, the affinity ligand increases the binding of the molecule of interest (e.g., antigen, adjuvant, immune modulator, and/or targeting moiety) to the moiety on the EV (e.g., scaffold moiety). In certain aspects, the binding of the molecule of interest to the moiety on the EV (e.g., scaffold moiety) is increased by at least about one-fold, at least about two-fold, at least about three-fold, at least about four-fold, at least about five-fold, at least about six-fold, at least about seven-fold, at least about eight-fold, at least about nine-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to a reference (e.g., binding of the molecule of interest to the moiety on the EV (e.g., scaffold moiety) without the use of an affinity ligand).

In some aspects, an affinity ligand that can be used with the present disclosure comprises a linear peptide. In certain aspects, an affinity ligand comprises at least about two, at least about three, at least about four, at least about five, at least about seven, at least about eight, at least about nine, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

Not to be bound by any one theory, using an affinity ligand (e.g., disclosed herein) to link or conjugate a molecule of interest (e.g., antigen, adjuvant, immune modulator, and/or targeting moiety) to a moiety expressed on the surface of an EV (e.g., scaffold moiety) can improve one or more properties of the EV disclosed herein. For instance, in some aspects, by increasing the binding of a molecule of interest to a moiety on the EVs (e.g., scaffold moiety), an affinity ligand disclosed herein can allow for increased expression of the molecule of interest on the surface of an EV (e.g., exterior surface). Accordingly, in certain aspects, a fusion protein comprising (i) a molecule of interest (e.g., antigen, adjuvant, immune modulator, and/or targeting moiety), (ii) an affinity ligand, and (iii) a scaffold moiety is present in the EV (e.g., exterior surface) at a higher density compared to a reference (e.g., corresponding fusion protein without the affinity ligand). In some aspects, the density of the fusion protein on the surface of the exosome is increased by at least about one-fold, at least about two-fold, at least about three-fold, at least about four-fold, at least about five-fold, at least about six-fold, at least about seven-fold, at least about eight-fold, at least about nine-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to the reference.

In some aspects, an improved binding of a molecule of interest (e.g., antigen, adjuvant, immune modulator, and/or targeting moiety) to a moiety expressed on the surface of an EV (e.g., scaffold moiety) can reduce the time required to produce an EV disclosed herein. Accordingly, in some aspects, an affinity ligand disclosed herein can reduce the time required for producing an engineered EV disclosed herein (e.g., comprising a molecule of interest and a scaffold moiety). In certain aspects, the time required to produce an engineered EV is reduced by at least about 5%, at least about 10%, at least about 20%, at least about 30%, at least about 40%, at least about 50%, at least about 60%, at least about 70%, at least about 80%, or at least about 90% or more, compared to a reference (e.g., time required to produce the corresponding EV without the affinity ligand).

In some aspects, an affinity ligand useful for the present disclosure comprises a cleavage site, such as a protease (e.g., thrombin) cleavage site.

A non-limiting example of an EV comprising an affinity ligand is described below. It will be apparent to those skilled in the art that an affinity ligand disclosed herein can be used in combination with other EVs disclosed herein.

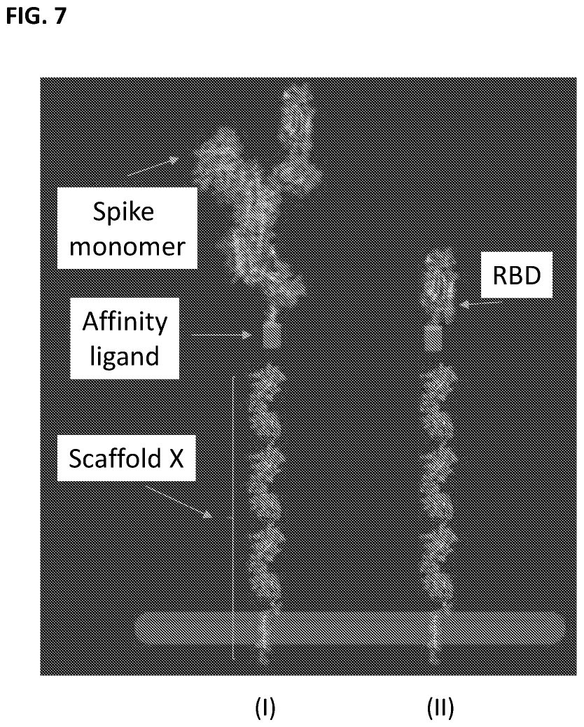

In some aspects, an EV comprises (i) an antigen, (ii) a scaffold moiety, and (iii) an affinity ligand, wherein the affinity ligand is used to link or conjugate the antigen to the scaffold moiety. As shown in FIG. 7, in some aspects, to produce such EVs, the affinity ligand is fused to an antigen (e.g., spike S protein), and then the antigen-affinity ligand fusion is linked or conjugated to the scaffold moiety via the affinity ligand. In certain aspects, the antigen comprises a receptor-binding domain (RBD) of a spike(S) protein of a coronavirus disclosed herein (e.g., COVID-19). In certain aspects, the scaffold moiety comprises a Scaffold X. Accordingly, in some aspects, an EV comprises (i) a RBD of a coronavirus S protein, (ii) a Scaffold X, and (iii) an affinity ligand, wherein the affinity ligand is used to link or conjugate the RBD of a coronavirus S protein to the Scaffold X. In some aspects, the affinity ligand can be used to link or conjugate a fragment of the RBD of a coronavirus S protein. In certain aspects, a fragment of the RBD of a coronavirus S protein that can be linked or conjugated to a Scaffold X using an affinity ligand disclosed herein is less than about 100 amino acids in length (e.g., less than about 90 amino acids, less than about 80 amino acids, less than about 70 amino acids, less than about 60 amino acids, less than about 50 amino acids, less than about 40 amino acids, less than about 30 amino acids, less than about 20 amino acids, less than about 10 amino acids, or more).

### III. Pharmaceutical Compositions

Provided herein are pharmaceutical compositions comprising an EV of the present disclosure having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and an exosome described herein. In certain aspects, the EVs are co-administered with of one or more additional therapeutic agents, in a pharmaceutically acceptable carrier. In some aspects, the pharmaceutical composition comprising the EV is administered prior to administration of the additional therapeutic agents. In other aspects, the pharmaceutical composition comprising the EV is administered after the administration of the additional therapeutic agents. In further aspects, the pharmaceutical composition comprising the EV is administered concurrently with the additional therapeutic agents.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs can be administered by parenteral, topical, intravenous, oral, subcutaneous, intra-arterial, intradermal, transdermal, rectal, intracranial, intraperitoneal, intranasal, intramuscular route or as inhalants. In certain aspects, the pharmaceutical composition comprising exosomes is administered intravenously, e.g. by injection. The EVs can optionally be administered in combination with other therapeutic agents that are at least partly effective in treating the disease, disorder or condition for which the EVs are intended.

Solutions or suspensions can include the following components: a sterile diluent such as water, saline solution, fixed oils, polyethylene glycols, glycerin, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs in an effective amount and in an appropriate solvent with one or a combination of ingredients enumerated herein, as desired. Generally, dispersions are prepared by incorporating the EVs into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The EVs can be administered in the form of a depot injection or implant preparation which can be formulated in such a manner to permit a sustained or pulsatile release of the EVs.

Systemic administration of compositions comprising exosomes can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects, the pharmaceutical composition comprising exosomes is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105 (46): 17908 (2008)), or by intramuscular injection, by subcutaneous administration, by direct injection into the thymus, or into the liver.

In certain aspects, the pharmaceutical composition comprising exosomes is administered as a liquid suspension. In certain aspects, the pharmaceutical composition is administered as a formulation that is capable of forming a depot following administration. In certain preferred aspects, the depot slowly releases the EVs into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs described herein. In some aspects, the dosage form is formulated as a liquid suspension for intravenous injection.

In certain aspects, the preparation of exosomes is subjected to radiation, e.g., X rays, gamma rays, beta particles, alpha particles, neutrons, protons, elemental nuclei, UV rays in order to damage residual replication-competent nucleic acids.

In certain aspects, the preparation of exosomes is subjected to gamma irradiation using an irradiation dose of more than 1, 5, 10, 15, 20, 25, 30, 35, 40, 50, 60, 70, 80, 90, 100, or more than 100 kGy.

In certain aspects, the preparation of exosomes is subjected to X-ray irradiation using an irradiation dose of more than 0.1, 0.5, 1, 5, 10, 15, 20, 25, 30, 35, 40, 50, 60, 70, 80, 90, 100, 200, 300, 400, 500, 600, 700, 800, 900, 1000, 2000, 3000, 4000, 5000, 6000, 7000, 8000, 9000, 10000, or greater than 10000 mSv.

### IV. Kits

Also provided herein are kits comprising one or more exosomes described herein. In some aspects, provided herein is a pharmaceutical pack or kit comprising one or more containers filled with one or more of the ingredients of the pharmaceutical compositions described herein, such as one or more exosomes provided herein, optional an instruction for use. In some aspects, the kits contain a pharmaceutical composition described herein and any prophylactic or therapeutic agent, such as those described herein.

### V. Methods of Producing EVs

EVs of the present disclosure differ from traditional vaccines in that the EVs can be rapidly engineered to express a moiety of interest (e.g., antigen, adjuvant, immune modulator, and/or targeting moiety). As described herein, the moieties of interest (i) can be directly linked to a surface of the EV (e.g., exterior surface and/or luminal surface), (ii) can be linked to a scaffold moiety (e.g., Scaffold X and/or Scaffold Y) and then expressed on a surface of the EV (e.g., exterior surface and/or luminal surface), (iii) can be expressed in the lumen of the EV, or (iv) combinations thereof. Such ability to rapidly engineer EVs is particularly useful in developing EV-based vaccines. For instance, a single EV engineered to express certain payloads and/or targeting moieties can be used in treating a wide range of diseases or disorders by simply “plugging” an antigen of interest into the EVs.

Accordingly, in some aspects, the present disclosure is directed to methods of producing such modular or “plug and play” EV vaccines. In certain aspects, a method of producing an EV-based vaccine comprises mixing an engineered EV with an antigen of interest, such that the antigen of interest is expressed in the engineered EV. As described herein, in some aspects, an antigen of interest that can be expressed in an EV comprises a full-length protein of a coronavirus (e.g., spike protein, envelope protein, and/or membrane protein). In certain aspects, the antigen of interest comprises a subunit of the full-length protein (e.g., receptor-binding domain of the spike protein). In some aspects, an EV can be engineered using methods disclosed herein to express multiple (e.g., two or more) coronavirus antigens, e.g., such as those described in the present disclosure. In some aspects, the engineered EV comprises one or more of the payloads disclosed herein (e.g., antigen, adjuvant, and/or immune modulator). In certain aspects, the engineered EV further comprises one or more scaffold moieties (e.g., Scaffold X and/or Scaffold Y). In some aspects, the engineered EV additionally comprises one or more targeting moieties. In some aspects, the engineered EV can be produced using any of the methods disclosed herein.

As will be apparent from the present disclosure, one of the features of the EV-based vaccine platform disclosed herein (e.g., modular or “plug and play” EVs) is that EVs can be isolated from a producer cell and stored indefinitely until they are to be used with the methods disclosed herein. As used herein, EVs that have been “isolated from a producer cell” refer to EVs that exist independent of the cells from which they are produced. In some aspects, once produced, the EVs that are useful for the present disclosure are purified or extracted from a culture containing the producer cells, and stored in a separate container until they are ready for further use (e.g., to add one or more antigens disclosed herein). Such EVs are also referred to herein as “base EVs” or “base exosomes.” As described herein, a base EV can differ from a naturally existing EV. For example, the base EVs can be genetically modified (e.g., by introducing a moiety of interest into the producer cells during production) or they can be modified after the EVs are produced and isolated from the producer cells.

For instance, as described herein, in producing the base EVs, they can be initially produced to comprise one or more moieties of interest, such as those that could be beneficial in a wide range of diseases or disorders (e.g., adjuvant and/or targeting moiety). Then, when desired, the base EVs can be rapidly modified by simply plugging or clipping on a specific antigen of interest, such as those useful to treat a neurological disorder, and thereby, produce or manufacture a vaccine that can be used to treat a disease or disorder described herein (e.g., neurological disorder). Such antigens can be added to the base EVs at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 2 years, at least about 3 years, at least about 4 years, or at least about 5 year or more after isolating the base EV from the producer cell.

Not to be bound by any one theory, the use of such base EVs can greatly improve one or more aspects of producing vaccines, particularly at a large manufacturing scale. While many traditional vaccines (e.g., peptide-based) have been used to treat and/or prevent certain diseases or disorders, they are generally poorly immunogenic and require repeated administrations and/or high doses. See, e.g., Hos, B. J., et al., Front Immunol 9:884 (2018), which is incorporated herein by reference in its entirety. Additionally, because of manufacturing complexities, compounded by the need for different formulations for different countries and age groups, it often takes multiple years to develop and manufacture a safe and efficacious vaccine. See, e.g., Smith, J., et al., Lancet 378 (9789): 428-438 (July 2011), which is incorporated herein by reference in its entirety. Accordingly, the ability to simply “plug” or “clip on” an antigen of interest to a base EV can be greatly advantageous when seeking to treat a disease or disorder that is more regional and/or more prevalent in certain individual or subsets of individuals (e.g., age group).

Accordingly, in some aspects, with the methods disclosed herein, the time required for manufacturing or producing a vaccine (“manufacturing time”) is reduced compared to a reference manufacturing time. In certain aspects, the reference manufacturing time refers to the time required to manufacture or produce a non-EV-based vaccine. In some aspects, the reference manufacturing time refers to the time required to manufacture or produce an EV-based vaccine wherein the antigen is not added to EVs that have been isolated from the producer cell (e.g., by introducing the antigen into the producer cell, such that when the EVs are produced, they comprise the antigen). In certain aspects, the manufacturing time is reduced by at least about 10%, at least about 20%, at least about 30%, at least about 40%, at least about 50%, at least about 60%, at least about 70%, at least about 80%, or at least about 90% or more, compared to the reference manufacturing time. In some aspects, the manufacturing time is less than about 12 months, less than about 11 months, less than about 10 months, less than about 9 months, less than about 8 months, less than about 7 months, less than about 6 months, less than about 5 months, less than about 4 months, less than about 3 months, less than about 2 months, or less than about 1 month. In some aspects, the manufacturing time is less than about 6 months.

In some aspects, EVs that can be produced or manufactured using the methods described herein are regionalized vaccines. As used herein, the term “regionalized vaccines” or “regional vaccines” refer to vaccines that are tailored to certain regions of the world. For instance, geographic isolation of certain genetic subtypes/serotypes of an infectious pathogen (e.g. virus) could require a more customized vaccine as opposed to a vaccine designed to address the extensive diversity of the pathogen worldwide. In some aspects, the methods disclosed herein can be used to produce or manufacture such regionalized vaccines by adding an antigen to an EV that has been isolated from a producer cell, wherein the antigen has been determined to be associated with a particular pathogen (or genetic subtype/serotype of a pathogen) prevalent within a certain region of the world. Non-limiting examples of such pathogens are provided elsewhere in the present disclosure.

In some aspects, EV-based vaccines that can be produced or manufactured using the methods described herein are individualized vaccines. As used herein, the term “individualized vaccines” and “personalized vaccines” can be used interchangeably and refer to vaccines that are tailored to a specific individual or subsets of individuals. Such a personalized vaccine could be of particular interest, e.g., for a cancer vaccine using neoantigens, since many neoantigens are specific for the particular cancer cells of an individual or subsets of individuals (e.g., those who share certain genetic background). In some aspects, the methods disclosed herein can be used to produce or manufacture such regionalized vaccines by adding an antigen to an EV that has been isolated from a producer cell, wherein the antigen has been determined to have (or likely to have) a therapeutic effect (e.g., induces an immune response) in the particular individual or subset of individuals.

As will be apparent to those skilled in the arts, methods of identifying regional- and/or individual-specific antigens are known in the art. See, e.g., US 2009/0169576; US 2014/0178438; Sakkhachornphop, S., et al., J Virol Methods 217:70-8 (June 2015); and Xu, K., et al., Sci Rep 8 (1): 1067 (January 2018), each of which is incorporated herein by reference in its entirety.

In some aspects, the present disclosure is also directed to methods of producing EVs described herein. In some aspects, the method comprises: obtaining the EV from a producer cell, wherein the producer cell contains two or more components of the EV (e.g., (i) antigen and adjuvant, (ii) antigen and immune modulator, (iii) antigen and targeting moiety, (iv) antigen, adjuvant, and targeting moiety, (v) antigen, immune modulator, and targeting moiety, (vi) antigen, adjuvant, and immune modulator, (vii) antigen, adjuvant, immune modulator, and targeting moiety); and optionally isolating the obtained EV. In some aspects, the method comprises: modifying a producer cell by introducing two or more components of an EV disclosed herein (e.g., (i) antigen and adjuvant, (ii) antigen and immune modulator, (iii) antigen and targeting moiety, (iv) antigen, adjuvant, and targeting moiety, (v) antigen, immune modulator, and targeting moiety, (vi) antigen, adjuvant, and immune modulator, (vii) antigen, adjuvant, immune modulator, and targeting moiety); obtaining the EV from the modified producer cell; and optionally isolating the obtained EV. In further aspects, the method comprises: obtaining an EV from a producer cell; isolating the obtained EV; and modifying the isolated EV (e.g., by inserting multiple exogenous biologically active molecules, e.g., antigens, adjuvants, and/or immune modulators, and/or targeting moieties). In certain aspects, the method further comprises formulating the isolated EV into a pharmaceutical composition.

As described herein, in some aspects, an EV that can be produced using the methods provided herein comprises one or more of the following features: (i) a luminal T cell antigen (e.g., attached to the luminal surface of the EV using a scaffold moiety); (ii) a surface B cell antigen (e.g., attached to the exterior surface of the EV using a scaffold moiety); and (iii) a STING agonist (e.g., loaded into the lumen of the EV). In addition to the scaffold moieties described herein (e.g., Scaffold X and Scaffold Y), any suitable method can be used to link an antigen or any other molecules of interest (e.g., adjuvant and/or targeting moiety) to an exterior surface and/or luminal surface of the EV. In certain aspects, the antigen or any other molecules of interest (e.g., adjuvant and/or targeting moiety) is linked to the exterior surface and/or the luminal surface of the EV by any suitable coupling strategies known in the art. In some aspects, the coupling strategy comprises: an anchoring moiety, affinity agent, chemical conjugation, cell penetrating peptide (CPP), split intein, Spy Tag/SpyCatcher, ALFA-tag, Streptavidin/Avitag, Sortase, SNAP-tag, ProA/Fc-binding peptide, or any combinations thereof. In some aspects, the anchoring moiety comprises a cholesterol, fatty acid (e.g., palmitate), tocopherol (e.g., vitamin E), alkyl chain, aromatic ring, or any combination thereof. In some aspects, the chemical conjugation comprises a maleimide moiety, copper-free, biorthogonal click chemistry (e.g., azide/strained alkyne (DIFO)), metal-catalyzed click chemistry (e.g., CUAAC, RUAAC), or any combination thereof. Additional description relating to the different approaches of linking an antigen or any other molecules of interest (e.g., adjuvant and/or targeting moiety) are provided elsewhere in the present disclosure.

In some aspects, to identify the T cell antigens that are useful for the present disclosure, an in silico structure-based network analysis can be used to determine one or more conserved T cell (e.g., CD8+ T cells) epitopes of a pathogen, e.g., coronavirus (e.g., SARS-CoV-1, SARS-CoV-2 (COVID-19), and/or MERS-CoV). Non-limiting examples of T cell epitopes of a coronavirus are provided in Table 2. In some aspects, the network analysis is applied to the spike, nucleocapsid, and/or non-structural proteins of a coronavirus (e.g., SARS-CoV-1, SARS-CoV-2 (COVID-19), and/or MERS-CoV). In some aspects, the T cell epitopes are CD8+ T cell epitopes and are conserved across different types of coronavirus (e.g., SARS-CoV-1, SARS-CoV-2 (COVID-19), and/or MERS-CoV). Additional disclosure relating to such an analysis is provided, e.g., in Gaiha et al., Science 364 (6439): 480-484 (May 2019), which is herein incorporated by reference in its entirety.

In some aspects, to identify the B cell antigens that are useful for the present disclosure, the spike protein of a coronavirus (e.g., SARS-CoV-1, SARS-CoV-2 (COVID-19), and/or MERS-CoV) can be assessed to determine B cell epitopes that are conserved across different types of coronavirus (e.g., SARS-CoV-1, SARS-CoV-2 (COVID-19), and/or MERS-CoV). As described herein, in certain aspects, the B cell antigen that can be attached to an EV (e.g., to the exterior surface of the EV using a scaffold moiety) comprises a receptor-binding domain (RBD) of a coronavirus spike protein. Additional disclosure relating to RBD is provided elsewhere herein.

### V.A Methods of Modifying a Producer Cell

As described supra, in some aspects, a method of producing an EV comprises modifying a producer cell with multiple (e.g., two or more) molecule of interest (e.g., exogenous biologically active molecules described herein (e.g., antigen, adjuvant, immune modulator), and/or targeting moiety). In some aspects, a producer cell disclosed herein can be further modified with a scaffold moiety disclosed herein (e.g., Scaffold X or Scaffold Y).

In some aspects, the producer cell can be a mammalian cell line, a plant cell line, an insect cell line, a fungi cell line, or a prokaryotic cell line. In certain aspects, the producer cell is a mammalian cell line. Non-limiting examples of mammalian cell lines include: a human embryonic kidney (HEK) cell line, a Chinese hamster ovary (CHO) cell line, an HT-1080 cell line, a HeLa cell line, a PERC-6 cell line, a CEVEC cell line, a fibroblast cell line, an amniocyte cell line, an epithelial cell line, a mesenchymal stem cell (MSC) cell line, and combinations thereof. In certain aspects, the mammalian cell line comprises HEK-293 cells, BJ human foreskin fibroblast cells, fHDF fibroblast cells, AGE.HN® neuronal precursor cells, CAP® amniocyte cells, adipose mesenchymal stem cells, RPTEC/TERT1 cells, or combinations thereof. In some aspects, the producer cell is a primary cell. In certain aspects, the primary cell can be a primary mammalian cell, a primary plant cell, a primary insect cell, a primary fungi cell, or a primary prokaryotic cell.

In some aspects, the producer cell is not an immune cell, such an antigen presenting cell, a T cell, a B cell, a natural killer cell (NK cell), a macrophage, a T helper cell, or a regulatory T cell (Treg cell). In other aspects, the producer cell is not an antigen presenting cell (e.g., dendritic cells, macrophages, B cells, mast cells, neutrophils, Kupffer-Browicz cell, or a cell derived from any such cells). In some aspects, a producer cell is not a naturally-existing antigen-presenting cell (i.e., has been modified). In some aspects, a producer cell is not a naturally-existing dendritic cell, a B cell, a mast cell, a macrophage, a neutrophil, Kupffer-Browicz cell, cell derived from any of these cells, or any combination thereof.

In some aspects, the one or more moieties (e.g., payload and/or targeting moiety) can be a transgene or mRNA, and introduced into the producer cell by transfection, viral transduction, electroporation, extrusion, sonication, cell fusion, or other methods that are known to the skilled in the art.

In some aspects, the one or more moieties (e.g., payload and/or targeting moiety) is introduced to the producer cell by transfection. In some aspects, the one or more moieties (e.g., payload and/or targeting moiety) can be introduced into suitable producer cells using synthetic macromolecules, such as cationic lipids and polymers (Papapetrou et al., Gene Therapy 12: S118-S130 (2005)). In some aspects, the cationic lipids form complexes with the one or more moieties (e.g., payload and/or targeting moiety) through charge interactions. In some of these aspects, the positively charged complexes bind to the negatively charged cell surface and are taken up by the cell by endocytosis. In some other aspects, a cationic polymer can be used to transfect producer cells. In some of these aspects, the cationic polymer is polyethylenimine (PEI). In certain aspects, chemicals such as calcium phosphate, cyclodextrin, or polybrene, can be used to introduce the one or more moieties (e.g., payload and/or targeting moiety) to the producer cells. The one or more moieties (e.g., payload and/or targeting moiety) can also be introduced into a producer cell using a physical method such as particle-mediated transfection, “gene gun”, biolistics, or particle bombardment technology (Papapetrou et al., Gene Therapy 12: S118-S130 (2005)). A reporter gene such as, for example, beta-galactosidase, chloramphenicol acetyltransferase, luciferase, or green fluorescent protein can be used to assess the transfection efficiency of the producer cell.

In certain aspects, the one or more moieties (e.g., payload and/or targeting moiety) are introduced to the producer cell by viral transduction. A number of viruses can be used as gene transfer vehicles, including moloney murine leukemia virus (MMLV), adenovirus, adeno-associated virus (AAV), herpes simplex virus (HSV), lentiviruses, and spumaviruses. The viral mediated gene transfer vehicles comprise vectors based on DNA viruses, such as adenovirus, adeno-associated virus and herpes virus, as well as retroviral based vectors.

In certain aspects, the one or more moieties (e.g., payload and/or targeting moiety) are introduced to the producer cell by electroporation. Electroporation creates transient pores in the cell membrane, allowing for the introduction of various molecules into the cell. In some aspects, DNA and RNA as well as polypeptides and non-polypeptide therapeutic agents can be introduced into the producer cell by electroporation.

In certain aspects, the one or more moieties (e.g., payload and/or targeting moiety) introduced to the producer cell by microinjection. In some aspects, a glass micropipette can be used to inject the one or more moieties (e.g., payload and/or targeting moiety) into the producer cell at the microscopic level.

In certain aspects, the one or more moieties (e.g., payload and/or targeting moiety) are introduced to the producer cell by extrusion.

In certain aspects, the one or more moieties (e.g., payload and/or targeting moiety) are introduced to the producer cell by sonication. In some aspects, the producer cell is exposed to high intensity sound waves, causing transient disruption of the cell membrane allowing loading of the one or more moieties (e.g., payload and/or targeting moiety).

In certain aspects, the one or more moieties (e.g., payload and/or targeting moiety) are introduced to the producer cell by cell fusion. In some aspects, the one or more moieties (e.g., payload and/or targeting moiety) are introduced by electrical cell fusion. In other aspects, polyethylene glycol (PEG) is used to fuse the producer cells. In further aspects, sendai virus is used to fuse the producer cells.

In some aspects, the one or more moieties (e.g., payload and/or targeting moiety) are introduced to the producer cell by hypotonic lysis. In such aspects, the producer cell can be exposed to low ionic strength buffer causing them to burst allowing loading of the one or more moieties (e.g., payload and/or targeting moiety). In other aspects, controlled dialysis against a hypotonic solution can be used to swell the producer cell and to create pores in the producer cell membrane. The producer cell is subsequently exposed to conditions that allow resealing of the membrane.

In some aspects, the one or more moieties (e.g., payload and/or targeting moiety) are introduced to the producer cell by detergent treatment. In certain aspects, producer cell is treated with a mild detergent which transiently compromises the producer cell membrane by creating pores allowing loading of the one or more moieties (e.g., payload and/or targeting moiety). After producer cells are loaded, the detergent is washed away thereby resealing the membrane.

In some aspects, the one or more moieties (e.g., payload and/or targeting moiety) introduced to the producer cell by receptor mediated endocytosis. In certain aspects, producer cells have a surface receptor which upon binding of the one or more moieties (e.g., payload and/or targeting moiety) induces internalization of the receptor and the associated moieties.

In some aspects, the one or more moieties (e.g., payload and/or targeting moiety) are introduced to the producer cell by filtration. In certain aspects, the producer cells and the one or more moieties (e.g., payload and/or targeting moiety) can be forced through a filter of pore size smaller than the producer cell causing transient disruption of the producer cell membrane and allowing the one or more moieties (e.g., payload and/or targeting moiety) to enter the producer cell.

In some aspects, the producer cell is subjected to several freeze thaw cycles, resulting in cell membrane disruption allowing loading of the one or more moieties (e.g., payload and/or targeting moiety).

### V.B Methods of Modifying an EV

In some aspects, a method of producing an EV comprises modifying the isolated EV by directly introducing one or more moieties (e.g., payload and/or targeting moiety) into the EVs. In certain aspects, the one or more moieties comprise an antigen, adjuvant, immune modulator, targeting moiety, or combinations thereof. In some aspects, the one or more moieties comprise a scaffold moiety disclosed herein (e.g., Scaffold X or Scaffold Y).

In certain aspects, the one or more moieties (e.g., payload and/or targeting moiety) are introduced to the EV by transfection. In some aspects, the one or more moieties (e.g., payload and/or targeting moiety) can be introduced into the EV using synthetic macromolecules such as cationic lipids and polymers (Papapetrou et al., Gene Therapy 12: S118-S130 (2005)). In certain aspects, chemicals such as calcium phosphate, cyclodextrin, or polybrene, can be used to introduce the one or more moieties (e.g., payload and/or targeting moiety) to the EV.

In certain aspects, the one or more moieties (e.g., payload and/or targeting moiety) are introduced to the EV by electroporation. In some aspects, EVs are exposed to an electrical field which causes transient holes in the EV membrane, allowing loading of the one or more moieties (e.g., payload and/or targeting moiety).

In certain aspects, the one or more moieties (e.g., payload and/or targeting moiety) are introduced to the EV by microinjection. In some aspects, a glass micropipette can be used to inject the one or more moieties (e.g., payload and/or targeting moiety) directly into the EV at the microscopic level.

In certain aspects, the one or more moieties (e.g., payload and/or targeting moiety) are introduced to the EV by extrusion.

In certain aspects, the one or more moieties (e.g., payload and/or targeting moiety) are introduced to the EV by sonication. In some aspects, EVs are exposed to high intensity sound waves, causing transient disruption of the EV membrane allowing loading of the one or more moieties (e.g., payload and/or targeting moiety).

In some aspects, one or more moieties (e.g., payload and/or targeting moiety) can be conjugated to the surface of the EV (i.e., conjugated or linked directly to the exterior surface of the EV or to the luminal surface of the EV). Conjugation can be achieved chemically or enzymatically, by methods known in the art.

In some aspects, the EV comprises one or more moieties (e.g., payload and/or targeting moiety) that are chemically conjugated. Chemical conjugation can be accomplished by covalent bonding of the one or more moieties (e.g., payload and/or targeting moiety) to another molecule, with or without use of a linker or affinity ligand disclosed herein. The formation of such conjugates is within the skill of artisans and various techniques are known for accomplishing the conjugation, with the choice of the particular technique being guided by the materials to be conjugated. In certain aspects, polypeptides are conjugated to the EV. In some aspects, non-polypeptides, such as lipids, carbohydrates, nucleic acids, and small molecules, are conjugated to the EV.

In some aspects, the one or more moieties (e.g., payload and/or targeting moiety) are introduced to the EV by hypotonic lysis. In such aspects, the EVs can be exposed to low ionic strength buffer causing them to burst allowing loading of the one or more moieties (e.g., payload and/or targeting moiety). In other aspects, controlled dialysis against a hypotonic solution can be used to swell the EV and to create pores in the EV membrane. The EV is subsequently exposed to conditions that allow rescaling of the membrane.

In some aspects, the one or more moieties (e.g., payload and/or targeting moiety) are introduced to the EV by detergent treatment. In certain aspects, extracellular vesicles are treated with a mild detergent which transiently compromises the EV membrane by creating pores allowing loading of the one or more moieties (e.g., payload and/or targeting moiety). After EVs are loaded, the detergent is washed away thereby resealing the membrane.

In some aspects, the one or more moieties (e.g., payload and/or targeting moiety) are introduced to the EV by receptor mediated endocytosis. In certain aspects, EVs have a surface receptor which upon binding of the one or more moieties (e.g., payload and/or targeting moiety) induces internalization of the receptor and the associated moieties.

In some aspects, the one or more moieties (e.g., payload and/or targeting moiety) are introduced to the EV by mechanical firing. In certain aspects, extracellular vesicles can be bombarded with one or more moieties (e.g., payload and/or targeting moiety) attached to a heavy or charged particle such as gold microcarriers. In some of these aspects, the particle can be mechanically or electrically accelerated such that it traverses the EV membrane.

In some aspects, extracellular vesicles are subjected to several freeze thaw cycles, resulting in EV membrane disruption allowing loading of the one or more moieties (e.g., payload and/or targeting moiety).

### V.C Methods of Isolating an EV

In some aspects, methods of producing EVs disclosed herein comprises isolating the EV from the producer cells. In certain aspects, the EVs released by the producer cell into the cell culture medium. It is contemplated that all known manners of isolation of EVs are deemed suitable for use herein. For example, physical properties of EVs can be employed to separate them from a medium or other source material, including separation on the basis of electrical charge (e.g., electrophoretic separation), size (e.g., filtration, molecular sieving, etc.), density (e.g., regular or gradient centrifugation), Svedberg constant (e.g., sedimentation with or without external force, etc.). Alternatively, or additionally, isolation can be based on one or more biological properties, and include methods that can employ surface markers (e.g., for precipitation, reversible binding to solid phase, FACS separation, specific ligand binding, non-specific ligand binding, affinity purification etc.).

Isolation and enrichment can be done in a general and non-selective manner, typically including serial centrifugation. Alternatively, isolation and enrichment can be done in a more specific and selective manner, such as using EV or producer cell-specific surface markers. For example, specific surface markers can be used in immunoprecipitation, FACS sorting, affinity purification, and magnetic separation with bead-bound ligands.

In some aspects, size exclusion chromatography can be utilized to isolate the EVs. Size exclusion chromatography techniques are known in the art. Exemplary, non-limiting techniques are provided herein. In some aspects, a void volume fraction is isolated and comprises the EVs of interest. Further, in some aspects, the EVs can be further isolated after chromatographic separation by centrifugation techniques (of one or more chromatography fractions), as is generally known in the art. In some aspects, for example, density gradient centrifugation can be utilized to further isolate the extracellular vesicles. In certain aspects, it can be desirable to further separate the producer cell-derived EVs from EVs of other origin. For example, the producer cell-derived EVs can be separated from non-producer cell-derived EVs by immunosorbent capture using an antigen antibody specific for the producer cell.

In some aspects, the isolation of EVs can involve combinations of methods that include, but are not limited to, differential centrifugation, size-based membrane filtration, immunoprecipitation, FACS sorting, and magnetic separation.

### VII. Methods of Treatment

Present disclosure also provides methods of preventing and/or treating an infectious disease or disorder, e.g., coronavirus infection, in a subject in need thereof, comprising administering an EV disclosed herein to the subject. Not to be bound by any one theory, in some aspects, an EV disclosed herein can treat and/or prevent these infectious diseases or disorders by inducing neutralizing antibodies that can specifically bind to a molecule associated with the infectious disease or disorder (e.g., S protein, M protein, and/or E protein).

EVs of the present disclosure can be administered to a subject by any useful method and/or route known in the art. In some aspects, the EVs are administered intravenously to the circulatory system of the subject. In some aspects, the EVs are infused in suitable liquid and administered into a vein of the subject.

In some aspects, the EVs are administered intra-arterially to the circulatory system of the subject. In some aspects, the EVs are infused in suitable liquid and administered into an artery of the subject.

In some aspects, the EVs are administered to the subject by intranasal administration. In some aspects, the EVs can be insufflated through the nose in a form of either topical administration or systemic administration. In certain aspects, the EVs are administered as nasal spray. In some aspects, intranasal administration can allow for the effective delivery of an EV disclosed herein to the gastrointestinal tissues. Such EVs delivered to the gastrointestinal tissues could be useful in providing protection against various gut-associated pathogens.

In some aspects, the EVs are administered to the subject by intraperitoneal administration. In some aspects, the EVs are infused in suitable liquid and injected into the peritoneum of the subject. In some aspects, the intraperitoneal administration results in distribution of the EVs to the lymphatics. In some aspects, the intraperitoneal administration results in distribution of the EVs to the thymus, spleen, and/or bone marrow. In some aspects, the intraperitoneal administration results in distribution of the EVs to one or more lymph nodes. In some aspects, the intraperitoneal administration results in distribution of the EVs to one or more of the cervical lymph node, the inguinal lymph node, the mediastinal lymph node, or the sternal lymph node. In some aspects, the intraperitoneal administration results in distribution of the EVs to the pancreas.

Non-limiting examples of other routes of administration that can be used to administer the EVs disclosed herein include parenteral, topical, oral, subcutaneous, intradermal, transdermal, rectal, intraperitoneal, intramuscular, sublingual, or combinations thereof.

As disclosed herein, in some aspects, EVs disclosed herein can be administered to a subject in combination with one or more additional therapeutic agents. In certain aspects, the one or more additional therapeutic agents and the EVs are administered concurrently. In some aspects, the one or more additional therapeutic agents and the EVs are administered sequentially. In some aspects, the EVs are administered to the subject prior to administering the one or more additional therapeutic agents. In certain aspects, the EVs are administered to the subject after administering the one or more additional therapeutic agents. As used herein, the term “therapeutic agents” refers to any agents that can be used in treating an infectious disease or disorder disclosed herein). In some aspects, the one or more additional therapeutic agents that can be used in combination with the EVs of the present disclosure include a payload (e.g., antigen, adjuvant, and/or immune modulator) which is not expressed in an EV. For instance, a treatment method disclosed herein can comprise administering to a subject in need thereof (i) an antigen-less EV and (ii) an antigen that is not expressed in an EV (e.g., soluble antigen).

In some aspects, a subject that can be treated with the present disclosure is a human. In some aspects, a subject is a non-human mammal (e.g., non-human primates, dogs, cats, guinea pigs, rabbits, rats, mice, horses, cattle, chickens, birds, and bears). Accordingly, in some aspects, the EVs disclosed herein can be used to improve the health of an animal (i.e., non-human mammal).

In some aspects, the present disclosure is directed to a method of vaccinating a subject in need thereof, comprising (i) administering a priming dose which comprises an extracellular vesicle comprising an adjuvant and an antigen to the subject and (ii) administering a boosting dose which comprises an extracellular vesicle comprising the antigen to the subject.

In some aspects, the antigen is derived from a coronavirus.

In some aspects, the priming dose is administered subcutaneously. In some aspects, the boost dose is administered intranasally.

In some aspects, the adjuvant is a STING agonist. In some aspects, the antigen is linked to a scaffold moiety. In some aspects, the scaffold moiety is Scaffold X.

In some aspects, the EV in the boosting dose does not contain any adjuvant.

In any of the administering methods described herein, in some aspects, an EV can be administered to a subject using a “prime-pull” dosing regimen. As used herein, the term “prime-pull” dosing regimen refers to an administration schedule in which a subject is first immunized with a first dosing regimen (also referred to herein as the “priming dose”) and then subsequently receives a second dosing regimen (also referred to herein as the “boosting dose”). In certain aspects, the first dosing regimen comprises a first EV and the second dosing regimen comprises a second EV, wherein the first and second EVs differ in their composition. For instance, in certain aspects, the first EV comprises an antigen and one or more of the other moieties described herein (e.g., adjuvant, immunomodulatory, and/or targeting moiety), and the second EV comprises the antigen but not the one or more of the other moieties present in the first EV. In some aspects, the first dosing regimen and the second dosing regimen are administered to the subject by different routes of administration (e.g., any combination of routes of administration that are known in the art and/or disclosed herein).

Accordingly, in some aspects, an administration (or vaccinating) method described herein comprises (i) administering a priming dose to a subject, wherein the priming dose comprises a first EV, which comprises an antigen and an adjuvant, and (ii) administering a boosting dose to the subject, wherein the boosting dose comprises a second EV, which comprises the antigen but not the adjuvant present in the first EV. In some aspects, the second EV does not comprise any adjuvant. Not to be bound by any one theory, in some aspects, the prime-pull dosing regimens can further improve the safety of the EV-based vaccines described herein, e.g., by not requiring the use of adjuvants when administering the boosting dose to the subject and thereby, avoid the risk of non-specific inflammation that can occur with certain adjuvants.

Moreover, in some aspects, the use of prime-pull dosing regimen can enhance the migration of immune cells to a particular tissue of interest (e.g., lung or other site of coronavirus infection). For instance, in certain aspects, a subject receives a first dosing regimen to prime or activate one or more immune cells, and then subsequently receives a second dosing regimen, wherein the second dosing regimen is capable of promoting the migration of the primed immune cells to the particular tissue of interest. In some aspects, this can be achieved by (i) administering the second dosing regimen using a tissue-specific route of administration, (ii) modifying the EVs of the second dosing regimen to comprise one or more tissue-specific targeting moieties, or (iii) both (i) and (ii). Non-limiting examples of such routes of administration and targeting moieties are provided throughout the present disclosure.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1: Generation of Engineered-Exosomes

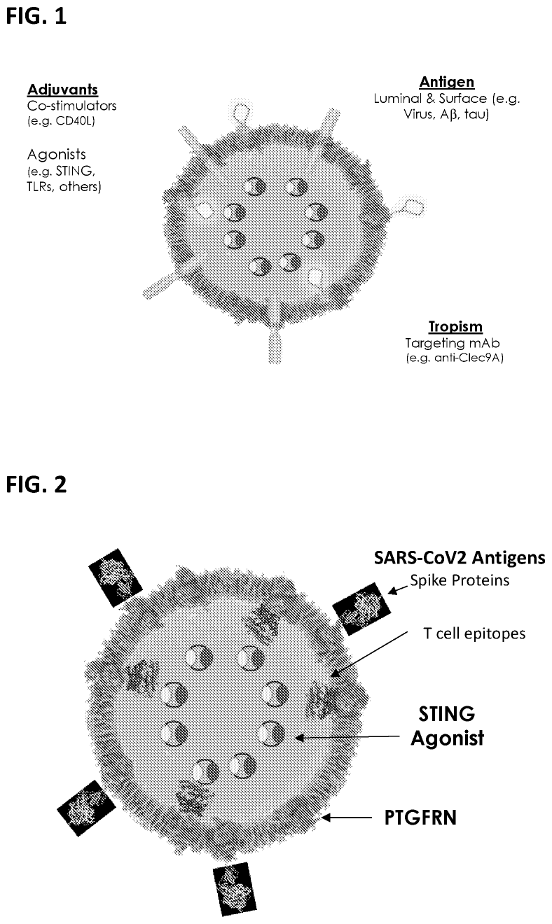

To generate exosomes described herein, human embryonic kidney (HEK) cell line (HEK293SF) was used. The cells were then stably transfected with Scaffold X and/or Scaffold Y linked to an agent of interest (e.g., antigen, adjuvant, or immune modulator). See FIGS. 1A, 1B, and 2. For example, CD40L-expressing exosomes were generated by transfecting HEK293SF cells with CD40L-GFP PTGFRN fusion molecules, which were expressed as a monomer (pCB-518 to pCB-526) or as a forced trimer (pCB-607 and pCB-527). An example of a trimeric CD40L-GFP PTGFRN fusion molecule is shown in FIG. 1A. Similarly, to generate chicken ovalbumin (OVA)-expressing exosomes, ovalbumin was stably expressed in HEK293SF cells as a fusion to amino acids 1-10 of BASP1 (“BASP1 (1-10)-OVA”).

Upon transfection, HEK293SF cells were grown to high density in chemically defined medium for 7 days. Conditioned cell culture media was collected and centrifuged at 300-800×g for 5 minutes at room temperature to remove cells and large debris. Media supernatant was then supplemented with 1000 U/L BENZONASE® and incubated at 37° C. for 1 hour in a water bath. Supernatant was collected and centrifuged at 16,000×g for 30 minutes at 4° C. to remove residual cell debris and other large contaminants. Supernatant was then ultracentrifuged at 133,900×g for 3 hours at 4° C. to pellet the exosomes. Supernatant was discarded and any residual media was aspirated from the bottom of the tube. The pellet was resuspended in 200-1000 μL PBS (—Ca—Mg).

To further enrich exosome populations, the pellet was processed via density gradient purification (sucrose or OPTIPREP™).

The gradient was spun at 200,000×g for 16 hours at 4° C. in a 12 mL Ultra-Clear (344059) tube placed in a SW 41 Ti rotor to separate the exosome fraction.

The exosome layer was gently removed from the top layer and diluted in ˜32.5 mL PBS in a 38.5 mL Ultra-Clear (344058) tube and ultracentrifuged again at 133,900×g for 3 hours at 4° C. to pellet the purified exosomes. The resulting pellet was resuspended in a minimal volume of PBS (˜200 μL) and stored at 4° C.

For OPTIPREP™ gradient, a 3-tier sterile gradient is prepared with equal volumes of 10%, 30%, and 45% OPTIPREP™ in a 12 mL Ultra-Clear (344059) tube for a SW 41 Ti rotor. The pellet was added to the OPTIPREP™ gradient and ultracentrifuged at 200,000×g for 16 hours at 4° C. to separate the exosome fraction. The exosome layer was then gently collected from the top ˜3 mL of the tube.

The exosome fraction was diluted in ˜32 mL PBS in a 38.5 mL Ultra-Clear (344058) tube and ultracentrifuged at 133,900×g for 3 hours at 4° C. to pellet the purified exosomes. The pelleted exosomes were then resuspended in a minimal volume of PBS (˜200 μL) and stored at 4° C. until ready to be used.

### Example 2: Efficacy of Engineered-Exosomes to Induce Antigen-Specific T Cell Responses

Virus-specific CD8 T cells are required for pathogen clearance following primary SARS-CoV infection. SARS-CoV-specific memory CD8 T cells protect susceptible hosts from lethal SARS-CoV infection. Ability of the exoVACC platform to generate robust antigen specific CD8+ T-cell response and the ability to expand the tissue resident memory T-cell response provides a unique opportunity to develop a CD8 T-cell based vaccine for SARS-Cov2.

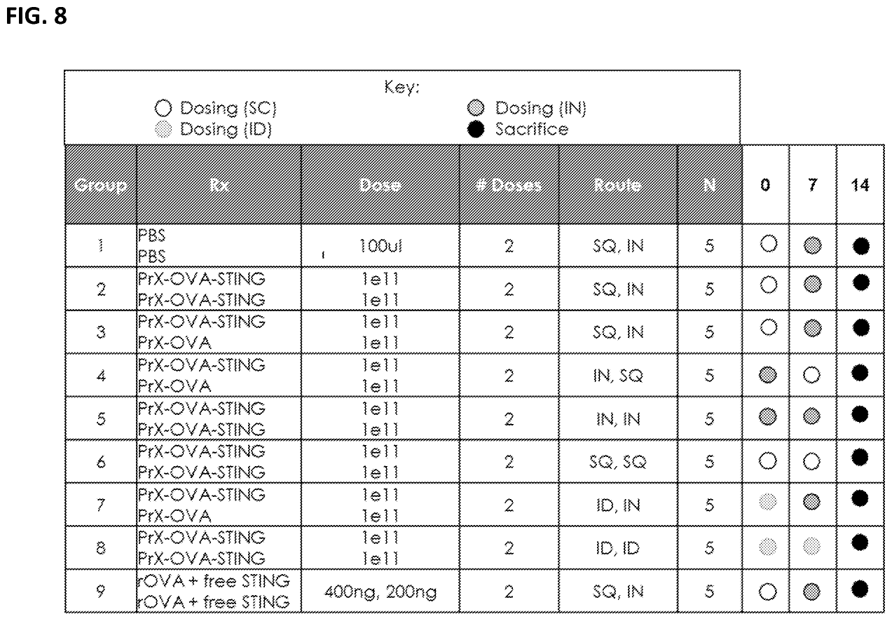

To demonstrate that the EVs described herein are capable of inducing antigen-specific T cell responses, EVs comprising ovalbumin (OVA) were constructed using methods described herein. OVA was fused to the N-terminus of PTGFRN and displayed on the exterior surface of the EV (“PrX-OVA”). Some of the EVs were further loaded with a STING agonist in the lumen of the EV (“PrX-OVA-STING”). Then, the EVs were administered to mice using the prime-and-pull administration strategy described herein. As shown in FIG. 8, animals were vaccinated twice (i.e., at days 0 and 7 post initial administration) via subcutaneous (SQ), intranasal (IN), or intradermal (ID) administration. Animals were sacrificed at day 14 post initial administration, and T cell immune responses were observed in the animals.

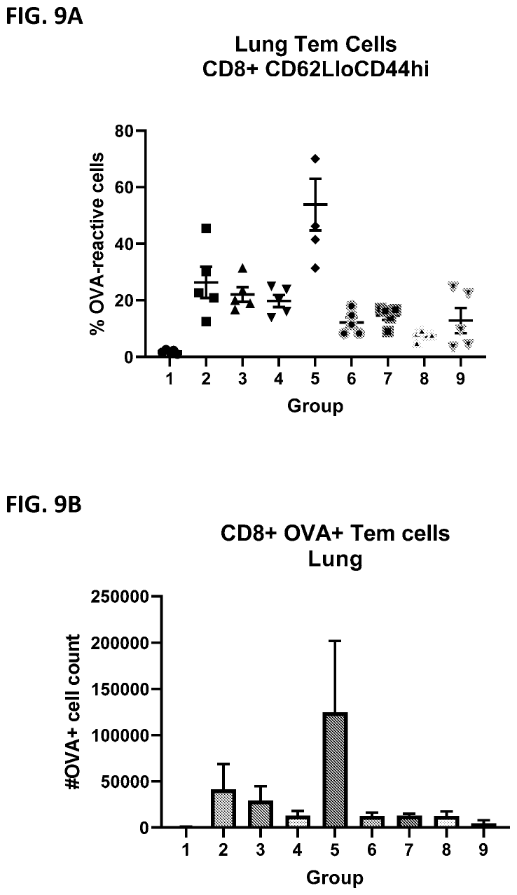

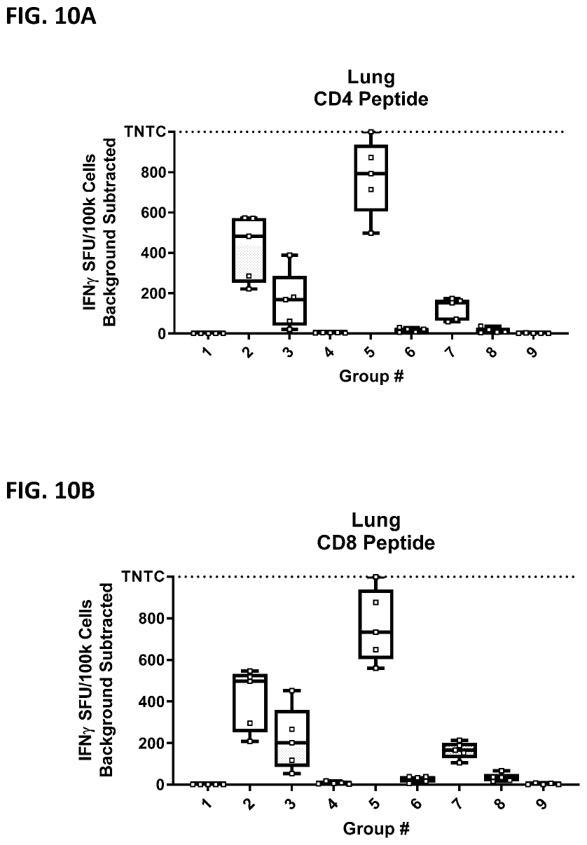

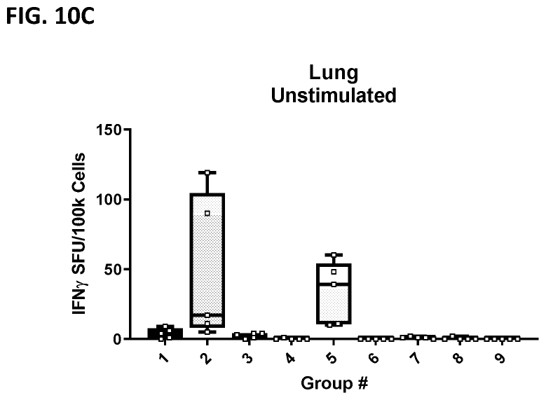

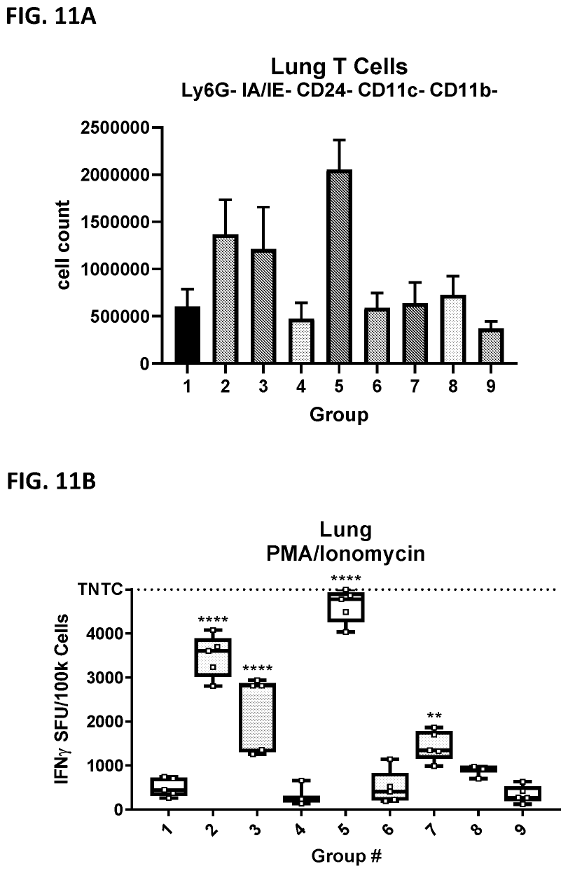

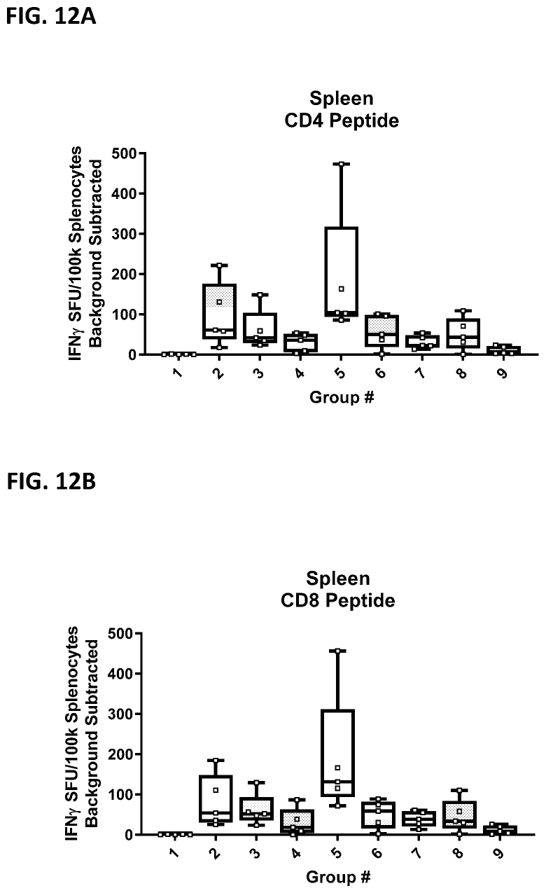

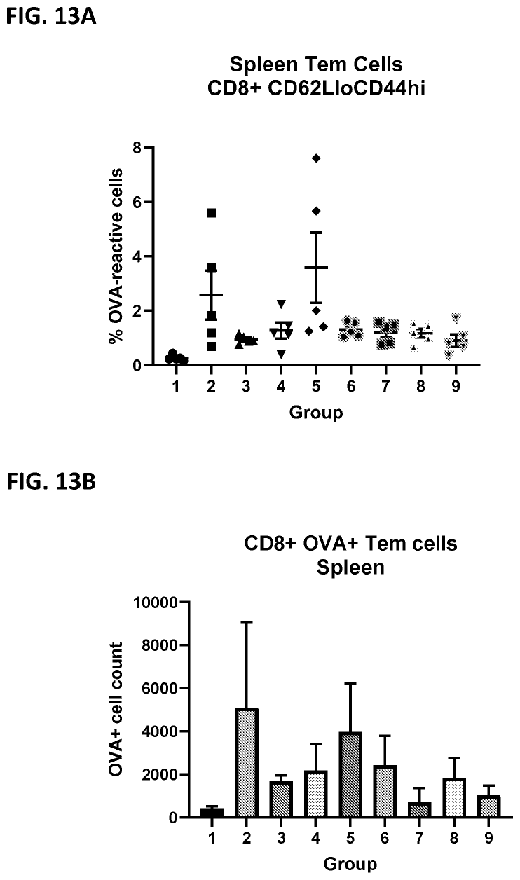

As shown in FIGS. 9A, 9B, 10A, 10B, 10C, 11A, and 11B, animals that received an EV described herein had significantly greater number of total T cells in the lung (i.e., groups 2-8) compared to animals treated with recombinant OVA+soluble STING agonist (i.e., group 9), demonstrating the potency of the EVs described herein. The increase in total T cells correlated with increases in both OVA-specific CD4+ and CD8+ T cells, including OVA-specific effector memory CD8+ T cells. The different combinations of administration routes appeared to have minimal effect. Additionally, OVA-specific effector memory CD8+ T cells were also observed in the lung where the EV used for the boost did not comprise the STING agonist (i.e., groups 3, 4, and 7), suggesting that an adjuvant is not necessary for the boost using EVs described herein. Moreover, as shown in FIG. 10C, the prime-pull administration strategy described herein did not lead to non-specific T cell inflammatory activation in the lungs (groups 3 and 4) compared to groups 2 and 5, where an adjuvanted boosting dose was administered intranasally. Similar results were observed in the spleen (see FIGS. 12A, 12B, 13A, and 13B).

The above results demonstrate the safety of the EVs described herein and the effectiveness of the prime-and-pull regimen of the present disclosure.

### Example 3: Development of a Coronavirus Extracellular Vesicle Vaccine

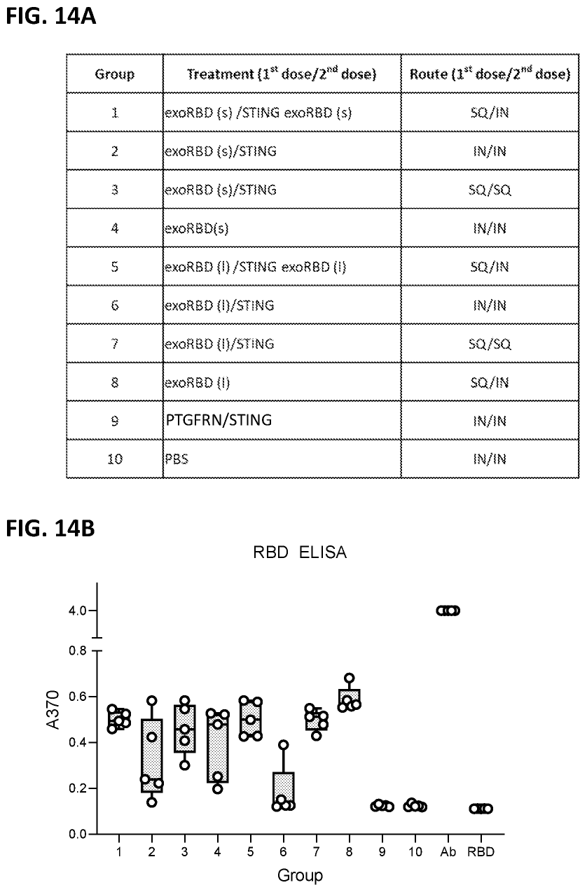

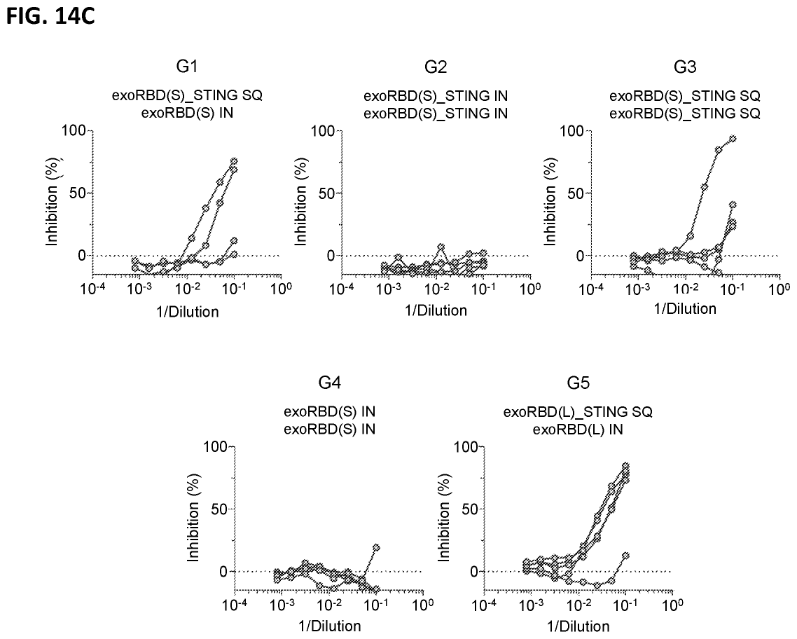

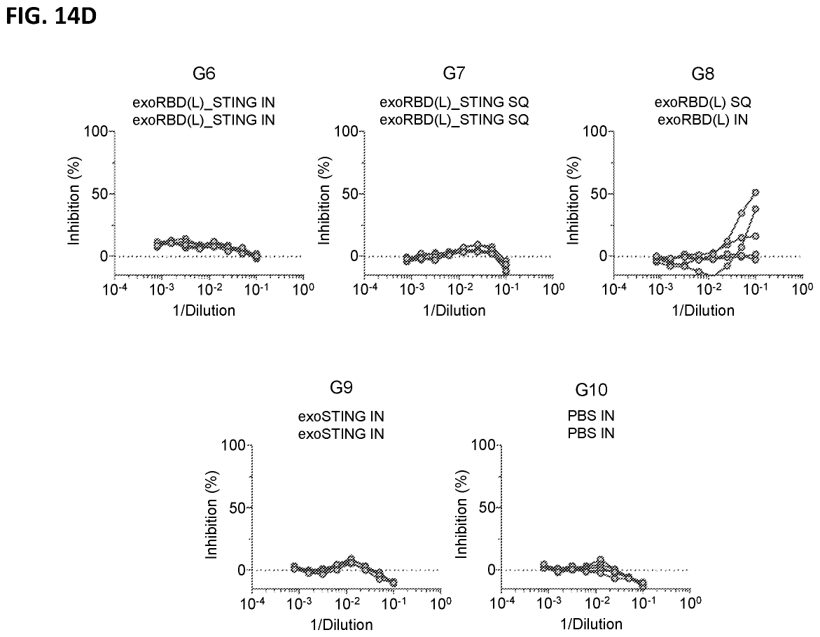

To assess the efficacy of EVs described herein in treating a coronavirus, EVs comprising the RBD of a coronavirus spike protein was constructed using the methods described herein (“exoRBD”). The RBD protein was fused to the N-terminus of either the full-length PTGFRN (“exoRBD (1)” or a PTGFRN fragment (“exoRBD(s)”). Some of the EVs were further loaded with a STING agonist in the lumen of the EV (“STING exoRBD”). Then, as shown in FIG. 14A, the different EVs were used to vaccinate mice. Each of the animals received two doses (at day 0 and at day 14) either via subcutaneous (SQ) or intranasal (IN) administration. Then, the sera was collected from the animals at day 28 post initial administration, and the anti-RBD antibody response was assessed using an ELISA assay.

As shown in FIG. 14B, in all animals that were vaccinated with the exoRBD (with or without the STING agonist) had significant levels of anti-RBD antibodies in the sera. Again, the particular combination of administration routes did not appear to have a significant effect. And, also as observed in Example 2, significant level of anti-RBD antibody was observed even where the STING agonist was not included as part of the 2^{nd} dose (either as soluble STING or loaded in the lumen of the EV). Moreover, the antibodies observed in animals from some of the EV vaccinated groups were also neutralizing (see, e.g., groups 1, 3, 5, and 8 in FIGS. 14C and 14D).

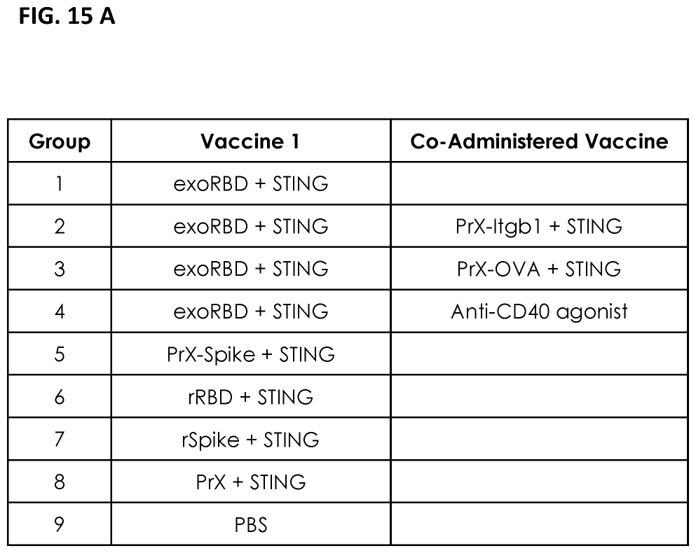

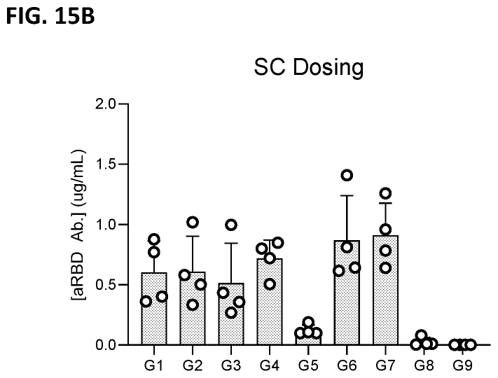

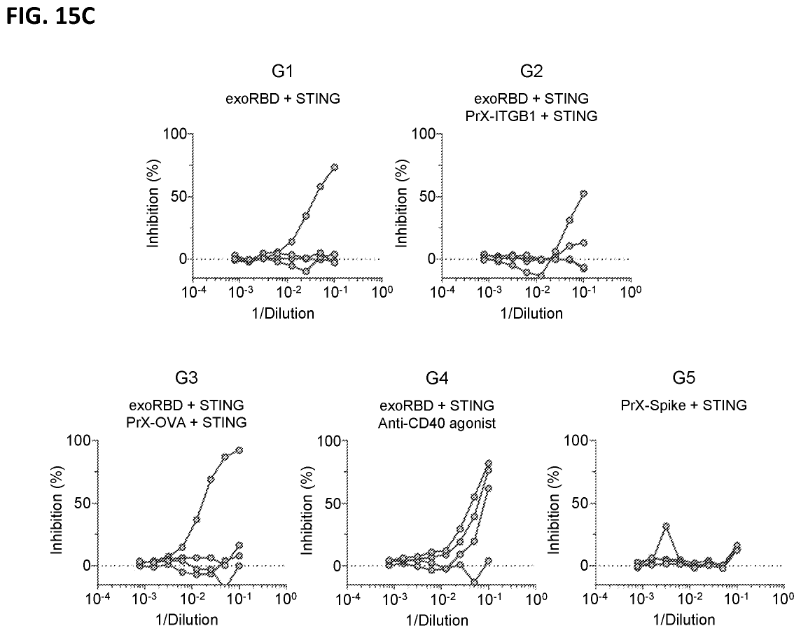

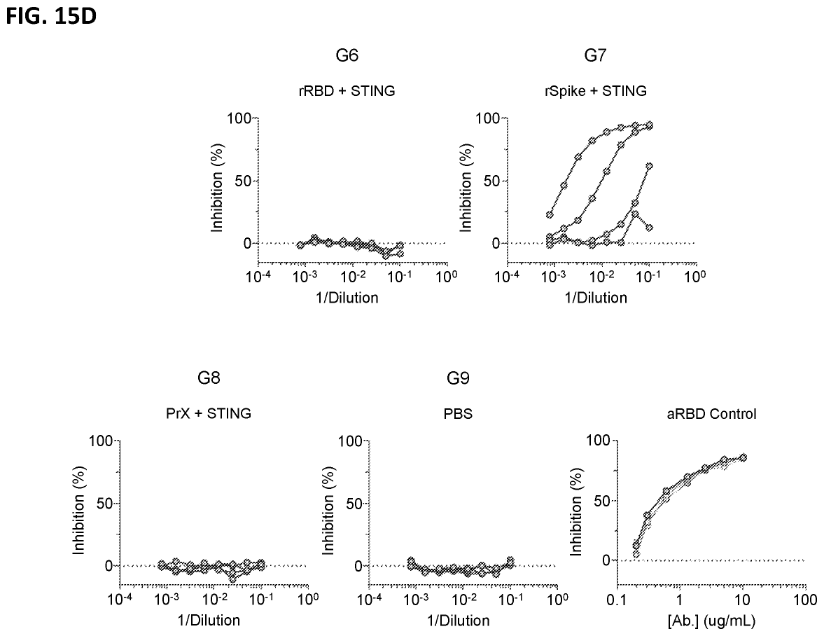

Next, to assess whether the addition of T helper epitopes or B cell immunomodulators can further enhance the anti-RBD antibody levels, EVs comprising either T helper epitopes Itgb1 or ovalbumin (OVA) fused to PTGFRN on the exterior surface (“PrX-Itgb1”) or (“PrX-OVA”) were co-administered with EVs with RBD fused to PTGFRN on the exterior surface or alternately EVs with RBD fused to PTGFRN on the exterior surface were administered with a B cell co-stimulator (anti-CD40 agonist antibody). Mice were vaccinated (at days 0 and 14) with the different EVs as shown in FIG. 15. Then, at day 35 post initial administration, the animals were sacrificed and the anti-RBD antibody levels and neutralizing antibody activity were observed in the animals.

As shown in FIGS. 15B-15G, B cell costimulation through CD40 activation enhanced anti-RBD antibody levels and neutralization activity.

### Example 4: Further Characterization of EVs Comprising Coronavirus Antigens

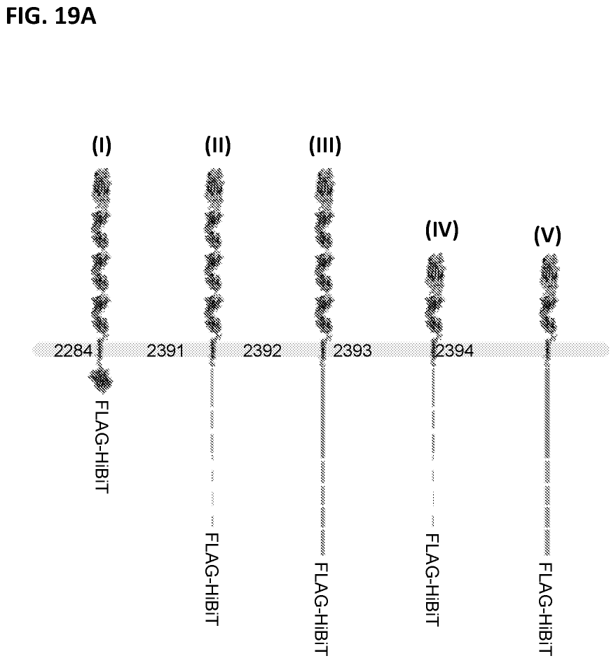

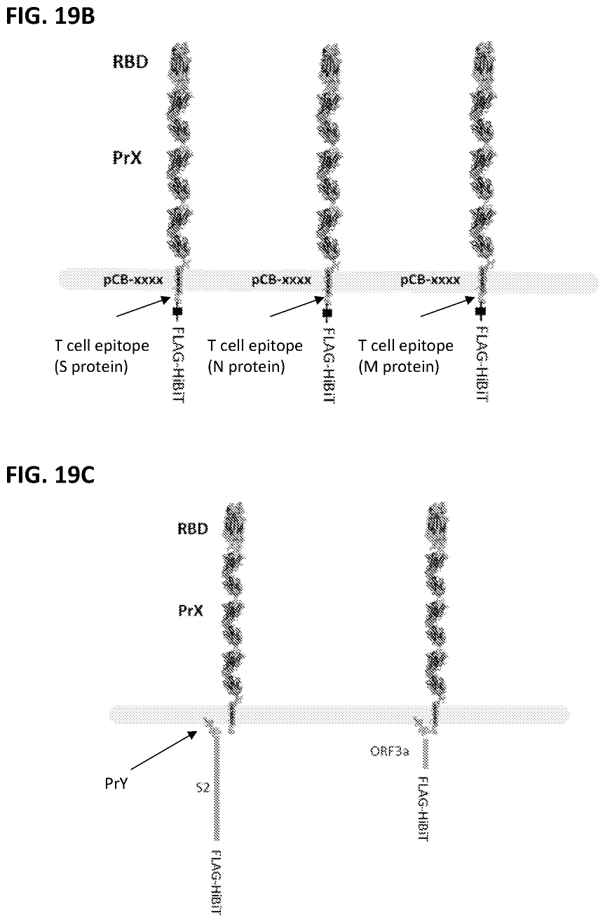

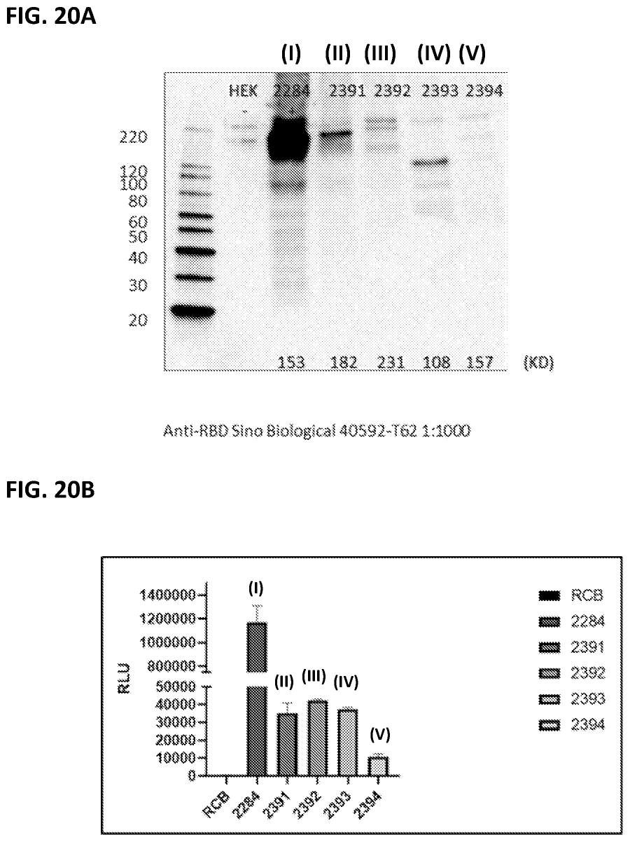

To further demonstrate that the EVs described herein can be engineered to express antigens that would be useful in treating an infectious disease, such as a coronavirus infection, EVs were engineered to comprise both B and T cell antigens of a coronavirus. Specifically, as described in Example 3, the RBD protein of a coronavirus spike protein was fused to the N-terminus of PTGFRN (both full-length and fragment) and displayed on the exterior surface of the EV. Additionally, the EVs were also engineered to comprise T cell epitopes of coronavirus (e.g., spike protein, nucleocapsid, membrane protein, and/or ORF3a), which were expressed on the luminal surface of the EVs (fused to PTGFRN or to BASP1), as a single peptide, as concatemer peptide antigens, or concatemer protein antigens (see FIGS. 19A, 19B, and 19C). Expression of the antigens was confirmed by both Western blot and HiBiT assay (see FIGS. 20A and 20B, respectively).

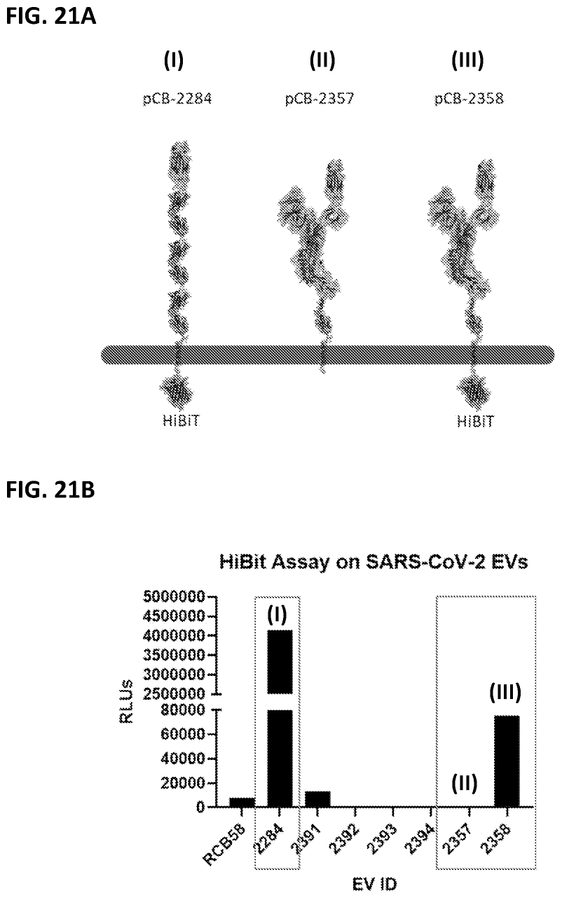

To assess the expression level of the coronavirus antigens, EVs were engineered to comprise either RBD protein or the entire spike protein of coronavirus fused to the N-terminus of PTGFRN and displayed on the exterior surface of the EV (see FIG. 21A). As shown in FIG. 21B, each of the EVs constructed expressed multiple copies of the coronavirus antigens on the exterior surface.

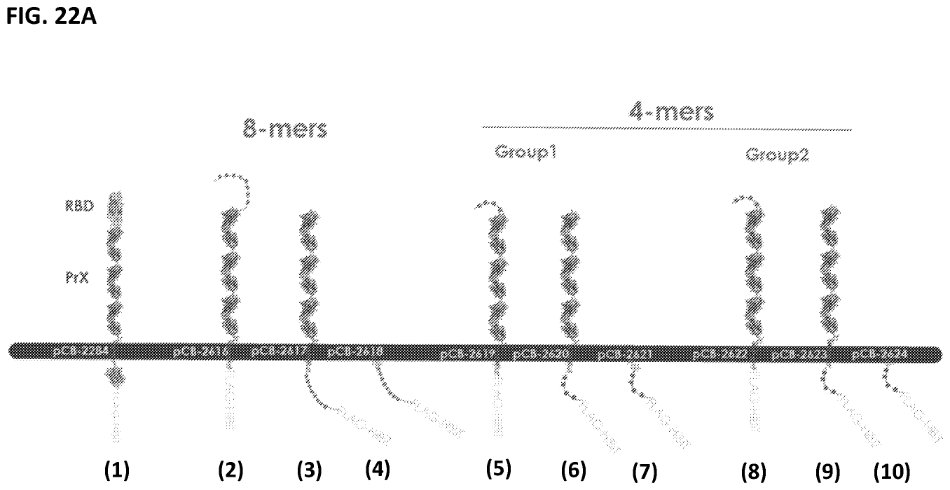

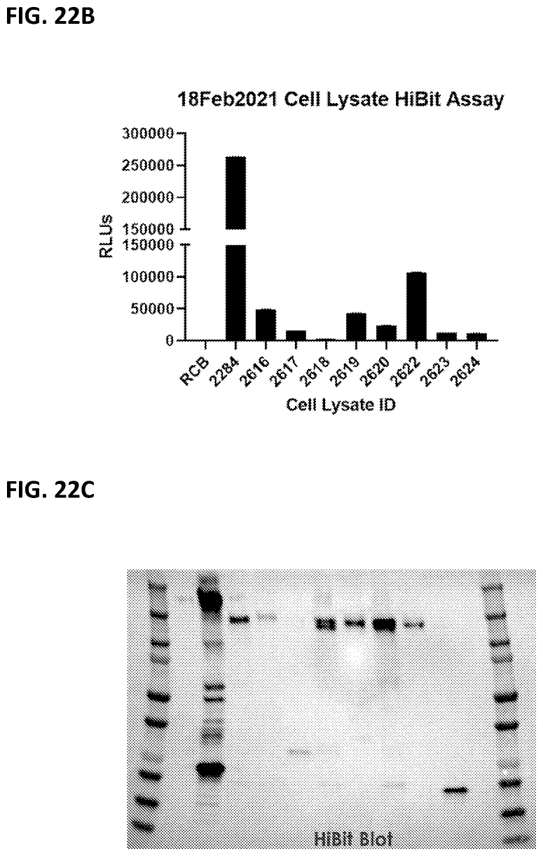

Next, to further assess the expression of the T cell epitopes of coronavirus in the EVs, EVs were modified to comprise the concatemer T cell epitopes of coronavirus either on the exterior surface (fused to PTGFRN) or on the luminal surface (fused to PTGFRN or BASP-1) (see FIG. 22A). Expression was confirmed by both Western blot and HiBiT assay (see FIGS. 22B and 22C).

The above results demonstrate the versatility of the EVs described herein, as they can be engineered to comprise multiple antigens (both B and T cell antigens) which can be useful in treating the different diseases or disorders disclosed herein, e.g., coronavirus infection.

### Example 5. exoVACC™: A Novel Exosome-Based Vaccine Platform that Induces Robust, Tunable B and T Cell Responses

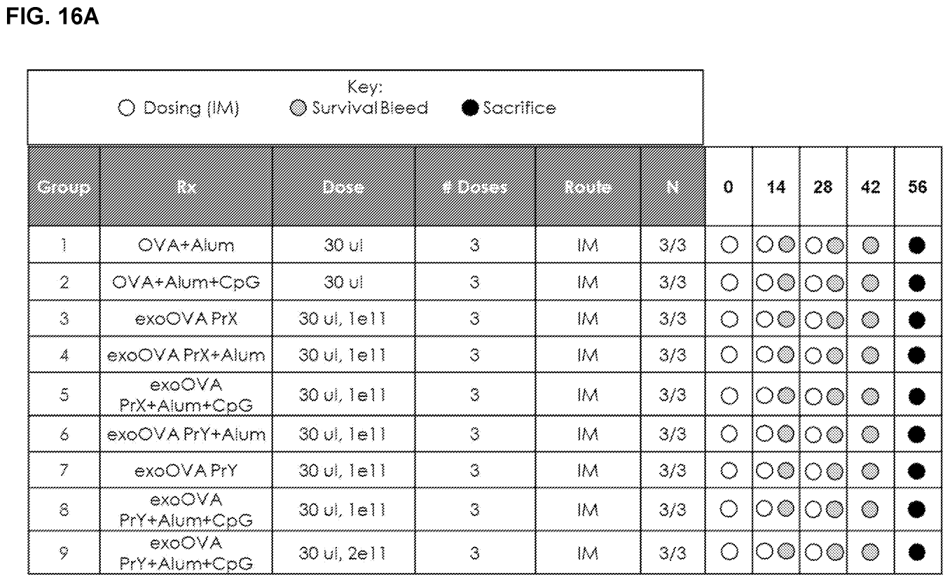

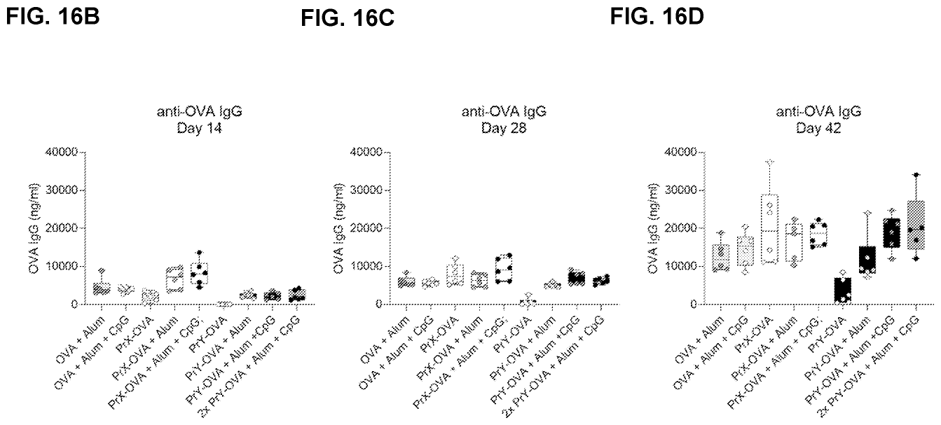

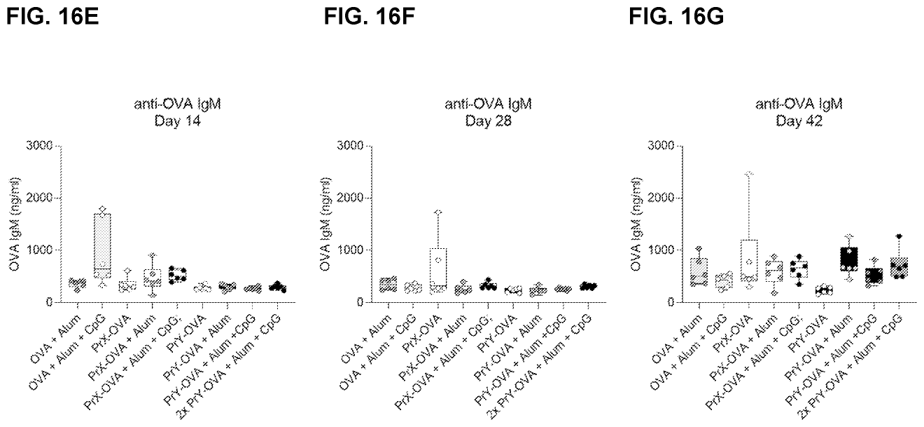

Background: exoVACC™ is a novel exosome-based vaccine platform that is designed to selectively deliver exogenous antigens, adjuvants and immunomodulators to antigen presenting cells to induce robust cellular and humoral immune responses. Exosomes are natural extracellular delivery vesicles but are inherently non-immunogenic. We investigated different antigen-adjuvant designs to better understand the properties that influence exoVACC™ immunogenicity. Using exosome scaffold proteins PTGFRN and BASP1, we selectively expressed the model antigen OVA either on the exosome surface, by fusion with PTGFRN, or in the lumen, by fusion with BASP1. We co-loaded different adjuvants and assessed both humoral and cellular immune responses in mice immunized through different routes of administration. The different treatment groups are described in FIG. 16A.

Results: Immunization with OVA expressed on the exosome surface induced anti-OVA IgG antibody responses as early as 14 days after a single immunization which increased following bi-weekly boost immunizations. See FIGS. 16B-16G. Antibody responses were induced without an adjuvant and at higher levels than in mice immunized with traditional soluble OVA and adjuvants. Notably, antibody responses were not enhanced when alum, CpG or both alum and CpG adjuvants were loaded onto exosomes. In contrast, mice immunized with exosomes expressing OVA in the lumen had very poor antibody responses even after several boost immunizations. Anti-OVA IgG antibody levels were significantly lower than in mice immunized with the same amount of soluble OVA. However, loading an adjuvant onto exosomes expressing luminal OVA induced antibody responses comparable to mice immunized with exosomes expressing surface OVA. Similarly, immunization of exosomes expressing luminal OVA without adjuvant also failed to induce antigen-specific T cell responses even after multiple administrations and via multiple routes of administration (RoA).

As demonstrated earlier (see, e.g., Example 2), loading an adjuvant induced robust T effector memory (TEM) and tissue resident memory T cell (TRM) responses after a single immunization via multiple RoA. Notably, the induction of antigen-specific TRM was markedly superior compared to immunization with soluble OVA and adjuvant. Exosomes expressing OVA on the surface also induced robust T cell responses. OVA-specific lung TRM and TEM were induced via IN immunization of STING adjuvanted exosomes but also through a “prime-pull” regimen where mice were first immunized SC with adjuvanted exosomes followed by an unadjuvanted IN boost. In contrast mice immunized with soluble OVA and STING adjuvant using the prime-push regimen did not elicit robust T cell responses.

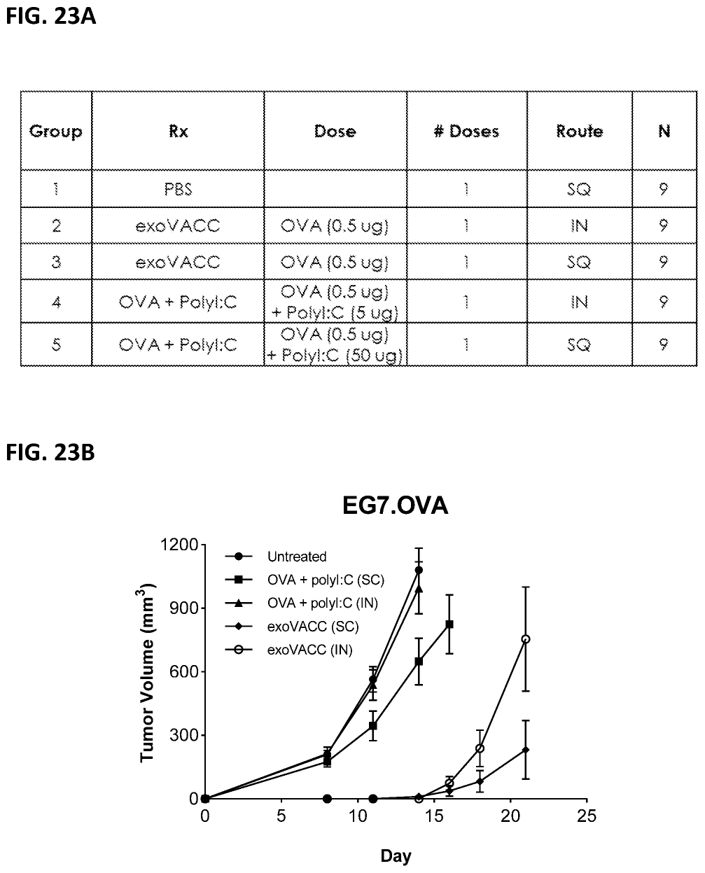

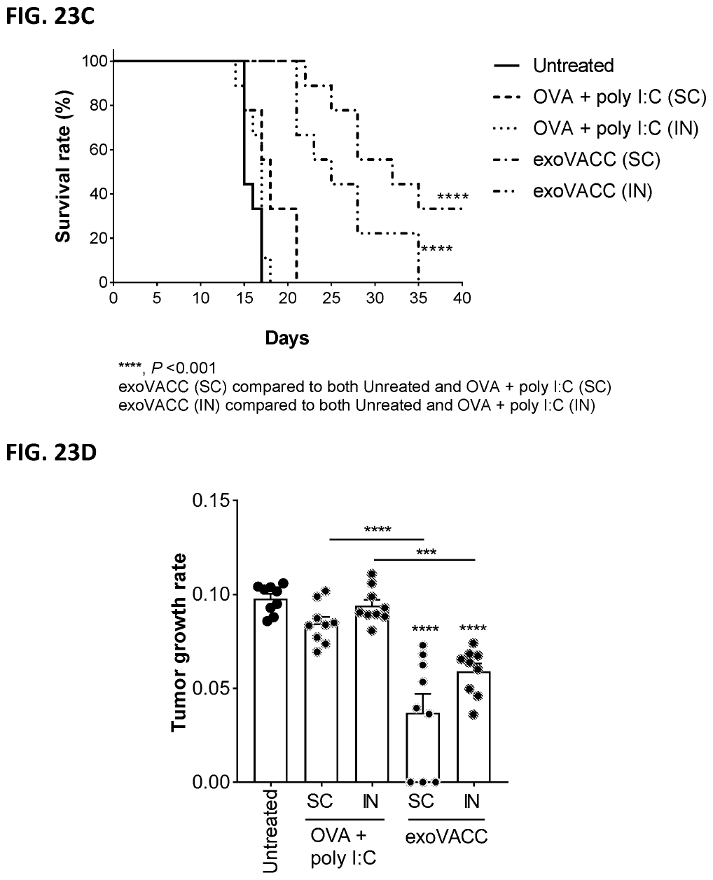

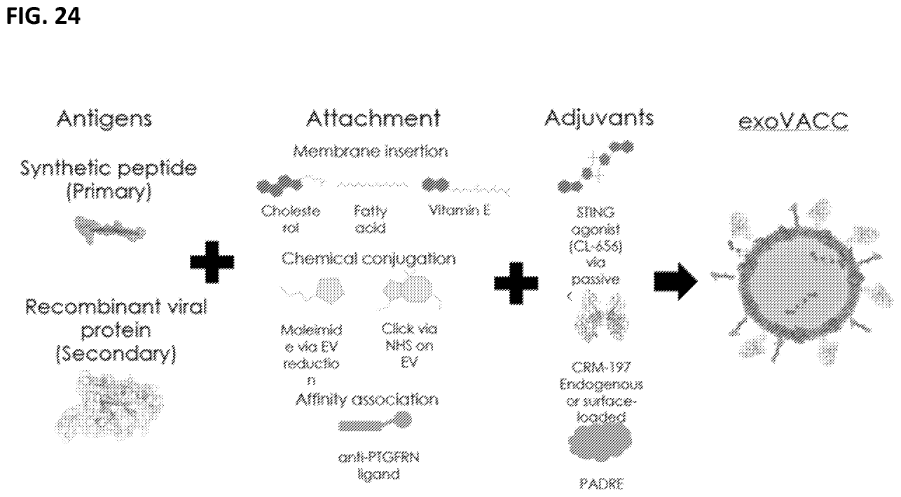

Finally, we assessed the efficacy of exosomes expressing OVA in the lumen and loaded with a STING agonist adjuvant (“exoVACC”) in the E.G7-OVA tumor model. As shown in FIG. 23A, mice received a single administration one of one of the following: (1) PBS alone via subcutaneous administration; (2) exoVACC via intranasal administration; (3) exoVACC via subcutaneous administration; (4) soluble OVA+PolyI:C via intranasal administration; and (5) soluble OVA+PolyI:C via subcutaneous administration. A single prophylactic immunization administered either subcutaneously (SC) or intranasally (IN) significantly reduced tumor growth rate (FIGS. 23B and 23D) and improved survival (FIG. 23C) compared to mice immunized with soluble OVA and polyI:C. 33% of mice immunized SC with the OVA exosomes had complete responses.

Conclusions: exoVACC™ is a versatile vaccine platform that enables antigen-specific immune responses to be modulated through antigen orientation and adjuvant loading. exoVACC™ induced superior systemic and tissue resident immune responses via multiple routes of administration compared to conventional vaccine formulations in different animal models.

### Example 6. Vaccination of Exo-VACC™ with Coronavirus Antigen

Exo-VACC™ with a coronavirus antigen alone and Exo-VACC™ with a coronavirus antigen and an adjuvant, e.g., STING agonist, were made according to the present disclosure, e.g., Examples 3 and 4. After the exosomes were prepared, the exosomes were administered to a subject using a vaccination regimen, e.g., prime-pull regimen. For example, in certain instances, a subject was subcutaneously administered a priming dose comprising exosomes which comprises a coronavirus antigen and an adjuvant, e.g., STING agonist. The subject was then be intranasally administered a boosting dose comprising exosomes which comprises the coronavirus antigen, but not any adjuvant.

As further described in Example 3, such a prime-pull administration regimen was effective in inducing coronavirus-specific immune responses in vivo (see, e.g., FIGS. 14C, 14D, and 15B-15E). Because an adjuvant is not necessary as part of the boosting dose, the results provided here suggest that the prime-pull administration strategy with the EVs described herein can be safe and effective in treating coronavirus.

### Example 7. Construction of EVs Comprising RBD Using the ALFA Plug-and-Play System

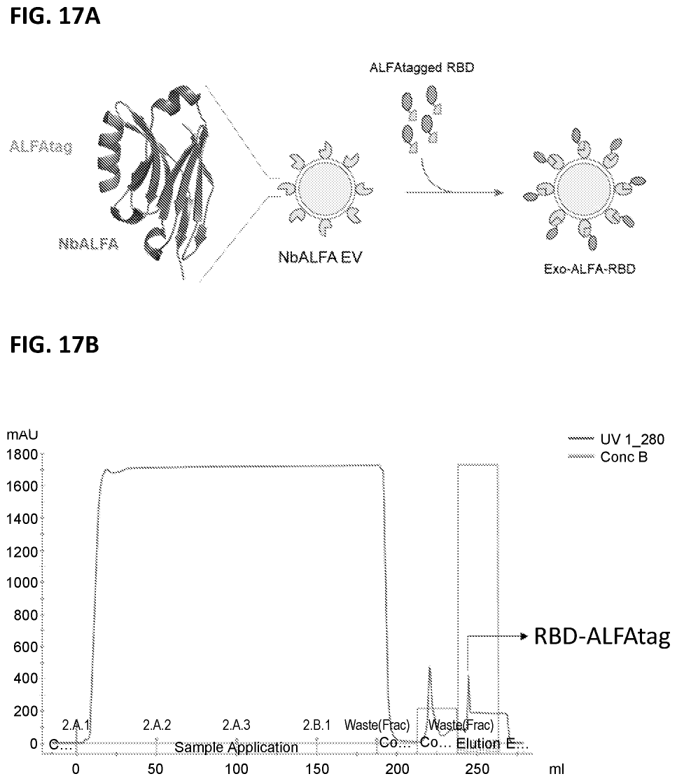

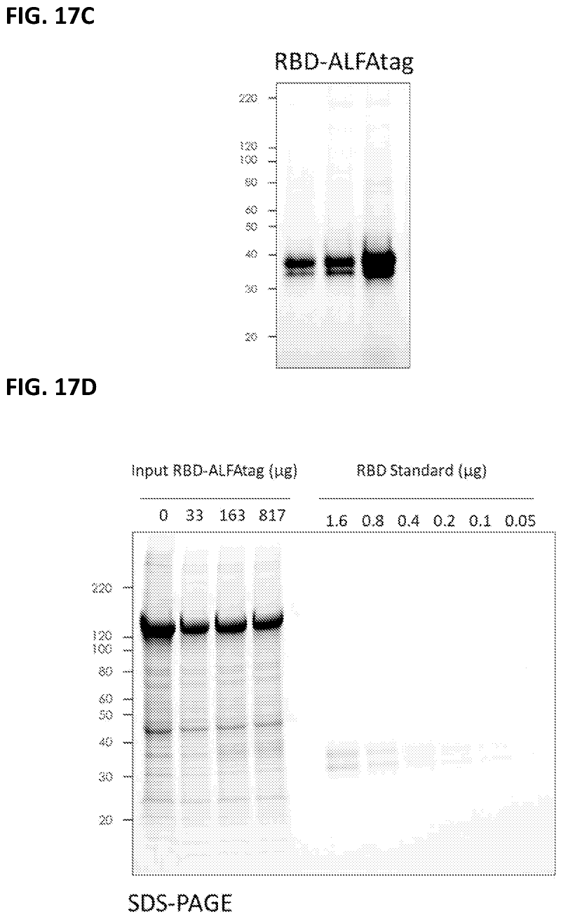

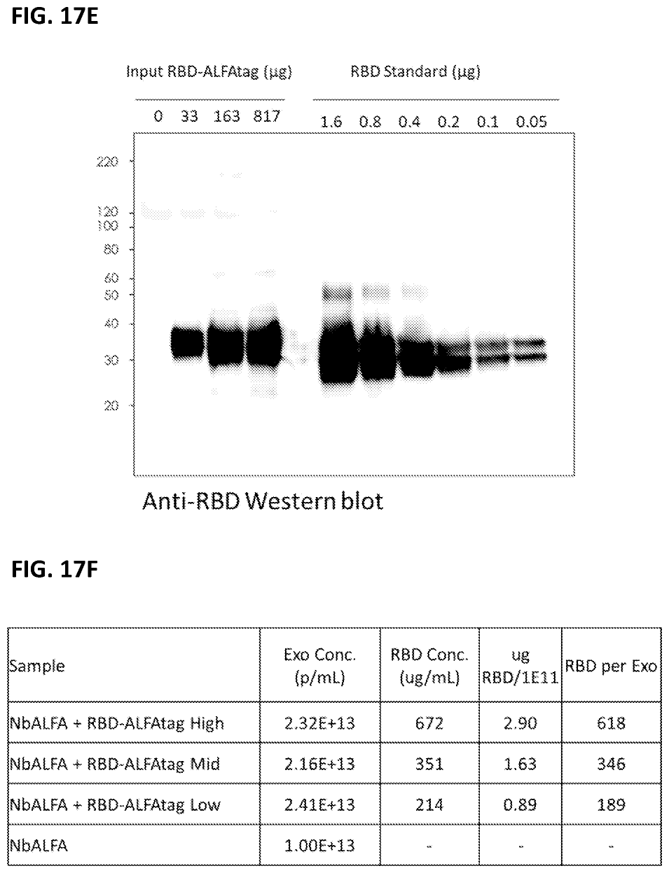

As further demonstration that the EVs described herein can be used to treat a coronavirus, the ALFA plug-and-play system described herein was used to modify the EVs to comprise the RBD protein of a coronavirus spike protein. Briefly, NbALFA nanobody was expressed on the EV surface as a fusion to the surface display scaffold PTGFRN. The RBD was expressed and purified using conventional transient transfection and column chromatography, respectively, and were fused to the ALFAtag. FIG. 17A. RBD-ALFAtag was conjugated to NbALFA EVs by mixing at room temperature for 30 minutes. Unbound ALFAtagged RBD was removed by ultracentrifugation or size exclusion chromatography, resulting in stable association of the ALFAtagged RBD on the surface of NbALFA EVs. FIGS. 17B-17F show the successful purification and loading of the ALFAtagged RBD to the EVs using both SDS-PAGE and Western blot.

Then, the EVs were administered to mice and the anti-RBD antibody response observed. Specifically, the animals received a single subcutaneous administration of one of the following: (i) “exoRBD”=RBD fused directly to PTGFRN; (ii) “rRBD+STING”=recombinant RBD protein+soluble STING agonist; (iii) “rRBD+Exo”=recombinant RBD protein+EV alone (i.e., RBD not associated with the EV); and (iv) PBS alone. In FIG. 17B, the animals were vaccinated with one of the following: (i) “NbALFA Exo+RBD-ALFAtag”=RBD conjugated to PTGFRN using the ALFA plug-and-play system; (ii) “rRBD”=recombinant RBD protein alone; (iii) “rRBD”=recombinant RBD protein+soluble STING agonist; (iv) “Exo+rRBD”=recombinant RBD protein+EV alone (i.e., RBD not associated with the EV); and (v) PBS alone. Control animals were administered 8 μg recombinant RBD alone (rRBD), 20 μg rRBD adjuvanted with a cyclic dinucleotide STING agonist (rRBD+STING), 8 μg rRBD co-administered with exosomes (rRBD+Exo) or PBS alone. 14 days post-vaccination, non-lethal bleeds were taken from each animal and anti-RBD antibody titer was determined by conventional plate-based ELISA.

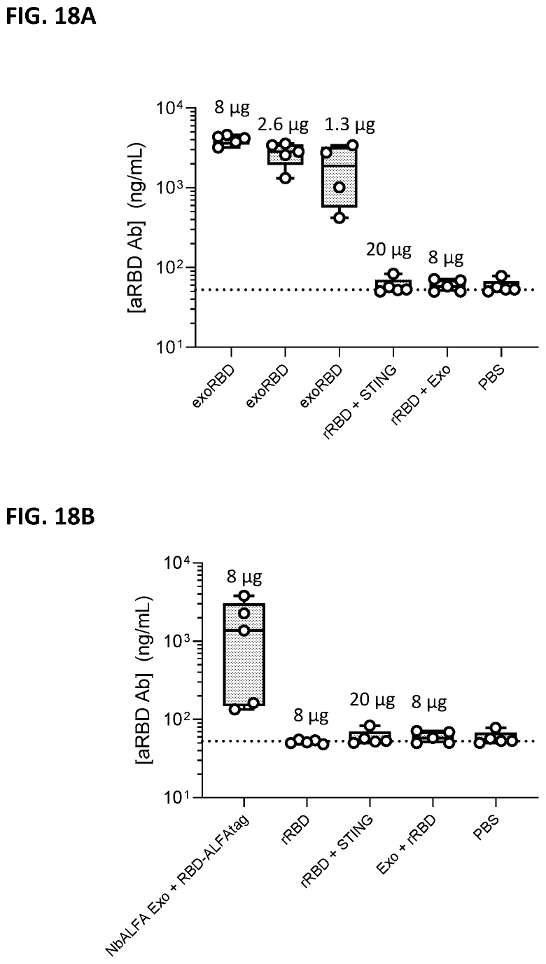

As shown in FIGS. 18A and 18B, superior immunogenicity was observed from exosomes displaying ALFAtagged RBD compared to rRBD, adjuvanted rRBD, or exosomes co-administered with rRBD, suggesting presentation of the antigen on the exosome surface was important for eliciting an immunogenic response. The data also suggest that the ALFA system was functioning as expected in an in vivo context.

Collectively, the data provided in the above examples demonstrate that EVs described herein (e.g., comprising a coronavirus antigen) can elicit potent coronavirus-specific immune responses, suggesting that they can be useful in treating a coronavirus, such as COVID-19.

### Example 8. Construction of EVs Comprising Acceptor Domain Fused to PTGFRN

To further demonstrate the versatility of the EVs described herein as a vaccine platform, several different “plug-and-play” or “clip-on” strategies were analyzed for their ability to quickly and efficiently attach a molecule to the surface of EVs.

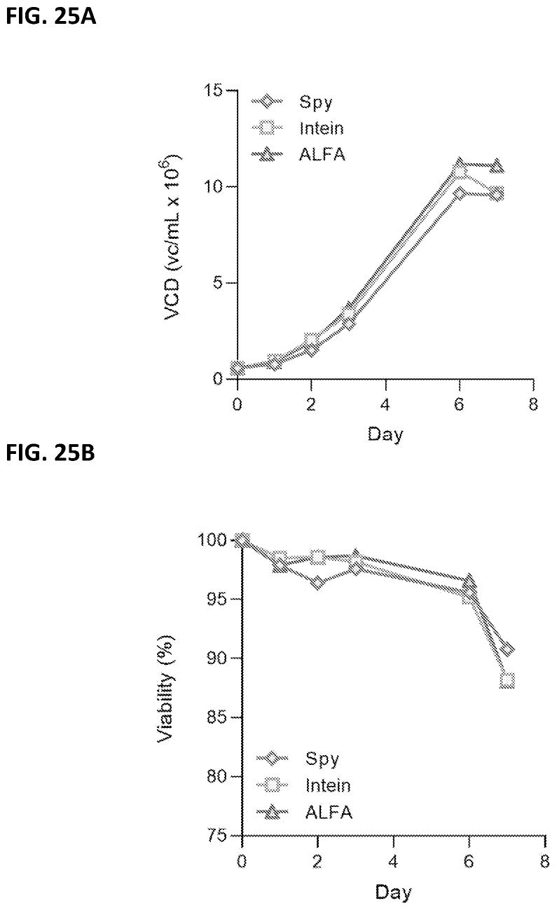

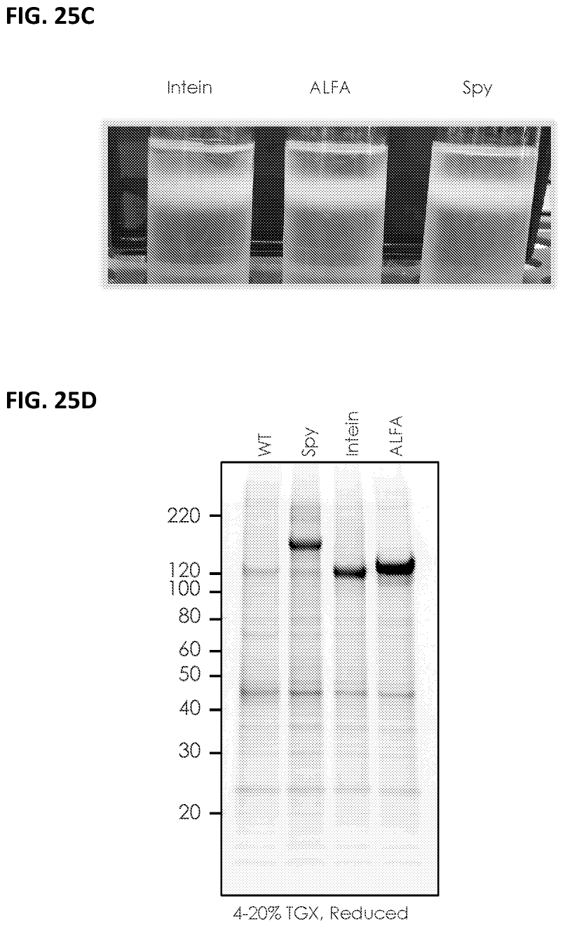

Briefly, HEK293 cells were transfected with constructs encoding an acceptor domain fused to the N-terminus of PTGFRN. Three different acceptor domains were analyzed: (1) SpyCatcher, (2) CfaC, and (3) ALFANb. As shown in FIGS. 25A and 25B, all of the PTGFRN fusion protein were stably expressed for at least a week post-transfection. Then EVs (e.g., exosomes) comprising the PTGFRN fusion proteins were successfully produced and purified from the producer cells (see FIGS. 25C and 25D).

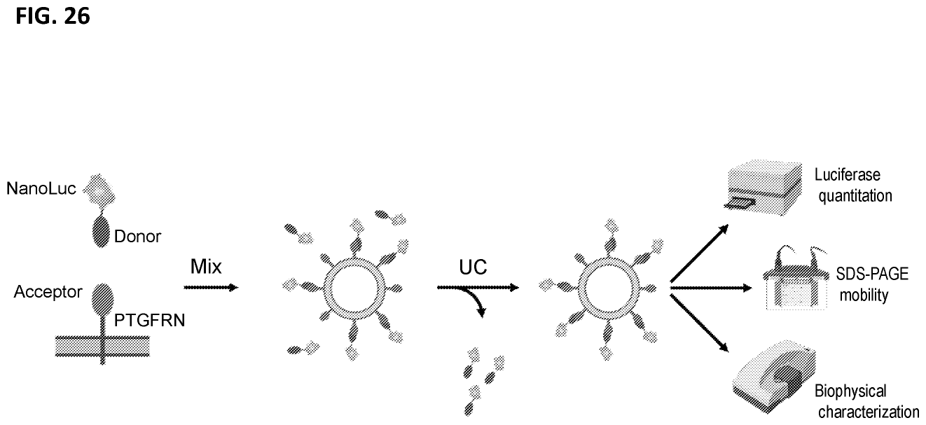

As described herein and further illustrated in FIG. 26, the EVs (e.g., exosomes) produced here could be useful as “base” EVs to which a moiety of interest (e.g., antigen) could rapidly be attached.

### Example 9. Construction of EVs Comprising NanoLuc Luciferase Fused to ALFAtag

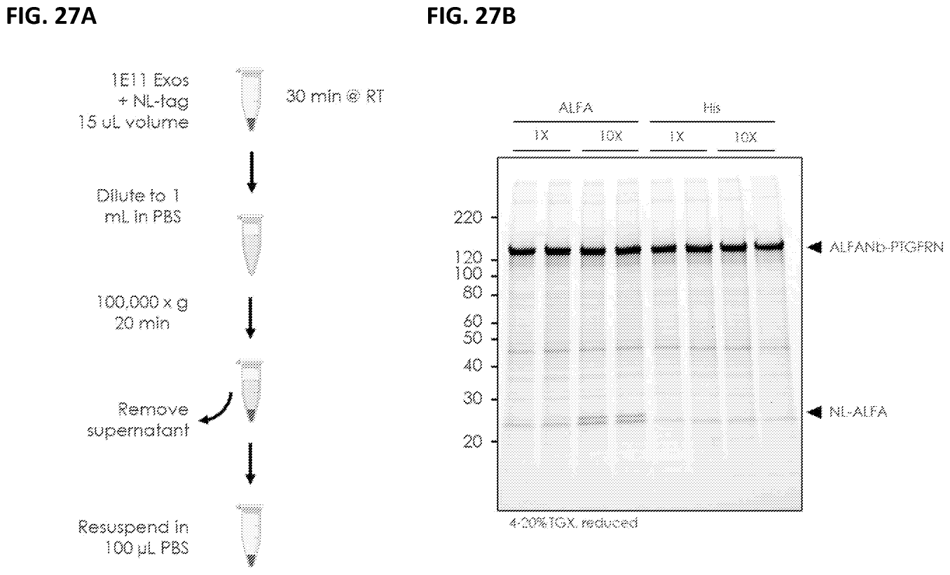

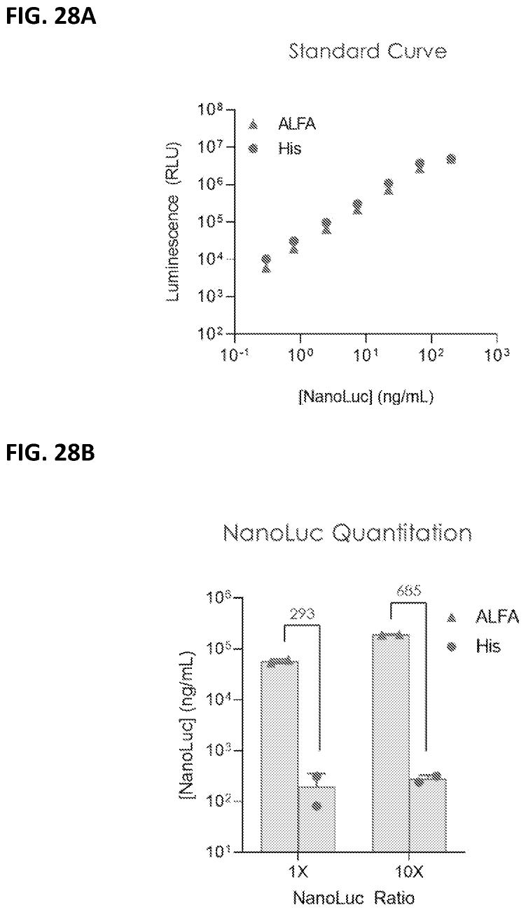

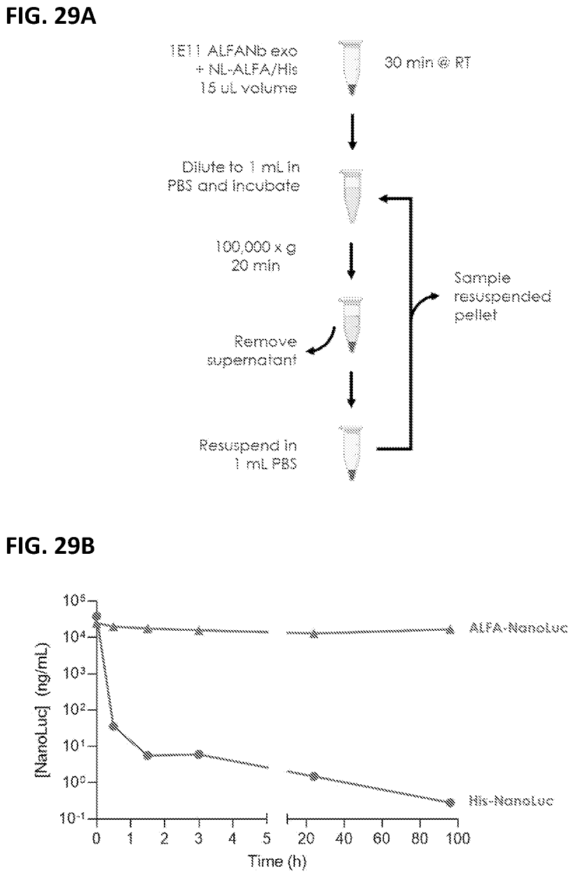

To further address the capability of the EV-based vaccines described herein, EVs overexpressing the ALFANb fused to PTGFRN were functionalized with solubly expressed NanoLuc fused to the ALFAtag peptide. The ALFAtag is capable of interacting with the ALFANb to form a stable, non-covalent interaction. FIG. 27A provides the general methods used. And, as shown FIG. 27B, SDS-PAGE analysis confirmed that the NanoLuc-ALFAtag was able to associate with the ALFANb-PTGFRN protein expressed on the exosome surface, at 1× or 10× molar excess of NanoLuc-ALFAtag. As shown in FIGS. 28A and 28B, compared to the control (i.e., exosomes mixed with NanoLuc fused to poly-histidine tag), loading of the NanoLuc-ALFAtag was significantly higher on the exosomes, indicating that the loading was due to the specific interaction between the ALFAtag and ALFANb-PTGFRN fusion on the exosomes. And, as shown in FIGS. 29A and 29B, the interaction between the ALFAtag and ALFANb-PTGFRN fusion remained stable for an extended period of time under physiologic conditions.

### Example 10. Construction of EVs Comprising NanoLuc Luciferase Fused to Cfa Split Intein

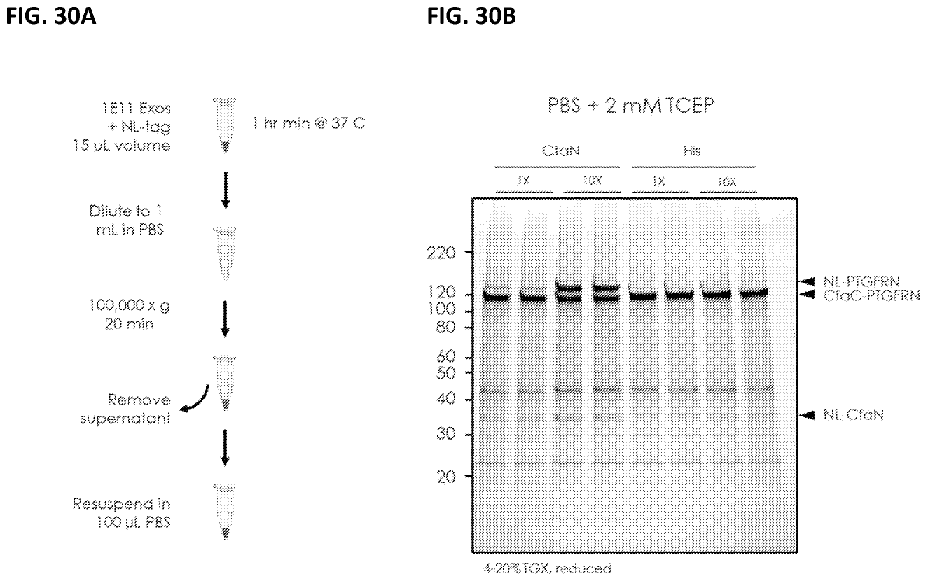

To confirm the results provided above, EVs overexpressing CfaC fused to PTGFRN were functionalized with solubly expressed NanoLuc fused to CfaN. Again, NanoLuc fused to a poly-histidine tag was used as a control. The general methods used are provided in FIG. 30A. Since intein trans-splicing results in covalent attachment of NanoLuc, a clear shift in molecular weight was visible by SDS-PAGE (NL-PTGFRN) (see FIG. 30B). The successful attachment of the NanoLuc was further confirmed when the expression of NanoLuc was measured on the exosomes. As shown in FIGS. 31A and 31B, compared to exosomes that were mixed with the NanoLuc-histidine control, exosomes that were mixed with the NanoLuc-CfaN had significantly higher NanoLuc expression.

### Example 11. Additional Construction of Modular EVs

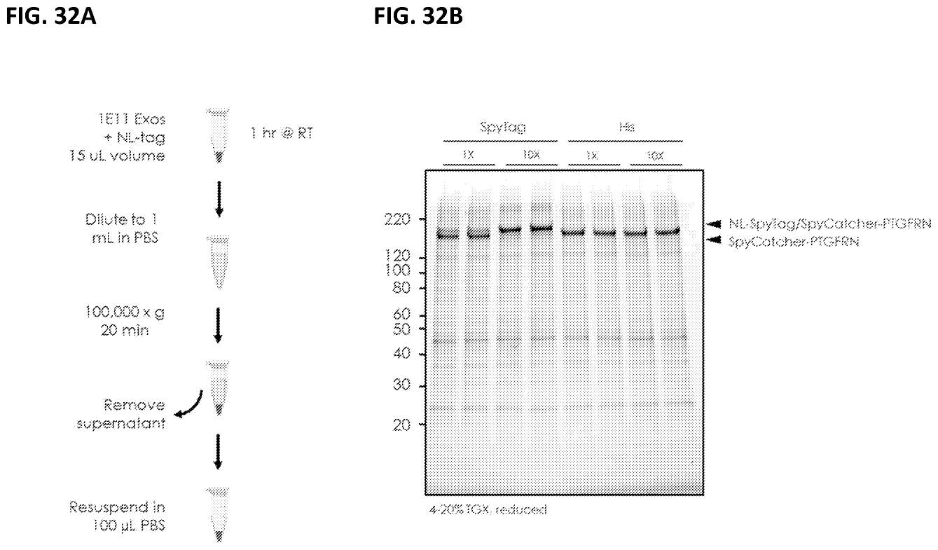

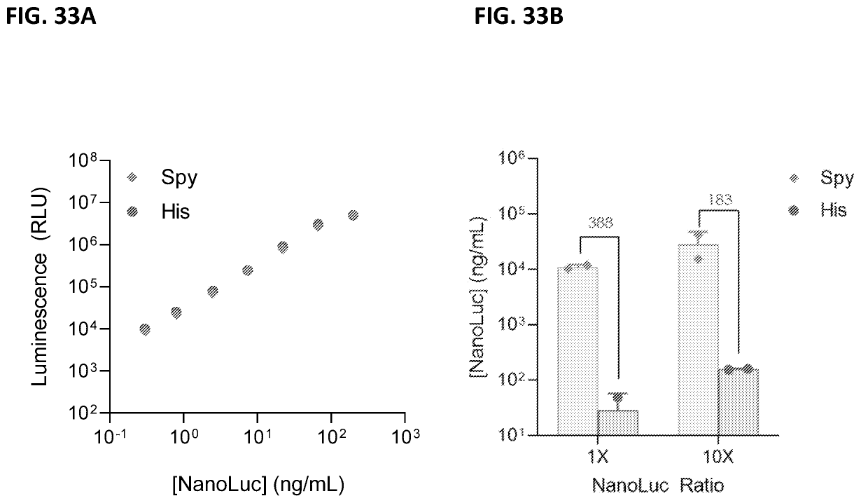

Further to the examples provided above, isolated EVs overexpressing SpyCatcher fused to PTGFRN were functionalized with solubly expressed NanoLuc fused to SpyTag, as described in FIG. 32A. The SDS-PAGE provided in FIG. 32B confirms the loading of NanoLuc-SpyTag onto SpyCatcher-PTGFRN overexpressing exosomes. Since SpyCatcher/Spy Tag forms a spontaneous isopeptide bond, covalent attachment of NanoLuc-SpyTag resulted in a clear shift in molecular weight is visible by SDS-PAGE (NL-SpyTag/SpyCatcher-PTGFRN) (see FIG. 32B). FIGS. 33A and 33B confirm the results quantitatively.

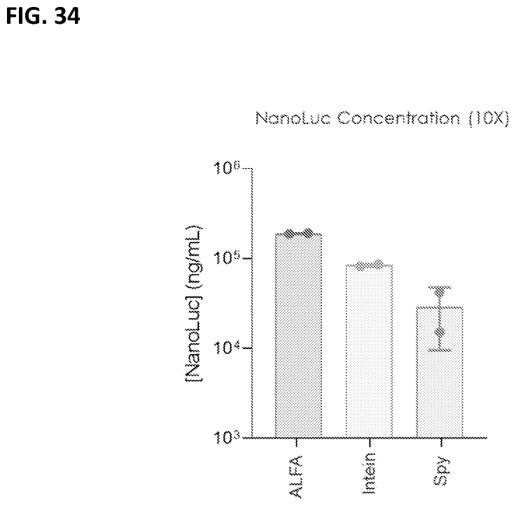

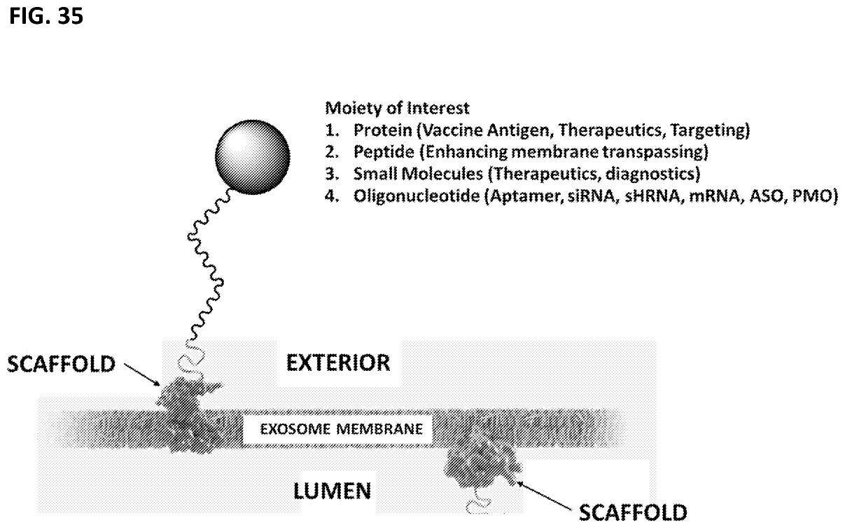

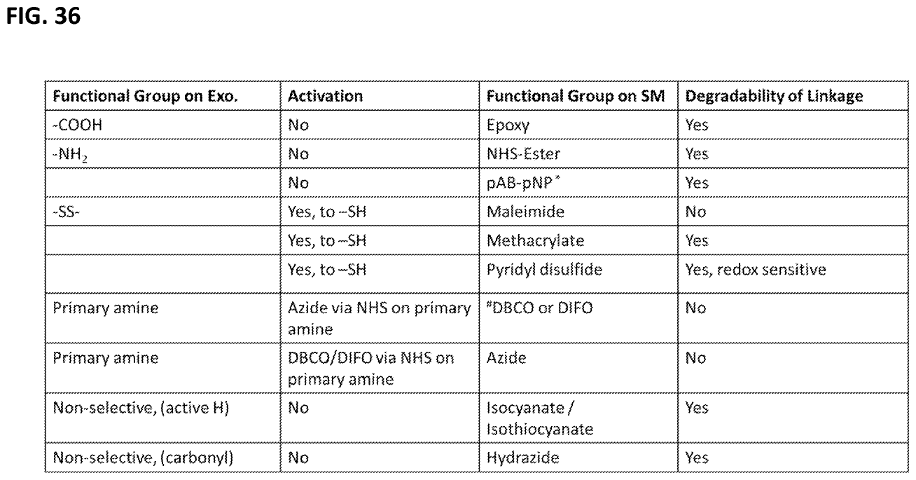

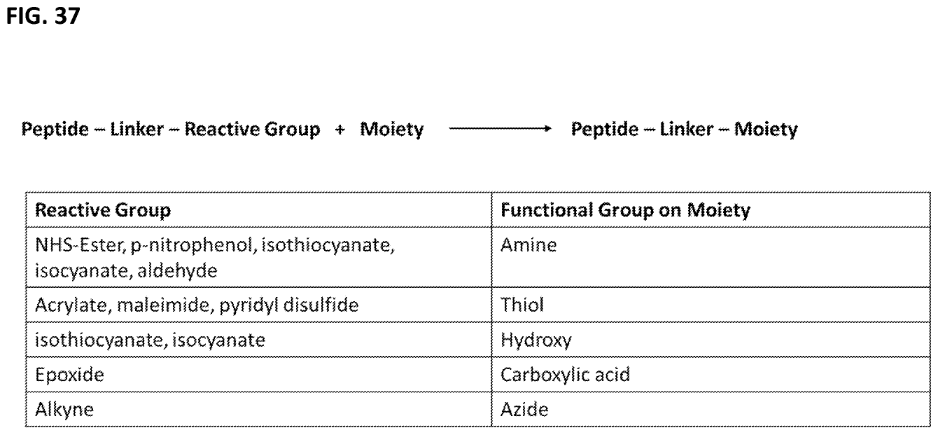

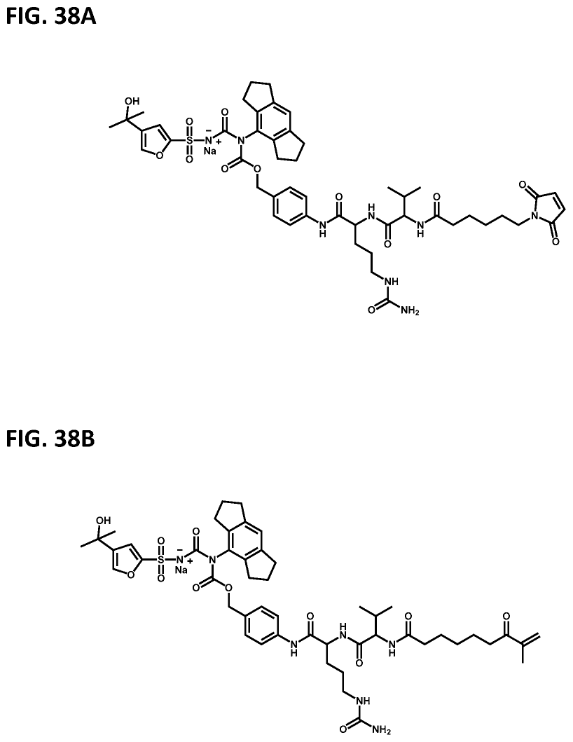

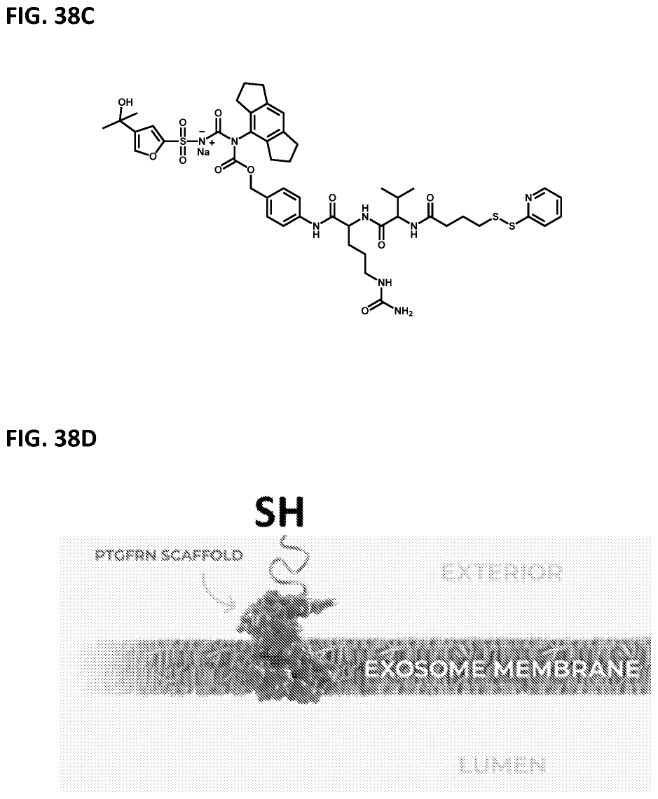

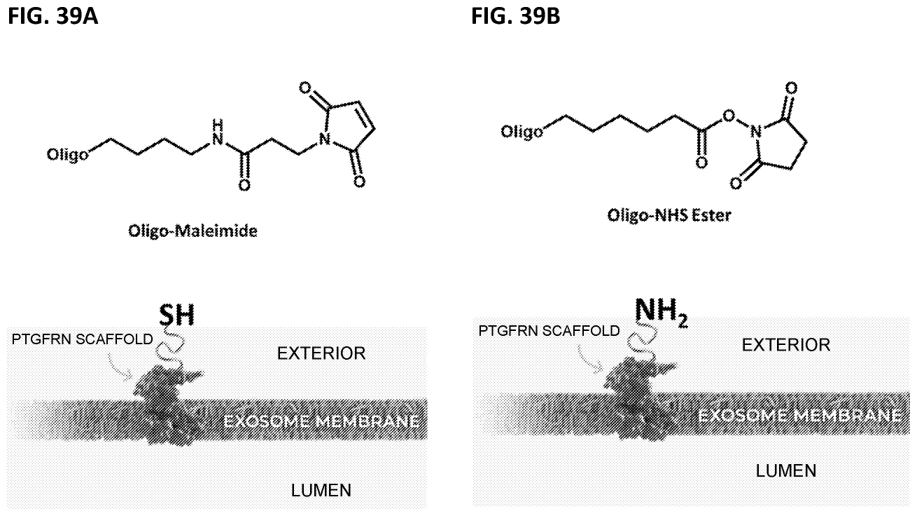

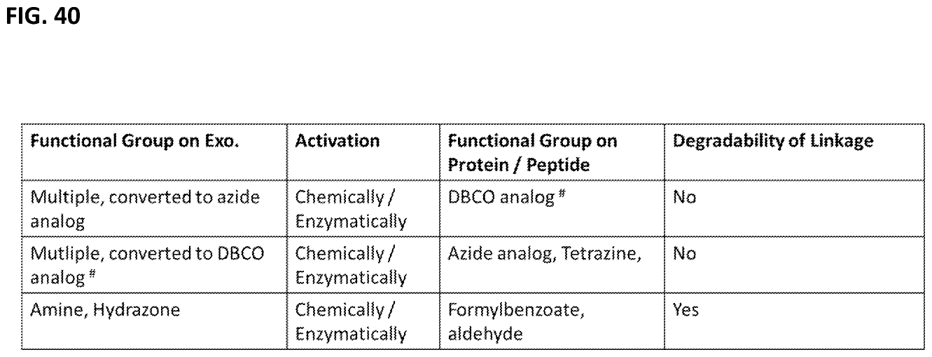

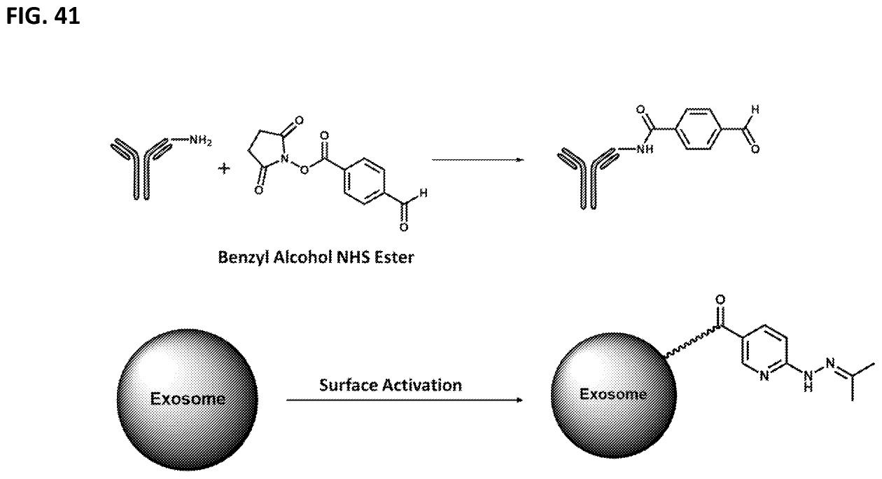

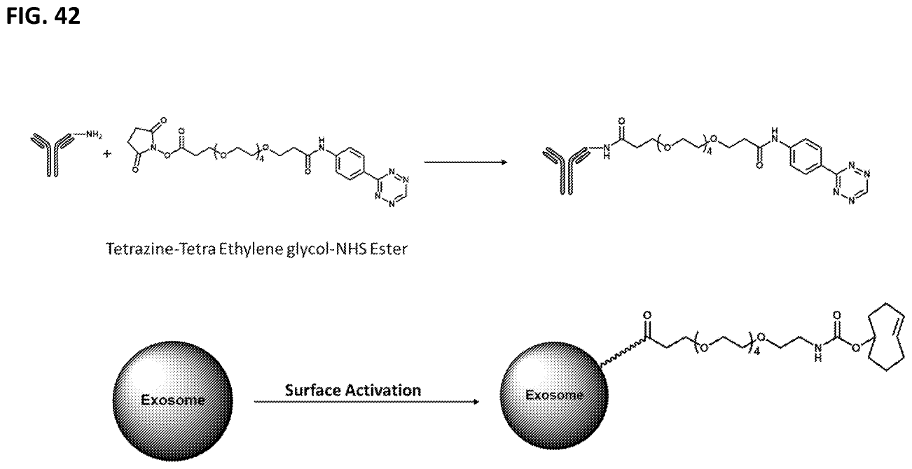

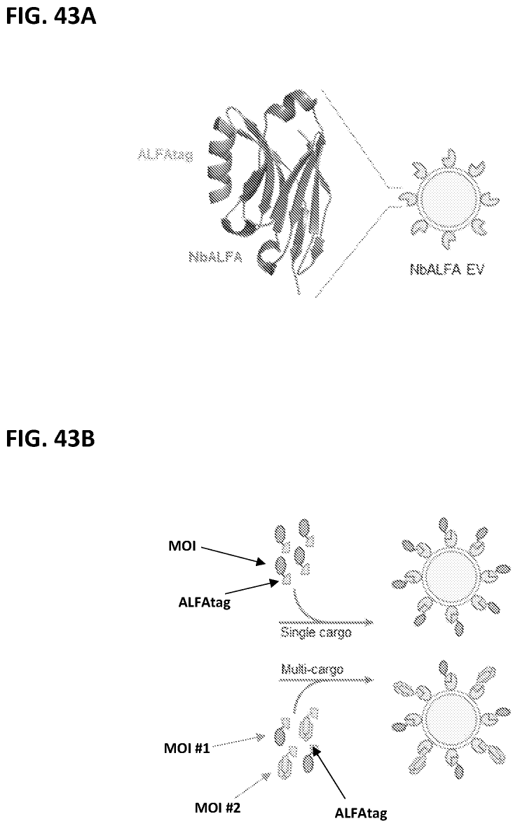

As shown in FIG. 34, similar results were observed with the earlier discussed acceptor domains (i.e., ALFANb, CfaC, and SpyCatcher).

Collectively, the above results confirm that the EVs (e.g., exosomes) described herein could be a useful vaccine platform for rapidly treating a wide range of diseases and disorders.

### Example 12. Analysis of the Loading of Multiple Payloads in EVs

Next, the ability of the EVs described herein to comprise multiple payloads (e.g., antigen, adjuvant, immunomodulatory, and/or targeting moiety) was assessed. Briefly, NANOLUC™ luciferase (Nluc) fused to the ALFAtag peptide (10 μg) (Nluc-ALFAtag) and molar equivalent of mouse IL-12 fused to ALFAtag peptide (mIL12-ALFAtag) were mixed with the following EVs individually or simultaneously: (1) native EVs; or (2) engineered-EVs overexpressing ALFA-specific nanobody (NbALFA) fused to PTGFRN (NbALFA-EVs). The mixture was incubated for 30 minutes at room temperature. Then, unbound Nluc-ALFAtag and/or mIL12-ALFAtag was removed by ultracentrifugation (20 minutes at 100,000×g). The EV pellets were resuspended in PBS and analyzed by SDS-PAGE and Western blot.

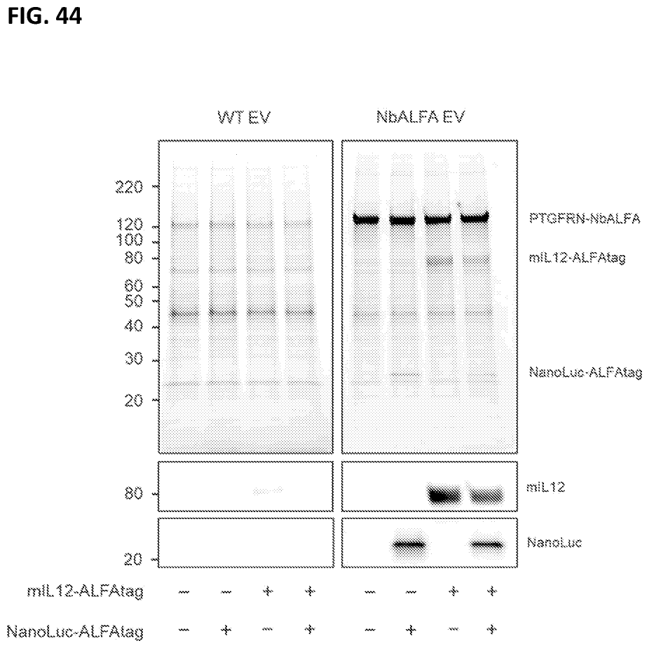

As shown in FIG. 44, in native EVs, no meaningful loading was observed for either Nluc-ALFAtag or mIL12-ALFAtag. However, in the NbALFA-EVs, significant loading of Nluc-ALFAtag and mIL12-ALFAtag was observed, as measured by both Western blot and SDS-PAGE. Similar results were observed whether Nluc-ALFAtag and mIL12-ALFAtag were loaded individually or simultaneously.

The above results further demonstrate that the EVs of the present disclosure can be readily modified to simultaneously comprise multiple payloads. As described herein, such EVs can be useful in treating various diseases and disorders, such as those disclosed herein.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the disclosure(s). Many variations will become apparent to those skilled in the art upon review of this specification.

1. An isolated extracellular vesicle (EV) comprising (i) at least one antigen derived from a coronavirus, (ii) at least a first scaffold moiety, and (iii) at least one targeting moiety.

2-30. (canceled)

31. The EV of claim 1, wherein a first antigen is linked to the first scaffold moiety.

32. The EV of claim 1, wherein a second antigen is linked to the first scaffold moiety.

33. The EV of claim 1, wherein the EV further comprises a second scaffold moiety.

34. The EV of claim 33, wherein a first antigen is linked to the first scaffold moiety, and a second antigen is linked to the second scaffold moiety.

35. The EV of claim 33, wherein the first scaffold moiety and the second scaffold moiety are the same.

36. (canceled)

37. The EV of claim 1, wherein the first scaffold moiety is a Scaffold X, wherein the Scaffold X is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), and any combination thereof.

38. The EV of claim 1, wherein the first scaffold moiety is a Scaffold Y, wherein the Scaffold Y is selected from the group consisting of myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), and any combination thereof.

39. The EV of claim 33, wherein the second scaffold moiety is a Scaffold X or a Scaffold Y.

40. (canceled)

41. The EV of claim 34, wherein the first scaffold moiety or the second scaffold moiety is capable of: (i) anchoring the first antigen on the luminal surface of the EV; (ii) anchoring the first antigen on the exterior surface of the EV; (iii) anchoring the second antigen on the luminal surface of the EV; (iv) anchoring the second antigen on the exterior surface of the EV; or (v) any combinations thereof.

42-66. (canceled)

67. The EV of claim 33, wherein the first scaffold moiety or the second scaffold moiety comprises an amino acid sequence at least 90%, at least 95%, at least 96%, at least 97%, at least 98%, at least 99%, or about 100% identical to SEQ ID NO: 1, 3 or 33.

68-70. (canceled)

71. The EV of claim 33, wherein the first scaffold moiety or the second scaffold moiety comprises an amino acid sequence set forth in any one of SEQ ID NOs: 50-155.

72-73. (canceled)

74. The EV of claim 1, wherein the EV further comprises an adjuvant, wherein the adjuvant is a Stimulator of Interferon Genes (STING) agonist, a toll-like receptor (TLR) agonist, an inflammatory mediator, RIG-I agonists, alpha-gal-cer (NKT agonist), heat shock proteins (e.g., HSP65 and HSP70), C-type lectin agonists (e.g., beta glucan (Dectin 1), chitin, and curdlan), or any combination thereof.

75-86. (canceled)

87. The EV of claim 1, wherein the EV is an exosome.

88. (canceled)

89. The EV of claim 1, wherein the targeting moiety specifically binds to a marker for a dendritic cell or a T cell.

90-101. (canceled)

102. A pharmaceutical composition comprising the EV of claim 1 and a pharmaceutically acceptable carrier.

103-108. (canceled)

109. A method of preventing or treating a disease in a subject in need thereof, comprising administering the EV of claim 1, wherein the disease is an infection and is associated with the antigen.

110-120. (canceled)

121. The EV of claim 74, wherein the antigen, adjuvant, immune modulator, and/or targeting moiety is linked to a surface of the EV by an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof.

122-131. (canceled)

132. The method of preparing an EV for a vaccine, comprising loading an antigen to an EV that has been isolated from a producer cell, wherein the antigen is derived from a coronavirus, an influenza virus, an Ebola virus, a Chikungunya virus (CHIKV), a Crimean-Congo hemorrhagic fever (CCGF) virus, a Hendra virus, a Lassa virus, a Marburg virus, a monkeypox virus, a Nipah virus, a Hendra virus, a Rift Valley fever (RVF) virus, a Variola virus, a yellow fever virus, a Zika virus, a measles virus, a human immunodeficiency virus (HIV), a hepatitis C virus (HCV), a dengue fever virus (DENV), a parvovirus (e.g., B19 virus), a norvovirus, a respiratory syncytial virus (RSV), a lentivirus, an adenovirus, a flavivirus, a filovirus, a rhinovirus, a human papillomavirus (HPV), or any combination thereof.

133-134. (canceled)

135. A method of manufacturing a vaccine for a disease or disorder, comprising loading an antigen to the EV of that has been isolated from a producer cell, wherein the time required for manufacturing the vaccine (“manufacturing time) is reduced compared to a reference manufacturing time (e.g., manufacturing time of a method wherein the loading of the antigen occurs by introducing the antigen into the producer cell, or manufacturing time of a method for producing a vaccine that does not comprise an EV, such as a traditional peptide vaccine), wherein the manufacturing time is reduced by at least about 10%, at least about 20%, at least about 30%, at least about 40%, at least about 50%, at least about 60%, at least about 70%, at least about 80%, or at least about 90% or more, compared to the reference manufacturing time.

136-150. (canceled)
